(19) United States
(12) Patent Application Publication
(10) Pub. No.: US 2018/0363062 A1
(43) Pub. Date: Dec. 20, 2018

(54) Methods for Diagnosis, Prognosis and Monitoring of Breast Cancer and Reagents Therefor

(71) Applicant: Garvan Institute of Medical Research, New South Wales (AU)

(72) Inventors: Andrew Stone, New South Wales (AU); Elena Zotenko, New South Wales (AU); Susan Clark, New South Wales (AU)

(21) Appl. No.: 15/742,924

(22) PCT Filed: Jul. 14, 2016
(86) PCT No.: PCT/AU16/50615

§ 371 Date: Jan. 9, 2018

Related U.S. Application Data
(60) Provisional application No. 62/192,450, filed on Jul. 14, 2015.

(51) Int. Cl. C12Q 1/6886; C12Q 1/48

(52) U.S. Cl. CPC C12Q 1/6886; C12Q 1/48; C12Q 2600/158; C12Q 2600/106; C12Q 2600/154

(57) ABSTRACT

The present disclosure generally relates to methods and reagents for the diagnosis, prognosis or the monitoring of estrogen receptor 1 (ESR1) positive breast cancer, for example, ESR1 positive breast cancer which is responsive to endocrine therapy and/or ESR1 positive breast cancer which is refractory to endocrine therapy. The present disclosure also relates generally to treatment management of ESR1 positive breast cancer.

### TECHNICAL FIELD

The present disclosure generally relates to methods and reagents for the diagnosis, prognosis or the monitoring of estrogen receptor 1 (ESR1) positive breast cancer, for example, ESR1 positive breast cancer which is responsive to endocrine therapy and/or ESR1 positive breast cancer which is refractory to endocrine therapy. The present disclosure also relates generally to treatment management of ESR1 positive breast cancer.

### BACKGROUND

Cancer is a leading cause of disease worldwide. Breast cancer is one of the most common forms of cancer, affecting both females and males globally. Various subtypes of breast cancer have been distinguished based on a number of factors including the histopathological type of tumor, the grade of the tumor, the stage of the tumor, and the expression of genes which are characteristic of particular subtypes of breast cancer. Determination of the particular subtype of cancer in a patient is often of critical importance in determining the most appropriate course of treatment for the patient.

Estrogen receptor (ER) negative (ER-ve) breast cancer and ER positive (ER+ve) breast cancer are two recognised subtypes of breast cancer, defined by the presence or absence of expression of the estrogen receptor gene. The steroid hormone estrogen activates the estrogen receptor (ESR1) to mediate a variety of functions that are central to the normal development and maintenance of multiple tissues, including breast tissue. Inappropriate activation of the ESR1-signalling network in mammary epithelial cells initiates neoplastic transformation and drives ESR1-positive breast cancer. Patients with this disease commonly receive adjuvant endocrine therapy, which serves to inhibit ESR1-signalling. Although endocrine therapy reduces the risk of disease recurrence and breast cancer-related mortality, a third of patients with ESR1-positive breast cancer acquire drug resistance and experience disease relapse. Currently, no tests exist which can predict resistance to endocrine therapy with certainty. Thus, there is a need to identify a robust method by which ESR1-positive breast cancer patients can be stratified according to their responsiveness or resistance to endocrine therapy to enable more informed disease management.

Previous efforts to stratify early breast cancer prognosis have primarily focused on multi-gene expression signatures. In addition to multi-gene expression assays, DNA methylation signatures are being assessed as potential molecular biomarkers of cancer. Despite growing interest in the prognostic significance of DNA methylation in breast cancer, there have been no studies specifically investigating the DNA methylation profile of human ESR1-positive breast cancer and its association with disease outcome in response to treatment.

There is a need in the art for improved methods for the diagnosis of breast cancer, as well as for the diagnosis of specific subtypes of breast cancer, including ESR1-positive breast cancer which is responsive to endocrine therapy and ESR1-positive breast cancer which is resistant to endocrine therapy. There is also a need for methods of prognosis, and predicting responsiveness to treatment, in patients diagnosed with breast cancer and undergoing treatment.

### SUMMARY

The present inventors performed a genome-wide DNA methylation profiling analysis from ESR1-positive endocrine therapy sensitive breast cancer cells and ESR1-positive endocrine resistant cells. In doing so, the inventors identified significant enrichment of hypermethylated probes in enhancer regions of the genome for ESR1-positive endocrine resistant cells in comparison to ESR1-positive endocrine therapy sensitive breast cancer cells. The inventors also identified a subset of 856 ESR1 binding sites that overlap enhancer regions that contain hypermethylated loci in the ESR1-positive endocrine resistant cells, 617 of which were identified as being intragenic. Furthermore, using RNA-seq and HM450 methylation data derived from a TCGA breast cancer cohort, the inventors identified that out of the 856 ESR1 binding sites which overlap enhancer regions identified, hypermethylation of 328 of those sites correlated with reduced expression of the genes with which they were most closely associated, representing 291 unique genes. These markers have been demonstrated to have significant value in the diagnosis and prognosis of ESR1-positive breast cancer which is resistant or responsive to endocrine therapy (i.e., whether the ESR1-positive cancer is in an endocrine responsive state), including determining whether a subject has acquired resistance to endocrine therapy during treatment of ESR1-positive breast cancer. The inventors have also shown that the methylation profile at the 856 ESR1 binding sites is indicative of the particular subtypes of ESR1-positive breast cancer e.g., luminal A breast cancer subtype or a luminal B breast cancer subtype.

Particular examples of enhancer regions of the disclosure which harbour CpG dinucleotide sequences identified as having significant value in the diagnosis and/or prognosis of ESR1-positive breast cancer which is, or is likely to be, resistant or responsive to endocrine therapy include those located within DAXX, MSI2, NCOR2, RXRA, C8orf46, GATA3, ITPK1, ESR1 and GET4.

Accordingly, the present disclosure provides a method for predicting response to endocrine therapy in a subject suffering from estrogen receptor 1 (ESR1) positive breast cancer, said method comprising:

(i) determining the methylation status of one or more CpG dinucleotide sequences within one or more estrogen responsive enhancers in the subject; and

(ii) identifying differential methylation of said one or more CpG dinucleotide sequences in the subject relative to a reference level of methylation for the corresponding one or more CpG dinucleotide sequences;

wherein differential methylation of said one or more CpG dinucleotide sequences in the subject relative to the reference level is indicative of the subject's likely response to endocrine therapy.

For example, increased methylation at the one or more CpG dinucleotide sequences within the one or more estrogen responsive enhancers relative to the reference level is indicative of the ESR1-positive breast cancer being refractory to endocrine therapy.

The present disclosure also provides a method for diagnosing estrogen receptor 1 (ESR1) positive breast cancer which is refractory to endocrine therapy in a subject suffering from ESR1 positive breast cancer, said method comprising:

(i) determining the methylation status of one or more CpG dinucleotide sequences within one or more estrogen responsive enhancers in the subject; and

(ii) identifying differential methylation of said one or more CpG dinucleotide sequences in the subject relative to a reference level of methylation for the corresponding one or more CpG dinucleotide sequences,

wherein differential methylation of said one or more CpG dinucleotide sequences in the subject relative to the reference level is indicative of the subject having breast cancer which is refractory to endocrine therapy.

The present disclosure also provides a method for predicting the therapeutic outcome of and/or monitoring the progression of estrogen receptor 1 (ESR1) positive breast cancer in a subject receiving or about to receive endocrine therapy, said method comprising:

(i) determining the methylation status of one or more CpG dinucleotide sequences within one or more estrogen responsive enhancers in the subject; and

(ii) identifying differential methylation of said one or more CpG dinucleotide sequences in the subject relative to a reference level of methylation for the corresponding one or more CpG dinucleotide sequences;

wherein differential methylation identified at (ii) is indicative of the likely therapeutic outcome and/or of the progression of the ESR1 positive breast cancer.

For example, increased methylation at the one or more CpG dinucleotide sequences within the one or more estrogen responsive enhancers relative to the reference level is indicative of the ESR1-positive breast cancer being refractory to endocrine therapy and/or that the subject is not responding to the endocrine therapy.

In one example, the method comprises determining whether the ESR1-positive breast cancer is a luminal A breast cancer subtype or a luminal B breast cancer subtype.

The present disclosure also provides a method for detecting differential methylation at one or more CpG dinucleotide sequences within one or more estrogen responsive enhancers in a subject suffering from estrogen receptor 1 (ESR1) positive breast cancer, said method comprising:

(i) performing an assay on a sample from the subject configured to determine methylation status at one or more CpG dinucleotide sequences within one or more estrogen responsive enhancers in the subject; and

(ii) detecting differential methylation at the one or more CpG dinucleotide sequences within the one or more estrogen responsive enhancers in the subject relative to a reference level of methylation for the corresponding one or more CpG dinucleotide sequences.

In one example, detecting differential methylation at the one or more CpG dinucleotide sequences at (ii) comprises comparing a level of methylation at the one or more CpG dinucleotide sequences as determined at (i) to the reference level of methylation for the corresponding one or more CpG dinucleotide sequences, and determining whether methylation at the one or more CpG dinucleotide sequences in the subject differs to the corresponding reference level(s) of methylation.

In any of the methods disclosed herein, methylation status may be determined for one or more CpG dinucleotide sequences within one or more ESR1 binding sites. In one example, the one or more CpG dinucleotide sequences are within one or more ESR1-binding sites as defined in Table 1. In another example, the one or more CpG dinucleotide sequences are within one or more ESR1-binding sites as defined in Table 2. In another example, the one or more CpG dinucleotide sequences are within one or more ESR1-binding sites as defined in Table 3.

In one example of the methods disclosed herein, methylation status is determined at one or more CpG dinucleotide sequences within an estrogen responsive enhancer of a gene selected from DAXX, ESR1, RXRA, GET4, NCOR2, GATA3, MSI2, C8orf46 and/or ITPK1. For example, the methylation status may be determined at one or more CpG dinucleotide sequences within an estrogen responsive enhancer within a gene selected from DAXX, ESR1, RXRA, GET4, NCOR2, GATA3, MSI2, C8orf46 and/or ITPK1. Exemplary CpG dinucleotide sequences for which methylation status may be determined in accordance with the methods of the disclosure are selected from those defined in rows 57, 111-113, 256-258, 288-289, 469-470, 805, 821-822 and 824-826 of Table 1.

In another example of the methods disclosed herein, methylation status is determined at one or more CpG dinucleotide sequences within an estrogen responsive enhancer of a gene selected from DAXX, RXRA, NCOR2, MSI2, and/or C8orf46. For example, the methylation status may be determined at one or more CpG dinucleotide sequences within an estrogen responsive enhancer within a gene selected from DAXX, RXRA, NCOR2, MSI2, and/or C8orf46.

In yet another example of the methods disclosed herein, methylation status is determined at one or more CpG dinucleotide sequences within an estrogen responsive enhancer of a gene selected from FOXA1, ESR1 and/or GATA3.

In any of the methods disclosed herein, methylation status of one or more CpG dinucleotide sequences may be determined according to any suitable method known in the art. For example, methylation status of one or more CpG dinucleotide sequences within the one or more estrogen responsive enhancers may be determined by one or more techniques selected from the group consisting of a nucleic acid amplification, polymerase chain reaction (PCR), methylation specific PCR, bisulfite pyrosequencing, single-strand conformation polymorphism (SSCP) analysis, restriction analysis, microarray technology, and proteomics. For example, methylation status of one or more CpG dinucleotide sequences within the one or more estrogen responsive enhancers in the subject is determined by one or more of the following:

(i) performing methylation-sensitive endonuclease digestion of DNA from the subject;

(ii) treating nucleic acid from the subject with an amount of a compound that selectively mutates non-methylated cytosine residues in nucleic acid under conditions sufficient to induce mutagenesis thereof and produce a mutant nucleic acid and amplifying the mutant nucleic acid using at least one primer that selectively hybridizes to the mutant nucleic acid;

(iii) treating nucleic acid from the subject with an amount of a compound that selectively mutates non-methylated cytosine residues in nucleic acid under conditions sufficient to induce mutagenesis thereof and produce a mutant nucleic acid, hybridizing a nucleic acid probe or primer capable of specifically hybridizing to the mutant nucleic acid and detecting the hybridized probe or primer;

(iv) treating nucleic acid from the subject with an amount of a compound that selectively mutates non-methylated cytosine residues in nucleic acid under conditions sufficient to induce mutagenesis thereof and produce a mutant nucleic acid, amplifying the mutant nucleic acid with promoter-tagged primers, transcribing the mutant nucleic acid in vitro to produce a transcript, subjecting the transcript to an enzymatic base-specific cleavage, and determining differences in mass and/or size of any cleaved fragments resulting from mutated cysteine residues, such as by MALDI-TOF mass spectrometry;

(v) treating nucleic acid from the subject with an amount of a compound that selectively mutates non-methylated cytosine residues in nucleic acid under conditions sufficient to induce mutagenesis thereof, thereby producing a mutant nucleic acid, and determining the nucleotide sequence of the mutant nucleic acid; and

(vi) performing methylation DNA capture or immunoprecipitation on DNA from the subject to detect and/or capture methylated DNA from the subject, and optionally determining the nucleotide sequence of the DNA fragments detected and/or captured.

The method used for methylation DNA capture or immunoprecipitation may be methylated DNA immunoprecipitation (MeDIP) or capture of methylated DNA by methyl-CpG binding domain-based (MBD) proteins (MBDCap).

The compound that selectively mutates non-methylated cytosine residues may be any compound suitable for that purpose, including, for example, a salt of bisulphite.

The methods disclosed herein may be performed on any test sample taken from a subject. For example, the methylation status of one or more CpG dinucleotide sequences within the one or more estrogen responsive enhancers may be determined in a test sample from the subject comprising tissue and/or a body fluid comprising, or suspected of comprising, a breast cancer cell or components of a breast cancer cell. The sample may comprise tissue, a cell and/or an extract thereof taken from a breast or lymph node. When the sample comprises a body fluid, the body fluid may be selected from the group consisting of whole blood, a fraction of blood such as blood serum or plasma, urine, saliva, breast milk, pleural fluid, sweat, tears and mixtures thereof.

In any of the methods disclosed herein, the reference level of methylation is a level of methylation determined for one or more CpG dinucleotide sequences within a corresponding estrogen responsive enhancer of a sample selected from the group consisting of:

(i) a sample from a normal or healthy tissue;

(ii) a sample comprising a non-cancerous cell;

(iii) a sample comprising a cancerous cell, other than a breast cancer cell characterized as being ESR1-negative subtype;

(iv) a sample comprising a cancerous cell, other than a breast cancer cell characterized as being a ESR1-positive subtype which is refractory to endocrine therapy;

(v) a sample comprising a breast cancer cell characterized as being a ESR1-positive subtype which is refractory to endocrine therapy;

(vi) a sample comprising a breast cancer cell characterized as being a ESR1-positive subtype which is responsive to endocrine therapy;

(vii) an extract of any one of (i) to (vi);

(viii) a data set comprising levels of methylation for the one or more CpG dinucleotide sequences within the corresponding estrogen responsive enhancer in a normal or healthy individual or a population of normal or healthy individuals;

(ix) a data set comprising levels of methylation for the one or more CpG dinucleotide sequences within the corresponding estrogen responsive enhancer in an individual or a population of individuals having cancer other than ESR1-negative breast cancer subtype;

(x) a data set comprising levels of methylation for the one or more CpG dinucleotide sequences within the corresponding estrogen responsive enhancer in an individual or a population of individuals having cancer other than a ESR1-positive breast cancer subtype which is refractory to endocrine therapy;

(xi) a data set comprising levels of methylation for the one or more CpG dinucleotide sequences within the corresponding estrogen responsive enhancer in an individual or a population of individuals having breast cancer characterized as being a ESR1-positive breast cancer subtype which is refractory to endocrine therapy;

(xii) a data set comprising levels of methylation for the one or more CpG dinucleotide sequences within the corresponding estrogen responsive enhancer in an individual or a population of individuals having breast cancer characterized as being a ESR1-positive breast cancer subtype which is responsive to endocrine therapy; and

(xiii) a data set comprising levels of methylation for the one or more CpG dinucleotide sequences within the corresponding estrogen responsive enhancer in the subject being tested wherein the levels of methylation are determined for a matched sample having normal cells.

The cancerous cell in (iii) and/or (iv) above may be, for example, a breast cancer cell. Alternatively or in addition, the cancer in (ix) and/or (x) above may be, for example, breast cancer.

In any of the methods disclosed herein, the method may additionally provide a step of treating ESR1-positive breast cancer e.g., following performance of a diagnostic or prognostic method disclosed herein. In this way, the methods of the disclosure may comprise, for example, diagnosing ESR1-positive breast cancer using a method of the disclosure described in any one or more examples described herein and, based on whether the subject is determined as being responsive or resistant to endocrine therapy, administering a suitable therapeutic compound or performing surgery or recommending treatment with a suitable therapeutic compound or recommending performance of surgery. For example, if, after performing the method of diagnosis or prognosis of the disclosure, the subject is determined as being responsive to endocrine therapy, the method may comprise commencing endocrine therapy e.g., by administering a therapeutic compound suitable for endocrine therapy, or recommending that the subject commence endocrine therapy. In another example, if, after performing the method of diagnosis or prognosis of the disclosure, the subject is determined as being resistance/refractory to endocrine therapy, the method may comprise commencing treatment other than endocrine therapy e.g., chemotherapy or radiotherapy, and/or performing surgery, or recommending that the subject commences treatment other than endocrine therapy e.g., chemotherapy or radiotherapy, and/or recommending surgery.

Drugs suitable for use in endocrine therapy are well known in the art, and include, for example, anastrozole, exemestane, fulvestrant, goserelin, letrozole, leuprorelin, leuprolide acetate, megestrol, palbociclib, tamoxifen and toremifene.

Chemotherapeutic drugs suitable for treatment of breast cancer are known in the art, but may include, for example, docetaxel, paclitaxel, platinum agents (cisplatin, carboplatin), vinorelbine, capecitabine, liposomal doxorubicin, gemcitabine, mitoxantrone, ixabepilone, albumin-bound paclitaxel and eribulin.

The present disclosure also provides a method of treating a subject suffering from estrogen receptor 1 (ESR1) positive breast cancer, wherein the subject has been diagnosed as being refractory to endocrine therapy based on differential methylation at one or more CpG dinucleotide sequences within one or more estrogen responsive enhancers in the subject as determined relative to a reference level of methylation for the corresponding one or more CpG dinucleotide sequence(s), said method comprising administering chemotherapy and/or radiotherapy to the subject, and/or performing surgery on the subject to remove the cancer or a portion thereof.

In one example, the subject has been diagnosed as being refractory to endocrine therapy based on increased methylation at the one or more CpG dinucleotide sequences within the one or more estrogen responsive enhancers relative to the reference level of methylation for the corresponding one or more CpG dinucleotide sequence(s).

In accordance with an example in which the subject has been diagnosed as being refractory to endocrine therapy, the reference level of methylation is a level of methylation determined for one or more CpG dinucleotide sequences within a corresponding estrogen responsive enhancer of a sample selected from the group consisting of:

(i) a sample from a normal or healthy tissue;

(ii) a sample comprising a non-cancerous cell;

(iii) a sample comprising a cancerous cell, other than a breast cancer cell characterized as being ESR1-negative subtype;

(iv) a sample comprising a cancerous cell, other than a breast cancer cell characterized as being a ESR1-positive subtype which is refractory to endocrine therapy;

(v) a sample comprising a breast cancer cell characterized as being a ESR1-positive subtype which is responsive to endocrine therapy

(vi) an extract of any one of (i) to (v);

(vii) a data set comprising levels of methylation for the one or more CpG dinucleotide sequences within the corresponding estrogen responsive enhancer in a normal or healthy individual or a population of normal or healthy individuals;

(viii) a data set comprising levels of methylation for the one or more CpG dinucleotide sequences within the corresponding estrogen responsive enhancer in an individual or a population of individuals having cancer other than ESR1-negative breast cancer subtype;

(ix) a data set comprising levels of methylation for the one or more CpG dinucleotide sequences within the corresponding estrogen responsive enhancer in an individual or a population of individuals having cancer other than a ESR1-positive breast cancer subtype which is refractory to endocrine therapy;

(x) a data set comprising levels of methylation for the one or more CpG dinucleotide sequences within the corresponding estrogen responsive enhancer in an individual or a population of individuals having ESR1-positive breast cancer subtype which is responsive to endocrine therapy; and

(xi) a data set comprising levels of methylation for the one or more CpG dinucleotide sequences within the corresponding estrogen responsive enhancer in the subject being tested wherein the levels of methylation are determined for a matched sample having normal cells.

The cancerous cell in (iii) and/or (iv) above may be, for example, a breast cancer cell. Alternatively or in addition, the cancer in (viii) and/or (ix) above may be, for example, breast cancer.

In one example, the method comprises administering chemotherapy to the subject who has been diagnosed as being refractory to endocrine therapy. Alternatively, or in addition, the method comprises administering radiotherapy to the subject who has been diagnosed as being refractory to endocrine therapy. Alternatively, or in addition, the method comprises performing surgery to remove the cancer or a portion thereof from the subject who has been diagnosed as being refractory to endocrine therapy. For example, the subject may receive chemotherapy and radiotherapy, or chemotherapy and surgery, or radiotherapy and surgery, or chemotherapy, radiotherapy and surgery. According to an example in which more than one of chemotherapy, radiotherapy and surgery are performed on the subject who has been diagnosed as being refractory to endocrine therapy, the respective treatments may be performed in any particular order.

Chemotherapeutic drugs suitable for treatment of breast cancer are known in the art and described herein.

The present disclosure also provides a method of treating a subject suffering from estrogen receptor 1 (ESR1) positive breast cancer, wherein the subject has been diagnosed as being responsive to endocrine therapy based on a differential methylation at one or more CpG dinucleotide sequences within one or more estrogen responsive enhancers in the subject relative to a reference level of methylation for the corresponding one or more CpG dinucleotide sequence(s), said method comprising administering endocrine therapy to the subject.

In one example, the subject has been diagnosed as being responsive to endocrine therapy based on a decreased level methylation at the one or more CpG dinucleotide sequences within the one or more estrogen responsive enhancers relative to the reference level of methylation for the corresponding one or more CpG dinucleotide sequence(s), wherein the reference level of methylation is a level of methylation determined for one or more CpG dinucleotide sequences within a corresponding estrogen responsive enhancer of a sample selected from the group consisting of:

(i) a sample comprising a breast cancer cell characterized as being a ESR1-positive subtype which is refractive to endocrine therapy;

(ii) an extract of (i); and

(iii) a data set comprising levels of methylation for the one or more CpG dinucleotide sequences within the corresponding estrogen responsive enhancer in an individual or a population of individuals having ESR1-positive breast cancer subtype which is refractive to endocrine therapy.

In one example, the subject has been diagnosed as being responsive to endocrine therapy based on a level methylation at the one or more CpG dinucleotide sequences within the one or more estrogen responsive enhancers which corresponds or is equivalent to the reference level of methylation for the corresponding one or more CpG dinucleotide sequence(s), wherein the reference level of methylation is a level of methylation determined for one or more CpG dinucleotide sequences within a corresponding estrogen responsive enhancer of a sample selected from the group consisting of:

(i) a sample comprising a breast cancer cell characterized as being a ESR1-positive subtype which is responsive to endocrine therapy;

(ii) an extract of (i); and

(iii) a data set comprising levels of methylation for the one or more CpG dinucleotide sequences within the corresponding estrogen responsive enhancer in an individual or a population of individuals having ESR1-positive breast cancer subtype which is responsive to endocrine therapy.

Drugs suitable for use in endocrine therapy are well known in the art and are described herein.

In any of the methods of treatment disclosed herein, the one or more CpG dinucleotide sequences are within one or more ESR1-binding sites as defined in Table 1. In another example, the one or more CpG dinucleotide sequences are within one or more ESR1-binding sites as defined in Table 2. In another example, the one or more CpG dinucleotide sequences are within one or more ESR1-binding sites as defined in Table 3.

In one example of the methods disclosed herein, the subject has been diagnosed as being refractory to endocrine therapy or responsive to endocrine therapy based on differential methylation at one or more CpG dinucleotide sequences within an estrogen responsive enhancer of a gene selected from DAXX, ESR1, RXRA, GET4, NCOR2, GATA3, MSI2, C8orf46 and/or ITPK1. For example, the subject has been diagnosed as being refractory to endocrine therapy or responsive to endocrine therapy based on differential methylation at one or more CpG dinucleotide sequences within an estrogen responsive enhancer within a gene selected from DAXX, ESR1, RXRA, GET4, NCOR2, GATA3, MSI2, C8orf46 and/or ITPK1. In one particular example, the subject has been diagnosed as being refractory to endocrine therapy or responsive to endocrine therapy based on differential methylation at one or more CpG dinucleotide sequences selected from those defined in rows 57, 111-113, 256-258, 288-289, 469-470, 805, 821-822 and 824-826 of Table 1.

In another example of the methods disclosed herein, the subject has been diagnosed as being refractory to endocrine therapy or responsive to endocrine therapy based on differential methylation at one or more CpG dinucleotide sequences within an estrogen responsive enhancer of a gene selected from DAXX, RXRA, NCOR2, MSI2, and/or C8orf46. For example, the subject has been diagnosed as being refractory to endocrine therapy or responsive to endocrine therapy based on differential methylation at one or more CpG dinucleotide sequences within an estrogen responsive enhancer within a gene selected from DAXX, RXRA, NCOR2, MSI2, and/or C8orf46.

In yet another example of the methods disclosed herein, the subject has been diagnosed as being refractory to endocrine therapy or responsive to endocrine therapy based on differential methylation at one or more CpG dinucleotide sequences within an estrogen responsive enhancer within a gene selected from FOXA1, ESR1 and/or GATA3.

The present disclosure also provides a kit for predicting response to endocrine therapy in a subject suffering from estrogen receptor 1 (ESR1) positive breast cancer, said kit comprising:

(i) one or more reagents configured to determine the methylation status of one or more CpG dinucleotide sequences within one or more estrogen responsive enhancers in the subject; and

(ii) a reference material which provides a reference level of methylation of the corresponding one or more CpG dinucleotide sequences.

The present disclosure also provides a kit for predicting the therapeutic outcome of and/or monitoring the progression of estrogen receptor 1 (ESR1) positive breast cancer in a subject receiving or about to receive endocrine therapy, said kit comprising:

(i) one or more reagents configured to determine methylation status of one or more CpG dinucleotide sequences within one or more estrogen responsive enhancers in the subject; and

(ii) a reference material which provides a reference level of methylation of the corresponding one or more CpG dinucleotide sequences.

In any of the kits disclosed herein, the one or more reagents is/are configured to determine methylation status of one or more CpG dinucleotide sequences within one or more ESR1 binding sites as defined in Table 1. In another example, the one or more reagents is/are configured to determine methylation status of one or more CpG dinucleotide sequences within one or more ESR1 binding sites as defined in Table 2. In yet another example, the one or more reagents is/are configured to determine methylation status of one or more CpG dinucleotide sequences within one or more ESR1 binding sites as defined in Table 3.

Reagents which may be particularly useful in kits of the disclosure may be those that are configured to determine methylation status of one or more CpG dinucleotide sequences within an estrogen responsive enhancer of a gene selected from DAXX, ESR1, RXRA, GET4, NCOR2, GATA3, MSI2, C8orf46 and/or ITPK1. For example, the kit may comprise one or more reagents configured to determine methylation status of one or more CpG dinucleotide sequences within an estrogen responsive enhancer within a gene selected from DAXX, ESR1, RXRA, GET4, NCOR2, GATA3, MSI2, C8orf46 and/or ITPK1. Exemplary reagents for inclusion in a kit of the disclosure include those configured to determine methylation status of one or more CpG dinucleotide sequences within one or more genomic regions selected from those defined in rows 57, 111-113, 256-258, 288-289, 469-470, 805, 821-822 and 824-826 of Table 1.

In another example, kits of the disclosure may comprise one or more reagents configured to determine methylation status of one or more CpG dinucleotide sequences within an estrogen responsive enhancer within a gene selected from DAXX, RXRA, NCOR2, MSI2, and/or C8orf46 e.g., such as a reagent configured to determine methylation status at one or more CpG dinucleotide sequences within an estrogen responsive enhancer within a gene selected from DAXX, RXRA, NCOR2, MSI2, and/or C8orf46.

In yet another example, kits of the disclosure may comprise one or more reagents configured to determine methylation status of one or more CpG dinucleotide sequences within an estrogen responsive enhancer within a gene selected from FOXA1, ESR1 and/or GATA3.

In any of the kits disclosed herein, the reference level of methylation is a level of methylation determined for one or more CpG dinucleotide sequences within a corresponding genomic region of a sample selected from the group consisting of:

(i) a sample from a normal or healthy tissue;

(ii) a sample comprising a non-cancerous cell;

(iii) a sample comprising a cancerous cell, other than a breast cancer cell characterized as being ESR1-negative subtype;

(iv) a sample comprising a cancerous cell, other than a breast cancer cell characterized as being a ESR1-positive subtype which is refractory to endocrine therapy;

(v) an extract of any one of (i) to (iv);

(vi) a data set comprising levels of methylation for the one or more CpG dinucleotide sequences within the corresponding estrogen responsive enhancer in a normal or healthy individual or a population of normal or healthy individuals;

(vii) a data set comprising levels of methylation for the one or more CpG dinucleotide sequences within the corresponding estrogen responsive enhancer in an individual or a population of individuals having cancer other than ESR1-negative breast cancer subtype;

(viii) a data set comprising levels of methylation for the one or more CpG dinucleotide sequences within the corresponding estrogen responsive enhancer in an individual or a population of individuals having cancer other than a ESR1-positive breast cancer subtype which is refractory to endocrine therapy; and

(ix) a data set comprising levels of methylation for the one or more CpG dinucleotide sequences within the corresponding estrogen responsive enhancer in the subject being tested wherein the levels of methylation are determined for a matched sample having normal cells.

The cancerous cell in (iii) and/or (iv) above may be, for example, a breast cancer cell. Alternatively or in addition, the cancer in (vii) and/or (viii) above may be, for example, breast cancer.

The present disclosure also provides any one of the kits disclosed herein when used in any one or more of the methods disclosed herein.

In addition, the present disclosure provides use of one or more reagents in the preparation of a medicament for predicting response to endocrine therapy in a subject suffering from estrogen receptor 1 (ESR1) positive breast cancer, wherein the one or more reagents is/are configured to determine methylation status of one or more CpG dinucleotide sequences within one or more estrogen responsive enhancers in the subject.

The present disclosure also provides the use of one or more reagents in the preparation of a medicament for predicting the therapeutic outcome of and/or monitoring the progression of estrogen receptor 1 (ESR1) positive breast cancer in a subject receiving or about to receive endocrine therapy, wherein the one or more reagents is/are configured to determine methylation status of one or more CpG dinucleotide sequences within one or more estrogen responsive enhancers in the subject.

In one example, the one or more reagents is/are configured to determine methylation status of one or more CpG dinucleotide sequences within one or more ESR1 binding sites as defined in Table 1. In another example, the one or more reagents is/are configured to determine methylation status of one or more CpG dinucleotide sequences within one or more ESR1 binding sites as defined in Table 2. In yet another example, the one or more reagents is/are configured to determine methylation status of one or more CpG dinucleotide sequences within one or more ESR1 binding sites as defined in Table 3.

Reagents which may be particularly useful for in the preparation of medicaments as disclosed herein may be those that are configured to determine methylation status of one or more CpG dinucleotide sequences within an estrogen responsive enhancer of a gene selected from DAXX, ESR1, RXRA, GET4, NCOR2, GATA3, MSI2, C8orf46 and/or ITPK1. For example, the medicament may comprise one or more reagents configured to determine methylation status of one or more CpG dinucleotide sequences within an estrogen responsive enhancer within a gene selected from DAXX, ESR1, RXRA, GET4, NCOR2, GATA3, MSI2, C8orf46 and/or ITPK1. Exemplary reagents for inclusion in a medicament as described herein include those configured to determine methylation status of one or more CpG dinucleotide sequences within one or more genomic regions selected from those defined in rows 57, 111-113, 256-258, 288-289, 469-470, 805, 821-822 and 824-826 of Table 1.

In another example, reagents which are particularly useful for in the preparation of medicaments as disclosed herein include those that are configured to determine methylation status of one or more CpG dinucleotide sequences within an estrogen responsive enhancer of a gene selected from DAXX, RXRA, NCOR2, MSI2, and/or C8orf46 e.g., such as a reagent configured to determine methylation status at one or more CpG dinucleotide sequences within an estrogen responsive enhancer within a gene selected from DAXX, RXRA, NCOR2, MSI2, and/or C8orf46.

In yet another example, reagents which are particularly useful for in the preparation of medicaments as disclosed herein include those that are configured to determine methylation status of one or more CpG dinucleotide sequences within an estrogen responsive enhancer within a gene selected from FOXA1, ESR1 and/or GATA3.

In addition, any of the methods disclosed herein may further comprise a step of administering a therapeutic treatment to a subject. For example, the determination of the presence of a particular subtype of ESR1 positive breast cancer e.g., ESR1 positive breast cancer which is responsive to endocrine therapy or ESR1 positive breast cancer which is resistant to endocrine therapy, in a subject may lead to the administration of a particular therapeutic treatment to that subject, which therapeutic treatment is particularly tailored to that particular subtype of breast cancer.

Each feature of any particular aspect or embodiment or example of the present disclosure may be applied mutatis mutandis to any other aspect or embodiment or example of the present disclosure.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1. Genome-wide DNA methylation profiling of endocrine resistant MCF7 cell models. (a-c) A colorimetric density plot showing correlation between the HM450 methylation profile of the endocrine resistant MCF7X (a), TAMR (b) and FASR (c) cells and the parent (endocrine sensitive) MCF7 cells. The plots show that while the methylation profile of the endocrine resistant cell lines is strongly correlated with the parent MCF7 cells (MCF7X, r²=0.895; TAMR, r²=0.91; FASR, r²=0.848; Pearson's Coefficient), both the MCF7X and TAMR cells predominantly gain DNA methylation, whereas the FASR cells exhibit both hyper and hypomethylation events relative to parent MCF7 cells. (d) A Venn diagram showing the overlap of HM450 methylation probes that are more heavily methylated in multiple endocrine resistant cells compared to the parent MCF7 cells (FDR<0.01). (e) A bar plot showing the association of differentially methylated HM450 probes that were common to all endocrine resistant cell lines (compared to the parent MCF7 cells) across functional/regulatory regions of the genome as determined by MCF7 ChromHMM annotation¹³. The height of the bars represents the level of enrichment measured as a ratio between the frequency of hypermethylated (dark blue) or hypomethylated (light blue) probes overlapping a functional element over the expected frequency if such overlaps were to occur at random in the genome. Statistically significant enrichments (p-value <<0.0001; hyper-geometric test) are marked with an asterisk. The numbers of commonly hyper/hypomethylated probes located within each specific region are presented in the respective column.

FIG. 2. ESR1 regulation of enhancer sites commonly hypermethylated in endocrine resistant cell models. (a) A bar plot showing the association of HM450 probes that were more heavily methylated in endocrine resistant cell models (compared to MCF7 cells) and also specifically located in enhancer regions, across ESR1, FOXA1 and GATA3 binding sites in MCF7 cells. The height of the bars represents enrichment measured as a ratio between the frequency of hypermethylated probes in enhancers overlapping a transcription factor binding site over the expected frequency if such overlaps were to occur at random across the genome (*p-value <<0.0001; hyper-geometric test). The numbers of commonly hyper/hypomethylated probes located within each specific region are presented in the columns. (b) A Venn diagram showing the overlap of enhancer-specific HM450 methylation probes that are more heavily methylated in multiple endocrine resistant cell models (compared to MCF7 cells) across ESR1, FOXA1 and GATA3 binding sites. (c) A box plot showing the log fold change (log FC) in ESR1 binding signal at ESR1-enhancer sites that contain at least one commonly hypermethylated probe (yellow box) and all other ESR1-enhancer sites that overlap a HM450 probe (grey box) in TAMR cells compared to the parent MCF7 cells. The mean log FC in ESR1 binding at hypermethylated ER-enhancer sites is −2.29 and the mean log FC of all other ESR1-enhancer sites is −0.52 (* p<<0.0001; t-test). (The whiskers of the boxplot extend to the most extreme data point, which is no more than 1.5×IQR from the box). (d) IGV screen shots to illustrate the loss of ESR1 binding in TAMR cells compared to the parent MCF7 cells in enhancer regions that overlap methylation probes that are more heavily methylated in the endocrine resistant cell models. The MCF7 ChromHMM regions are colour coded as follows; blue=enhancer, yellow=transcribed, green=promoter, light blue=CTCF, burgundy=transcribed. The HM450 beta values are shown for the MCF7 (green), MCF7X (burgundy), TAMR (orange) and FASR cells (red) and are representative of biological duplicates. ESR1 ChIP data (blue) is presented in duplicate for both MCF7 and TAMR cells. The ESR1-enhancers that overlap regions of endocrine-resistant specific hypermethylation are highlighted by the blue boxes.

FIG. 3. Characterisation of genes whose expression is negatively affected by ESR1-enhancer hypermethylation in human breast cancer. (a) Hypergeometric testing of genes whose expression is negatively affected by ESR1-enhancer hypermethylation in human breast cancer (n=291) in the MSigDB C2 database. The height of the bars represents the level of enrichment measured as a ratio between the number of genes overlapping an MSigDB C2 gene set over the expected frequency if such overlaps were to occur at random in the genome (p-value <<0.0001; hyper-geometric test). (b) Unsupervised clustering of the gene set whose expression is negatively correlated with ESR1-enhancer methylation (n=291) in ESR1 positive (red) (n=174) and ESR1 negative (n=588) breast cancer patients (obtained from TGCA breast cohort RNA-seq data).

FIG. 4. Graphical representation of the correlation between the technical replicates presented in FIG. 5. Scatter plots showing the correlation between the technical replicates of multiplex bisulphite-PCR resequencing data presented in FIG. 5 (R=Pearson correlation).

FIG. 5. Association between ESR1 enhancer methylation and breast cancer subtype. (a) A boxplot showing the median methylation of all HM450 probes that overlap an enhancer region, an ESR1 binding site and demonstrate hypermethylation in endocrine resistant vs parental MCF7 cells (n=801 probes), in normal breast tissue (green) (n=97), luminal A (light blue) (n=301), luminal B (dark blue) (n=52) and ESR1-negative (red) (n=105) breast cancer (data obtained from TCGA breast cancer cohort) (* p<0.05, ** p<<0.0001; Mann-Whitney U test). (The whiskers of the boxplot extend to the most extreme data point, which is no more than 1.5×IQR from the box). (b) A heatmap showing the methylation profile of 801 ESR1-enhancer specific HM450 probes that are more heavily methylated in endocrine resistant vs parent MCF7 cells in normal breast tissue (green) (n=97), luminal A (light blue) (n=301), luminal B (dark blue) (n=52) and ESR1-negative (red) (n=105) breast cancer. Columns are patient samples and rows are HM450 probes. The level of methylation is represented by a colour scale—blue for low levels and red for high levels of methylation. (c) Boxplots showing distribution of methylation beta values in normal n=97 (green), luminal A (light blue) (n=301), luminal B (dark blue) (n=52) and ESR1-negative (red) (n=105) breast cancer samples across HM450 probes overlapping the ESR1-binding site located within the DAXX enhancer (Chr6: 33288112-33288670) (left panel) and the DAXX promoter region (1000 bp upstream and 100 bp downstream of the transcription start site) (Chr6: 33290693-33291793) (right panel). (The whiskers of the boxplots extend to the most extreme data point, which is no more than 1.5×IQR from the box).

FIG. 6. ESR1-Enhancer DNA hypermethylation in acquired endocrine resistance in human breast cancer. (a-d) (Left panel) A scatter plot showing the methylation of individual CpG sites across the ESR1-enhancer region of interest (a—GATA3—Chr10: 8103616-8103673; b—ITPK1—Chr14: 93412603-93412703; c—ESR1—Chr6: 152124782-152125008; d—GET4—Chr7: 922042-922114) in 3 primary luminal A breast cancers from patients that received adjuvant endocrine therapy and experienced relapse free survival (RFS) (green), 3 primary luminal A breast cancers from patients that relapsed following adjuvant endocrine therapy (n/RFS) (blue) and their matched local relapse (red). Each dot represents the % methylation at an individual CpG site for a single patient and the lines represent the average methylation for the region in primary RFS (green), primary n/RFS (blue) and matched recurrent tumours (red). (Right Panel) Box plots showing the distribution of methylation values across the ESR1-enhancer region depicted in the left panel for RFS (green), prognosis/RFS (blue) and matched recurrent tumours (red); p-values correspond to t-test comparison between RFS vs n/RFS, and n/RFS vs relapse tumours. (The whiskers of the boxplot extend to the most extreme data point, which is no more than 1.5×IQR from the box).

FIG. 7. ESR1-Enhancer DNA hypermethylation in cell models of acquired endocrine resistance. (a-I) (Left panel) A scatter plot showing the methylation of individual CpG sites across the ESR1-enhancer region of interest (a—DAXX—Chr6: 33288296-33288372; b—GET4—Chr7: 922042-922114; c—ESR1—Chr6: 152124782-152125008; d-NCOR2—Chr12: 124844786-124844883; e—GATA3—Chr10: 8103616-8103673; f—ITPK1—Chr14: 93412603-93412703; g—RXRA—Chr9: 137252867-137252967; h—MSI2—Chr17: 55371693-55371786; i—C8orf46—Chr8: 67425069-67425134) in the parental MCF7 cells (green), and the endocrine resistant derivatives, TAMR (orange), MCF7X (purple) and FASR (red). Each dot represents the % methylation at an individual CpG site and the lines represent the average methylation for the region. (Right Panel) Box plots showing the distribution of methylation values across the ESR1-enhancer region depicted in the left panel for the parental MCF7 cells (green), and the endocrine resistant derivatives, TAMR (orange), MCF7X (purple) and FASR (red) (mean±SD) (*p<0.05, **p<0.01, ***p<0.001; t-test). (The whiskers of the boxplot extend to the most extreme data point, which is no more than 1.5×IQR from the box.)

FIG. 8. Graphical representation of the correlation between the technical replicates presented in FIG. 7. Scatter plots showing the correlation between the technical replicates of multiplex bisulphite-PCR resequencing data presented in FIG. 7 (R=Pearson correlation).

FIG. 9. ESR1 enhancer DNA hypermethylation in acquired endocrine resistance in human breast cancer. (a-e) (Left panel) A scatter plot showing the methylation of individual CpG sites across the ESR1-enhancer region of interest (a—DAXX—Chr6: 33288296-33288372; b—MSI2—Chr17: 55371693-55371786; c—NCOR2—Chr12: 124844786-124844883; d—RXRA—Chr9: 137252867-137252967; e—C8orf46—Chr8: 67425069-67425134) in 3 primary luminal A breast cancers from patients that received adjuvant endocrine therapy and exhibited relapse free survival (RFS) (green), 3 primary luminal A breast cancers from patients that relapsed following adjuvant endocrine therapy, defined as no relapse free survival (n/RFS) (blue) and their matched local relapse (red). Each dot represents the % methylation at an individual CpG site for a single patient and the lines represent the average methylation for the region in primary RFS (green), primary n/RFS (blue) and matched recurrent tumours (red). (Right Panel) Box plots showing the distribution of methylation values across the ESR1-enhancer region depicted in the left panel for RFS (green), prognosis/RFS (blue) and matched recurrent tumours (red); p-values correspond to t-test comparison between RFS vs n/RFS, and n/RFS vs relapse tumours. (The whiskers of the boxplots extend to the most extreme data point, which is no more than 1.5×IQR from the box).

FIG. 10. This figure provides a flow-chart illustrating a computer system of the disclosure which may be used for predicting response to endocrine therapy in a subject suffering from ESR1 positive breast cancer.

### KEY TO THE SEQUENCE LISTING

SEQ ID NO: 1: DNA sequence for bisulphite-PCR primer designated GATA3_ct_f2

SEQ ID NO: 2: DNA sequence for bisulphite-PCR fusion primer designated GATA3_ct_f2

SEQ ID NO: 3: DNA sequence for bisulphite-PCR primer designated GATA3_ct_r2

SEQ ID NO: 4: DNA sequence for bisulphite-PCR fusion primer designated GATA3_ct_r2

SEQ ID NO: 5: DNA sequence for bisulphite-PCR primer designated ESR1_ct_f1

SEQ ID NO: 6: DNA sequence for bisulphite-PCR fusion primer designated ESR1_ct_f1

SEQ ID NO: 7: DNA sequence for bisulphite-PCR primer designated ESR1_ct_r1

SEQ ID NO: 8: DNA sequence for bisulphite-PCR fusion primer designated ESR1_ct_r1

SEQ ID NO: 9: DNA sequence for bisulphite-PCR primer designated ESR1_ct_f2

SEQ ID NO: 10: DNA sequence for bisulphite-PCR fusion primer designated ESR1_ct_f2

SEQ ID NO: 11: DNA sequence for bisulphite-PCR primer designated ESR1_ct_r2

SEQ ID NO: 12: DNA sequence for bisulphite-PCR fusion primer designated ESR1_ct_r2

SEQ ID NO: 13: DNA sequence for bisulphite-PCR primer designated GET4_ct_f1

SEQ ID NO: 14: DNA sequence for bisulphite-PCR fusion primer designated GET4_ct_f1

SEQ ID NO: 15: DNA sequence for bisulphite-PCR primer designated GET4_ct_r1

SEQ ID NO: 16: DNA sequence for bisulphite-PCR fusion primer designated GET4_ct_r1

SEQ ID NO: 17: DNA sequence for bisulphite-PCR primer designated ITPK1_ct_f1

SEQ ID NO: 18: DNA sequence for bisulphite-PCR fusion primer designated ITPK1_ct_f1

SEQ ID NO: 19: DNA sequence for bisulphite-PCR primer designated ITPK1_ct_r2

SEQ ID NO: 20: DNA sequence for bisulphite-PCR fusion primer designated ITPK1_ct_r2

SEQ ID NO: 21: DNA sequence for bisulphite-PCR primer designated MSI2_ct_f2

SEQ ID NO: 22: DNA sequence for bisulphite-PCR fusion primer designated MSI2_ct_f2

SEQ ID NO: 23: DNA sequence for bisulphite-PCR primer designated MSI2_ct_r2

SEQ ID NO: 24: DNA sequence for bisulphite-PCR fusion primer designated MSI2_ct_r2

SEQ ID NO: 25: DNA sequence for bisulphite-PCR primer designated C8orf46_ga_f1

SEQ ID NO: 26: DNA sequence for bisulphite-PCR fusion primer designated C8orf46_ga_f1

SEQ ID NO: 27: DNA sequence for bisulphite-PCR primer designated C8orf46_ga_r1

SEQ ID NO: 28: DNA sequence for bisulphite-PCR fusion primer designated C8orf46_ga_r1

SEQ ID NO: 29: DNA sequence for bisulphite-PCR primer designated DAXX_ga_f2

SEQ ID NO: 30: DNA sequence for bisulphite-PCR fusion primer designated DAXX_ga_f2

SEQ ID NO: 31: DNA sequence for bisulphite-PCR primer designated DAXX_ga_r2

SEQ ID NO: 32: DNA sequence for bisulphite-PCR fusion primer designated DAXX_ga_r2

SEQ ID NO: 33: DNA sequence for bisulphite-PCR primer designated NCOR2_ga_f1

SEQ ID NO: 34: DNA sequence for bisulphite-PCR fusion primer designated NCOR2_ga_f1

SEQ ID NO: 35: DNA sequence for bisulphite-PCR primer designated NCOR2ga_r1

SEQ ID NO: 36: DNA sequence for bisulphite-PCR fusion primer designated NCOR2_ga_r1

SEQ ID NO: 37: DNA sequence for bisulphite-PCR primer designated RXRA_ga_f1

SEQ ID NO: 38: DNA sequence for bisulphite-PCR fusion primer designated RXRAga_f1

SEQ ID NO: 39: DNA sequence for bisulphite-PCR primer designated RXRA_ga_r1

SEQ ID NO: 40: DNA sequence for bisulphite-PCR fusion primer designated RXRAga_r1

### DETAILED DESCRIPTION

General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise.

The term “and/or”, e.g., “X and/or Y” shall be understood to mean either “X and Y” or

“X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

As used herein, the term “diagnosis”, and variants thereof, such as, but not limited to “diagnose” or “diagnosing” shall include, but not be limited to, a primary diagnosis of a clinical state or any primary diagnosis of a clinical state. A diagnostic method described herein is also useful for assessing responsiveness of a subject to a particular form of therapy, such as determining whether a subject having cancer will be responsive to endocrine therapy. A diagnostic method described herein is also useful for assessing the remission of a subject, or monitoring disease recurrence, or tumor recurrence, such as following surgery, radiation therapy, adjuvant therapy or chemotherapy, or determining the appearance of metastases of a primary tumor. All such uses of the assays described herein are encompassed by the present disclosure.

As used herein, the term “prognosis”, and variants thereof, such as, but not limited to “prognosing” shall refer to the prediction of the likelihood that a cancer patient e.g., a breast cancer patient, will have a cancer-attributable death, or that the cancer will progress to a worsening stage in the subject, such as recurrence or metastatic spread, or that the cancer will have or develop drug resistance, such as resistance to endocrine therapy.

As used herein, the term “cancer” shall be taken to include a disease that is characterized by uncontrolled growth of cells within a subject. The term “cancer” shall not be limited to cancer of a specific tissue or cell type. Those skilled in the art will be aware that as a cancer progresses, metastases occur in organs and tissues outside the site of the primary cancer. Accordingly, the term “cancer” as used herein shall be taken to include a metastasis of a cancer in addition to a primary tumor. A particularly preferred cancer in the context of the present disclosure is breast cancer.

As used herein, the term “breast cancer” shall be understood to include a disease that is characterized by uncontrolled growth of cells from breast tissue of a subject.

As used herein, the term “estrogen receptor 1 (ESR1) positive breast cancer” shall be understood to refer to a breast cancer which is characterised by increased expression of the ESR1 gene when compared to a non-cancerous sample or an ESR1 negative cancerous sample, or which is characterised by a level of expression of the ESR1 gene which is different from the level of expression of a housekeeping gene.

As used herein, the term “estrogen receptor 1 (ESR1) negative breast cancer” shall be understood to refer to a breast cancer which is characterised by reduced expression of the ESR1 gene when compared to a non-cancerous sample, or an ESR1 positive cancerous sample, or which is characterised by a level of expression of the ESR1 gene which is not significantly different from the level of expression of a housekeeping gene, or which is characterised by the absence of a detectable level of expression of the ESR1 gene, or which is characterised by the absence of expression of the ESR1 gene.

As used herein, the term “estrogen responsive enhancer”, “estrogen responsive enhancers”, or similar, refers to a region or regions of the genome to which estrogen-bound estrogen receptor protein, including estrogen receptor 1 (ESR1) protein bound to estrogen, binds to activate transcription of a gene. It will be appreciated that an “estrogen responsive enhancer” may be located within the gene it activates or may be cis-acting and located away from the gene it activates e.g., upstream or downstream from the gene's start site or in an unrelated part of the genome. For example, an “estrogen responsive enhancer” may be defined according to the means described in Example 2 herein, and in particular, using the ChromHMM segmentation program as described in Taberlay et al., (2014).

The term “estrogen receptor 1 binding site”, “ESR1 binding site”, or similar, as used herein refers to a region of the genome to which the ESR1 protein binds e.g., including free ESR1 protein or ESR1 protein bound to estrogen. An “estrogen responsive enhancer” may comprise one or more “estrogen receptor 1 binding sites”.

The term “endocrine therapy” is given to those treatments which target the estrogen receptor e.g., ESR1, by blocking receptor binding with an antagonist or by depriving a cancer e.g., breast cancer, of estrogen. In the context of the present disclosure, the term “endocrine therapy” shall include therapy or treatment with an agent or compound which inhibits estrogen e.g., from acting on breast cancer cells. Such therapy is routine in treatment of breast cancer which is determined to be estrogen receptor positive i.e., expresses estrogen receptor protein, such as breast cancer which is ESR1 positive. Drugs suitable for use in endocrine therapy are well known in the art, and include, for example, anastrozole, exemestane, fulvestrant, goserelin, letrozole, leuprorelin, leuprolide acetate, megestrol, palbociclib, tamoxifen and/or toremifene.

As used herein, breast cancer which is characterised as being “resistant” or “refractory” or as having “resistance” to endocrine therapy, refers to a breast cancer which does not or will not respond to treatment with endocrine therapy.

The term “tumor” as used herein, refers to all neoplastic cell growth and proliferation, whether malignant or benign, and all pre-cancerous and cancerous cells and tissues. It will also be understood that the term “tumor sample” or similar in the context of a patient having cancer refers to a sample comprising tumor material obtained from a cancer patient. The term encompasses tumor tissue samples, for example, tissue obtained by surgical resection and tissue obtained by biopsy, such as for example, a core biopsy or a fine needle biopsy. In a particular embodiment, the tumor sample is a fixed, wax-embedded tissue sample, such as a formalin-fixed, paraffin-embedded tissue sample. Additionally, the term “tumor sample” encompasses a sample comprising tumor cells obtained from sites other than the primary tumor, e.g., circulating tumor cells.

The term “test sample” as used herein is taken to mean any tissue or body fluid sample taken from a subject having or suspected of having breast cancer. The presence of breast cancer in the subject may therefore already have been determined. Thus, the methods of the present disclosure may be used to determine a particular subtype of breast cancer (such as ESR1-positive breast cancer which is responsive to endocrine therapy or ESR1-positive breast cancer which is resistance to endocrine therapy) in a subject known to have ESR1-positive breast cancer. Thus, the “test sample” may be a “tumor sample” as defined herein. Alternatively, the methods of the present disclosure may be used to determine the presence of breast cancer e.g., such as ESR1 positive breast cancer, in a subject in whom the presence of breast cancer has not previously been determined.

As used herein, the term “methylation” will be understood to mean the presence of a methyl group added by the action of a DNA methyl transferase enzyme to a cytosine base or bases in a region of nucleic acid e.g. genomic DNA. Accordingly, the term, “methylation status” as used herein refers to the presence or absence of methylation in a specific nucleic acid region e.g., genomic region. In particular, the present disclosure relates to detection of methylated cytosine (5-methylcytosine). A nucleic acid sequence may comprise one or more CpG methylation sites.

As used herein, the term “differential methylation” shall be taken to mean a change in the relative amount of methylation of a nucleic acid e.g., genomic DNA, in a biological sample e.g., such as a cell or a cell extract, or a body fluid (such as blood), obtained from a subject. In one example, the term “differential methylation” is an increased level of methylation of a nucleic acid. In another example, the term “differential methylation” is a decreased level of methylation of a nucleic acid. In the present disclosure, “differential methylation” is generally determined with reference to a baseline level of methylation for a given genomic region, such as a non-cancerous sample, including a non-cancerous matched sample from a subject known to have cancer e.g., breast cancer. For example, the level of differential methylation may be at least 2% greater or less than a baseline level of methylation, for example at least 5% greater or less than a baseline level of methylation, or at least 10% greater or less than a baseline level of methylation, or at least 15% greater or less than a baseline level of methylation, or at least 20% greater or less than a baseline level of methylation, or at least 25% greater or less than a baseline level of methylation, or at least 30% greater or less than a baseline level of methylation, or at least 40% greater or less than a baseline level of methylation, or at least 50% greater or less than a baseline level of methylation, or at least 60% greater or less than a baseline level of methylation, or at least 70% greater or less than a baseline level of methylation, or at least 80% greater or less than a baseline level of methylation, or at least 90% greater or less than a baseline level of methylation. Thus, the level of differential methylation may be at least 10%, at least 15%, at least 20%, or at least 25% greater than or less than a baseline level of methylation. For example, the level of differential methylation may be at least 10%, at least 15%, at least 20%, or at least 25% greater than a baseline level of methylation.

As used herein, a “CpG dinucleotide”, “CpG methylation site” or equivalent, shall be taken to denote a cytosine linked to a guanine by a phosphodiester bond. CpG dinucleotides are targets for methylation of the cytosine residue and may reside within coding or non-coding nucleic acids. Non-coding nucleic acids are understood in the art to include introns, 5′-untranslated regions, 3′ untranslated regions, promoter regions of a genomic gene, or intergenic regions.

As used herein, a “reference level of methylation” shall be understood to include a level of methylation detected in a corresponding nucleic acid from a normal or healthy cell or tissue or body fluid, or a data set produced using information from a normal or healthy cell or tissue or body fluid. A “reference level of methylation” can also include a level of methylation detected in a corresponding nucleic acid from a cell or tissue or body fluid from a subject suffering from ESR1-positive breast cancer who is refractory to endocrine therapy, or a data set produced using information from a cell or tissue or body fluid from a subject suffering from ESR1-positive breast cancer who is refractory to endocrine therapy i.e., to provide a baseline level of methylation in a subject who is refractive to endocrine therapy. A “reference level of methylation” can also include a level of methylation detected in a corresponding nucleic acid from a cell or tissue or body fluid from a subject suffering from ESR1-positive breast cancer who is responsive to endocrine therapy, or a data set produced using information from a cell or tissue or body fluid from a subject suffering from ESR1-positive breast cancer who is responsive to endocrine therapy i.e., to provide a baseline level of methylation in a subject who is responsive to endocrine therapy. For example, a “reference level of methylation” may be a level of methylation in a corresponding nucleic acid from:

(i) a sample from a normal or healthy tissue;

(ii) a sample comprising a non-cancerous cell;

(iii) a sample comprising a cancerous cell, other than a breast cancer cell characterized as being ESR1-negative subtype;

(iv) a sample comprising a cancerous cell, other than a breast cancer cell characterized as being a ESR1-positive subtype which is refractory to endocrine therapy;

(v) a sample comprising a breast cancer cell characterized as being a ESR1-positive subtype which is refractory to endocrine therapy;

(vi) a sample comprising a breast cancer cell characterized as being a ESR1-positive subtype which is responsive to endocrine therapy;

(vii) an extract of any one of (i) to (vi);

(viii) a data set comprising levels of methylation for the one or more CpG dinucleotide sequences within the corresponding estrogen responsive enhancer in a normal or healthy individual or a population of normal or healthy individuals;

(ix) a data set comprising levels of methylation for the one or more CpG dinucleotide sequences within the corresponding estrogen responsive enhancer in an individual or a population of individuals having cancer other than ESR1-negative breast cancer subtype;

(x) a data set comprising levels of methylation for the one or more CpG dinucleotide sequences within the corresponding estrogen responsive enhancer in an individual or a population of individuals having cancer other than a ESR1-positive breast cancer subtype which is refractory to endocrine therapy;

(xi) a data set comprising levels of methylation for the one or more CpG dinucleotide sequences within the corresponding estrogen responsive enhancer in an individual or a population of individuals having breast cancer characterized as being a ESR1-positive breast cancer subtype which is refractory to endocrine therapy;

(xii) a data set comprising levels of methylation for the one or more CpG dinucleotide sequences within the corresponding estrogen responsive enhancer in an individual or a population of individuals having breast cancer characterized as being a ESR1-positive breast cancer subtype which is responsive to endocrine therapy; and

(xiii) a data set comprising levels of methylation for the one or more CpG dinucleotide sequences within the corresponding estrogen responsive enhancer in the subject being tested wherein the levels of methylation are determined for a matched sample having normal cells.

Preferably, the non-cancerous sample is (i) or (ii) or (viii) or (xi).

In one example, the reference level of methylation may be a level of methylation determined for one or more CpG dinucleotide sequences within a corresponding genomic region of a healthy breast epithelial cell. Thus, the normal or healthy cell or tissue may comprise a breast epithelial cell. In addition, the “non-cancerous cell” may be a breast epithelial cell. The extract of the normal or healthy cell or tissue, or of the non-cancerous cell may be an extract from a breast epithelial cell.

As used herein, the term “subject” or “patient” shall be taken to mean any animal including a human, preferably a mammal. Exemplary subjects include but are not limited to humans, primates, livestock (e.g. sheep, cows, horses, donkeys, pigs), companion animals (e.g. dogs, cats), laboratory test animals (e.g. mice, rabbits, rats, guinea pigs, hamsters), captive wild animals (e.g. fox, deer). Preferably the mammal is a human or primate. More preferably the mammal is a human.

DNA Methylation Biomarkers

The present disclosure provides a method for detecting differential methylation at one or more CpG dinucleotide sequences within one or more estrogen responsive enhancers in a subject suffering from ESR1 positive breast cancer, said method comprising performing an assay on a sample from the subject configured to determine methylation status at one or more CpG dinucleotide sequences within one or more estrogen responsive enhancers in the subject, and detecting differential methylation at the one or more CpG dinucleotide sequences within the one or more estrogen responsive enhancers in the subject relative to a reference level of methylation for the corresponding one or more CpG dinucleotide sequences. For example, detecting differential methylation at the one or more CpG dinucleotide sequences may comprise comparing a level of methylation at the one or more CpG dinucleotide sequences in the subject to the reference level of methylation for the corresponding one or more CpG dinucleotide sequences, and determining whether methylation at the one or more CpG dinucleotide sequences in the subject differs to the corresponding reference level(s) of methylation.

The present disclosure also provides a method for predicting response to endocrine therapy in a subject suffering from ESR1 positive breast cancer, comprising detecting the methylation status of one or more CpG dinucleotides within one or more estrogen responsive enhancers in the subject, and determining differential methylation at said one or more CpG dinucleotides in the subject relative to a reference level of methylation for the corresponding one or more CpG dinucleotide sequences, wherein differential methylation at said one or more CpG dinucleotides in the subject relative to the reference level is indicative of the subject's likely response to endocrine therapy. For example, determining increased methylation at the one or more CpG dinucleotide sequences within the one or more estrogen responsive enhancers relative to the reference level may be indicative of the ESR1-positive breast cancer being refractory to endocrine therapy.

The present disclosure also provides a method for diagnosing ESR1 positive breast cancer which is refractory to endocrine therapy in a subject suffering from ESR1 positive breast cancer, comprising detecting the methylation status of one or more CpG dinucleotides within one or more estrogen responsive enhancers in the subject, and determining differential methylation at said one or more CpG dinucleotides in the subject relative to a reference level of methylation for the corresponding one or more CpG dinucleotide sequences, wherein differential methylation at said one or more CpG dinucleotides in the subject relative to the reference level is indicative of the subject having breast cancer which is refractory to endocrine therapy.

The present disclosure also provides a method for predicting the therapeutic outcome of and/or monitoring the progression of ESR1 positive breast cancer in a subject receiving or about to receive endocrine therapy, comprising detecting the methylation status of one or more CpG dinucleotides within one or more estrogen responsive enhancers in the subject, and determining differential methylation at said one or more CpG dinucleotides in the subject relative to a reference level of methylation for the corresponding one or more CpG dinucleotide sequences, wherein differential methylation at said one or more CpG dinucleotides in the subject relative to the reference level is indicative of the likely therapeutic outcome and/or of the progression of the ESR1 positive breast cancer. For example, increased methylation at the one or more CpG dinucleotide sequences within the one or more estrogen responsive enhancers relative to the reference level may be indicative of the ESR1-positive breast cancer being refractory to endocrine therapy and/or that the subject is not responding to the endocrine therapy.

In any one of the foregoing methods, identifying differential methylation at the one or more CpG dinucleotides in the subject relative to the reference level may be used to determine whether the ESR1-positive breast cancer is a luminal A breast cancer subtype or a luminal B breast cancer subtype. Accordingly, methods of the disclosure may also comprise determining whether the ESR1-positive breast cancer is a luminal A breast cancer subtype or a luminal B breast cancer subtype.

The one or more CpG dinucleotide sequences may be within one or more ESR1 binding sites which are within one or more estrogen responsive enhancers. For example, the one or more CpG dinucleotide sequences may be within one or more of the ESR1 binding sites set forth in Table 1. The ESR1 binding sites set forth in Table 1 are defined with reference to human genome assembly version 19 (“hg19”). As used herein, “hg19” refers to the February 2009 human reference sequence (Genome Reference Consortium GRCh37), which was produced by the International Human Genome Sequencing Consortium. Further information about this assembly is provided under the reference Genome Reference Consortium GRCh37 in the NCBI Assembly database. Thus, the nucleotide sequences of each of the regions identified in Table 1 (or in any of the Tables disclosed herein) can be identified by reference to hg19, using the “start” and “end” positions described in Table 1 (or in any of the Tables disclosed herein).

The 856 genomic regions listed in Table 1 encompass ESR1 binding sites that overlap estrogen responsive enhancer regions containing hypermethylated CpG dinucleotides in multiple models of endocrine resistance (i.e., MCF7-derived cell lines, tamoxifen-resistant (TAMR)10, fulvestrant-resistant (FASR)11 and estrogen deprivation resistant (MCF7X)12 cells) relative to ESR1-positive hormone sensitive MCF7 cells. Increased methylation at the 856 genomic regions listed in Table 1 was found to be associated with a reduction in ESR1 binding. For each of the ESR1 binding sites set forth in Table 1, the following information is provided:

(i) chromosome ID (Column 2);

(ii) genomic coordinates of ESR1-binding site with respect to hg19 (Columns 3-4);

(iii) ESR1-binding site name (Column 5);

(iv) name of gene within which ESR1-binding site is located (Column 6); and

(v) whether hypermethylation resulted in loss of ESR1 binding in TAMR cells

TABLE 1
Hypermethylated ESR1 enhancer binding sites

| | | | | | | Loss of ESR1 |
|---|---|---|---|---|---|---|
| | | | | | | binding in TAMR |
| Row No. | Chromosome | Start | End | ESR1 site name | Gene | Cells |
| 1 | 7 | 27210940 | 27211286 | ER_100307 | HOXA-AS4 | No |
| 2 | 7 | 43288514 | 43288831 | ER_100892 | HECW1 | Yes |
| 3 | 7 | 44224085 | 44224425 | ER_100934 | GCK | No |
| 4 | 7 | 47611478 | 47611980 | ER_101074 | TNS3 | No |
| 5 | 7 | 55620324 | 55620676 | ER_101291 | VOPP1 | No |
| 6 | 7 | 73866691 | 73867043 | ER_101653 | GTF2IRD1 | Yes |
| 7 | 7 | 74020804 | 74021327 | ER_101674 | GTF2IRD1 | No |
| 8 | 7 | 74024605 | 74024980 | ER_101675 | GTF2IRD1 | No |
| 9 | 7 | 75279353 | 75279713 | ER_101692 | HIP1 | Yes |
| 10 | 7 | 75362309 | 75362707 | ER_101699 | HIP1 | Yes |
| 11 | 1 | 223854199 | 223854500 | ER_10205 | CAPN8 | Yes |
| 12 | 7 | 87855579 | 87855879 | ER_102057 | SRI | No |
| 13 | 1 | 224400118 | 224400678 | ER_10231 | NVL | Yes |
| 14 | 7 | 98989800 | 98990147 | ER_102479 | ARPC1B | Yes |
| 15 | 7 | 99719664 | 99719969 | ER_102522 | CNPY4 | Yes |
| 16 | 7 | 100463589 | 100463889 | ER_102585 | SLC12A9 | No |
| 17 | 7 | 100464090 | 100464390 | ER_102586 | SLC12A9 | Yes |
| 18 | 7 | 100769916 | 100770235 | ER_102602 | SERPINE1 | Yes |
| 19 | 7 | 100800324 | 100800624 | ER_102605 | AP1S1 | Yes |
| 20 | 7 | 101017180 | 101017480 | ER_102622 | EMID2 | No |
| 21 | 7 | 101180514 | 101180818 | ER_102635 | EMID2 | Yes |
| 22 | 7 | 101273902 | 101274202 | ER_102645 | MYL10 | No |
| 23 | 7 | 101768479 | 101768882 | ER_102709 | CUX1 | Yes |
| 24 | 7 | 101959130 | 101959535 | ER_102722 | SH2B2 | Yes |
| 25 | 7 | 102080614 | 102081003 | ER_102733 | ORAI2 | No |
| 26 | 7 | 105313870 | 105314183 | ER_102843 | ATXN7L1 | Yes |
| 27 | 1 | 27240488 | 27241066 | ER_1029 | NR0B2 | No |
| 28 | 7 | 107886705 | 107887046 | ER_103011 | NRCAM | No |
| 29 | 1 | 228598287 | 228598587 | ER_10389 | TRIM17 | No |
| 30 | 7 | 138560635 | 138560935 | ER_104150 | KIAA1549 | Yes |
| 31 | 7 | 139345059 | 139345423 | ER_104192 | HIPK2 | Yes |
| 32 | 7 | 140206668 | 140207069 | ER_104247 | DENND2A | No |
| 33 | 7 | 143106427 | 143106737 | ER_104387 | EPHA1-AS1 | Yes |
| 34 | 7 | 144104415 | 144104715 | ER_104415 | NOBOX | No |
| 35 | 7 | 148901363 | 148901733 | ER_104483 | ZNF282 | No |
| 36 | 7 | 149569864 | 149570164 | ER_104518 | ATP6V0E2 | Yes |
| 37 | 7 | 151418719 | 151419146 | ER_104616 | PRKAG2 | Yes |
| 38 | 7 | 151424787 | 151425151 | ER_104617 | PRKAG2 | No |
| 39 | 7 | 151442252 | 151442633 | ER_104619 | PRKAG2 | No |
| 40 | 7 | 151493516 | 151493829 | ER_104624 | PRKAG2 | Yes |
| 41 | 7 | 151553656 | 151553979 | ER_104631 | PRKAG2 | No |
| 42 | 7 | 155600847 | 155601325 | ER_104760 | SHH | Yes |
| 43 | 1 | 230882258 | 230882814 | ER_10488 | CAPN9 | Yes |
| 44 | 7 | 157069948 | 157070302 | ER_104899 | UBE3C | Yes |
| 45 | 7 | 157916425 | 157916765 | ER_104936 | PTPRN2 | Yes |
| 46 | 1 | 230895945 | 230896482 | ER_10494 | CAPN9 | Yes |
| 47 | 1 | 231113407 | 231113970 | ER_10502 | TTC13 | Yes |
| 48 | 8 | 1946769 | 1947125 | ER_105037 | KBTBD11 | No |
| 49 | 8 | 17394868 | 17395391 | ER_105282 | SLC7A2 | Yes |
| 50 | 8 | 21948482 | 21948782 | ER_105469 | FAM160B2 | No |
| 51 | 8 | 28223166 | 28223486 | ER_105673 | ZNF395 | Yes |
| 52 | 1 | 28012971 | 28013583 | ER_1057 | IFI6 | Yes |
| 53 | 8 | 37700545 | 37700845 | ER_105921 | GPR124 | No |
| 54 | 8 | 48739114 | 48739414 | ER_106145 | PRKDC | Yes |
| 55 | 8 | 56791360 | 56791707 | ER_106342 | LYN | Yes |
| 56 | 8 | 62567898 | 62568253 | ER_106497 | ASPH | Yes |
| 57 | 8 | 67425037 | 67425360 | ER_106600 | C8orf46 | Yes |
| 58 | 8 | 74002442 | 74002818 | ER_106800 | SBSPON | No |
| 59 | 8 | 90772144 | 90772507 | ER_107685 | RIPK2 | No |
| 60 | 8 | 98881668 | 98882017 | ER_108508 | MATN2 | No |
| 61 | 8 | 101017572 | 101017872 | ER_108722 | RGS22 | Yes |
| 62 | 1 | 246868690 | 246869132 | ER_10884 | SCCPDH | Yes |
| 63 | 1 | 249106283 | 249106588 | ER_10898 | SH3BP5L | Yes |
| 64 | 8 | 102526632 | 102526954 | ER_109025 | GRHL2 | Yes |
| 65 | 8 | 103588959 | 103589377 | ER_109184 | ODF1 | Yes |
| 66 | 10 | 416280 | 416842 | ER_10930 | DIP2C | Yes |
| 67 | 10 | 579019 | 579393 | ER_10949 | DIP2C | Yes |
| 68 | 8 | 124172628 | 124172928 | ER_110725 | WDR67 | No |
| 69 | 8 | 124179711 | 124180074 | ER_110727 | FAM83A | No |
| 70 | 8 | 124193498 | 124193984 | ER_110732 | FAM83A | No |
| 71 | 8 | 124194626 | 124194965 | ER_110733 | FAM83A | No |
| 72 | 8 | 126082161 | 126082738 | ER_111167 | KIAA0196 | Yes |
| 73 | 8 | 126398383 | 126398799 | ER_111257 | NSMCE2 | No |
| 74 | 10 | 5538429 | 5538782 | ER_11143 | CALML5 | Yes |
| 75 | 8 | 129103245 | 129103607 | ER_111781 | PVT1 | No |
| 76 | 8 | 134224471 | 134224833 | ER_112110 | WISP1 | No |
| 77 | 8 | 134249814 | 134250165 | ER_112114 | NDRG1 | No |
| 78 | 8 | 135490722 | 135491122 | ER_112226 | ZFAT | Yes |
| 79 | 8 | 142139549 | 142139849 | ER_112470 | DENND3 | Yes |
| 80 | 8 | 142247703 | 142248043 | ER_112484 | SLC45A4 | Yes |
| 81 | 8 | 143395413 | 143395760 | ER_112563 | TSNARE1 | Yes |
| 82 | 8 | 143625782 | 143626082 | ER_112589 | BAI1 | Yes |
| 83 | 8 | 143690473 | 143690892 | ER_112597 | ARC | No |
| 84 | 8 | 143763120 | 143763420 | ER_112608 | PSCA | No |
| 85 | 8 | 143823681 | 143823981 | ER_112619 | SLURP1 | No |
| 86 | 8 | 143851193 | 143851559 | ER_112624 | LYNX1 | Yes |
| 87 | 8 | 143867136 | 143867477 | ER_112628 | LY6D | Yes |
| 88 | 8 | 144129484 | 144129962 | ER_112655 | C8orf31 | No |
| 89 | 10 | 7984711 | 7985286 | ER_11272 | TAF3 | No |
| 90 | 8 | 145048592 | 145048911 | ER_112777 | PLEC | Yes |
| 91 | 8 | 145086414 | 145086732 | ER_112784 | SPATC1 | Yes |
| 92 | 8 | 145721323 | 145721690 | ER_112829 | PPP1R16A | Yes |
| 93 | 8 | 145728596 | 145728968 | ER_112830 | GPT | Yes |
| 94 | 9 | 33443026 | 33443378 | ER_113615 | AQP3 | No |
| 95 | 9 | 34372740 | 34373087 | ER_113641 | KIAA1161 | No |
| 96 | 9 | 71660820 | 71661159 | ER_113876 | FXN | Yes |
| 97 | 9 | 79629665 | 79630009 | ER_114134 | FOXB2 | No |
| 98 | 9 | 95834504 | 95834841 | ER_115179 | SUSD3 | Yes |
| 99 | 1 | 3388133 | 3388514 | ER_116 | ARHGEF16 | Yes |
| 100 | 9 | 124979962 | 124980333 | ER_116729 | LHX6 | No |
| 101 | 1 | 32039497 | 32039797 | ER_1168 | TINAGL1 | No |
| 102 | 1 | 3398747 | 3399296 | ER_117 | ARHGEF16 | Yes |
| 103 | 9 | 129433798 | 129434130 | ER_117042 | LMX1B | Yes |
| 104 | 9 | 130484264 | 130484588 | ER_117145 | TTC16 | Yes |
| 105 | 9 | 130487106 | 130487456 | ER_117146 | TTC16 | No |
| 106 | 9 | 130524258 | 130524602 | ER_117150 | SH2D3C | Yes |
| 107 | 9 | 130727796 | 130728104 | ER_117181 | FAM102A | Yes |
| 108 | 9 | 131934253 | 131934574 | ER_117307 | IER5L | Yes |
| 109 | 9 | 133974376 | 133974700 | ER_117471 | AIF1L | Yes |
| 110 | 9 | 136728378 | 136728709 | ER_117634 | VAV2 | Yes |
| 111 | 9 | 137252760 | 137253089 | ER_117681 | RXRA | Yes |
| 112 | 9 | 137258516 | 137258927 | ER_117685 | RXRA | Yes |
| 113 | 9 | 137302122 | 137302482 | ER_117702 | RXRA | Yes |
| 114 | 9 | 138997255 | 138997607 | ER_117799 | C9orf69 | Yes |
| 115 | 9 | 139069147 | 139069469 | ER_117809 | LHX3 | Yes |
| 116 | 9 | 139734802 | 139735102 | ER_117864 | RABL6 | Yes |
| 117 | 9 | 139949038 | 139949338 | ER_117897 | ENTPD2 | Yes |
| 118 | 9 | 140188799 | 140189099 | ER_117923 | NRARP | No |
| 119 | 9 | 140374065 | 140374394 | ER_117940 | PNPLA7 | Yes |
| 120 | 9 | 140408341 | 140408692 | ER_117943 | PNPLA7 | Yes |
| 121 | 10 | 24496562 | 24497177 | ER_11835 | KIAA1217 | Yes |
| 122 | X | 16889511 | 16889834 | ER_118379 | RBBP7 | Yes |
| 123 | X | 18708777 | 18709167 | ER_118438 | PPEF1 | No |
| 124 | X | 40007163 | 40007510 | ER_118823 | BCOR | Yes |
| 125 | 1 | 32404117 | 32404574 | ER_1191 | PTP4A2 | No |
| 126 | X | 133792539 | 133792870 | ER_120294 | PLAC1 | Yes |
| 127 | 10 | 30316774 | 30317298 | ER_12062 | KIAA1462 | No |
| 128 | X | 153586498 | 153586870 | ER_120632 | FLNA | Yes |
| 129 | 1 | 3471648 | 3472248 | ER_122 | MEGF6 | Yes |
| 130 | 1 | 37321565 | 37322055 | ER_1299 | GRIK3 | Yes |
| 131 | 1 | 931045 | 931617 | ER_13 | HES4 | Yes |
| 132 | 1 | 37503121 | 37503497 | ER_1301 | GRIK3 | Yes |
| 133 | 10 | 71213510 | 71214074 | ER_13175 | TSPAN15 | Yes |
| 134 | 10 | 72202041 | 72202482 | ER_13208 | NODAL | Yes |
| 135 | 10 | 73828553 | 73829113 | ER_13269 | SPOCK2 | Yes |
| 136 | 10 | 74020315 | 74020712 | ER_13281 | DDIT4 | Yes |
| 137 | 10 | 74021102 | 74021625 | ER_13282 | DDIT4 | Yes |
| 138 | 1 | 3614361 | 3614966 | ER_133 | TP73 | Yes |
| 139 | 10 | 76858883 | 76859398 | ER_13395 | DUSP13 | Yes |
| 140 | 10 | 77872231 | 77872922 | ER_13427 | C10orf11 | No |
| 141 | 10 | 82189362 | 82189690 | ER_13657 | FAM213A | Yes |
| 142 | 10 | 88475369 | 88475919 | ER_13815 | LDB3 | No |
| 143 | 10 | 88703091 | 88703639 | ER_13821 | MMRN2 | Yes |
| 144 | 10 | 95228052 | 95228774 | ER_14079 | MYOF | Yes |
| 145 | 10 | 99331269 | 99331862 | ER_14202 | UBTD1 | No |
| 146 | 10 | 102792785 | 102793085 | ER_14340 | SFXN3 | No |
| 147 | 10 | 105238778 | 105239360 | ER_14449 | CALHM3 | No |
| 148 | 10 | 112259180 | 112259480 | ER_14629 | DUSP5 | No |
| 149 | 1 | 42420202 | 42420763 | ER_1481 | HIVEP3 | Yes |
| 150 | 10 | 119102102 | 119102657 | ER_14914 | PDZD8 | No |
| 151 | 10 | 121079102 | 121079402 | ER_15001 | GRK5 | No |
| 152 | 10 | 121415148 | 121415773 | ER_15018 | BAG3 | Yes |
| 153 | 10 | 123900770 | 123901156 | ER_15113 | TACC2 | No |
| 154 | 10 | 124888975 | 124889540 | ER_15133 | HMX3 | No |
| 155 | 10 | 126211691 | 126211991 | ER_15194 | LHPP | Yes |
| 156 | 10 | 126315723 | 126316102 | ER_15199 | FAM53B | Yes |
| 157 | 10 | 126700346 | 126700820 | ER_15221 | CTBP2 | Yes |
| 158 | 1 | 44300618 | 44300993 | ER_1532 | ST3GAL3 | Yes |
| 159 | 10 | 128606764 | 128607329 | ER_15340 | DOCK1 | Yes |
| 160 | 10 | 134079116 | 134079490 | ER_15434 | STK32C | Yes |
| 161 | 10 | 134224980 | 134225280 | ER_15443 | PWWP2B | Yes |
| 162 | 10 | 134261296 | 134261809 | ER_15450 | C10orf91 | No |
| 163 | 10 | 134332284 | 134332680 | ER_15463 | INPP5A | Yes |
| 164 | 10 | 134346912 | 134347484 | ER_15465 | INPP5A | Yes |
| 165 | 10 | 134420347 | 134420647 | ER_15478 | INPP5A | No |
| 166 | 10 | 134498671 | 134499284 | ER_15480 | INPP5A | Yes |
| 167 | 10 | 134728825 | 134729477 | ER_15487 | TTC40 | Yes |
| 168 | 10 | 134729867 | 134730225 | ER_15488 | TTC40 | Yes |
| 169 | 10 | 135178534 | 135178842 | ER_15502 | ECHS1 | Yes |
| 170 | 10 | 135337035 | 135337690 | ER_15505 | CYP2E1 | No |
| 171 | 11 | 569384 | 569784 | ER_15531 | MIR210HG | No |
| 172 | 11 | 849066 | 849366 | ER_15551 | TSPAN4 | No |
| 173 | 11 | 1224796 | 1225357 | ER_15568 | MUC5B | Yes |
| 174 | 11 | 1275364 | 1275883 | ER_15574 | MUC5B | No |
| 175 | 11 | 1460055 | 1460355 | ER_15581 | BRSK2 | Yes |
| 176 | 11 | 1507075 | 1507659 | ER_15583 | MOB2 | Yes |
| 177 | 11 | 1777371 | 1777938 | ER_15595 | CTSD | Yes |
| 178 | 11 | 1990361 | 1990661 | ER_15612 | MRPL23 | No |
| 179 | 11 | 2004135 | 2004733 | ER_15613 | MRPL23-AS1 | No |
| 180 | 11 | 2011314 | 2011614 | ER_15617 | MRPL23-AS1 | No |
| 181 | 11 | 2181411 | 2181715 | ER_15620 | INS | Yes |
| 182 | 11 | 2181411 | 2181715 | ER_15620 | INS-IGF2 | Yes |
| 183 | 11 | 2212159 | 2212517 | ER_15626 | MIR4686 | Yes |
| 184 | 11 | 2431263 | 2431688 | ER_15638 | TRPM5 | No |
| 185 | 11 | 10764241 | 10764827 | ER_15808 | CTR9 | No |
| 186 | 11 | 12309323 | 12309637 | ER_15849 | MICALCL | Yes |
| 187 | 11 | 18616740 | 18617317 | ER_16065 | SPTY2D1-AS1 | Yes |
| 188 | 11 | 20063465 | 20064029 | ER_16131 | NAV2 | No |
| 189 | 11 | 33744676 | 33745230 | ER_16397 | CD59 | Yes |
| 190 | 11 | 45928675 | 45929091 | ER_16775 | C11orf94 | Yes |
| 191 | 11 | 46373841 | 46374290 | ER_16803 | DGKZ | No |
| 192 | 11 | 61466759 | 61467374 | ER_17096 | DAGLA | Yes |
| 193 | 11 | 62437396 | 62437722 | ER_17173 | C11orf48 | Yes |
| 194 | 11 | 62647962 | 62648262 | ER_17194 | SLC3A2 | No |
| 195 | 11 | 62690015 | 62690315 | ER_17200 | CHRM1 | No |
| 196 | 11 | 64007699 | 64008267 | ER_17271 | FKBP2 | No |
| 197 | 11 | 64034904 | 64035204 | ER_17276 | PLCB3 | No |
| 198 | 11 | 64053773 | 64054355 | ER_17285 | GPR137 | Yes |
| 199 | 11 | 64322980 | 64323660 | ER_17306 | SLC22A11 | Yes |
| 200 | 11 | 64618305 | 64618873 | ER_17326 | EHD1 | Yes |
| 201 | 11 | 65121625 | 65122198 | ER_17368 | TIGD3 | Yes |
| 202 | 11 | 65548866 | 65549271 | ER_17431 | AP5B1 | No |
| 203 | 11 | 65582282 | 65582857 | ER_17439 | OVOL1 | Yes |
| 204 | 11 | 66659547 | 66660139 | ER_17525 | PC | No |
| 205 | 11 | 67165633 | 67165963 | ER_17593 | PPP1CA | No |
| 206 | 11 | 67186225 | 67186704 | ER_17595 | CARNS1 | Yes |
| 207 | 11 | 67810737 | 67811051 | ER_17643 | TCIRG1 | No |
| 208 | 11 | 67914354 | 67914874 | ER_17656 | SUV420H1 | Yes |
| 209 | 11 | 69061731 | 69062031 | ER_17743 | MYEOV | No |
| 210 | 11 | 69515571 | 69515871 | ER_17789 | FGF19 | Yes |
| 211 | 11 | 70917115 | 70917490 | ER_17848 | SHANK2 | No |
| 212 | 11 | 71276995 | 71277590 | ER_17878 | KRTAP5-10 | Yes |
| 213 | 11 | 73000242 | 73000542 | ER_17968 | P2RY6 | Yes |
| 214 | 11 | 76371803 | 76372137 | ER_18134 | LRRC32 | Yes |
| 215 | 1 | 59058083 | 59058687 | ER_1844 | TACSTD2 | No |
| 216 | 11 | 85468982 | 85469571 | ER_18506 | SYTL2 | No |
| 217 | 1 | 6330275 | 6330725 | ER_187 | ACOT7 | Yes |
| 218 | 11 | 117300693 | 117300993 | ER_19145 | DSCAML1 | No |
| 219 | 11 | 118013408 | 118013932 | ER_19187 | SCN4B | Yes |
| 220 | 1 | 6445555 | 6445855 | ER_192 | ACOT7 | No |
| 221 | 11 | 118758401 | 118758701 | ER_19225 | CXCR5 | No |
| 222 | 11 | 119994594 | 119994894 | ER_19291 | TRIM29 | No |
| 223 | 11 | 119995602 | 119996221 | ER_19292 | TRIM29 | No |
| 224 | 11 | 120106109 | 120106716 | ER_19293 | POU2F3 | No |
| 225 | 12 | 2021757 | 2022057 | ER_19684 | CACNA2D4 | Yes |
| 226 | 12 | 48340415 | 48340727 | ER_21318 | TMEM106C | Yes |
| 227 | 12 | 48396469 | 48396769 | ER_21327 | COL2A1 | No |
| 228 | 12 | 49759304 | 49759746 | ER_21394 | SPATS2 | Yes |
| 229 | 12 | 51236349 | 51236665 | ER_21514 | TMPRSS12 | No |
| 230 | 12 | 52542519 | 52542858 | ER_21611 | KRT80 | Yes |
| 231 | 12 | 52579425 | 52579733 | ER_21620 | KRT80 | Yes |
| 232 | 12 | 53300210 | 53300676 | ER_21729 | KRT8 | Yes |
| 233 | 12 | 53552932 | 53553480 | ER_21786 | CSAD | Yes |
| 234 | 12 | 56123942 | 56124242 | ER_21950 | CD63 | Yes |
| 235 | 12 | 56652692 | 56653098 | ER_22003 | ANKRD52 | No |
| 236 | 12 | 57559601 | 57559901 | ER_22058 | LRP1 | No |
| 237 | 12 | 58160686 | 58160986 | ER_22088 | CYP27B1 | No |
| 238 | 12 | 58209624 | 58209992 | ER_22094 | AVIL | No |
| 239 | 12 | 63193501 | 63193816 | ER_22367 | PPM1H | Yes |
| 240 | 12 | 65043934 | 65044256 | ER_22510 | RASSF3 | Yes |
| 241 | 12 | 65066737 | 65067037 | ER_22517 | RASSF3 | Yes |
| 242 | 12 | 68633903 | 68634289 | ER_22744 | IL22 | No |
| 243 | 1 | 7705733 | 7706033 | ER_240 | CAMTA1 | No |
| 244 | 12 | 103344027 | 103344491 | ER_24357 | ASCL1 | No |
| 245 | 12 | 109162594 | 109162897 | ER_24689 | SSH1 | Yes |
| 246 | 12 | 111029308 | 111029650 | ER_24787 | PPTC7 | No |
| 247 | 12 | 111840744 | 111841077 | ER_24815 | SH2B3 | Yes |
| 248 | 12 | 112207002 | 112207577 | ER_24835 | ALDH2 | No |
| 249 | 12 | 113547314 | 113547643 | ER_24903 | RASAL1 | No |
| 250 | 12 | 117471560 | 117471860 | ER_25133 | FBXW8 | No |
| 251 | 12 | 120703585 | 120704001 | ER_25278 | PXN | Yes |
| 252 | 12 | 122019200 | 122019628 | ER_25371 | KDM2B | No |
| 253 | 12 | 122458756 | 122459117 | ER_25409 | BCL7A | No |
| 254 | 12 | 123446240 | 123446640 | ER_25491 | ABCB9 | Yes |
| 255 | 12 | 123449086 | 123449400 | ER_25493 | ABCB9 | No |
| 256 | 12 | 124844847 | 124845161 | ER_25578 | NCOR2 | Yes |
| 257 | 12 | 124879925 | 124880326 | ER_25592 | NCOR2 | Yes |
| 258 | 12 | 125004808 | 125005149 | ER_25616 | NCOR2 | Yes |
| 259 | 12 | 131438531 | 131438856 | ER_25787 | GPR133 | Yes |
| 260 | 12 | 131622396 | 131622696 | ER_25800 | GPR133 | No |
| 261 | 12 | 132285353 | 132285666 | ER_25837 | SFSWAP | Yes |
| 262 | 12 | 132348290 | 132348625 | ER_25849 | MMP17 | Yes |
| 263 | 12 | 132671184 | 132671527 | ER_25874 | GALNT9 | Yes |
| 264 | 12 | 132694663 | 132695050 | ER_25875 | GALNT9 | No |
| 265 | 13 | 20965643 | 20965996 | ER_25987 | CRYL1 | No |
| 266 | 13 | 25691436 | 25692026 | ER_26197 | PABPC3 | Yes |
| 267 | 13 | 34208323 | 34209020 | ER_26636 | STARD13 | Yes |
| 268 | 13 | 41590103 | 41590403 | ER_26934 | ELF1 | Yes |
| 269 | 13 | 51854732 | 51855052 | ER_27435 | FAM124A | No |
| 270 | 13 | 113651709 | 113652104 | ER_28798 | MCF2L | Yes |
| 271 | 13 | 113677094 | 113677394 | ER_28802 | MCF2L | Yes |
| 272 | 13 | 113979939 | 113980263 | ER_28813 | GRTP1 | Yes |
| 273 | 14 | 21494973 | 21495345 | ER_28907 | NDRG2 | No |
| 274 | 1 | 8823899 | 8824414 | ER_290 | RERE | No |
| 275 | 14 | 23623250 | 23623562 | ER_29019 | SLC7A8 | Yes |
| 276 | 14 | 23623676 | 23623976 | ER_29020 | SLC7A8 | Yes |
| 277 | 14 | 38054124 | 38054508 | ER_29794 | FOXA1 | Yes |
| 278 | 14 | 64932503 | 64932803 | ER_31140 | AKAP5 | No |
| 279 | 14 | 68871364 | 68871773 | ER_31488 | RAD51B | Yes |
| 280 | 14 | 69263206 | 69263506 | ER_31542 | ZFP36L1 | Yes |
| 281 | 14 | 74214079 | 74214466 | ER_31871 | C14orf43 | Yes |
| 282 | 14 | 74238204 | 74238510 | ER_31884 | C14orf43 | Yes |
| 283 | 1 | 1141942 | 1142242 | ER_32 | TNFRSF18 | Yes |
| 284 | 14 | 76447292 | 76447654 | ER_32084 | TGFB3 | No |
| 285 | 14 | 88942632 | 88943092 | ER_32519 | PTPN21 | No |
| 286 | 14 | 91723710 | 91724098 | ER_32707 | GPR68 | Yes |
| 287 | 14 | 91730894 | 91731408 | ER_32708 | CCDC88C | No |
| 288 | 14 | 93412574 | 93412953 | ER_32818 | ITPK1 | Yes |
| 289 | 14 | 93473727 | 93474077 | ER_32832 | ITPK1 | Yes |
| 290 | 14 | 94392599 | 94392913 | ER_32902 | FAM181A-AS1 | Yes |
| 291 | 14 | 95047627 | 95047927 | ER_32949 | SERPINA5 | No |
| 292 | 14 | 95078366 | 95078753 | ER_32959 | SERPINA3 | Yes |
| 293 | 14 | 95615683 | 95616180 | ER_32995 | DICER1 | No |
| 294 | 1 | 1143577 | 1143920 | ER_33 | TNFRSF18 | Yes |
| 295 | 14 | 96691960 | 96692332 | ER_33117 | BDKRB2 | No |
| 296 | 14 | 100055296 | 100055707 | ER_33296 | CCDC85C | No |
| 297 | 14 | 100196895 | 100197241 | ER_33310 | CYP46A1 | No |
| 298 | 14 | 100618272 | 100618577 | ER_33370 | DEGS2 | No |
| 299 | 14 | 103541236 | 103541566 | ER_33566 | CDC42BPB | Yes |
| 300 | 14 | 103566340 | 103566640 | ER_33567 | EXOC3L4 | Yes |
| 301 | 14 | 103576070 | 103576413 | ER_33570 | EXOC3L4 | No |
| 302 | 14 | 104094520 | 104094858 | ER_33611 | KLC1 | Yes |
| 303 | 14 | 104165149 | 104165544 | ER_33615 | XRCC3 | Yes |
| 304 | 14 | 104637698 | 104638043 | ER_33656 | KIF26A | Yes |
| 305 | 14 | 105131984 | 105132284 | ER_33687 | MIR4710 | Yes |
| 306 | 14 | 105181841 | 105182228 | ER_33695 | INF2 | Yes |
| 307 | 14 | 105215618 | 105215967 | ER_33701 | ADSSL1 | Yes |
| 308 | 14 | 105285893 | 105286336 | ER_33720 | LINC00638 | Yes |
| 309 | 14 | 105434204 | 105434522 | ER_33730 | AHNAK2 | Yes |
| 310 | 14 | 105446288 | 105446760 | ER_33737 | AHNAK2 | Yes |
| 311 | 14 | 105823215 | 105823515 | ER_33780 | PACS2 | Yes |
| 312 | 14 | 105992102 | 105992449 | ER_33807 | TMEM121 | No |
| 313 | 15 | 28352967 | 28353510 | ER_33941 | HERC2 | Yes |
| 314 | 15 | 32951599 | 32952135 | ER_33988 | SCG5 | Yes |
| 315 | 15 | 41258295 | 41258881 | ER_34198 | CHAC1 | Yes |
| 316 | 15 | 50519199 | 50519766 | ER_34615 | SLC27A2 | Yes |
| 317 | 1 | 1173574 | 1173890 | ER_35 | B3GALT6 | Yes |
| 318 | 15 | 53095569 | 53095869 | ER_35206 | ONECUT1 | No |
| 319 | 1 | 10075638 | 10076128 | ER_353 | RBP7 | No |
| 320 | 15 | 63074443 | 63074894 | ER_35498 | TLN2 | No |
| 321 | 15 | 63345507 | 63345850 | ER_35518 | TPM1 | Yes |
| 322 | 15 | 63671577 | 63672181 | ER_35532 | CA12 | Yes |
| 323 | 15 | 63672612 | 63673165 | ER_35533 | CA12 | Yes |
| 324 | 15 | 69588677 | 69589124 | ER_35794 | PAQR5 | Yes |
| 325 | 15 | 70384192 | 70384714 | ER_35866 | TLE3 | Yes |
| 326 | 15 | 73667419 | 73667968 | ER_36062 | HCN4 | Yes |
| 327 | 15 | 74232754 | 74233252 | ER_36102 | LOXL1 | Yes |
| 328 | 15 | 74495260 | 74495560 | ER_36118 | STRA6 | Yes |
| 329 | 15 | 74537511 | 74538135 | ER_36123 | CCDC33 | Yes |
| 330 | 15 | 75974558 | 75974909 | ER_36216 | CSPG4 | Yes |
| 331 | 15 | 79223130 | 79223938 | ER_36351 | CTSH | Yes |
| 332 | 15 | 80878455 | 80879031 | ER_36421 | ARNT2 | No |
| 333 | 15 | 81596013 | 81596551 | ER_36480 | IL16 | No |
| 334 | 15 | 83781576 | 83782122 | ER_36550 | TM6SF1 | Yes |
| 335 | 15 | 86219506 | 86219806 | ER_36659 | AKAP13 | Yes |
| 336 | 15 | 89027778 | 89028410 | ER_36781 | MRPS11 | No |
| 337 | 15 | 89630906 | 89631554 | ER_36798 | ABHD2 | Yes |
| 338 | 15 | 90328102 | 90328625 | ER_36856 | ANPEP | Yes |
| 339 | 15 | 90349924 | 90350224 | ER_36858 | ANPEP | Yes |
| 340 | 15 | 90649755 | 90650347 | ER_36890 | IDH2 | Yes |
| 341 | 15 | 93573071 | 93573371 | ER_37056 | CHD2 | No |
| 342 | 15 | 96866722 | 96867218 | ER_37215 | NR2F2 | Yes |
| 343 | 15 | 99236506 | 99236926 | ER_37297 | IGF1R | No |
| 344 | 15 | 99271960 | 99272504 | ER_37301 | IGF1R | Yes |
| 345 | 15 | 101548985 | 101549285 | ER_37458 | LRRK1 | No |
| 346 | 16 | 374320 | 374831 | ER_37511 | AXIN1 | Yes |
| 347 | 16 | 585341 | 586026 | ER_37534 | MIR5587 | Yes |
| 348 | 16 | 615200 | 615500 | ER_37536 | C16orf11 | Yes |
| 349 | 16 | 633459 | 633759 | ER_37539 | PIGQ | Yes |
| 350 | 16 | 635443 | 635747 | ER_37540 | PIGQ | Yes |
| 351 | 16 | 701631 | 702182 | ER_37550 | WDR90 | Yes |
| 352 | 16 | 757105 | 757566 | ER_37561 | FBXL16 | Yes |
| 353 | 16 | 760308 | 760829 | ER_37562 | METRN | Yes |
| 354 | 16 | 832843 | 833202 | ER_37573 | RPUSD1 | Yes |
| 355 | 16 | 854207 | 854703 | ER_37576 | PRR25 | Yes |
| 356 | 16 | 863331 | 863631 | ER_37577 | PRR25 | No |
| 357 | 16 | 1098718 | 1099027 | ER_37589 | SSTR5-AS1 | Yes |
| 358 | 16 | 1132936 | 1133498 | ER_37595 | SSTR5 | No |
| 359 | 16 | 1138312 | 1138612 | ER_37596 | C1QTNF8 | No |
| 360 | 16 | 1209882 | 1210346 | ER_37604 | CACNA1H | Yes |
| 361 | 16 | 1232244 | 1232599 | ER_37605 | CACNA1H | Yes |
| 362 | 16 | 1240629 | 1241133 | ER_37606 | CACNA1H | No |
| 363 | 16 | 1275504 | 1275804 | ER_37610 | TPSG1 | No |
| 364 | 16 | 1305610 | 1305910 | ER_37611 | TPSD1 | No |
| 365 | 16 | 1350736 | 1351036 | ER_37623 | UBE2I | No |
| 366 | 16 | 1353340 | 1353767 | ER_37624 | UBE2I | Yes |
| 367 | 16 | 1430147 | 1430478 | ER_37632 | UNKL | Yes |
| 368 | 16 | 1479242 | 1479542 | ER_37638 | CCDC154 | No |
| 369 | 16 | 1827853 | 1828153 | ER_37665 | SPSB3 | No |
| 370 | 16 | 2004491 | 2004791 | ER_37679 | RPL3L | No |
| 371 | 16 | 2047651 | 2048147 | ER_37687 | ZNF598 | No |
| 372 | 16 | 2285522 | 2286213 | ER_37716 | E4F1 | Yes |
| 373 | 16 | 2294337 | 2294666 | ER_37717 | ECl1 | Yes |
| 374 | 16 | 2879820 | 2880369 | ER_37757 | ZG16B | Yes |
| 375 | 16 | 3009266 | 3009645 | ER_37769 | KREMEN2 | Yes |
| 376 | 16 | 3199417 | 3199873 | ER_37797 | ZNF213 | Yes |
| 377 | 16 | 3704538 | 3704838 | ER_37830 | DNASE1 | No |
| 378 | 16 | 3707018 | 3707318 | ER_37831 | DNASE1 | No |
| 379 | 16 | 4367762 | 4368278 | ER_37883 | GLIS2 | Yes |
| 380 | 16 | 4421377 | 4421694 | ER_37887 | CORO7 | Yes |
| 381 | 16 | 4425919 | 4426536 | ER_37892 | VASN | Yes |
| 382 | 16 | 4478392 | 4478714 | ER_37899 | DNAJA3 | Yes |
| 383 | 16 | 4741141 | 4741534 | ER_37916 | MGRN1 | Yes |
| 384 | 16 | 4746859 | 4747392 | ER_37919 | ANKS3 | Yes |
| 385 | 16 | 4838430 | 4839026 | ER_37923 | Sep-12 | Yes |
| 386 | 16 | 11422282 | 11422834 | ER_38071 | RMI2 | Yes |
| 387 | 16 | 14030414 | 14030714 | ER_38174 | ERCC4 | No |
| 388 | 16 | 14580747 | 14581084 | ER_38229 | PARN | No |
| 389 | 16 | 15602986 | 15603303 | ER_38266 | C16orf45 | Yes |
| 390 | 16 | 16090418 | 16090901 | ER_38297 | ABCC1 | Yes |
| 391 | 16 | 16108606 | 16109211 | ER_38301 | ABCC1 | No |
| 392 | 16 | 22103014 | 22103632 | ER_38518 | VWA3A | No |
| 393 | 16 | 24747980 | 24748466 | ER_38621 | TNRC6A | Yes |
| 394 | 16 | 24855743 | 24856361 | ER_38625 | SLC5A11 | No |
| 395 | 16 | 28511293 | 28511661 | ER_38732 | IL27 | Yes |
| 396 | 16 | 28608240 | 28608616 | ER_38739 | SULT1A2 | Yes |
| 397 | 16 | 30906082 | 30906783 | ER_38861 | BCL7C | No |
| 398 | 16 | 68321665 | 68322263 | ER_39511 | SLC7A6 | No |
| 399 | 16 | 69358300 | 69358626 | ER_39598 | VPS4A | Yes |
| 400 | 16 | 70472947 | 70473558 | ER_39656 | ST3GAL2 | No |
| 401 | 16 | 70687883 | 70688183 | ER_39667 | IL34 | No |
| 402 | 16 | 72994910 | 72995462 | ER_39877 | ZFHX3 | Yes |
| 403 | 16 | 78991185 | 78991807 | ER_40213 | WWOX | Yes |
| 404 | 16 | 81248258 | 81248721 | ER_40354 | PKD1L2 | Yes |
| 405 | 16 | 83847710 | 83848298 | ER_40473 | HSBP1 | No |
| 406 | 16 | 84400643 | 84401271 | ER_40523 | ATP2C2 | Yes |
| 407 | 16 | 84552526 | 84553023 | ER_40542 | KIAA1609 | Yes |
| 408 | 16 | 84628881 | 84629206 | ER_40553 | COTL1 | Yes |
| 409 | 16 | 84840044 | 84840619 | ER_40577 | CRISPLD2 | No |
| 410 | 16 | 84871395 | 84871718 | ER_40585 | CRISPLD2 | No |
| 411 | 16 | 85669299 | 85669599 | ER_40724 | KIAA0182 | No |
| 412 | 16 | 85723351 | 85723651 | ER_40737 | GINS2 | No |
| 413 | 16 | 85786954 | 85787520 | ER_40750 | C16orf74 | Yes |
| 414 | 1 | 109373005 | 109373612 | ER_4085 | AKNAD1 | No |
| 415 | 16 | 87739399 | 87739859 | ER_40900 | KLHDC4 | No |
| 416 | 16 | 87778638 | 87778938 | ER_40904 | KLHDC4 | No |
| 417 | 16 | 87812661 | 87813207 | ER_40908 | KLHDC4 | No |
| 418 | 16 | 87868043 | 87868626 | ER_40912 | SLC7A5 | No |
| 419 | 16 | 87890385 | 87890763 | ER_40918 | SLC7A5 | Yes |
| 420 | 16 | 87914990 | 87915446 | ER_40927 | CA5A | Yes |
| 421 | 16 | 88110906 | 88111273 | ER_40942 | BANP | Yes |
| 422 | 16 | 88111279 | 88111579 | ER_40943 | BANP | No |
| 423 | 16 | 88548864 | 88549438 | ER_40969 | ZFPM1 | Yes |
| 424 | 16 | 88704888 | 88705257 | ER_40987 | IL17C | Yes |
| 425 | 16 | 88988838 | 88989268 | ER_41018 | CBFA2T3 | Yes |
| 426 | 16 | 88991561 | 88992120 | ER_41020 | CBFA2T3 | No |
| 427 | 16 | 89004344 | 89004862 | ER_41024 | CBFA2T3 | No |
| 428 | 16 | 89043365 | 89043665 | ER_41029 | CBFA2T3 | Yes |
| 429 | 16 | 89638485 | 89638811 | ER_41092 | CPNE7 | Yes |
| 430 | 16 | 89648350 | 89648952 | ER_41093 | CPNE7 | Yes |
| 431 | 16 | 89665706 | 89666006 | ER_41094 | CPNE7 | Yes |
| 432 | 1 | 11020446 | 11021019 | ER_411 | C1orf127 | No |
| 433 | 16 | 89900078 | 89900645 | ER_41112 | SPIRE2 | Yes |
| 434 | 16 | 89927268 | 89927731 | ER_41118 | SPIRE2 | Yes |
| 435 | 16 | 90012005 | 90012305 | ER_41125 | DEF8 | No |
| 436 | 17 | 151960 | 152279 | ER_41149 | RPH3AL | Yes |
| 437 | 17 | 1634282 | 1634616 | ER_41197 | WDR81 | Yes |
| 438 | 17 | 1901327 | 1901683 | ER_41216 | RTN4RL1 | No |
| 439 | 17 | 1987743 | 1988066 | ER_41224 | SMG6 | Yes |
| 440 | 17 | 3635535 | 3635874 | ER_41267 | ITGAE | Yes |
| 441 | 1 | 109826384 | 109826898 | ER_4128 | PSRC1 | No |
| 442 | 17 | 3870398 | 3870698 | ER_41284 | ATP2A3 | No |
| 443 | 17 | 4400749 | 4401049 | ER_41310 | SPNS2 | Yes |
| 444 | 17 | 4436972 | 4437323 | ER_41315 | SPNS2 | No |
| 445 | 17 | 4455826 | 4456126 | ER_41316 | MYBBP1A | No |
| 446 | 17 | 7283657 | 7283982 | ER_41423 | TNK1 | No |
| 447 | 17 | 7959803 | 7960158 | ER_41460 | ALOX15B | No |
| 448 | 17 | 14109320 | 14109680 | ER_41695 | COX10 | No |
| 449 | 17 | 16322433 | 16322733 | ER_41788 | TRPV2 | No |
| 450 | 17 | 16954969 | 16955288 | ER_41817 | MPRIP | Yes |
| 451 | 17 | 17628505 | 17628831 | ER_41872 | RAI1 | No |
| 452 | 17 | 17718324 | 17718694 | ER_41889 | SREBF1 | Yes |
| 453 | 17 | 18139202 | 18139554 | ER_41939 | LLGL1 | Yes |
| 454 | 17 | 18280720 | 18281075 | ER_41950 | EVPLL | No |
| 455 | 17 | 19627818 | 19628193 | ER_42003 | SLC47A2 | No |
| 456 | 17 | 26578307 | 26578607 | ER_42148 | PPY2 | Yes |
| 457 | 17 | 27295913 | 27296324 | ER_42201 | SEZ6 | Yes |
| 458 | 17 | 29649808 | 29650164 | ER_42279 | NF1 | Yes |
| 459 | 1 | 111006535 | 111007108 | ER_4230 | PROK1 | No |
| 460 | 17 | 39577357 | 39577695 | ER_42572 | KRT37 | Yes |
| 461 | 17 | 39662933 | 39663289 | ER_42574 | KRT13 | No |
| 462 | 17 | 39677996 | 39678528 | ER_42579 | KRT19 | No |
| 463 | 17 | 39685761 | 39686063 | ER_42583 | KRT19 | No |
| 464 | 17 | 39694022 | 39694351 | ER_42588 | KRT19 | No |
| 465 | 17 | 40931971 | 40932360 | ER_42648 | WNK4 | Yes |
| 466 | 17 | 44896572 | 44896908 | ER_42790 | WNT3 | Yes |
| 467 | 17 | 48048352 | 48048668 | ER_42976 | DLX4 | Yes |
| 468 | 17 | 48261929 | 48262229 | ER_43003 | COL1A1 | Yes |
| 469 | 17 | 55371434 | 55371787 | ER_43290 | MSI2 | Yes |
| 470 | 17 | 55673703 | 55674091 | ER_43343 | MSI2 | Yes |
| 471 | 1 | 12192916 | 12193471 | ER_444 | TNFRSF8 | No |
| 472 | 17 | 58498280 | 58498903 | ER_44815 | C17orf64 | No |
| 473 | 1 | 1609934 | 1610467 | ER_45 | SLC35E2B | No |
| 474 | 17 | 59484187 | 59484499 | ER_45541 | TBX2 | Yes |
| 475 | 1 | 114218048 | 114218488 | ER_4610 | MAGI3 | No |
| 476 | 17 | 64940732 | 64941081 | ER_46841 | CACNG4 | Yes |
| 477 | 17 | 64954251 | 64954635 | ER_46847 | CACNG4 | Yes |
| 478 | 17 | 65487266 | 65487622 | ER_46920 | PITPNC1 | No |
| 479 | 17 | 66291281 | 66291581 | ER_46984 | ARSG | Yes |
| 480 | 17 | 70636643 | 70636952 | ER_47135 | LINC00511 | Yes |
| 481 | 17 | 71612589 | 71612889 | ER_47199 | SDK2 | No |
| 482 | 17 | 72439108 | 72439488 | ER_47235 | GPRC5C | Yes |
| 483 | 17 | 72732636 | 72732936 | ER_47245 | RAB37 | No |
| 484 | 17 | 72740901 | 72741201 | ER_47248 | RAB37 | No |
| 485 | 17 | 73500571 | 73500871 | ER_47351 | CASKIN2 | Yes |
| 486 | 17 | 73641528 | 73641923 | ER_47388 | RECQL5 | Yes |
| 487 | 17 | 73696463 | 73696795 | ER_47396 | SAP30BP | Yes |
| 488 | 17 | 73761501 | 73761882 | ER_47406 | GALK1 | Yes |
| 489 | 17 | 73805905 | 73806251 | ER_47422 | UNK | Yes |
| 490 | 17 | 73872405 | 73872705 | ER_47428 | TRIM47 | No |
| 491 | 17 | 74494140 | 74494511 | ER_47474 | RHBDF2 | Yes |
| 492 | 17 | 74684239 | 74684546 | ER_47498 | MXRA7 | No |
| 493 | 17 | 75181779 | 75182110 | ER_47526 | SEC14L1 | No |
| 494 | 17 | 75473604 | 75473943 | ER_47548 | Sep-09 | Yes |
| 495 | 17 | 76498871 | 76499229 | ER_47612 | DNAH17 | No |
| 496 | 17 | 76522848 | 76523165 | ER_47614 | DNAH17 | Yes |
| 497 | 17 | 76588325 | 76588770 | ER_47620 | DNAH17 | No |
| 498 | 17 | 76858208 | 76858508 | ER_47631 | TIMP2 | No |
| 499 | 17 | 76973124 | 76973427 | ER_47643 | LGALS3BP | Yes |
| 500 | 17 | 77782827 | 77783366 | ER_47666 | CBX8 | No |
| 501 | 17 | 77785183 | 77785533 | ER_47667 | CBX8 | No |
| 502 | 17 | 77818303 | 77818633 | ER_47681 | CBX4 | Yes |
| 503 | 17 | 78522351 | 78522735 | ER_47743 | RPTOR | Yes |
| 504 | 17 | 78667839 | 78668195 | ER_47754 | RPTOR | Yes |
| 505 | 17 | 78791604 | 78791917 | ER_47760 | RPTOR | Yes |
| 506 | 17 | 78793449 | 78793890 | ER_47761 | RPTOR | Yes |
| 507 | 17 | 78796720 | 78797088 | ER_47762 | RPTOR | Yes |
| 508 | 17 | 79018691 | 79019077 | ER_47773 | BAIAP2 | No |
| 509 | 17 | 79251153 | 79251492 | ER_47785 | SLC38A10 | No |
| 510 | 17 | 79447600 | 79447963 | ER_47803 | BAHCC1 | Yes |
| 511 | 17 | 79961758 | 79962058 | ER_47823 | ASPSCR1 | No |
| 512 | 17 | 80162846 | 80163196 | ER_47840 | CCDC57 | No |
| 513 | 17 | 80174646 | 80174968 | ER_47845 | CCDC57 | Yes |
| 514 | 17 | 80419732 | 80420082 | ER_47870 | NARF | No |
| 515 | 17 | 80662180 | 80662480 | ER_47885 | RAB40B | Yes |
| 516 | 17 | 81025415 | 81025759 | ER_47909 | METRNL | Yes |
| 517 | 17 | 81031364 | 81031696 | ER_47911 | METRNL | Yes |
| 518 | 18 | 3446546 | 3446906 | ER_48111 | TGIF1 | Yes |
| 519 | 1 | 114521332 | 114522214 | ER_4835 | OLFML3 | No |
| 520 | 18 | 74536197 | 74536522 | ER_49880 | ZNF236 | Yes |
| 521 | 19 | 930678 | 930978 | ER_49971 | ARID3A | Yes |
| 522 | 19 | 1169031 | 1169402 | ER_49996 | SBNO2 | No |
| 523 | 19 | 1496339 | 1496639 | ER_50033 | REEP6 | No |
| 524 | 19 | 1907761 | 1908143 | ER_50049 | SCAMP4 | Yes |
| 525 | 19 | 2167382 | 2167682 | ER_50060 | DOT1L | No |
| 526 | 19 | 2624590 | 2624934 | ER_50099 | GNG7 | Yes |
| 527 | 19 | 2723050 | 2723398 | ER_50102 | DIRAS1 | Yes |
| 528 | 19 | 2727038 | 2727338 | ER_50103 | SLC39A3 | Yes |
| 529 | 19 | 3374719 | 3375065 | ER_50124 | NFIC | Yes |
| 530 | 19 | 6276448 | 6276748 | ER_50272 | MLLT1 | Yes |
| 531 | 19 | 7684955 | 7685267 | ER_50326 | XAB2 | Yes |
| 532 | 19 | 7714289 | 7714687 | ER_50328 | STXBP2 | No |
| 533 | 19 | 11617772 | 11618120 | ER_50514 | ECSIT | Yes |
| 534 | 19 | 14066552 | 14066906 | ER_50640 | DCAF15 | Yes |
| 535 | 19 | 14544916 | 14545316 | ER_50670 | PKN1 | Yes |
| 536 | 19 | 15590207 | 15590541 | ER_50728 | PGLYRP2 | No |
| 537 | 19 | 15618423 | 15618788 | ER_50733 | CYP4F22 | Yes |
| 538 | 19 | 15622532 | 15622947 | ER_50734 | CYP4F22 | Yes |
| 539 | 19 | 16045737 | 16046329 | ER_50746 | CYP4F11 | No |
| 540 | 19 | 16603746 | 16604192 | ER_50800 | CALR3 | Yes |
| 541 | 19 | 17407033 | 17407362 | ER_50855 | ABHD8 | Yes |
| 542 | 19 | 18385725 | 18386025 | ER_50903 | KIAA1683 | Yes |
| 543 | 19 | 33167485 | 33167785 | ER_51224 | RGS9BP | No |
| 544 | 19 | 33624481 | 33624781 | ER_51258 | WDR88 | Yes |
| 545 | 19 | 33726540 | 33726840 | ER_51263 | SLC7A10 | Yes |
| 546 | 19 | 35531859 | 35532247 | ER_51354 | HPN | No |
| 547 | 19 | 35800367 | 35800739 | ER_51381 | MAG | Yes |
| 548 | 19 | 35801186 | 35801551 | ER_51382 | MAG | No |
| 549 | 19 | 35940219 | 35940897 | ER_51391 | FFAR2 | Yes |
| 550 | 19 | 38793369 | 38793685 | ER_51493 | YIF1B | Yes |
| 551 | 19 | 39222375 | 39222739 | ER_51519 | CAPN12 | Yes |
| 552 | 19 | 41633774 | 41634080 | ER_51655 | CYP2F1 | Yes |
| 553 | 19 | 45843797 | 45844097 | ER_51894 | KLC3 | No |
| 554 | 19 | 45848214 | 45848514 | ER_51895 | KLC3 | Yes |
| 555 | 19 | 49059555 | 49059855 | ER_52131 | SULT2B1 | No |
| 556 | 19 | 50458158 | 50458475 | ER_52226 | SIGLEC11 | Yes |
| 557 | 19 | 50969943 | 50970265 | ER_52248 | FAM71E1 | Yes |
| 558 | 19 | 51568129 | 51568429 | ER_52287 | KLK13 | No |
| 559 | 19 | 54600186 | 54600532 | ER_52363 | OSCAR | No |
| 560 | 19 | 55874846 | 55875146 | ER_52410 | FAM71E2 | Yes |
| 561 | 19 | 55880474 | 55880774 | ER_52411 | IL11 | No |
| 562 | 19 | 56047757 | 56048080 | ER_52419 | SBK2 | No |
| 563 | 2 | 3452679 | 3453034 | ER_52628 | TRAPPC12 | Yes |
| 564 | 2 | 8833486 | 8833786 | ER_52806 | ID2 | No |
| 565 | 2 | 19555335 | 19555655 | ER_53364 | OSR1 | Yes |
| 566 | 2 | 25094778 | 25095111 | ER_53566 | ADCY3 | Yes |
| 567 | 2 | 25562754 | 25563086 | ER_53585 | DNMT3A | Yes |
| 568 | 2 | 26199821 | 26200281 | ER_53612 | KIF3C | No |
| 569 | 2 | 26947077 | 26947447 | ER_53662 | KCNK3 | No |
| 570 | 2 | 27319112 | 27319450 | ER_53686 | KHK | No |
| 571 | 2 | 28549119 | 28549437 | ER_53751 | BRE | Yes |
| 572 | 2 | 28569483 | 28570005 | ER_53759 | BRE | Yes |
| 573 | 2 | 45998277 | 45998784 | ER_54551 | PRKCE | Yes |
| 574 | 2 | 46361686 | 46362029 | ER_54598 | PRKCE | Yes |
| 575 | 2 | 47236009 | 47236309 | ER_54678 | TTC7A | Yes |
| 576 | 2 | 54760101 | 54760460 | ER_54825 | SPTBN1 | Yes |
| 577 | 1 | 16061307 | 16061646 | ER_549 | PLEKHM2 | Yes |
| 578 | 1 | 16074063 | 16074363 | ER_550 | TMEM82 | No |
| 579 | 1 | 115211543 | 115212151 | ER_5516 | DENND2C | Yes |
| 580 | 2 | 74152981 | 74153296 | ER_55613 | DGUOK | Yes |
| 581 | 2 | 85280957 | 85281340 | ER_55852 | KCMF1 | Yes |
| 582 | 2 | 85621930 | 85622230 | ER_55867 | CAPG | No |
| 583 | 1 | 16251091 | 16251574 | ER_559 | SPEN | No |
| 584 | 2 | 95719289 | 95719611 | ER_56072 | MAL | Yes |
| 585 | 2 | 97508396 | 97508742 | ER_56156 | ANKRD23 | No |
| 586 | 2 | 102012642 | 102013116 | ER_56390 | RFX8 | No |
| 587 | 1 | 16403340 | 16403801 | ER_564 | FAM131C | Yes |
| 588 | 2 | 106007690 | 106008035 | ER_56517 | FHL2 | No |
| 589 | 2 | 113875147 | 113875494 | ER_56808 | IL1RN | No |
| 590 | 2 | 121036360 | 121036660 | ER_56956 | RALB | Yes |
| 591 | 2 | 121036666 | 121036969 | ER_56957 | RALB | Yes |
| 592 | 2 | 121071534 | 121071901 | ER_56964 | RALB | No |
| 593 | 2 | 128458070 | 128458447 | ER_57147 | SFT2D3 | No |
| 594 | 2 | 129066951 | 129067320 | ER_57178 | HS6ST1 | No |
| 595 | 1 | 116219030 | 116219646 | ER_5750 | VANGL1 | No |
| 596 | 2 | 175499224 | 175499524 | ER_59117 | WIPF1 | No |
| 597 | 1 | 16950505 | 16950951 | ER_599 | CROCCP2 | Yes |
| 598 | 2 | 197158771 | 197159125 | ER_59918 | HECW2 | No |
| 599 | 2 | 197466173 | 197466509 | ER_59929 | HECW2 | Yes |
| 600 | 1 | 17035334 | 17035751 | ER_603 | ESPNP | No |
| 601 | 2 | 208494280 | 208494580 | ER_60350 | METTL21A | No |
| 602 | 2 | 216478034 | 216478350 | ER_60685 | LINC00607 | No |
| 603 | 2 | 220007041 | 220007402 | ER_60937 | NHEJ1 | No |
| 604 | 1 | 17047514 | 17047814 | ER_610 | ESPNP | No |
| 605 | 2 | 224624781 | 224625214 | ER_61179 | AP1S3 | No |
| 606 | 2 | 236447665 | 236448023 | ER_61661 | AGAP1 | Yes |
| 607 | 2 | 239169600 | 239169933 | ER_61869 | PER2 | No |
| 608 | 2 | 240186821 | 240187208 | ER_61904 | HDAC4 | Yes |
| 609 | 2 | 240241009 | 240241336 | ER_61909 | HDAC4 | Yes |
| 610 | 2 | 241807610 | 241808275 | ER_61956 | AGXT | Yes |
| 611 | 2 | 241832846 | 241833163 | ER_61959 | C2orf54 | No |
| 612 | 2 | 241936626 | 241937048 | ER_61974 | SNED1 | Yes |
| 613 | 2 | 241975925 | 241976252 | ER_61978 | SNED1 | Yes |
| 614 | 2 | 242138256 | 242138571 | ER_61990 | ANO7 | Yes |
| 615 | 2 | 242500199 | 242500499 | ER_62006 | BOK | Yes |
| 616 | 20 | 17595352 | 17595711 | ER_62469 | RRBP1 | Yes |
| 617 | 20 | 18035798 | 18036353 | ER_62490 | OVOL2 | No |
| 618 | 20 | 30432800 | 30433405 | ER_62916 | FOXS1 | Yes |
| 619 | 20 | 32446738 | 32447321 | ER_63011 | CHMP4B | No |
| 620 | 20 | 32888452 | 32889022 | ER_63021 | AHCY | Yes |
| 621 | 20 | 34205164 | 34205464 | ER_63067 | SPAG4 | No |
| 622 | 20 | 35093680 | 35094313 | ER_63095 | DLGAP4 | No |
| 623 | 20 | 35493191 | 35493491 | ER_63113 | SOGA1 | No |
| 624 | 20 | 36767493 | 36768090 | ER_63162 | TGM2 | No |
| 625 | 1 | 118727703 | 118728260 | ER_6319 | SPAG17 | Yes |
| 626 | 1 | 17634485 | 17634785 | ER_634 | PADI4 | Yes |
| 627 | 20 | 43343181 | 43343741 | ER_63515 | WISP2 | No |
| 628 | 20 | 44048102 | 44048440 | ER_63559 | PIGT | No |
| 629 | 20 | 44330526 | 44330841 | ER_63571 | WFDC13 | Yes |
| 630 | 20 | 47278241 | 47278894 | ER_65136 | PREX1 | No |
| 631 | 1 | 144989293 | 144989659 | ER_6529 | PDE4DIP | No |
| 632 | 20 | 47448122 | 47448615 | ER_65321 | PREX1 | No |
| 633 | 20 | 49345259 | 49346053 | ER_65723 | PARD6B | No |
| 634 | 20 | 49346308 | 49346885 | ER_65724 | PARD6B | No |
| 635 | 20 | 49411019 | 49411779 | ER_65780 | BCAS4 | No |
| 636 | 20 | 52205856 | 52206621 | ER_66116 | ZNF217 | Yes |
| 637 | 1 | 17887921 | 17888496 | ER_662 | ARHGEF10L | Yes |
| 638 | 1 | 18006751 | 18007051 | ER_669 | ARHGEF10L | No |
| 639 | 20 | 55200110 | 55200410 | ER_67539 | TFAP2C | No |
| 640 | 20 | 58325746 | 58326345 | ER_68282 | PHACTR3 | No |
| 641 | 20 | 60510103 | 60510565 | ER_68459 | CDH4 | Yes |
| 642 | 20 | 60924954 | 60925254 | ER_68486 | LAMA5 | No |
| 643 | 20 | 60932217 | 60932572 | ER_68489 | LAMA5 | Yes |
| 644 | 20 | 61451571 | 61451871 | ER_68537 | COL9A3 | Yes |
| 645 | 20 | 62184027 | 62184327 | ER_68582 | C20orf195 | No |
| 646 | 20 | 62282532 | 62282832 | ER_68587 | STMN3 | No |
| 647 | 1 | 150333685 | 150334193 | ER_6892 | RPRD2 | Yes |
| 648 | 1 | 2036450 | 2036863 | ER_69 | PRKCZ | Yes |
| 649 | 21 | 37802129 | 37802579 | ER_69231 | CHAF1B | No |
| 650 | 21 | 42212292 | 42212834 | ER_69514 | DSCAM | No |
| 651 | 21 | 43107677 | 43108088 | ER_69583 | LINC00111 | No |
| 652 | 21 | 43135959 | 43136350 | ER_69588 | LINC00479 | No |
| 653 | 21 | 43735388 | 43735910 | ER_69642 | TFF3 | Yes |
| 654 | 21 | 44816541 | 44817155 | ER_69713 | SIK1 | No |
| 655 | 21 | 44897853 | 44898245 | ER_69724 | LINC00313 | Yes |
| 656 | 21 | 46172674 | 46173357 | ER_69814 | UBE2G2 | Yes |
| 657 | 21 | 46321203 | 46321769 | ER_69832 | ITGB2 | No |
| 658 | 21 | 46325777 | 46326117 | ER_69834 | ITGB2 | Yes |
| 659 | 21 | 46331748 | 46332340 | ER_69835 | ITGB2 | No |
| 660 | 21 | 46409729 | 46410331 | ER_69844 | LINC00163 | Yes |
| 661 | 21 | 46953747 | 46954295 | ER_69890 | SLC19A1 | Yes |
| 662 | 22 | 18919539 | 18919839 | ER_69990 | PRODH | Yes |
| 663 | 22 | 19718480 | 19718835 | ER_70025 | GP1BB | No |
| 664 | 22 | 19755289 | 19755589 | ER_70033 | TBX1 | Yes |
| 665 | 22 | 19879093 | 19879435 | ER_70044 | TXNRD2 | No |
| 666 | 22 | 24384589 | 24384947 | ER_70184 | GSTT1 | No |
| 667 | 1 | 151554445 | 151555180 | ER_7042 | TUFT1 | Yes |
| 668 | 22 | 35695235 | 35695568 | ER_70647 | TOM1 | Yes |
| 669 | 22 | 35931960 | 35932283 | ER_70657 | RASD2 | Yes |
| 670 | 22 | 38092597 | 38092897 | ER_70759 | TRIOBP | No |
| 671 | 1 | 151818574 | 151819202 | ER_7079 | THEM5 | No |
| 672 | 22 | 38610003 | 38610303 | ER_70793 | MAFF | No |
| 673 | 22 | 39759998 | 39760357 | ER_70842 | SYNGR1 | No |
| 674 | 22 | 40404687 | 40405045 | ER_70880 | FAM83F | Yes |
| 675 | 22 | 43525157 | 43525457 | ER_70998 | BIK | Yes |
| 676 | 22 | 46921821 | 46922121 | ER_71163 | CELSR1 | Yes |
| 677 | 22 | 50450954 | 50451412 | ER_71287 | IL17REL | Yes |
| 678 | 22 | 50720235 | 50720566 | ER_71300 | PLXNB2 | No |
| 679 | 22 | 50738759 | 50739090 | ER_71305 | PLXNB2 | Yes |
| 680 | 22 | 50918140 | 50918446 | ER_71311 | ADM2 | No |
| 681 | 3 | 8693628 | 8694000 | ER_71572 | C3orf32 | Yes |
| 682 | 3 | 9757534 | 9757834 | ER_71643 | CPNE9 | No |
| 683 | 3 | 9996819 | 9997181 | ER_71662 | PRRT3-AS1 | No |
| 684 | 3 | 11550571 | 11550871 | ER_71746 | ATG7 | No |
| 685 | 3 | 11643071 | 11643504 | ER_71757 | VGLL4 | No |
| 686 | 3 | 11763408 | 11763780 | ER_71771 | VGLL4 | Yes |
| 687 | 3 | 12985852 | 12986194 | ER_71863 | IQSEC1 | No |
| 688 | 3 | 12994633 | 12995020 | ER_71864 | IQSEC1 | No |
| 689 | 3 | 13517660 | 13517983 | ER_71911 | HDAC11 | No |
| 690 | 3 | 14920746 | 14921046 | ER_72049 | FGD5 | No |
| 691 | 3 | 15310870 | 15311256 | ER_72070 | SH3BP5 | Yes |
| 692 | 3 | 15687178 | 15687551 | ER_72120 | BTD | No |
| 693 | 1 | 19663685 | 19664277 | ER_722 | CAPZB | Yes |
| 694 | 1 | 19664905 | 19665205 | ER_723 | CAPZB | Yes |
| 695 | 1 | 154166420 | 154166720 | ER_7253 | MIR190B | Yes |
| 696 | 1 | 154298772 | 154299072 | ER_7259 | ATP8B2 | No |
| 697 | 1 | 154377532 | 154377832 | ER_7267 | IL6R | No |
| 698 | 3 | 38067281 | 38067610 | ER_73005 | PLCD1 | No |
| 699 | 3 | 46734109 | 46734463 | ER_73287 | ALS2CL | Yes |
| 700 | 3 | 48589861 | 48590161 | ER_73356 | PFKFB4 | No |
| 701 | 1 | 155161639 | 155162099 | ER_7336 | MUC1 | No |
| 702 | 3 | 50638992 | 50639632 | ER_73426 | CISH | Yes |
| 703 | 3 | 52280070 | 52280428 | ER_73463 | PPM1M | No |
| 704 | 3 | 58028409 | 58028709 | ER_73666 | FLNB | No |
| 705 | 1 | 155912401 | 155912737 | ER_7372 | RXFP4 | Yes |
| 706 | 1 | 156095999 | 156096299 | ER_7395 | LMNA | No |
| 707 | 3 | 61793488 | 61793816 | ER_73972 | PTPRG | No |
| 708 | 1 | 156679461 | 156679823 | ER_7437 | CRABP2 | Yes |
| 709 | 1 | 156822285 | 156822644 | ER_7451 | INSRR | No |
| 710 | 1 | 160079100 | 160079716 | ER_7493 | ATP1A2 | Yes |
| 711 | 1 | 161646681 | 161647242 | ER_7530 | FCGR2B | No |
| 712 | 1 | 2174671 | 2174976 | ER_76 | SKI | Yes |
| 713 | 3 | 63922231 | 63922573 | ER_76144 | ATXN7 | No |
| 714 | 3 | 66519928 | 66520259 | ER_77324 | LRIG1 | Yes |
| 715 | 3 | 66543250 | 66543644 | ER_77334 | LRIG1 | No |
| 716 | 3 | 69942380 | 69942719 | ER_77420 | MITF | Yes |
| 717 | 3 | 99721769 | 99722069 | ER_78150 | FILIP1L | Yes |
| 718 | 1 | 170043984 | 170044284 | ER_7816 | KIFAP3 | No |
| 719 | 3 | 99833502 | 99833835 | ER_78166 | C3orf26 | Yes |
| 720 | 1 | 171226439 | 171226888 | ER_7821 | FMO1 | Yes |
| 721 | 3 | 112359914 | 112360214 | ER_78589 | CCDC80 | No |
| 722 | 3 | 122057821 | 122058174 | ER_78781 | CSTA | Yes |
| 723 | 1 | 172608570 | 172609176 | ER_7888 | FASLG | No |
| 724 | 3 | 124493174 | 124493574 | ER_78936 | ITGB5 | No |
| 725 | 3 | 126200679 | 126200979 | ER_79014 | UROC1 | No |
| 726 | 3 | 126678959 | 126679283 | ER_79028 | CHCHD6 | Yes |
| 727 | 3 | 129295579 | 129295977 | ER_79235 | PLXND1 | No |
| 728 | 3 | 133174831 | 133175237 | ER_79397 | BFSP2 | Yes |
| 729 | 3 | 160787663 | 160788036 | ER_80595 | PPM1L | No |
| 730 | 3 | 168870086 | 168870443 | ER_80932 | MECOM | Yes |
| 731 | 3 | 169758098 | 169758589 | ER_80993 | GPR160 | No |
| 732 | 3 | 183959578 | 183959923 | ER_81693 | VWA5B2 | Yes |
| 733 | 3 | 184052187 | 184052556 | ER_81701 | EIF4G1 | No |
| 734 | 3 | 195531202 | 195531699 | ER_82319 | MUC4 | Yes |
| 735 | 3 | 195603560 | 195603892 | ER_82326 | TNK2 | Yes |
| 736 | 3 | 196388403 | 196388727 | ER_82377 | LRRC33 | No |
| 737 | 4 | 680920 | 681252 | ER_82472 | MFSD7 | Yes |
| 738 | 4 | 686579 | 687011 | ER_82474 | MFSD7 | No |
| 739 | 4 | 757427 | 757729 | ER_82479 | PCGF3 | Yes |
| 740 | 4 | 965412 | 965736 | ER_82491 | DGKQ | Yes |
| 741 | 4 | 987571 | 987878 | ER_82493 | IDUA | Yes |
| 742 | 4 | 1239191 | 1239531 | ER_82503 | CTBP1 | No |
| 743 | 4 | 1729872 | 1730230 | ER_82525 | TACC3 | Yes |
| 744 | 4 | 1986291 | 1986591 | ER_82549 | WHSC2 | Yes |
| 745 | 4 | 2798112 | 2798444 | ER_82594 | SH3BP2 | Yes |
| 746 | 4 | 3341090 | 3341444 | ER_82632 | RGS12 | Yes |
| 747 | 4 | 3772680 | 3772980 | ER_82661 | ADRA2C | Yes |
| 748 | 4 | 6928789 | 6929118 | ER_82798 | TBC1D14 | Yes |
| 749 | 4 | 7219719 | 7220019 | ER_82822 | SORCS2 | No |
| 750 | 4 | 8062466 | 8062766 | ER_82882 | ABLIM2 | Yes |
| 751 | 4 | 8130159 | 8130480 | ER_82884 | ABLIM2 | Yes |
| 752 | 4 | 8412544 | 8412844 | ER_82916 | ACOX3 | No |
| 753 | 4 | 48908995 | 48909295 | ER_83945 | OCIAD2 | Yes |
| 754 | 1 | 22773934 | 22774523 | ER_859 | ZBTB40 | No |
| 755 | 4 | 119910484 | 119910799 | ER_85984 | SYNPO2 | No |
| 756 | 4 | 184644416 | 184644716 | ER_87970 | TRAPPC11 | Yes |
| 757 | 5 | 373069 | 373625 | ER_88159 | AHRR | Yes |
| 758 | 5 | 429733 | 430313 | ER_88163 | AHRR | Yes |
| 759 | 5 | 610077 | 610670 | ER_88173 | CEP72 | Yes |
| 760 | 5 | 672776 | 673076 | ER_88176 | TPPP | No |
| 761 | 5 | 1201313 | 1201673 | ER_88195 | SLC6A19 | No |
| 762 | 5 | 1207117 | 1207709 | ER_88196 | SLC6A19 | Yes |
| 763 | 5 | 1443109 | 1443488 | ER_88204 | SLC6A3 | No |
| 764 | 5 | 7851676 | 7852193 | ER_88404 | C5orf49 | Yes |
| 765 | 5 | 37840402 | 37840702 | ER_89386 | GDNF | No |
| 766 | 5 | 43603889 | 43604448 | ER_89613 | NNT | Yes |
| 767 | 5 | 52385969 | 52386507 | ER_89737 | ITGA2 | Yes |
| 768 | 1 | 201095792 | 201096337 | ER_8996 | TMEM9 | No |
| 769 | 1 | 201252202 | 201252787 | ER_9013 | PKP1 | No |
| 770 | 5 | 95226771 | 95227393 | ER_91036 | ELL2 | No |
| 771 | 1 | 203096814 | 203097402 | ER_9205 | ADORA1 | No |
| 772 | 1 | 203122931 | 203123270 | ER_9215 | ADORA1 | No |
| 773 | 5 | 138299601 | 138299901 | ER_92405 | SIL1 | Yes |
| 774 | 1 | 203297838 | 203298428 | ER_9241 | FMOD | Yes |
| 775 | 5 | 138731543 | 138732087 | ER_92433 | SPATA24 | Yes |
| 776 | 5 | 139033837 | 139034156 | ER_92444 | CXXC5 | Yes |
| 777 | 5 | 139069265 | 139069565 | ER_92451 | CXXC5 | Yes |
| 778 | 5 | 140012702 | 140013307 | ER_92479 | CD14 | No |
| 779 | 1 | 203488468 | 203489054 | ER_9257 | OPTC | Yes |
| 780 | 1 | 203594651 | 203595113 | ER_9266 | ATP2B4 | No |
| 781 | 5 | 148513872 | 148514255 | ER_92755 | ABLIM3 | No |
| 782 | 5 | 159687745 | 159688359 | ER_93120 | CCNJL | Yes |
| 783 | 5 | 176239632 | 176240113 | ER_93644 | UNC5A | Yes |
| 784 | 5 | 176816546 | 176817069 | ER_93670 | SLC34A1 | No |
| 785 | 5 | 176874917 | 176875279 | ER_93675 | PRR7-AS1 | Yes |
| 786 | 5 | 177029224 | 177029524 | ER_93689 | B4GALT7 | Yes |
| 787 | 5 | 177547872 | 177548392 | ER_93712 | N4BP3 | No |
| 788 | 5 | 180022475 | 180023061 | ER_93819 | SCGB3A1 | Yes |
| 789 | 5 | 180648505 | 180649224 | ER_93834 | MIR4638 | No |
| 790 | 6 | 379290 | 379637 | ER_93850 | IRF4 | Yes |
| 791 | 6 | 3139405 | 3139938 | ER_93950 | BPHL | Yes |
| 792 | 6 | 3723005 | 3723548 | ER_93997 | PXDC1 | No |
| 793 | 6 | 10420655 | 10421048 | ER_94256 | TFAP2A | No |
| 794 | 1 | 205633387 | 205633954 | ER_9471 | SLC45A3 | Yes |
| 795 | 6 | 30698796 | 30699096 | ER_95075 | FLOT1 | No |
| 796 | 6 | 31477979 | 31478310 | ER_95117 | MICB | No |
| 797 | 6 | 31743735 | 31744048 | ER_95131 | VWA7 | Yes |
| 798 | 6 | 31744689 | 31744989 | ER_95132 | VWA7 | Yes |
| 799 | 6 | 31833129 | 31833429 | ER_95141 | SLC44A4 | Yes |
| 800 | 6 | 31868596 | 31868896 | ER_95144 | C2 | No |
| 801 | 6 | 32050977 | 32051543 | ER_95152 | TNXB | Yes |
| 802 | 6 | 32078224 | 32078792 | ER_95153 | TNXB | Yes |
| 803 | 6 | 32135005 | 32135578 | ER_95158 | EGFL8 | Yes |
| 804 | 6 | 33173123 | 33173423 | ER_95186 | HSD17B8 | No |
| 805 | 6 | 33288112 | 33288670 | ER_95195 | DAXX | Yes |
| 806 | 6 | 33993601 | 33993995 | ER_95239 | GRM4 | Yes |
| 807 | 6 | 34511677 | 34512101 | ER_95268 | SPDEF | Yes |
| 808 | 6 | 40363002 | 40363510 | ER_95568 | LRFN2 | No |
| 809 | 6 | 42108932 | 42109278 | ER_95663 | C6orf132 | Yes |
| 810 | 6 | 43463227 | 43463527 | ER_95722 | TJAP1 | No |
| 811 | 6 | 43603250 | 43603772 | ER_95728 | MAD2L1BP | No |
| 812 | 6 | 52268606 | 52268913 | ER_96019 | PAQR8 | Yes |
| 813 | 6 | 56580975 | 56581555 | ER_96201 | RNU6-71 | No |
| 814 | 6 | 64281501 | 64282055 | ER_96269 | PTP4A1 | No |
| 815 | 1 | 25070145 | 25070811 | ER_965 | CLIC4 | No |
| 816 | 6 | 106576993 | 106577649 | ER_97203 | PRDM1 | Yes |
| 817 | 6 | 109267245 | 109267600 | ER_97322 | ARMC2 | Yes |
| 818 | 6 | 138425688 | 138426269 | ER_98290 | PERP | No |
| 819 | 6 | 149806486 | 149806786 | ER_98712 | ZC3H12D | Yes |
| 820 | 6 | 151937170 | 151937602 | ER_98798 | CCDC170 | Yes |
| 821 | 6 | 152124722 | 152125100 | ER_98841 | ESR1 | Yes |
| 822 | 6 | 152432244 | 152432773 | ER_98857 | ESR1 | No |
| 823 | 6 | 155052906 | 155053506 | ER_98929 | SCAF8 | Yes |
| 824 | 7 | 921963 | 922279 | ER_99284 | GET4 | Yes |
| 825 | 7 | 923724 | 924024 | ER_99285 | GET4 | No |
| 826 | 7 | 927894 | 928206 | ER_99286 | GET4 | Yes |
| 827 | 7 | 940841 | 941164 | ER_99287 | ADAP1 | Yes |
| 828 | 7 | 955121 | 955452 | ER_99289 | ADAP1 | Yes |
| 829 | 7 | 966854 | 967230 | ER_99290 | ADAP1 | Yes |
| 830 | 7 | 1004933 | 1005233 | ER_99293 | COX19 | No |
| 831 | 7 | 1026106 | 1026406 | ER_99296 | CYP2W1 | No |
| 832 | 7 | 1027843 | 1028143 | ER_99297 | CYP2W1 | Yes |
| 833 | 7 | 1054343 | 1054643 | ER_99302 | C7orf50 | Yes |
| 834 | 7 | 1139221 | 1139554 | ER_99313 | C7orf50 | Yes |
| 835 | 7 | 1286406 | 1286773 | ER_99325 | UNCX | No |
| 836 | 7 | 1501011 | 1501415 | ER_99342 | MICALL2 | Yes |
| 837 | 7 | 1552985 | 1553309 | ER_99348 | INTS1 | No |
| 838 | 1 | 217886370 | 217887036 | ER_9936 | SPATA17 | Yes |
| 839 | 7 | 1659555 | 1659913 | ER_99368 | TFAMP1 | No |
| 840 | 7 | 1753468 | 1753819 | ER_99371 | ELFN1 | No |
| 841 | 7 | 1782517 | 1782817 | ER_99372 | ELFN1 | No |
| 842 | 7 | 1891218 | 1891549 | ER_99377 | MAD1L1 | No |
| 843 | 7 | 1931906 | 1932296 | ER_99382 | MAD1L1 | No |
| 844 | 7 | 1959815 | 1960115 | ER_99385 | MAD1L1 | No |
| 845 | 7 | 1961938 | 1962358 | ER_99386 | MAD1L1 | Yes |
| 846 | 7 | 1967307 | 1967639 | ER_99387 | MAD1L1 | Yes |
| 847 | 7 | 1968165 | 1968519 | ER_99388 | MAD1L1 | No |
| 848 | 7 | 2191489 | 2191835 | ER_99406 | MAD1L1 | Yes |
| 849 | 7 | 2673205 | 2673630 | ER_99460 | TTYH3 | No |
| 850 | 7 | 2702609 | 2702937 | ER_99466 | TTYH3 | No |
| 851 | 7 | 2731506 | 2731863 | ER_99473 | AMZ1 | Yes |
| 852 | 7 | 2760404 | 2760837 | ER_99477 | AMZ1 | Yes |
| 853 | 7 | 4876684 | 4876984 | ER_99580 | RADIL | No |
| 854 | 7 | 5436737 | 5437105 | ER_99601 | TNRC18 | Yes |
| 855 | 7 | 5526272 | 5526875 | ER_99612 | FBXL18 | Yes |
| 856 | 7 | 6312965 | 6313265 | ER_99654 | CYTH3 | No |

In one example, the method of detecting differential methylation comprises detecting differential methylation at one or more CpG dinucleotide sequences within one or more regions set forth in rows 1-856 of Table 1 in a subject suffering from ESR1 positive breast cancer relative to a reference level of methylation for the corresponding one or more CpG dinucleotide sequences. For example, the method may comprise detecting differential methylation at a single CpG dinucleotide sequence within any one of the genomic regions defined in Table 1 in a subject suffering from ESR1 positive breast cancer relative to a reference level of methylation for the corresponding CpG dinucleotide sequence.

In another example, the method of detecting differential methylation comprises detecting differential methylation at two or more CpG dinucleotides within any genomic region defined in Table 1 in a subject suffering from ESR1 positive breast cancer relative to a reference level of methylation for the corresponding two or more CpG dinucleotide sequences. For example, the method may comprise detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 1 in a subject suffering from ESR1 positive breast cancer relative to a reference level of methylation for the corresponding CpG dinucleotide sequences. The two or more CpG dinucleotides may be consecutive (i.e., contiguous) within a genomic region. Alternatively, the two or more CpG dinucleotides may not be consecutive (i.e., may not be contiguous) within any genomic region.

In yet another example, the method of detecting differential methylation comprises detecting differential methylation of at least one CpG dinucleotide within two or more different genomic regions set forth in Table 1 in a subject suffering from ESR1 positive breast cancer relative to a reference level of methylation for the corresponding CpG dinucleotide sequences. For example, the method may comprise detecting differential methylation at a CpG dinucleotide within two or more, or three or more, or four or more, or five or more, or six or more, or seven or more, or eight or more, or nine or more, or 10 or more different genomic regions set forth in Table 1 in a subject suffering from ESR1 positive breast cancer relative to a reference level of methylation for the corresponding CpG dinucleotide sequences.

In one example, identifying differential methylation at one or more CpG dinucleotide sequences within one or more regions set forth in rows 1-856 of Table 1 in a subject relative to a reference level of methylation for the corresponding one or more CpG dinucleotide sequences is indicative of the subject's likely response to endocrine therapy.

Detecting differential methylation at a single CpG dinucleotide sequence within any one of the genomic regions defined in Table 1 may be indicative of the subject's likely response to endocrine therapy.

Alternatively, detecting differential methylation at two or more CpG dinucleotides within any genomic region defined in Table 1 may be indicative of the subject's likely response to endocrine therapy. For example, detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 1 may be indicative of the subject's likely response to endocrine therapy. The two or more CpG dinucleotides may be consecutive (i.e., contiguous) within a genomic region. Alternatively, the two or more CpG dinucleotides may not be consecutive (i.e., may not be contiguous) within any genomic region.

Detecting differential methylation of at least one CpG dinucleotide within two or more different genomic regions set forth in Table 1 may be indicative of the subject's likely response to endocrine therapy. For example, detecting differential methylation at a CpG dinucleotide within two or more, or three or more, or four or more, or five or more, or six or more, or seven or more, or eight or more, or nine or more, or 10 or more different genomic regions set forth in Table 1 may be indicative of the subject's likely response to endocrine therapy.

In one example, identifying differential methylation at one or more CpG dinucleotide sequences within one or more regions set forth in rows 1-856 of Table 1 in a subject relative to a reference level of methylation for the corresponding one or more CpG dinucleotide sequences is indicative of the subject having breast cancer which is refractory to endocrine therapy.

Detecting differential methylation at a single CpG dinucleotide sequence within any one of the genomic regions defined in Table 1 may be indicative of the subject having breast cancer which is refractory to endocrine therapy.

Alternatively, detecting differential methylation at two or more CpG dinucleotides within any genomic region defined in Table 1 may be indicative of the subject having breast cancer which is refractory to endocrine therapy. For example, detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 1 may be indicative of the subject having breast cancer which is refractory to endocrine therapy. The two or more CpG dinucleotides may be consecutive (i.e., contiguous) within a genomic region. Alternatively, the two or more CpG dinucleotides may not be consecutive (i.e., may not be contiguous) within any genomic region.

Detecting differential methylation of at least one CpG dinucleotide within two or more different genomic regions set forth in Table 1 may be indicative of the subject having breast cancer which is refractory to endocrine therapy. For example, detecting differential methylation at a CpG dinucleotide within two or more, or three or more, or four or more, or five or more, or six or more, or seven or more, or eight or more, or nine or more, or 10 or more different genomic regions set forth in Table 1 may be indicative of the subject having breast cancer which is refractory to endocrine therapy.

In one example, identifying differential methylation at one or more CpG dinucleotide sequences within one or more regions set forth in rows 1-856 of Table 1 in a subject relative to a reference level of methylation for the corresponding one or more CpG dinucleotide sequences is indicative of the likely therapeutic outcome and/or of the progression of the ESR1 positive breast cancer.

Detecting differential methylation at a single CpG dinucleotide sequence within any one of the genomic regions defined in Table 1 may be indicative of the likely therapeutic outcome and/or of the progression of the ESR1 positive breast cancer.

Alternatively, detecting differential methylation at two or more CpG dinucleotides within any genomic region defined in Table 1 may be indicative of the likely therapeutic outcome and/or of the progression of the ESR1 positive breast cancer. For example, detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 1 may be indicative of the likely therapeutic outcome and/or of the progression of the ESR1 positive breast cancer. The two or more CpG dinucleotides may be consecutive (i.e., contiguous) within a genomic region. Alternatively, the two or more CpG dinucleotides may not be consecutive (i.e., may not be contiguous) within any genomic region.

Detecting differential methylation of at least one CpG dinucleotide within two or more different genomic regions set forth in Table 1 may be indicative of the likely therapeutic outcome and/or of the progression of the ESR1 positive breast cancer. For example, detecting differential methylation at a CpG dinucleotide within two or more, or three or more, or four or more, or five or more, or six or more, or seven or more, or eight or more, or nine or more, or 10 or more different genomic regions set forth in Table 1 may be indicative of the likely therapeutic outcome and/or of the progression of the ESR1 positive breast cancer.

In another example, the methods of the disclosure may comprise determining methylation status of one or more CpG dinucleotide sequences within one or more ESR1 binding sites which is/are intragenic. For example, the one or more CpG dinucleotide sequences may be within one or more of the 617 intragenic ESR1 binding sites set forth in Table 2. The 617 genomic regions listed in Table 2 encompass intragenic ESR1 binding sites that overlap estrogen responsive enhancer regions and which contain hypermethylated CpG dinucleotides in multiple models of endocrine resistance (i.e., MCF7-derived cell lines, tamoxifen-resistant (TAMR)10, fulvestrant-resistant (FASR)11 and estrogen deprivation resistant (MCF7X)12 cells), relative to ESR1-positive hormone sensitive MCF7 cells.

The ESR1 binding sites set forth in Table 2 are also defined with reference to hg19. Thus, the nucleotide sequences of each of the regions identified in Table 2 (or in any of the Tables disclosed herein) can be identified by reference to hg19, using the “start” and “end” positions described in Table 2 (or in any of the Tables disclosed herein). For each of the ESR1 binding sites set forth in Table 2, the following information is provided:

### (i) Chromosome ID (Column 2);

(ii) genomic coordinates of ESR1-binding site with respect to hg19 (Columns 3-4);
(iii) ESR1-binding site name (Column 5);
(iv) name of gene which ESR1-binding site is most closely associated (Column 6);
(v) inverse correlation between methylation and gene expression in TCGA (Column 7);
(vi) spearman's RHO (Column 8);
(vii) correlation P-value (Column 9); and
(viii) number of HM450K probes per ESR1 binding site (Column 10).

TABLE 2
Hypermethylated ESR1 binding sites in estrogen enhancer regions which are intragenic

| | | | | | | Inverse | | | No. |
|---|---|---|---|---|---|---|---|---|---|
| | | | | | | correlation | | | statistically |
| | | | | | | between | | | significant |
| | | | | | | methylation and | | Correlation | probes |
| Row | Chromo- | | | ESR1 | | Expression | | P-value | per ESR1 |
| No. | some | Start | End | site name | Gene | in TCGA | Spearman's RHO | (P > 0.001) | binding site |
| 1 | 7 | 27210940 | 27211286 | ER_100307 | HOXA-AS4 | No | n/a | n/a | n/a |
| 2 | 7 | 43288514 | 43288831 | ER_100892 | HECW1 | No | n/a | n/a | n/a |
| 3 | 7 | 44224085 | 44224425 | ER_100934 | GCK | No | n/a | n/a | n/a |
| 4 | 7 | 47611478 | 47611980 | ER_101074 | TNS3 | No | n/a | n/a | n/a |
| 5 | 7 | 55620324 | 55620676 | ER_101291 | VOPP1 | No | n/a | n/a | n/a |
| 6 | 7 | 75279353 | 75279713 | ER_101692 | HIP1 | No | n/a | n/a | n/a |
| 7 | 7 | 75362309 | 75362707 | ER_101699 | HIP1 | Yes | −0.25958627 | 2.59613E−08 | 1 |
| 8 | 7 | 87855579 | 87855879 | ER_102057 | SRI | No | n/a | n/a | n/a |
| 9 | 1 | 224400118 | 224400678 | ER_10231 | NVL | No | n/a | n/a | n/a |
| 10 | 7 | 98989800 | 98990147 | ER_102479 | ARPC1B | No | n/a | n/a | n/a |
| 11 | 7 | 99719664 | 99719969 | ER_102522 | CNPY4 | No | n/a | n/a | n/a |
| 12 | 7 | 100463589 | 100463889 | ER_102585 | SLC12A9 | No | n/a | n/a | n/a |
| 13 | 7 | 100464090 | 100464390 | ER_102586 | SLC12A9 | No | n/a | n/a | n/a |
| 14 | 7 | 100800324 | 100800624 | ER_102605 | AP1S1 | Yes | −0.308429704 | 2.87107E−11 | 1 |
| 15 | 7 | 101017180 | 101017480 | ER_102622 | EMID2 | No | 0.325866479 | 1.36233E−12 | 1 |
| 16 | 7 | 101180514 | 101180818 | ER_102635 | EMID2 | No | 0.471143615 | 3.03374E−26 | 1 |
| 17 | 7 | 101768479 | 101768882 | ER_102709 | CUX1 | No | n/a | n/a | n/a |
| 18 | 7 | 101959130 | 101959535 | ER_102722 | SH2B2 | Yes | −0.285866301 | 7.82483E−10 | 1 |
| 19 | 7 | 102080614 | 102081003 | ER_102733 | ORAI2 | Yes | −0.18527387 | 7.91545E−05 | 1 |
| 20 | 7 | 105313870 | 105314183 | ER_102843 | ATXN7L1 | No | n/a | n/a | n/a |
| 21 | 1 | 27240488 | 27241066 | ER_1029 | NR0B2 | Yes | −0.224802698 | 1.45501E−06 | 1 |
| 22 | 7 | 107886705 | 107887046 | ER_103011 | NRCAM | No | n/a | n/a | n/a |
| 23 | 1 | 228598287 | 228598587 | ER_10389 | TRIM17 | Yes | −0.249438828 | 8.24599E−08 | 1 |
| 24 | 7 | 138560635 | 138560935 | ER_104150 | KIAA1549 | No | n/a | n/a | n/a |
| 25 | 7 | 139345059 | 139345423 | ER_104192 | HIPK2 | Yes | −0.216367357 | 3.82399E−06 | 1 |
| 26 | 7 | 143106427 | 143106737 | ER_104387 | EPHA1-AS1 | No | n/a | n/a | n/a |
| 27 | 7 | 144104415 | 144104715 | ER_104415 | NOBOX | No | n/a | n/a | n/a |
| 28 | 7 | 148901363 | 148901733 | ER_104483 | ZNF282 | No | n/a | n/a | n/a |
| 29 | 7 | 149569864 | 149570164 | ER_104518 | ATP6V0E2 | No | n/a | n/a | n/a |
| 30 | 7 | 151418719 | 151419146 | ER_104616 | PRKAG2 | No | n/a | n/a | n/a |
| 31 | 7 | 151424787 | 151425151 | ER_104617 | PRKAG2 | No | n/a | n/a | n/a |
| 32 | 7 | 151442252 | 151442633 | ER_104619 | PRKAG2 | No | n/a | n/a | n/a |
| 33 | 7 | 151493516 | 151493829 | ER_104624 | PRKAG2 | No | n/a | n/a | n/a |
| 34 | 7 | 151553656 | 151553979 | ER_104631 | PRKAG2 | No | n/a | n/a | n/a |
| 35 | 7 | 155600847 | 155601325 | ER_104760 | SHH | No | n/a | n/a | n/a |
| 36 | 7 | 157916425 | 157916765 | ER_104936 | PTPRN2 | Yes | −0.190965163 | 0.000045562 | 1 |
| 37 | 1 | 230895945 | 230896482 | ER_10494 | CAPN9 | Yes | −0.361156359 | 2.60574E−15 | 1 |
| 38 | 1 | 231113407 | 231113970 | ER_10502 | TTC13 | Yes | −0.215328471 | 0.000004263 | 1 |
| 39 | 8 | 1946769 | 1947125 | ER_105037 | KBTBD11 | No | n/a | n/a | n/a |
| 40 | 8 | 17394868 | 17395391 | ER_105282 | SLC7A2 | Yes | −0.584122654 | 0 | 3 |
| 41 | 8 | 21948482 | 21948782 | ER_105469 | FAM160B2 | Yes | −0.316480707 | 8.22104E−12 | 1 |
| 42 | 8 | 28223166 | 28223486 | ER_105673 | ZNF395 | Yes | −0.30437003 | 5.3156E−11 | 1 |
| 43 | 8 | 37700545 | 37700845 | ER_105921 | GPR124 | No | n/a | n/a | n/a |
| 44 | 8 | 48739114 | 48739414 | ER_106145 | PRKDC | No | n/a | n/a | n/a |
| 45 | 8 | 62567898 | 62568253 | ER_106497 | ASPH | Yes | −0.174035493 | 0.000211725 | 1 |
| 46 | 8 | 67425037 | 67425360 | ER_106600 | C8orf46 | Yes | −0.446493874 | 1.96242E−23 | 1 |
| 47 | 8 | 74002442 | 74002818 | ER_106800 | SBSPON | No | n/a | n/a | n/a |
| 48 | 8 | 90772144 | 90772507 | ER_107685 | RIPK2 | No | n/a | n/a | n/a |
| 49 | 8 | 98881668 | 98882017 | ER_108508 | MATN2 | Yes | −0.199715554 | 2.05104E−05 | 1 |
| 50 | 8 | 101017572 | 101017872 | ER_108722 | RGS22 | Yes | −0.424259141 | 4.35447E−21 | 1 |
| 51 | 1 | 249106283 | 249106588 | ER_10898 | SH3BP5L | No | n/a | n/a | n/a |
| 52 | 8 | 102526632 | 102526954 | ER_109025 | GRHL2 | Yes | −0.470255952 | 0 | 1 |
| 53 | 10 | 416280 | 416842 | ER_10930 | DIP2C | Yes | −0.266239142 | 1.1071E−08 | 1 |
| 54 | 10 | 579019 | 579393 | ER_10949 | DIP2C | Yes | −0.190689534 | 4.82507E−05 | 1 |
| 55 | 8 | 124193498 | 124193984 | ER_110732 | FAM83A | Yes | −0.43367174 | 4.64354E−22 | 1 |
| 56 | 8 | 124194626 | 124194965 | ER_110733 | FAM83A | Yes | −0.211481781 | 6.03929E−06 | 1 |
| 57 | 8 | 126082161 | 126082738 | ER_111167 | KIAA0196 | Yes | −0.361311348 | 3.0008E−15 | 1 |
| 58 | 8 | 129103245 | 129103607 | ER_111781 | PVT1 | Yes | −0.35335898 | 1.45588E−14 | 1 |
| 59 | 8 | 134224471 | 134224833 | ER_112110 | WISP1 | No | n/a | n/a | n/a |
| 60 | 8 | 134249814 | 134250165 | ER_112114 | NDRG1 | No | n/a | n/a | n/a |
| 61 | 8 | 135490722 | 135491122 | ER_112226 | ZFAT | No | n/a | n/a | n/a |
| 62 | 8 | 142139549 | 142139849 | ER_112470 | DENND3 | Yes | −0.208632932 | 8.48193E−06 | 1 |
| 63 | 8 | 142247703 | 142248043 | ER_112484 | SLC45A4 | Yes | −0.18946273 | 5.40406E−05 | 2 |
| 64 | 8 | 143395413 | 143395760 | ER_112563 | TSNARE1 | Yes | −0.175513031 | 0.000186653 | 1 |
| 65 | 8 | 143625782 | 143626082 | ER_112589 | BAI1 | No | n/a | n/a | n/a |
| 66 | 8 | 143763120 | 143763420 | ER_112608 | PSCA | No | n/a | n/a | n/a |
| 67 | 8 | 143823681 | 143823981 | ER_112619 | SLURP1 | Yes | −0.621291369 | 2.14036E−49 | 4 |
| 68 | 8 | 143851193 | 143851559 | ER_112624 | LYNX1 | Yes | −0.204416812 | 1.29375E−05 | 1 |
| 69 | 8 | 143867136 | 143867477 | ER_112628 | LY6D | No | n/a | n/a | n/a |
| 70 | 8 | 144129484 | 144129962 | ER_112655 | C8orf31 | No | 0.206690735 | 9.86107E−06 | 1 |
| 71 | 10 | 7984711 | 7985286 | ER_11272 | TAF3 | No | n/a | n/a | n/a |
| 72 | 8 | 145048592 | 145048911 | ER_112777 | PLEC | Yes | −0.233518914 | 5.8926E−07 | 1 |
| 73 | 8 | 145086414 | 145086732 | ER_112784 | SPATC1 | Yes | −0.27978868 | 1.54356E−09 | 1 |
| 74 | 9 | 33443026 | 33443378 | ER_113615 | AQP3 | Yes | −0.375082083 | 1.00675E−16 | 1 |
| 75 | 9 | 34372740 | 34373087 | ER_113641 | KIAA1161 | Yes | −0.200056757 | 1.98428E−05 | 1 |
| 76 | 9 | 71660820 | 71661159 | ER_113876 | FXN | No | n/a | n/a | n/a |
| 77 | 9 | 95834504 | 95834841 | ER_115179 | SUSD3 | Yes | −0.290895198 | 3.83898E−10 | 1 |
| 78 | 1 | 3388133 | 3388514 | ER_116 | ARHGEF16 | No | n/a | n/a | n/a |
| 79 | 9 | 124979962 | 124980333 | ER_116729 | LHX6 | No | 0.267574721 | 9.30418E−09 | 2 |
| 80 | 9 | 129433798 | 129434130 | ER_117042 | LMX1B | Yes | −0.571087528 | 0 | 1 |
| 81 | 9 | 130484264 | 130484588 | ER_117145 | TTC16 | No | n/a | n/a | n/a |
| 82 | 9 | 130487106 | 130487456 | ER_117146 | TTC16 | No | n/a | n/a | n/a |
| 83 | 9 | 130524258 | 130524602 | ER_117150 | SH2D3C | No | 0.368556421 | 6.03173E−16 | 1 |
| 84 | 9 | 130727796 | 130728104 | ER_117181 | FAM102A | No | n/a | n/a | n/a |
| 85 | 9 | 133974376 | 133974700 | ER_117471 | AIF1L | No | n/a | n/a | n/a |
| 86 | 9 | 136728378 | 136728709 | ER_117634 | VAV2 | Yes | −0.239045592 | 3.12752E−07 | 1 |
| 87 | 9 | 137252760 | 137253089 | ER_117681 | RXRA | Yes | −0.244453224 | 1.65813E−07 | 1 |
| 88 | 9 | 137258516 | 137258927 | ER_117685 | RXRA | Yes | −0.22430142 | 1.63893E−06 | 1 |
| 89 | 9 | 137302122 | 137302482 | ER_117702 | RXRA | No | n/a | n/a | n/a |
| 90 | 9 | 139734802 | 139735102 | ER_117864 | RABL6 | No | n/a | n/a | n/a |
| 91 | 9 | 140374065 | 140374394 | ER_117940 | PNPLA7 | Yes | −0.227579527 | 1.14457E−06 | 1 |
| 92 | 9 | 140408341 | 140408692 | ER_117943 | PNPLA7 | No | n/a | n/a | n/a |
| 93 | 10 | 24496562 | 24497177 | ER_11835 | KIAA1217 | Yes | −0.283127061 | 1.14663E−09 | 1 |
| 94 | X | 18708777 | 18709167 | ER_118438 | PPEF1 | Yes | −0.26765505 | 9.20712E−09 | 2 |
| 95 | X | 40007163 | 40007510 | ER_118823 | BCOR | No | n/a | n/a | n/a |
| 96 | 10 | 30316774 | 30317298 | ER_12062 | KIAA1462 | No | n/a | n/a | n/a |
| 97 | X | 153586498 | 153586870 | ER_120632 | FLNA | No | n/a | n/a | n/a |
| 98 | 1 | 3471648 | 3472248 | ER_122 | MEGF6 | No | 0.163319391 | 0.000512684 | 1 |
| 99 | 1 | 37321565 | 37322055 | ER_1299 | GRIK3 | No | 0.530788416 | 4.53213E−34 | 1 |
| 100 | 10 | 71213510 | 71214074 | ER_13175 | TSPAN15 | Yes | −0.424739404 | 3.89108E−21 | 1 |
| 101 | 10 | 73828553 | 73829113 | ER_13269 | SPOCK2 | No | 0.440229269 | 0 | 1 |
| 102 | 1 | 3614361 | 3614966 | ER_133 | TP73 | No | 0.428857492 | 0 | 1 |
| 103 | 10 | 76858883 | 76859398 | ER_13395 | DUSP13 | Yes | −0.374994214 | 1.79367E−16 | 6 |
| 104 | 10 | 77872231 | 77872922 | ER_13427 | C10orf11 | No | 0.192369608 | 4.12673E−05 | 2 |
| 105 | 10 | 82189362 | 82189690 | ER_13657 | FAM213A | No | n/a | n/a | n/a |
| 106 | 10 | 88475369 | 88475919 | ER_13815 | LDB3 | Yes | −0.20457813 | 1.27322E−05 | 1 |
| 107 | 10 | 88703091 | 88703639 | ER_13821 | MMRN2 | No | n/a | n/a | n/a |
| 108 | 10 | 95228052 | 95228774 | ER_14079 | MYOF | Yes | −0.293781665 | 2.53507E−10 | 1 |
| 109 | 10 | 102792785 | 102793085 | ER_14340 | SFXN3 | Yes | −0.550035375 | 0 | 1 |
| 110 | 10 | 105238778 | 105239360 | ER_14449 | CALHM3 | Yes | −0.200511144 | 1.82534E−05 | 4 |
| 111 | 10 | 112259180 | 112259480 | ER_14629 | DUSP5 | Yes | −0.47604403 | 0 | 1 |
| 112 | 1 | 42420202 | 42420763 | ER_1481 | HIVEP3 | No | n/a | n/a | n/a |
| 113 | 10 | 119102102 | 119102657 | ER_14914 | PDZD8 | No | n/a | n/a | n/a |
| 114 | 10 | 121079102 | 121079402 | ER_15001 | GRK5 | No | n/a | n/a | n/a |
| 115 | 10 | 121415148 | 121415773 | ER_15018 | BAG 3 | Yes | −0.449812197 | 0 | 1 |
| 116 | 10 | 123900770 | 123901156 | ER_15113 | TACC2 | Yes | −0.165628275 | 0.000425606 | 1 |
| 117 | 10 | 126211691 | 126211991 | ER_15194 | LHPP | No | n/a | n/a | n/a |
| 118 | 10 | 126315723 | 126316102 | ER_15199 | FAM53B | Yes | −0.247233418 | 1.18974E−07 | 1 |
| 119 | 10 | 126700346 | 126700820 | ER_15221 | CTBP2 | Yes | −0.267400497 | 9.51812E−09 | 1 |
| 120 | 1 | 44300618 | 44300993 | ER_1532 | ST3GAL3 | Yes | −0.203979608 | 1.35099E−05 | 1 |
| 121 | 10 | 128606764 | 128607329 | ER_15340 | DOCK1 | Yes | −0.286907293 | 6.76003E−10 | 1 |
| 122 | 10 | 134079116 | 134079490 | ER_15434 | STK32C | No | n/a | n/a | n/a |
| 123 | 10 | 134224980 | 134225280 | ER_15443 | PWWP2B | Yes | −0.185156404 | 7.99969E−05 | 2 |
| 124 | 10 | 134261296 | 134261809 | ER_15450 | C10orf91 | Yes | −0.370354575 | 4.46339E−16 | 3 |
| 125 | 10 | 134420347 | 134420647 | ER_15478 | INPP5A | Yes | −0.177054833 | 0.000163475 | 1 |
| 126 | 10 | 134498671 | 134499284 | ER_15480 | INPP5A | Yes | −0.207945257 | 9.09185E−06 | 1 |
| 127 | 10 | 134728825 | 134729477 | ER_15487 | TTC40 | No | n/a | n/a | n/a |
| 128 | 10 | 134729867 | 134730225 | ER_15488 | TTC40 | No | n/a | n/a | n/a |
| 129 | 10 | 135178534 | 135178842 | ER_15502 | ECHS1 | No | n/a | n/a | n/a |
| 130 | 11 | 849066 | 849366 | ER_15551 | TSPAN4 | No | 0.165317491 | 0.000436466 | 2 |
| 131 | 11 | 1275364 | 1275883 | ER_15574 | MUC5B | No | 0.374119444 | 1.36781E−16 | 2 |
| 132 | 11 | 1460055 | 1460355 | ER_15581 | BRSK2 | No | 0.227080381 | 1.13056E−06 | 1 |
| 133 | 11 | 1507075 | 1507659 | ER_15583 | MOB2 | Yes | −0.155896803 | 0.000917668 | 1 |
| 134 | 11 | 1777371 | 1777938 | ER_15595 | CTSD | Yes | −0.389507306 | 0 | 1 |
| 135 | 11 | 2004135 | 2004733 | ER_15613 | MRPL23 | No | n/a | n/a | n/a |
| | | | | | [symbol] | | | | |
| 136 | 11 | 2181411 | 2181715 | ER_15620 | INS | No | n/a | n/a | n/a |
| 137 | 11 | 2181411 | 2181715 | ER_15620 | INS-IGF2 | No | n/a | n/a | n/a |
| 138 | 11 | 2431263 | 2431688 | ER_15638 | TRPM5 | No | n/a | n/a | n/a |
| 139 | 11 | 12309323 | 12309637 | ER_15849 | MICALCL | No | 0.262835668 | 1.71706E−08 | 1 |
| 140 | 11 | 20063465 | 20064029 | ER_16131 | NAV2 | No | n/a | n/a | n/a |
| 141 | 11 | 33744676 | 33745230 | ER_16397 | CD59 | Yes | −0.304313667 | 5.36089E−11 | 2 |
| 142 | 11 | 45928675 | 45929091 | ER_16775 | C11orf94 | No | n/a | n/a | n/a |
| 143 | 11 | 46373841 | 46374290 | ER_16803 | DGKZ | Yes | −0.292581725 | 3.01408E−10 | 1 |
| 144 | 11 | 61466759 | 61467374 | ER_17096 | DAGLA | Yes | −0.222108817 | 2.07767E−06 | 1 |
| 145 | 11 | 62437396 | 62437722 | ER_17173 | C11orf48 | Yes | −0.162346349 | 0.000554124 | 2 |
| 146 | 11 | 62647962 | 62648262 | ER_17194 | SLC3A2 | No | n/a | n/a | n/a |
| 147 | 11 | 64034904 | 64035204 | ER_17276 | PLCB3 | Yes | −0.229382466 | 9.37378E−07 | 2 |
| 148 | 11 | 64053773 | 64054355 | ER_17285 | GPR137 | No | n/a | n/a | n/a |
| 149 | 11 | 64322980 | 64323660 | ER_17306 | SLC22A11 | No | n/a | n/a | n/a |
| 150 | 11 | 66659547 | 66660139 | ER_17525 | PC | No | n/a | n/a | n/a |
| 151 | 11 | 67165633 | 67165963 | ER_17593 | PPP1CA | No | n/a | n/a | n/a |
| 152 | 11 | 67186225 | 67186704 | ER_17595 | CARNS1 | No | n/a | n/a | n/a |
| 153 | 11 | 67810737 | 67811051 | ER_17643 | TCIRG1 | No | n/a | n/a | n/a |
| 154 | 11 | 69061731 | 69062031 | ER_17743 | MYEOV | Yes | −0.161570899 | 0.000580565 | 1 |
| 155 | 11 | 69515571 | 69515871 | ER_17789 | FGF19 | No | n/a | n/a | n/a |
| 156 | 11 | 70917115 | 70917490 | ER_17848 | SHANK2 | Yes | −0.315797247 | 9.15588E−12 | 3 |
| 157 | 11 | 71276995 | 71277590 | ER_17878 | KRTAP5-10 | No | n/a | n/a | n/a |
| 158 | 11 | 73000242 | 73000542 | ER_17968 | P2RY6 | No | 0.351070507 | 2.24479E−14 | 1 |
| 159 | 11 | 76371803 | 76372137 | ER_18134 | LRRC32 | No | 0.228162509 | 1.07319E−06 | 2 |
| 160 | 11 | 85468982 | 85469571 | ER_18506 | SYTL2 | Yes | −0.459623471 | 0 | 3 |
| 161 | 1 | 6330275 | 6330725 | ER_187 | ACOT7 | No | n/a | n/a | n/a |
| 162 | 11 | 117300693 | 117300993 | ER_19145 | DSCAML1 | No | n/a | n/a | n/a |
| 163 | 11 | 118013408 | 118013932 | ER_19187 | SCN4B | No | 0.166779885 | 0.000387521 | 1 |
| 164 | 1 | 6445555 | 6445855 | ER_192 | ACOT7 | Yes | −0.346507797 | 3.85637E−14 | 1 |
| 165 | 11 | 118758401 | 118758701 | ER_19225 | CXCR5 | No | 0.327604866 | 1.01939E−12 | 1 |
| 166 | 11 | 119994594 | 119994894 | ER_19291 | TRIM29 | No | n/a | n/a | n/a |
| 167 | 11 | 119995602 | 119996221 | ER_19292 | TRIM29 | No | n/a | n/a | n/a |
| 168 | 12 | 2021757 | 2022057 | ER_19684 | CACNA2D4 | No | n/a | n/a | n/a |
| 169 | 12 | 48396469 | 48396769 | ER_21327 | COL2A1 | No | n/a | n/a | n/a |
| 170 | 12 | 52579425 | 52579733 | ER_21620 | KRT80 | Yes | −0.156387208 | 0.000883714 | 1 |
| 171 | 12 | 53300210 | 53300676 | ER_21729 | KRT8 | Yes | −0.358090657 | 5.78223E−15 | 1 |
| 172 | 12 | 53552932 | 53553480 | ER_21786 | CSAD | Yes | −0.462318596 | 0 | 1 |
| 173 | 12 | 57559601 | 57559901 | ER_22058 | LRP1 | No | n/a | n/a | n/a |
| 174 | 12 | 58160686 | 58160986 | ER_22088 | CYP27B1 | Yes | −0.255597575 | 4.28052E−08 | 2 |
| 175 | 12 | 58209624 | 58209992 | ER_22094 | AVIL | No | n/a | n/a | n/a |
| 176 | 12 | 63193501 | 63193816 | ER_22367 | PPM1H | Yes | −0.167030751 | 0.000379657 | 1 |
| 177 | 12 | 65043934 | 65044256 | ER_22510 | RASSF3 | No | n/a | n/a | n/a |
| 178 | 12 | 65066737 | 65067037 | ER_22517 | RASSF3 | No | n/a | n/a | n/a |
| 179 | 1 | 7705733 | 7706033 | ER_240 | CAMTA1 | No | n/a | n/a | n/a |
| 180 | 12 | 112207002 | 112207577 | ER_24835 | ALDH2 | No | n/a | n/a | n/a |
| 181 | 12 | 113547314 | 113547643 | ER_24903 | RASAL1 | No | 0.398144188 | 1.51701E−18 | 1 |
| 182 | 12 | 123446240 | 123446640 | ER_25491 | ABCB9 | Yes | −0.188629804 | 5.83395E−05 | 1 |
| 183 | 12 | 123449086 | 123449400 | ER_25493 | ABCB9 | Yes | −0.416828462 | 0 | 1 |
| 184 | 12 | 124844847 | 124845161 | ER_25578 | NCOR2 | Yes | −0.158414149 | 0.000755335 | 2 |
| 185 | 12 | 124879925 | 124880326 | ER_25592 | NCOR2 | Yes | −0.219796114 | 2.66153E−06 | 3 |
| 186 | 12 | 125004808 | 125005149 | ER_25616 | NCOR2 | Yes | −0.17339707 | 0.000223506 | 1 |
| 187 | 12 | 131438531 | 131438856 | ER_25787 | GPR133 | No | n/a | n/a | n/a |
| 188 | 12 | 131622396 | 131622696 | ER_25800 | GPR133 | No | 0.383647195 | 0 | 2 |
| 189 | 12 | 132694663 | 132695050 | ER_25875 | GALNT9 | No | 0.176229524 | 0.000171659 | 1 |
| 190 | 13 | 34208323 | 34209020 | ER_26636 | STARD13 | Yes | −0.292609379 | 3.0021E−10 | 1 |
| 191 | 13 | 41590103 | 41590403 | ER_26934 | ELF1 | Yes | −0.28858071 | 4.43501E−10 | 1 |
| 192 | 13 | 51854732 | 51855052 | ER_27435 | FAM124A | No | 0.197644104 | 2.50444E−05 | 1 |
| 193 | 13 | 113651709 | 113652104 | ER_28798 | MCF2L | Yes | −0.416344509 | 0 | 1 |
| 194 | 13 | 113677094 | 113677394 | ER_28802 | MCF2L | Yes | −0.233597269 | 5.84054E−07 | 1 |
| 195 | 13 | 113979939 | 113980263 | ER_28813 | GRTP1 | Yes | −0.561680883 | 8.91715E−39 | 2 |
| 196 | 14 | 21494973 | 21495345 | ER_28907 | NDRG2 | No | n/a | n/a | n/a |
| 197 | 1 | 8823899 | 8824414 | ER_290 | RERE | Yes | −0.425083186 | 0 | 1 |
| 198 | 14 | 23623250 | 23623562 | ER_29019 | SLC7A8 | Yes | −0.354356515 | 1.20238E−14 | 1 |
| 199 | 14 | 23623676 | 23623976 | ER_29020 | SLC7A8 | Yes | −0.296853548 | 1.62175E−10 | 4 |
| 200 | 14 | 64932503 | 64932803 | ER_31140 | AKAP5 | Yes | −0.214691628 | 4.5555E−06 | 2 |
| 201 | 14 | 68871364 | 68871773 | ER_31488 | RAD51B | No | n/a | n/a | n/a |
| 202 | 14 | 74214079 | 74214466 | ER_31871 | C14orf43 | Yes | −0.432667091 | 0 | 1 |
| 203 | 14 | 74238204 | 74238510 | ER_31884 | C14orf43 | Yes | −0.195733971 | 3.00548E−05 | 1 |
| 204 | 1 | 1141942 | 1142242 | ER_32 | TNFRSF18 | No | n/a | n/a | n/a |
| 205 | 14 | 76447292 | 76447654 | ER_32084 | TGFB3 | Yes | −0.303016542 | 6.51353E−11 | 3 |
| 206 | 14 | 88942632 | 88943092 | ER_32519 | PTPN21 | Yes | −0.168486692 | 0.000336879 | 1 |
| 207 | 14 | 93412574 | 93412953 | ER_32818 | ITPK1 | Yes | −0.361876948 | 2.66671E−15 | 2 |
| 208 | 14 | 93473727 | 93474077 | ER_32832 | ITPK1 | Yes | −0.290394125 | 4.12381E−10 | 1 |
| 209 | 14 | 94392599 | 94392913 | ER_32902 | FAM181A- | No | n/a | n/a | n/a |
| | | | | | [symbol] | | | | |
| 210 | 14 | 95047627 | 95047927 | ER_32949 | SERPINA5 | Yes | −0.671013091 | 0 | 2 |
| 211 | 14 | 95078366 | 95078753 | ER_32959 | SERPINA3 | Yes | −0.324660895 | 2.21327E−12 | 2 |
| 212 | 14 | 95615683 | 95616180 | ER_32995 | DICER1 | Yes | −0.296402122 | 1.73236E−10 | 1 |
| 213 | 14 | 96691960 | 96692332 | ER_33117 | BDKRB2 | Yes | −0.167100809 | 0.000377488 | 1 |
| 214 | 14 | 100055296 | 100055707 | ER_33296 | CCDC85C | Yes | −0.175193161 | 0.000191832 | 1 |
| 215 | 14 | 100618272 | 100618577 | ER_33370 | DEGS2 | Yes | −0.394516582 | 0 | 1 |
| 216 | 14 | 103566340 | 103566640 | ER_33567 | EXOC3L4 | No | n/a | n/a | n/a |
| 217 | 14 | 103576070 | 103576413 | ER_33570 | EXOC3L4 | No | n/a | n/a | n/a |
| 218 | 14 | 104165149 | 104165544 | ER_33615 | XRCC3 | No | n/a | n/a | n/a |
| 219 | 14 | 104637698 | 104638043 | ER_33656 | KIF26A | No | 0.430282487 | 0 | 1 |
| 220 | 14 | 105181841 | 105182228 | ER_33695 | INF2 | Yes | −0.165915618 | 0.00041579 | 1 |
| 221 | 14 | 105434204 | 105434522 | ER_33730 | AHNAK2 | Yes | −0.17137013 | 0.000265106 | 1 |
| 222 | 14 | 105823215 | 105823515 | ER_33780 | PACS2 | No | n/a | n/a | n/a |
| 223 | 15 | 32951599 | 32952135 | ER_33988 | SCG5 | No | n/a | n/a | n/a |
| 224 | 15 | 50519199 | 50519766 | ER_34615 | SLC27A2 | Yes | −0.342165772 | 1.1351E−13 | 1 |
| 225 | 1 | 10075638 | 10076128 | ER_353 | RBP7 | No | n/a | n/a | n/a |
| 226 | 15 | 63074443 | 63074894 | ER_35498 | TLN2 | No | n/a | n/a | n/a |
| 227 | 15 | 63345507 | 63345850 | ER_35518 | TPM1 | No | n/a | n/a | n/a |
| 228 | 15 | 63671577 | 63672181 | ER_35532 | CA12 | Yes | −0.663688479 | 0 | 1 |
| 229 | 15 | 63672612 | 63673165 | ER_35533 | CA12 | Yes | −0.553637039 | 0 | 1 |
| 230 | 15 | 70384192 | 70384714 | ER_35866 | TLE3 | Yes | −0.624063592 | 0 | 1 |
| 231 | 15 | 74232754 | 74233252 | ER_36102 | LOXL1 | Yes | −0.219977053 | 2.6107E−06 | 1 |
| 232 | 15 | 74495260 | 74495560 | ER_36118 | STRA6 | No | n/a | n/a | n/a |
| 233 | 15 | 74537511 | 74538135 | ER_36123 | CCDC33 | No | n/a | n/a | n/a |
| 234 | 15 | 75974558 | 75974909 | ER_36216 | CSPG4 | No | n/a | n/a | n/a |
| 235 | 15 | 79223130 | 79223938 | ER_36351 | CTSH | No | 0.30157074 | 8.08343E−11 | 1 |
| 236 | 15 | 80878455 | 80879031 | ER_36421 | ARNT2 | Yes | −0.270986754 | 5.94211E−09 | 1 |
| 237 | 15 | 81596013 | 81596551 | ER_36480 | IL16 | No | 0.445491253 | 0 | 1 |
| 238 | 15 | 83781576 | 83782122 | ER_36550 | TM6SF1 | No | n/a | n/a | n/a |
| 239 | 15 | 86219506 | 86219806 | ER_36659 | AKAP13 | No | 0.155234149 | 0.000965466 | 1 |
| 240 | 15 | 89630906 | 89631554 | ER_36798 | ABHD2 | No | 0.239107665 | 2.85507E−07 | 2 |
| 241 | 15 | 90328102 | 90328625 | ER_36856 | ANPEP | No | n/a | n/a | n/a |
| 242 | 15 | 90349924 | 90350224 | ER_36858 | ANPEP | No | n/a | n/a | n/a |
| 243 | 15 | 96866722 | 96867218 | ER_37215 | NR2F2 | Yes | −0.210236502 | 6.86762E−06 | 2 |
| 244 | 15 | 99236506 | 99236926 | ER_37297 | IGF1R | Yes | −0.537894014 | 0 | 1 |
| 245 | 15 | 99271960 | 99272504 | ER_37301 | IGF1R | Yes | −0.588333638 | 0 | 1 |
| 246 | 15 | 101548985 | 101549285 | ER_37458 | LRRK1 | No | 0.400507986 | 0 | 1 |
| 247 | 16 | 374320 | 374831 | ER_37511 | AXIN1 | No | n/a | n/a | n/a |
| 248 | 16 | 585341 | 586026 | ER_37534 | MIR5587 | No | n/a | n/a | n/a |
| 249 | 16 | 615200 | 615500 | ER_37536 | C16orf11 | No | n/a | n/a | n/a |
| 250 | 16 | 633459 | 633759 | ER_37539 | PIGQ | No | n/a | n/a | n/a |
| 251 | 16 | 701631 | 702182 | ER_37550 | WDR90 | No | 0.194088004 | 0.000035121 | 1 |
| 252 | 16 | 863331 | 863631 | ER_37577 | PRR25 | No | n/a | n/a | n/a |
| 253 | 16 | 1138312 | 1138612 | ER_37596 | C1QTNF8 | No | n/a | n/a | n/a |
| 254 | 16 | 1209882 | 1210346 | ER_37604 | CACNA1H | No | n/a | n/a | n/a |
| 255 | 16 | 1232244 | 1232599 | ER_37605 | CACNA1H | Yes | −0.232335435 | 6.7354E−07 | 1 |
| 256 | 16 | 1240629 | 1241133 | ER_37606 | CACNA1H | No | n/a | n/a | n/a |
| 257 | 16 | 1430147 | 1430478 | ER_37632 | UNKL | No | n/a | n/a | n/a |
| 258 | 16 | 1827853 | 1828153 | ER_37665 | SPSB3 | Yes | −0.168403597 | 0.000339194 | 1 |
| 259 | 16 | 2004491 | 2004791 | ER_37679 | RPL3L | No | n/a | n/a | n/a |
| 260 | 16 | 2047651 | 2048147 | ER_37687 | ZNF598 | No | n/a | n/a | n/a |
| 261 | 16 | 2285522 | 2286213 | ER_37716 | E4F1 | No | n/a | n/a | n/a |
| 262 | 16 | 2294337 | 2294666 | ER_37717 | ECI1 | No | n/a | n/a | n/a |
| 263 | 16 | 2879820 | 2880369 | ER_37757 | ZG16B | Yes | −0.331448616 | 7.19734E−13 | 5 |
| 264 | 16 | 3704538 | 3704838 | ER_37830 | DNASE1 | Yes | −0.225124178 | 1.40433E−06 | 1 |
| 265 | 16 | 3707018 | 3707318 | ER_37831 | DNASE1 | Yes | −0.190279859 | 4.85723E−05 | 1 |
| 266 | 16 | 4421377 | 4421694 | ER_37887 | CORO7 | No | n/a | n/a | n/a |
| 267 | 16 | 4425919 | 4426536 | ER_37892 | VASN | No | n/a | n/a | n/a |
| 268 | 16 | 4478392 | 4478714 | ER_37899 | DNAJA3 | No | n/a | n/a | n/a |
| 269 | 16 | 4746859 | 4747392 | ER_37919 | ANKS3 | No | n/a | n/a | n/a |
| 270 | 16 | 4838430 | 4839026 | ER_37923 | Sep-12 | Yes | −0.290559784 | 3.32936E−10 | 3 |
| 271 | 16 | 14030414 | 14030714 | ER_38174 | ERCC4 | Yes | −0.286422287 | 7.23732E−10 | 1 |
| 272 | 16 | 14580747 | 14581084 | ER_38229 | PARN | Yes | −0.328987106 | 1.0858E−12 | 1 |
| 273 | 16 | 15602986 | 15603303 | ER_38266 | C16orf45 | No | n/a | n/a | n/a |
| 274 | 16 | 16090418 | 16090901 | ER_38297 | ABCC1 | No | n/a | n/a | n/a |
| 275 | 16 | 16108606 | 16109211 | ER_38301 | ABCC1 | Yes | −0.199363421 | 2.12218E−05 | 1 |
| 276 | 16 | 24747980 | 24748466 | ER_38621 | TNRC6A | Yes | −0.363523046 | 1.88066E−15 | 1 |
| 277 | 16 | 28511293 | 28511661 | ER_38732 | IL27 | No | n/a | n/a | n/a |
| 278 | 16 | 28608240 | 28608616 | ER_38739 | SULT1A2 | Yes | −0.449317709 | 0 | 1 |
| 279 | 16 | 30906082 | 30906783 | ER_38861 | BCL7C | No | n/a | n/a | n/a |
| 280 | 16 | 68321665 | 68322263 | ER_39511 | SLC7A6 | No | n/a | n/a | n/a |
| 281 | 16 | 69358300 | 69358626 | ER_39598 | VPS4A | No | n/a | n/a | n/a |
| 282 | 16 | 70472947 | 70473558 | ER_39656 | ST3GAL2 | No | n/a | n/a | n/a |
| 283 | 16 | 70687883 | 70688183 | ER_39667 | IL34 | No | 0.574054983 | 0 | 3 |
| 284 | 16 | 72994910 | 72995462 | ER_39877 | ZFHX3 | Yes | −0.209502467 | 7.76642E−06 | 1 |
| 285 | 16 | 78991185 | 78991807 | ER_40213 | WWOX | No | n/a | n/a | n/a |
| 286 | 16 | 81248258 | 81248721 | ER_40354 | PKD1L2 | No | n/a | n/a | n/a |
| 287 | 16 | 84628881 | 84629206 | ER_40553 | COTL1 | No | 0.234534492 | 5.25106E−07 | 2 |
| 288 | 16 | 84871395 | 84871718 | ER_40585 | CRISPLD2 | No | n/a | n/a | n/a |
| 289 | 16 | 85669299 | 85669599 | ER_40724 | KIAA0182 | Yes | −0.459019682 | 0 | 2 |
| 290 | 16 | 85723351 | 85723651 | ER_40737 | GINS2 | Yes | −0.29702843 | 1.58078E−10 | 2 |
| 291 | 16 | 85786954 | 85787520 | ER_40750 | C16orf74 | Yes | −0.272675914 | 4.74847E−09 | 2 |
| 292 | 1 | 109373005 | 109373612 | ER_4085 | AKNAD1 | Yes | −0.246004825 | 1.25377E−07 | 1 |
| 293 | 16 | 87778638 | 87778938 | ER_40904 | KLHDC4 | No | n/a | n/a | n/a |
| 294 | 16 | 87812661 | 87813207 | ER_40908 | KLHDC4 | Yes | −0.172678581 | 0.000237498 | 1 |
| 295 | 16 | 87868043 | 87868626 | ER_40912 | SLC7A5 | No | n/a | n/a | n/a |
| 296 | 16 | 87890385 | 87890763 | ER_40918 | SLC7A5 | Yes | −0.483398404 | 0 | 1 |
| 297 | 16 | 88110906 | 88111273 | ER_40942 | BANP | No | n/a | n/a | n/a |
| 298 | 16 | 88548864 | 88549438 | ER_40969 | ZFPM1 | No | n/a | n/a | n/a |
| 299 | 16 | 88704888 | 88705257 | ER_40987 | IL17C | No | n/a | n/a | n/a |
| 300 | 16 | 88988838 | 88989268 | ER_41018 | CBFA2T3 | No | n/a | n/a | n/a |
| 301 | 16 | 88991561 | 88992120 | ER_41020 | CBFA2T3 | No | n/a | n/a | n/a |
| 302 | 16 | 89004344 | 89004862 | ER_41024 | CBFA2T3 | No | 0.289451767 | 4.71623E−10 | 1 |
| 303 | 16 | 89043365 | 89043665 | ER_41029 | CBFA2T3 | No | 0.168608164 | 0.000327491 | 2 |
| 304 | 16 | 89648350 | 89648952 | ER_41093 | CPNE7 | No | n/a | n/a | n/a |
| 305 | 1 | 11020446 | 11021019 | ER_411 | C1orf127 | Yes | −0.239407483 | 2.75616E−07 | 2 |
| 306 | 16 | 89900078 | 89900645 | ER_41112 | SPIRE2 | Yes | −0.231466954 | 7.42637E−07 | 2 |
| 307 | 16 | 89927268 | 89927731 | ER_41118 | SPIRE2 | No | 0.337950574 | 2.37482E−13 | 1 |
| 308 | 17 | 151960 | 152279 | ER_41149 | RPH3AL | Yes | −0.376348525 | 6.43879E−17 | 3 |
| 309 | 17 | 1634282 | 1634616 | ER_41197 | WDR81 | No | n/a | n/a | n/a |
| 310 | 17 | 1901327 | 1901683 | ER_41216 | RTN4RL1 | Yes | −0.201776075 | 1.67812E−05 | 2 |
| 311 | 17 | 1987743 | 1988066 | ER_41224 | SMG6 | Yes | −0.176716395 | 0.000168319 | 1 |
| 312 | 17 | 3635535 | 3635874 | ER_41267 | ITGAE | No | 0.203757714 | 1.38096E−05 | 1 |
| 313 | 17 | 4436972 | 4437323 | ER_41315 | SPNS2 | No | 0.441142392 | 0 | 1 |
| 314 | 17 | 4455826 | 4456126 | ER_41316 | MYBBP1A | No | n/a | n/a | n/a |
| 315 | 17 | 14109320 | 14109680 | ER_41695 | COX10 | Yes | −0.283019603 | 1.16385E−09 | 1 |
| 316 | 17 | 16322433 | 16322733 | ER_41788 | TRPV2 | Yes | −0.369616508 | 4.66012E−16 | 1 |
| 317 | 17 | 16954969 | 16955288 | ER_41817 | MPRIP | No | n/a | n/a | n/a |
| 318 | 17 | 17628505 | 17628831 | ER_41872 | RAI1 | No | n/a | n/a | n/a |
| 319 | 17 | 17718324 | 17718694 | ER_41889 | SREBF1 | Yes | −0.37884006 | 1.97175E−17 | 1 |
| 320 | 17 | 18139202 | 18139554 | ER_41939 | LLGL1 | Yes | −0.169416014 | 0.000311973 | 1 |
| 321 | 17 | 27295913 | 27296324 | ER_42201 | SEZ6 | Yes | −0.183419447 | 9.10278E−05 | 1 |
| 322 | 17 | 29649808 | 29650164 | ER_42279 | NF1 | Yes | −0.385262742 | 0 | 1 |
| 323 | 17 | 39577357 | 39577695 | ER_42572 | KRT37 | Yes | −0.51802934 | 2.90057E−32 | 1 |
| 324 | 17 | 48048352 | 48048668 | ER_42976 | DLX4 | No | n/a | n/a | n/a |
| 325 | 17 | 48261929 | 48262229 | ER_43003 | COL1A1 | No | n/a | n/a | n/a |
| 326 | 17 | 55371434 | 55371787 | ER_43290 | MSI2 | Yes | −0.576424707 | 0 | 1 |
| 327 | 17 | 55673703 | 55674091 | ER_43343 | MSI2 | Yes | −0.341475332 | 1.28245E−13 | 1 |
| 328 | 1 | 12192916 | 12193471 | ER_444 | TNFRSF8 | No | n/a | n/a | n/a |
| 329 | 1 | 1609934 | 1610467 | ER_45 | SLC35E2B | No | n/a | n/a | n/a |
| 330 | 17 | 59484187 | 59484499 | ER_45541 | TBX2 | No | n/a | n/a | n/a |
| 331 | 1 | 114218048 | 114218488 | ER_4610 | MAGI3 | Yes | −0.376987343 | 5.0018E−17 | 1 |
| 332 | 17 | 65487266 | 65487622 | ER_46920 | PITPNC1 | No | 0.211843351 | 6.11506E−06 | 1 |
| 333 | 17 | 66291281 | 66291581 | ER_46984 | ARSG | Yes | −0.356520016 | 7.89327E−15 | 1 |
| 334 | 17 | 71612589 | 71612889 | ER_47199 | SDK2 | No | n/a | n/a | n/a |
| 335 | 17 | 72439108 | 72439488 | ER_47235 | GPRC5C | No | n/a | n/a | n/a |
| 336 | 17 | 72732636 | 72732936 | ER_47245 | RAB37 | No | 0.157959167 | 0.000782549 | 1 |
| 337 | 17 | 72740901 | 72741201 | ER_47248 | RAB37 | No | 0.231474987 | 7.41968E−07 | 1 |
| 338 | 17 | 73500571 | 73500871 | ER_47351 | CASKIN2 | No | n/a | n/a | n/a |
| 339 | 17 | 73641528 | 73641923 | ER_47388 | RECQL5 | Yes | −0.357417469 | 6.61148E−15 | 1 |
| 340 | 17 | 73696463 | 73696795 | ER_47396 | SAP30BP | Yes | −0.156918701 | 0.000848229 | 1 |
| 341 | 17 | 73805905 | 73806251 | ER_47422 | UNK | No | n/a | n/a | n/a |
| 342 | 17 | 73872405 | 73872705 | ER_47428 | TRIM47 | Yes | −0.203451408 | 1.42337E−05 | 2 |
| 343 | 17 | 74494140 | 74494511 | ER_47474 | RHBDF2 | No | 0.255975124 | 4.08406E−08 | 2 |
| 344 | 17 | 74684239 | 74684546 | ER_47498 | MXRA7 | Yes | −0.158712948 | 0.000737942 | 1 |
| 345 | 17 | 75181779 | 75182110 | ER_47526 | SEC14L1 | No | 0.183371901 | 9.38792E−05 | 1 |
| 346 | 17 | 75473604 | 75473943 | ER_47548 | Sep-09 | No | n/a | n/a | n/a |
| 347 | 17 | 76498871 | 76499229 | ER_47612 | DNAH17 | No | n/a | n/a | n/a |
| 348 | 17 | 76522848 | 76523165 | ER_47614 | DNAH17 | No | n/a | n/a | n/a |
| 349 | 17 | 76858208 | 76858508 | ER_47631 | TIMP2 | Yes | −0.172720063 | 0.000236668 | 1 |
| 350 | 17 | 76973124 | 76973427 | ER_47643 | LGALS3BP | Yes | −0.285188503 | 8.60401E−10 | 1 |
| 351 | 17 | 78522351 | 78522735 | ER_47743 | RPTOR | Yes | −0.211839663 | 6.11738E−06 | 1 |
| 352 | 17 | 78667839 | 78668195 | ER_47754 | RPTOR | Yes | −0.28719267 | 5.41578E−10 | 2 |
| 353 | 17 | 78791604 | 78791917 | ER_47760 | RPTOR | Yes | −0.235434315 | 4.73923E−07 | 1 |
| 354 | 17 | 78793449 | 78793890 | ER_47761 | RPTOR | No | n/a | n/a | n/a |
| 355 | 17 | 78796720 | 78797088 | ER_47762 | RPTOR | No | n/a | n/a | n/a |
| 356 | 17 | 79018691 | 79019077 | ER_47773 | BAIAP2 | Yes | −0.250472546 | 8.04182E−08 | 1 |
| 357 | 17 | 79251153 | 79251492 | ER_47785 | SLC38A10 | No | n/a | n/a | n/a |
| 358 | 17 | 79961758 | 79962058 | ER_47823 | ASPSCR1 | No | n/a | n/a | n/a |
| 359 | 17 | 80162846 | 80163196 | ER_47840 | CCDC57 | Yes | −0.224436269 | 1.61508E−06 | 1 |
| 360 | 17 | 80419732 | 80420082 | ER_47870 | NARF | No | n/a | n/a | n/a |
| 361 | 18 | 3446546 | 3446906 | ER_48111 | TGIF1 | No | n/a | n/a | n/a |
| 362 | 1 | 114521332 | 114522214 | ER_4835 | OLFML3 | No | n/a | n/a | n/a |
| 363 | 18 | 74536197 | 74536522 | ER_49880 | ZNF236 | Yes | −0.317765849 | 6.70861E−12 | 1 |
| 364 | 19 | 930678 | 930978 | ER_49971 | ARID3A | No | n/a | n/a | n/a |
| 365 | 19 | 1169031 | 1169402 | ER_49996 | SBNO2 | No | n/a | n/a | n/a |
| 366 | 19 | 1496339 | 1496639 | ER_50033 | REEP6 | Yes | −0.24403709 | 1.74201E−07 | 3 |
| 367 | 19 | 1907761 | 1908143 | ER_50049 | SCAMP4 | Yes | −0.311880585 | 1.68785E−11 | 2 |
| 368 | 19 | 2167382 | 2167682 | ER_50060 | DOT1L | No | n/a | n/a | n/a |
| 369 | 19 | 2624590 | 2624934 | ER_50099 | GNG7 | No | n/a | n/a | n/a |
| 370 | 19 | 3374719 | 3375065 | ER_50124 | NFIC | Yes | −0.335512035 | 2.66239E−13 | 1 |
| 371 | 19 | 6276448 | 6276748 | ER_50272 | MLLT1 | No | n/a | n/a | n/a |
| 372 | 19 | 7684955 | 7685267 | ER_50326 | XAB2 | No | n/a | n/a | n/a |
| 373 | 19 | 11617772 | 11618120 | ER_50514 | ECSIT | No | n/a | n/a | n/a |
| 374 | 19 | 14066552 | 14066906 | ER_50640 | DCAF15 | No | n/a | n/a | n/a |
| 375 | 19 | 14544916 | 14545316 | ER_50670 | PKN1 | Yes | −0.210598637 | 6.94619E−06 | 1 |
| 376 | 19 | 15590207 | 15590541 | ER_50728 | PGLYRP2 | Yes | −0.526755523 | 1.7199E−33 | 4 |
| 377 | 19 | 15622532 | 15622947 | ER_50734 | CYP4F22 | No | n/a | n/a | n/a |
| 378 | 19 | 16603746 | 16604192 | ER_50800 | CALR3 | No | n/a | n/a | n/a |
| 379 | 19 | 17407033 | 17407362 | ER_50855 | ABHD8 | No | n/a | n/a | n/a |
| 380 | 19 | 33167485 | 33167785 | ER_51224 | RGS9BP | No | n/a | n/a | n/a |
| 381 | 19 | 33624481 | 33624781 | ER_51258 | WDR88 | No | n/a | n/a | n/a |
| 382 | 19 | 35531859 | 35532247 | ER_51354 | HPN | Yes | −0.269976176 | 5.90098E−09 | 3 |
| 383 | 19 | 35800367 | 35800739 | ER_51381 | MAG | Yes | −0.511574789 | 2.22575E−31 | 1 |
| 384 | 19 | 35801186 | 35801551 | ER_51382 | MAG | Yes | −0.160341682 | 0.000640132 | 1 |
| 385 | 19 | 35940219 | 35940897 | ER_51391 | FFAR2 | No | n/a | n/a | n/a |
| 386 | 19 | 39222375 | 39222739 | ER_51519 | CAPN12 | No | n/a | n/a | n/a |
| 387 | 19 | 41633774 | 41634080 | ER_51655 | CYP2F1 | No | n/a | n/a | n/a |
| 388 | 19 | 45843797 | 45844097 | ER_51894 | KLC3 | Yes | −0.288529952 | 5.37546E−10 | 2 |
| 389 | 19 | 45848214 | 45848514 | ER_51895 | KLC3 | No | n/a | n/a | n/a |
| 390 | 19 | 49059555 | 49059855 | ER_52131 | SULT2B1 | No | n/a | n/a | n/a |
| 391 | 19 | 50458158 | 50458475 | ER_52226 | SIGLEC11 | No | n/a | n/a | n/a |
| 392 | 19 | 50969943 | 50970265 | ER_52248 | FAM71E1 | No | n/a | n/a | n/a |
| 393 | 19 | 51568129 | 51568429 | ER_52287 | KLK13 | Yes | −0.255470367 | 3.89102E−08 | 3 |
| 394 | 19 | 54600186 | 54600532 | ER_52363 | OSCAR | No | n/a | n/a | n/a |
| 395 | 19 | 55880474 | 55880774 | ER_52411 | IL11 | No | n/a | n/a | n/a |
| 396 | 19 | 56047757 | 56048080 | ER_52419 | SBK2 | Yes | −0.328477521 | 8.80677E−13 | 2 |
| 397 | 2 | 3452679 | 3453034 | ER_52628 | TRAPPC12 | No | n/a | n/a | n/a |
| 398 | 2 | 19555335 | 19555655 | ER_53364 | OSR1 | No | 0.211392327 | 6.09547E−06 | 1 |
| 399 | 2 | 25094778 | 25095111 | ER_53566 | ADCY3 | No | n/a | n/a | n/a |
| 400 | 2 | 25562754 | 25563086 | ER_53585 | DNMT3A | No | n/a | n/a | n/a |
| 401 | 2 | 26199821 | 26200281 | ER_53612 | KIF3C | No | n/a | n/a | n/a |
| 402 | 2 | 26947077 | 26947447 | ER_53662 | KCNK3 | Yes | −0.3517283 | 1.49994E−14 | 3 |
| 403 | 2 | 27319112 | 27319450 | ER_53686 | KHK | No | n/a | n/a | n/a |
| 404 | 2 | 28549119 | 28549437 | ER_53751 | BRE | No | n/a | n/a | n/a |
| 405 | 2 | 45998277 | 45998784 | ER_54551 | PRKCE | Yes | −0.165247828 | 0.000438935 | 1 |
| 406 | 2 | 46361686 | 46362029 | ER_54598 | PRKCE | Yes | −0.154901111 | 0.000990347 | 1 |
| 407 | 2 | 47236009 | 47236309 | ER_54678 | TTC7A | No | 0.203893879 | 0.000013625 | 1 |
| 408 | 2 | 54760101 | 54760460 | ER_54825 | SPTBN1 | No | n/a | n/a | n/a |
| 409 | 1 | 16074063 | 16074363 | ER_550 | TMEM82 | Yes | −0.15474423 | 0.000990014 | 1 |
| 410 | 1 | 115211543 | 115212151 | ER_5516 | DENND2C | No | n/a | n/a | n/a |
| 411 | 2 | 85280957 | 85281340 | ER_55852 | KCMF1 | No | n/a | n/a | n/a |
| 412 | 2 | 85621930 | 85622230 | ER_55867 | CAPG | No | n/a | n/a | n/a |
| 413 | 1 | 16251091 | 16251574 | ER_559 | SPEN | No | n/a | n/a | n/a |
| 414 | 2 | 95719289 | 95719611 | ER_56072 | MAL | No | 0.264684405 | 1.18981E−08 | 1 |
| 415 | 2 | 97508396 | 97508742 | ER_56156 | ANKRD23 | No | n/a | n/a | n/a |
| 416 | 2 | 106007690 | 106008035 | ER_56517 | FHL2 | Yes | −0.20727681 | 9.72463E−06 | 1 |
| 417 | 2 | 113875147 | 113875494 | ER_56808 | IL1RN | No | n/a | n/a | n/a |
| 418 | 2 | 121036360 | 121036660 | ER_56956 | RALB | No | n/a | n/a | n/a |
| 419 | 2 | 121036666 | 121036969 | ER_56957 | RALB | No | n/a | n/a | n/a |
| 420 | 2 | 129066951 | 129067320 | ER_57178 | HS6ST1 | No | n/a | n/a | n/a |
| 421 | 1 | 116219030 | 116219646 | ER_5750 | VANGL1 | Yes | −0.289419108 | 4.73818E−10 | 1 |
| 422 | 2 | 175499224 | 175499524 | ER_59117 | WIPF1 | Yes | −0.320473352 | 4.35639E−12 | 2 |
| 423 | 1 | 16950505 | 16950951 | ER_599 | CROCCP2 | No | n/a | n/a | n/a |
| 424 | 2 | 197158771 | 197159125 | ER_59918 | HECW2 | No | n/a | n/a | n/a |
| 425 | 1 | 17035334 | 17035751 | ER_603 | ESPNP | No | n/a | n/a | n/a |
| 426 | 2 | 216478034 | 216478350 | ER_60685 | LINC00607 | No | n/a | n/a | n/a |
| 427 | 2 | 220007041 | 220007402 | ER_60937 | NHEJ1 | No | n/a | n/a | n/a |
| 428 | 2 | 224624781 | 224625214 | ER_61179 | AP1S3 | Yes | −0.292051154 | 3.25296E−10 | 1 |
| 429 | 2 | 236447665 | 236448023 | ER_61661 | AGAP1 | Yes | −0.18315725 | 9.56943E−05 | 1 |
| 430 | 2 | 239169600 | 239169933 | ER_61869 | PER2 | No | n/a | n/a | n/a |
| 431 | 2 | 240186821 | 240187208 | ER_61904 | HDAC4 | No | 0.178834332 | 0.000140089 | 1 |
| 432 | 2 | 240241009 | 240241336 | ER_61909 | HDAC4 | No | n/a | n/a | n/a |
| 433 | 2 | 241807610 | 241808275 | ER_61956 | AGXT | Yes | −0.350729914 | 1.79926E−14 | 6 |
| 434 | 2 | 241832846 | 241833163 | ER_61959 | C2orf54 | Yes | −0.342330286 | 8.10744E−14 | 1 |
| 435 | 2 | 241975925 | 241976252 | ER_61978 | SNED1 | No | 0.321003133 | 4.00127E−12 | 3 |
| 436 | 2 | 242138256 | 242138571 | ER_61990 | ANO7 | Yes | −0.164860403 | 0.000452908 | 1 |
| 437 | 2 | 242500199 | 242500499 | ER_62006 | BOK | Yes | −0.326941071 | 1.52312E−12 | 1 |
| 438 | 20 | 17595352 | 17595711 | ER_62469 | RRBP1 | No | n/a | n/a | n/a |
| 439 | 20 | 18035798 | 18036353 | ER_62490 | OVOL2 | Yes | −0.264697876 | 1.18771E−08 | 1 |
| 440 | 20 | 30432800 | 30433405 | ER_62916 | FOXS1 | No | n/a | n/a | n/a |
| 441 | 20 | 32888452 | 32889022 | ER_63021 | AHCY | Yes | −0.255937198 | 4.10339E−08 | 1 |
| 442 | 20 | 34205164 | 34205464 | ER_63067 | SPAG4 | Yes | −0.253362831 | 5.64449E−08 | 1 |
| 443 | 20 | 35093680 | 35094313 | ER_63095 | DLGAP4 | Yes | −0.226710124 | 1.25956E−06 | 2 |
| 444 | 20 | 36767493 | 36768090 | ER_63162 | TGM2 | No | n/a | n/a | n/a |
| 445 | 1 | 118727703 | 118728260 | ER_6319 | SPAG17 | Yes | −0.468876303 | 5.62552E−26 | 9 |
| 446 | 1 | 17634485 | 17634785 | ER_634 | PADI4 | Yes | −0.304565675 | 4.10341E−11 | 4 |
| 447 | 20 | 44048102 | 44048440 | ER_63559 | PIGT | Yes | −0.483985863 | 0 | 1 |
| 448 | 20 | 44330526 | 44330841 | ER_63571 | WFDC13 | Yes | −0.372474463 | 2.94819E−16 | 1 |
| 449 | 20 | 47278241 | 47278894 | ER_65136 | PREX1 | No | 0.312514399 | 1.52976E−11 | 1 |
| 450 | 1 | 144989293 | 144989659 | ER_6529 | PDE4DIP | No | n/a | n/a | n/a |
| 451 | 20 | 49411019 | 49411779 | ER_65780 | BCAS4 | No | n/a | n/a | n/a |
| 452 | 1 | 17887921 | 17888496 | ER_662 | ARHGEF10L | No | n/a | n/a | n/a |
| 453 | 1 | 18006751 | 18007051 | ER_669 | ARHGEF10L | Yes | −0.203444297 | 1.42437E−05 | 1 |
| 454 | 20 | 58325746 | 58326345 | ER_68282 | PHACTR3 | No | n/a | n/a | n/a |
| 455 | 20 | 60510103 | 60510565 | ER_68459 | CDH4 | No | n/a | n/a | n/a |
| 456 | 20 | 60924954 | 60925254 | ER_68486 | LAMA5 | Yes | −0.172436407 | 0.000242396 | 1 |
| 457 | 20 | 60932217 | 60932572 | ER_68489 | LAMA5 | No | n/a | n/a | n/a |
| 458 | 20 | 61451571 | 61451871 | ER_68537 | COL9A3 | No | n/a | n/a | n/a |
| 459 | 20 | 62282532 | 62282832 | ER_68587 | STMN3 | No | n/a | n/a | n/a |
| 460 | 1 | 2036450 | 2036863 | ER_69 | PRKCZ | Yes | −0.217921669 | 3.24696E−06 | 2 |
| 461 | 21 | 42212292 | 42212834 | ER_69514 | DSCAM | No | n/a | n/a | n/a |
| 462 | 21 | 43107677 | 43108088 | ER_69583 | LINC00111 | No | n/a | n/a | n/a |
| 463 | 21 | 43735388 | 43735910 | ER_69642 | TFF3 | Yes | −0.64300849 | 7.33107E−54 | 3 |
| 464 | 21 | 44897853 | 44898245 | ER_69724 | LINC00313 | No | n/a | n/a | n/a |
| 465 | 21 | 46321203 | 46321769 | ER_69832 | ITGB2 | No | 0.678975995 | 0 | 2 |
| 466 | 21 | 46325777 | 46326117 | ER_69834 | ITGB2 | No | n/a | n/a | n/a |
| 467 | 21 | 46331748 | 46332340 | ER_69835 | ITGB2 | No | n/a | n/a | n/a |
| 468 | 21 | 46409729 | 46410331 | ER_69844 | LINC00163 | No | n/a | n/a | n/a |
| 469 | 21 | 46953747 | 46954295 | ER_69890 | SLC19A1 | No | n/a | n/a | n/a |
| 470 | 22 | 18919539 | 18919839 | ER_69990 | PRODH | Yes | −0.498286049 | 1.29155E−29 | 1 |
| 471 | 22 | 19755289 | 19755589 | ER_70033 | TBX1 | No | 0.390493517 | 0 | 1 |
| 472 | 22 | 19879093 | 19879435 | ER_70044 | TXNRD2 | No | n/a | n/a | n/a |
| 473 | 1 | 151554445 | 151555180 | ER_7042 | TUFT1 | Yes | −0.426720988 | 0 | 1 |
| 474 | 22 | 35695235 | 35695568 | ER_70647 | TOM1 | No | n/a | n/a | n/a |
| 475 | 22 | 38610003 | 38610303 | ER_70793 | MAFF | No | n/a | n/a | n/a |
| 476 | 22 | 39759998 | 39760357 | ER_70842 | SYNGR1 | Yes | −0.473134386 | 0 | 4 |
| 477 | 22 | 40404687 | 40405045 | ER_70880 | FAM83F | No | n/a | n/a | n/a |
| 478 | 22 | 43525157 | 43525457 | ER_70998 | BIK | No | n/a | n/a | n/a |
| 479 | 22 | 46921821 | 46922121 | ER_71163 | CELSR1 | Yes | −0.358860373 | 4.95469E−15 | 1 |
| 480 | 22 | 50450954 | 50451412 | ER_71287 | IL17REL | No | n/a | n/a | n/a |
| 481 | 22 | 50720235 | 50720566 | ER_71300 | PLXNB2 | Yes | −0.26103668 | 2.16012E−08 | 3 |
| 482 | 22 | 50738759 | 50739090 | ER_71305 | PLXNB2 | Yes | −0.165338693 | 0.000435717 | 1 |
| 483 | 3 | 8693628 | 8694000 | ER_71572 | C3orf32 | No | n/a | n/a | n/a |
| 484 | 3 | 9757534 | 9757834 | ER_71643 | CPNE9 | No | n/a | n/a | n/a |
| 485 | 3 | 11550571 | 11550871 | ER_71746 | ATG7 | No | n/a | n/a | n/a |
| 486 | 3 | 11643071 | 11643504 | ER_71757 | VGLL4 | Yes | −0.222133443 | 2.07217E−06 | 2 |
| 487 | 3 | 12985852 | 12986194 | ER_71863 | IQSEC1 | No | n/a | n/a | n/a |
| 488 | 3 | 12994633 | 12995020 | ER_71864 | IQSEC1 | Yes | −0.187690211 | 6.35759E−05 | 1 |
| 489 | 3 | 14920746 | 14921046 | ER_72049 | FGD5 | No | n/a | n/a | n/a |
| 490 | 3 | 15310870 | 15311256 | ER_72070 | SH3BP5 | No | n/a | n/a | n/a |
| 491 | 3 | 15687178 | 15687551 | ER_72120 | BTD | Yes | −0.367472893 | 7.79001E−16 | 1 |
| 492 | 1 | 154298772 | 154299072 | ER_7259 | ATP8B2 | No | n/a | n/a | n/a |
| 493 | 1 | 154377532 | 154377832 | ER_7267 | IL6R | Yes | −0.226436213 | 1.29803E−06 | 1 |
| 494 | 3 | 38067281 | 38067610 | ER_73005 | PLCD1 | No | n/a | n/a | n/a |
| 495 | 3 | 46734109 | 46734463 | ER_73287 | ALS2CL | No | n/a | n/a | n/a |
| 496 | 3 | 48589861 | 48590161 | ER_73356 | PFKFB4 | Yes | −0.248525803 | 1.01827E−07 | 1 |
| 497 | 1 | 155161639 | 155162099 | ER_7336 | MUC1 | Yes | −0.415072063 | 3.62028E−20 | 3 |
| 498 | 3 | 52280070 | 52280428 | ER_73463 | PPM1M | Yes | −0.1589276 | 0.000725678 | 4 |
| 499 | 3 | 58028409 | 58028709 | ER_73666 | FLNB | Yes | −0.538338329 | 0 | 1 |
| 500 | 1 | 155912401 | 155912737 | ER_7372 | RXFP4 | No | n/a | n/a | n/a |
| 501 | 1 | 156095999 | 156096299 | ER_7395 | LMNA | Yes | −0.275981939 | 3.04831E−09 | 1 |
| 502 | 3 | 61793488 | 61793816 | ER_73972 | PTPRG | No | n/a | n/a | n/a |
| 503 | 1 | 156822285 | 156822644 | ER_7451 | INSRR | No | 0.245053419 | 1.40657E−07 | 1 |
| 504 | 1 | 161646681 | 161647242 | ER_7530 | FCGR2B | No | 0.257178093 | 3.51452E−08 | 1 |
| 505 | 1 | 2174671 | 2174976 | ER_76 | SKI | No | n/a | n/a | n/a |
| 506 | 3 | 63922231 | 63922573 | ER_76144 | ATXN7 | No | n/a | n/a | n/a |
| 507 | 3 | 66519928 | 66520259 | ER_77324 | LRIG1 | Yes | −0.507588811 | 0 | 1 |
| 508 | 3 | 66543250 | 66543644 | ER_77334 | LRIG1 | Yes | −0.352144949 | 1.83363E−14 | 1 |
| 509 | 3 | 69942380 | 69942719 | ER_77420 | MITF | No | n/a | n/a | n/a |
| 510 | 3 | 99721769 | 99722069 | ER_78150 | FILIP1L | No | n/a | n/a | n/a |
| 511 | 3 | 99833502 | 99833835 | ER_78166 | C3orf26 | No | n/a | n/a | n/a |
| 512 | 1 | 171226439 | 171226888 | ER_7821 | FMO1 | No | 0.188726331 | 5.78252E−05 | 1 |
| 513 | 3 | 112359914 | 112360214 | ER_78589 | CCDC80 | No | n/a | n/a | n/a |
| 514 | 3 | 122057821 | 122058174 | ER_78781 | CSTA | No | n/a | n/a | n/a |
| 515 | 3 | 124493174 | 124493574 | ER_78936 | ITGB5 | No | n/a | n/a | n/a |
| 516 | 3 | 126200679 | 126200979 | ER_79014 | UROC1 | No | n/a | n/a | n/a |
| 517 | 3 | 126678959 | 126679283 | ER_79028 | CHCHD6 | No | n/a | n/a | n/a |
| 518 | 3 | 129295579 | 129295977 | ER_79235 | PLXND1 | No | n/a | n/a | n/a |
| 519 | 3 | 133174831 | 133175237 | ER_79397 | BFSP2 | Yes | −0.17660393 | 0.000166181 | 1 |
| 520 | 3 | 160787663 | 160788036 | ER_80595 | PPM1L | No | 0.259407701 | 2.65537E−08 | 1 |
| 521 | 3 | 168870086 | 168870443 | ER_80932 | MECOM | No | n/a | n/a | n/a |
| 522 | 3 | 169758098 | 169758589 | ER_80993 | GPR160 | Yes | −0.525559204 | 0 | 1 |
| 523 | 3 | 183959578 | 183959923 | ER_81693 | VWA5B2 | Yes | −0.24189682 | 2.05298E−07 | 1 |
| 524 | 3 | 184052187 | 184052556 | ER_81701 | EIF4G1 | No | n/a | n/a | n/a |
| 525 | 3 | 195531202 | 195531699 | ER_82319 | MUC4 | No | n/a | n/a | n/a |
| 526 | 3 | 195603560 | 195603892 | ER_82326 | TNK2 | No | n/a | n/a | n/a |
| 527 | 3 | 196388403 | 196388727 | ER_82377 | LRRC33 | No | 0.496219142 | 0 | 2 |
| 528 | 4 | 680920 | 681252 | ER_82472 | MFSD7 | Yes | −0.324124136 | 2.41555E−12 | 1 |
| 529 | 4 | 757427 | 757729 | ER_82479 | PCGF3 | Yes | −0.384333816 | 0 | 1 |
| 530 | 4 | 965412 | 965736 | ER_82491 | DGKQ | Yes | −0.182120866 | 0.000104932 | 1 |
| 531 | 4 | 987571 | 987878 | ER_82493 | IDUA | Yes | −0.316721564 | 7.91431E−12 | 2 |
| 532 | 4 | 1239191 | 1239531 | ER_82503 | CTBP1 | No | n/a | n/a | n/a |
| 533 | 4 | 1729872 | 1730230 | ER_82525 | TACC3 | No | n/a | n/a | n/a |
| 534 | 4 | 1986291 | 1986591 | ER_82549 | WHSC2 | Yes | −0.180201515 | 0.000124297 | 1 |
| 535 | 4 | 2798112 | 2798444 | ER_82594 | SH3BP2 | No | n/a | n/a | n/a |
| 536 | 4 | 3341090 | 3341444 | ER_82632 | RGS12 | Yes | −0.390930721 | 0 | 1 |
| 537 | 4 | 6928789 | 6929118 | ER_82798 | TBC1D14 | No | n/a | n/a | n/a |
| 538 | 4 | 7219719 | 7220019 | ER_82822 | SORCS2 | No | n/a | n/a | n/a |
| 539 | 4 | 8062466 | 8062766 | ER_82882 | ABLIM2 | No | 0.165497975 | 0.000422976 | 3 |
| 540 | 4 | 8130159 | 8130480 | ER_82884 | ABLIM2 | No | 0.307594935 | 3.26131E−11 | 1 |
| 541 | 4 | 8412544 | 8412844 | ER_82916 | ACOX3 | No | n/a | n/a | n/a |
| 542 | 4 | 119910484 | 119910799 | ER_85984 | SYNPO2 | No | n/a | n/a | n/a |
| 543 | 5 | 373069 | 373625 | ER_88159 | AHRR | No | n/a | n/a | n/a |
| 544 | 5 | 429733 | 430313 | ER_88163 | AHRR | No | n/a | n/a | n/a |
| 545 | 5 | 610077 | 610670 | ER_88173 | CEP72 | No | n/a | n/a | n/a |
| 546 | 5 | 672776 | 673076 | ER_88176 | TPPP | Yes | −0.173994887 | 0.000208036 | 1 |
| 547 | 5 | 1207117 | 1207709 | ER_88196 | SLC6A19 | No | 0.176046676 | 0.000174395 | 1 |
| 548 | 5 | 1443109 | 1443488 | ER_88204 | SLC6A3 | No | n/a | n/a | n/a |
| 549 | 5 | 37840402 | 37840702 | ER_89386 | GDNF | No | n/a | n/a | n/a |
| 550 | 5 | 43603889 | 43604448 | ER_89613 | NNT | Yes | −0.217287839 | 3.28333E−06 | 1 |
| 551 | 5 | 52385969 | 52386507 | ER_89737 | ITGA2 | Yes | −0.21285208 | 5.51202E−06 | 1 |
| 552 | 1 | 201252202 | 201252787 | ER_9013 | PKP1 | Yes | −0.160895926 | 0.000612605 | 5 |
| 553 | 5 | 95226771 | 95227393 | ER_91036 | ELL2 | Yes | −0.204999399 | 1.22105E−05 | 1 |
| 554 | 1 | 203096814 | 203097402 | ER_9205 | ADORA1 | Yes | −0.156376349 | 0.000873099 | 1 |
| 555 | 1 | 203122931 | 203123270 | ER_9215 | ADORA1 | Yes | −0.17524604 | 0.000186865 | 1 |
| 556 | 5 | 138299601 | 138299901 | ER_92405 | SIL1 | Yes | −0.199535932 | 2.08704E−05 | 1 |
| 557 | 5 | 139033837 | 139034156 | ER_92444 | CXXC5 | Yes | −0.689848806 | 0 | 1 |
| 558 | 5 | 140012702 | 140013307 | ER_92479 | CD14 | No | 0.172842269 | 0.00023424 | 1 |
| 559 | 5 | 159687745 | 159688359 | ER_93120 | CCNJL | No | n/a | n/a | n/a |
| 560 | 5 | 176239632 | 176240113 | ER_93644 | UNC5A | No | n/a | n/a | n/a |
| 561 | 5 | 176816546 | 176817069 | ER_93670 | SLC34A1 | Yes | −0.338284799 | 1.64743E−13 | 2 |
| 562 | 5 | 176874917 | 176875279 | ER_93675 | PRR7-AS1 | No | n/a | n/a | n/a |
| 563 | 5 | 177029224 | 177029524 | ER_93689 | B4GALT7 | Yes | −0.203926933 | 1.35805E−05 | 1 |
| 564 | 5 | 177547872 | 177548392 | ER_93712 | N4BP3 | Yes | −0.200270092 | 1.94359E−05 | 1 |
| 565 | 6 | 3139405 | 3139938 | ER_93950 | BPHL | Yes | −0.183369268 | 9.39013E−05 | 1 |
| 566 | 6 | 3723005 | 3723548 | ER_93997 | PXDC1 | No | n/a | n/a | n/a |
| 567 | 1 | 205633387 | 205633954 | ER_9471 | SLC45A3 | No | n/a | n/a | n/a |
| 568 | 6 | 30698796 | 30699096 | ER_95075 | FLOT1 | Yes | −0.158565326 | 0.000746488 | 1 |
| 569 | 6 | 31477979 | 31478310 | ER_95117 | MICB | Yes | −0.294476318 | 2.29255E−10 | 2 |
| 570 | 6 | 31743735 | 31744048 | ER_95131 | VWA7 | No | n/a | n/a | n/a |
| 571 | 6 | 31744689 | 31744989 | ER_95132 | VWA7 | No | n/a | n/a | n/a |
| 572 | 6 | 31833129 | 31833429 | ER_95141 | SLC44A4 | Yes | −0.191804667 | 4.35012E−05 | 3 |
| 573 | 6 | 31868596 | 31868896 | ER_95144 | C2 | No | 0.289384343 | 4.76165E−10 | 8 |
| 574 | 6 | 32050977 | 32051543 | ER_95152 | TNXB | No | 0.160713551 | 0.000630719 | 1 |
| 575 | 6 | 32135005 | 32135578 | ER_95158 | EGFL8 | Yes | −0.241470361 | 2.3573E−07 | 17 |
| 576 | 6 | 33173123 | 33173423 | ER_95186 | HSD17B8 | Yes | −0.254003888 | 5.21528E−08 | 2 |
| 577 | 6 | 33288112 | 33288670 | ER_95195 | DAXX | Yes | −0.24031322 | 2.69879E−07 | 13 |
| 578 | 6 | 33993601 | 33993995 | ER_95239 | GRM4 | Yes | −0.292034242 | 2.68505E−10 | 1 |
| 579 | 6 | 34511677 | 34512101 | ER_95268 | SPDEF | Yes | −0.522863026 | 0 | 1 |
| 580 | 6 | 40363002 | 40363510 | ER_95568 | LRFN2 | Yes | −0.408390902 | 1.62215E−19 | 1 |
| 581 | 6 | 42108932 | 42109278 | ER_95663 | C6orf132 | Yes | −0.240988721 | 2.28671E−07 | 1 |
| 582 | 6 | 43463227 | 43463527 | ER_95722 | TJAP1 | No | n/a | n/a | n/a |
| 583 | 6 | 43603250 | 43603772 | ER_95728 | MAD2L1BP | No | 0.180749995 | 0.000118445 | 1 |
| 584 | 6 | 52268606 | 52268913 | ER_96019 | PAQR8 | No | 0.418197621 | 0 | 1 |
| 585 | 6 | 56580975 | 56581555 | ER_96201 | RNU6-71 | No | n/a | n/a | n/a |
| 586 | 6 | 64281501 | 64282055 | ER_96269 | PTP4A1 | Yes | −0.259535604 | 2.31991E−08 | 5 |
| 587 | 6 | 109267245 | 109267600 | ER_97322 | ARMC2 | Yes | −0.189528837 | 5.37126E−05 | 1 |
| 588 | 6 | 138425688 | 138426269 | ER_98290 | PERP | No | n/a | n/a | n/a |
| 589 | 6 | 151937170 | 151937602 | ER_98798 | CCDC170 | No | n/a | n/a | n/a |
| 590 | 6 | 152124722 | 152125100 | ER_98841 | ESR1 | Yes | −0.636001231 | 0 | 1 |
| 591 | 7 | 921963 | 922279 | ER_99284 | GET4 | Yes | −0.206381595 | 0.000010638 | 1 |
| 592 | 7 | 923724 | 924024 | ER_99285 | GET4 | Yes | −0.309385232 | 2.48011E−11 | 1 |
| 593 | 7 | 927894 | 928206 | ER_99286 | GET4 | Yes | −0.265790218 | 1.17348E−08 | 2 |
| 594 | 7 | 940841 | 941164 | ER_99287 | ADAP1 | No | n/a | n/a | n/a |
| 595 | 7 | 955121 | 955452 | ER_99289 | ADAP1 | No | n/a | n/a | n/a |
| 596 | 7 | 966854 | 967230 | ER_99290 | ADAP1 | No | 0.267014125 | 1.00097E−08 | 3 |
| 597 | 7 | 1004933 | 1005233 | ER_99293 | COX19 | No | n/a | n/a | n/a |
| 598 | 7 | 1026106 | 1026406 | ER_99296 | CYP2W1 | No | n/a | n/a | n/a |
| 599 | 7 | 1027843 | 1028143 | ER_99297 | CYP2W1 | No | n/a | n/a | n/a |
| 600 | 7 | 1054343 | 1054643 | ER_99302 | C7orf50 | No | n/a | n/a | n/a |
| 601 | 7 | 1139221 | 1139554 | ER_99313 | C7orf50 | No | n/a | n/a | n/a |
| 602 | 1 | 217886370 | 217887036 | ER_9936 | SPATA17 | Yes | −0.496347538 | 0 | 1 |
| 603 | 7 | 1753468 | 1753819 | ER_99371 | ELFN1 | No | 0.31719091 | 5.63452E−12 | 3 |
| 604 | 7 | 1782517 | 1782817 | ER_99372 | ELFN1 | No | n/a | n/a | n/a |
| 605 | 7 | 1891218 | 1891549 | ER_99377 | MAD1L1 | No | n/a | n/a | n/a |
| 606 | 7 | 1931906 | 1932296 | ER_99382 | MAD1L1 | No | n/a | n/a | n/a |
| 607 | 7 | 1959815 | 1960115 | ER_99385 | MAD1L1 | No | n/a | n/a | n/a |
| 608 | 7 | 1961938 | 1962358 | ER_99386 | MAD1L1 | No | n/a | n/a | n/a |
| 609 | 7 | 1967307 | 1967639 | ER_99387 | MAD1L1 | No | n/a | n/a | n/a |
| 610 | 7 | 1968165 | 1968519 | ER_99388 | MAD1L1 | No | n/a | n/a | n/a |
| 611 | 7 | 2191489 | 2191835 | ER_99406 | MAD1L1 | No | n/a | n/a | n/a |
| 612 | 7 | 2673205 | 2673630 | ER_99460 | TTYH3 | Yes | −0.20153074 | 1.71888E−05 | 1 |
| 613 | 7 | 2702609 | 2702937 | ER_99466 | TTYH3 | No | 0.168197637 | 0.000344996 | 1 |
| 614 | 7 | 2731506 | 2731863 | ER_99473 | AMZ1 | No | n/a | n/a | n/a |
| 615 | 7 | 4876684 | 4876984 | ER_99580 | RADIL | No | n/a | n/a | n/a |
| 616 | 7 | 5436737 | 5437105 | ER_99601 | TNRC18 | No | n/a | n/a | n/a |
| 617 | 7 | 5526272 | 5526875 | ER_99612 | FBXL18 | No | n/a | n/a | n/a |

[symbol]

indicates data missing or illegible when filed

In one example, the method of detecting differential methylation comprises identifying differential methylation at one or more CpG dinucleotide sequences within one or more regions set forth in rows 1-617 of Table 2 in a subject suffering from ESR1 positive cancer relative to a reference level of methylation for the corresponding one or more CpG dinucleotide sequences. For example, the method may comprise detecting differential methylation at a single CpG dinucleotide sequence within any one of the genomic regions defined in Table 2 in a subject suffering from ESR1 positive breast cancer relative to a reference level of methylation for the corresponding CpG dinucleotide sequence.

In another example, the method of detecting differential methylation comprises detecting differential methylation at two or more CpG dinucleotides within any genomic region defined in Table 2 in a subject suffering from ESR1 positive breast cancer relative to a reference level of methylation for the corresponding CpG dinucleotide sequences. For example, the method may comprise detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 2 in a subject suffering from ESR1 positive breast cancer relative to a reference level of methylation for the corresponding CpG dinucleotide sequences. The two or more CpG dinucleotides may be consecutive (i.e., contiguous) within a genomic region. Alternatively, the two or more CpG dinucleotides may not be consecutive (i.e., may not be contiguous) within any genomic region.

In yet another example, the method of detecting differential methylation comprises detecting differential methylation of at least one CpG dinucleotide within two or more different genomic regions set forth in Table 2 in a subject suffering from ESR1 positive breast cancer relative to a reference level of methylation for the corresponding CpG dinucleotide sequences. For example, the method may comprise detecting differential methylation at a CpG dinucleotide within two or more, or three or more, or four or more, or five or more, or six or more, or seven or more, or eight or more, or nine or more, or 10 or more different genomic regions set forth in Table 2 in a subject suffering from ESR1 positive breast cancer relative to a reference level of methylation for the corresponding CpG dinucleotide sequences.

In one example, identifying differential methylation at one or more CpG dinucleotide sequences within one or more regions set forth in rows 1-617 of Table 2 in a subject relative to a reference level of methylation for the corresponding one or more CpG dinucleotide sequences is indicative of the subject's likely response to endocrine therapy.

Detecting differential methylation at a single CpG dinucleotide sequence within any one of the genomic regions defined in Table 2 may be indicative of the subject's likely response to endocrine therapy.

Alternatively, detecting differential methylation at two or more CpG dinucleotides within any genomic region defined in Table 2 may be indicative of the subject's likely response to endocrine therapy. For example, detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 2 may be indicative of the subject's likely response to endocrine therapy. The two or more CpG dinucleotides may be consecutive (i.e., contiguous) within a genomic region. Alternatively, the two or more CpG dinucleotides may not be consecutive (i.e., may not be contiguous) within any genomic region.

Detecting differential methylation of at least one CpG dinucleotide within two or more different genomic regions set forth in Table 2 may be indicative of the subject's likely response to endocrine therapy. For example, detecting differential methylation at a CpG dinucleotide within two or more, or three or more, or four or more, or five or more, or six or more, or seven or more, or eight or more, or nine or more, or 10 or more different genomic regions set forth in Table 2 may be indicative of the subject's likely response to endocrine therapy.

In one example, identifying differential methylation at one or more CpG dinucleotide sequences within one or more regions set forth in rows 1-617 of Table 2 in a subject relative to a reference level of methylation for the corresponding one or more CpG dinucleotide sequences is indicative of the subject having breast cancer which is refractory to endocrine therapy.

Detecting differential methylation at a single CpG dinucleotide sequence within any one of the genomic regions defined in Table 2 may be indicative of the subject having breast cancer which is refractory to endocrine therapy.

Alternatively, detecting differential methylation at two or more CpG dinucleotides within any genomic region defined in Table 2 may be indicative of the subject having breast cancer which is refractory to endocrine therapy. For example, detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 2 may be indicative of the subject having breast cancer which is refractory to endocrine therapy. The two or more CpG dinucleotides may be consecutive (i.e., contiguous) within a genomic region. Alternatively, the two or more CpG dinucleotides may not be consecutive (i.e., may not be contiguous) within any genomic region.

Detecting differential methylation of at least one CpG dinucleotide within two or more different genomic regions set forth in Table 2 may be indicative of the subject having breast cancer which is refractory to endocrine therapy. For example, detecting differential methylation at a CpG dinucleotide within two or more, or three or more, or four or more, or five or more, or six or more, or seven or more, or eight or more, or nine or more, or 10 or more different genomic regions set forth in Table 2 may be indicative of the subject having breast cancer which is refractory to endocrine therapy.

In one example, identifying differential methylation at one or more CpG dinucleotide sequences within one or more regions set forth in rows 1-617 of Table 2 in a subject relative to a reference level of methylation for the corresponding one or more CpG dinucleotide sequences is indicative of the likely therapeutic outcome and/or of the progression of the ESR1 positive breast cancer.

Detecting differential methylation at a single CpG dinucleotide sequence within any one of the genomic regions defined in Table 2 may be indicative of the likely therapeutic outcome and/or of the progression of the ESR1 positive breast cancer.

Alternatively, detecting differential methylation at two or more CpG dinucleotides within any genomic region defined in Table 2 may be indicative of the likely therapeutic outcome and/or of the progression of the ESR1 positive breast cancer. For example, detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 2 may be indicative of the likely therapeutic outcome and/or of the progression of the ESR1 positive breast cancer. The two or more CpG dinucleotides may be consecutive (i.e., contiguous) within a genomic region. Alternatively, the two or more CpG dinucleotides may not be consecutive (i.e., may not be contiguous) within any genomic region.

Detecting differential methylation of at least one CpG dinucleotide within two or more different genomic regions set forth in Table 2 may be indicative of the likely therapeutic outcome and/or of the progression of the ESR1 positive breast cancer. For example, detecting differential methylation at a CpG dinucleotide within two or more, or three or more, or four or more, or five or more, or six or more, or seven or more, or eight or more, or nine or more, or 10 or more different genomic regions set forth in Table 2 may be indicative of the likely therapeutic outcome and/or of the progression of the ESR1 positive breast cancer.

Of the ESR1 binding sites set forth in Table 1, hypermethylation of 328 of those sites correlated with reduced expression of the gene(s) with which the respective ESR1 binding sites were most closely associated (Table 3). These 328 ESR1 binding sites represented 291 unique genes. Accordingly, the method of the disclosure may comprise determining the methylation status of one or more CpG dinucleotide sequences within one or more of the ESR1 binding sites set forth in Table 3. The ESR1 binding sites set forth in Table 3 are also defined with reference to hg19. Thus, the nucleotide sequences of each of the regions identified in Table 3 (or in any of the Tables disclosed herein) can be identified by reference to hg19, using the “start” and “end” positions described in Table 3 (or in any of the Tables disclosed herein).

For each of the ESR1 binding sites set forth in Table 3, the following information is provided:

### (i) Chromosome ID (Column 2);

(ii) genomic coordinates of ESR1-binding site with respect to hg19 (Columns 3-4);
(iii) ESR1-binding site name (Column 5);
(iv) name of gene which ESR1-binding site is most closely associated (Column 6);
(v) inverse correlation between methylation and gene expression in TCGA (Column 7);
(vi) spearman's RHO (Column 8);
(vii) correlation P-value (Column 9); and
(viii) number of HM450K probes per ESR1 binding site (column 10).

TABLE 3
Hypermethylated ESR1 binding sites in estrogen enhancer regions correlating with reduced gene expression

| Row No. | Chromosome | Start | End | ESR1 site name | Gene | Probe name | Spearman'sRHO | Correlation P-value |
|---|---|---|---|---|---|---|---|---|
| 1 | 7 | 74024952 | 74024954 | ER_101675 | GTF2IRD1 | cg13446584 | −0.335580192 | 3.57E−13 |
| 2 | 7 | 75362553 | 75362555 | ER_101699 | HIP1 | cg27531731 | −0.25958627 | 2.60E−08 |
| 3 | 1 | 223854446 | 223854448 | ER_10205 | CAPN8 | cg02313130 | −0.510874625 | 2.77E−31 |
| 4 | 7 | 100800361 | 100800363 | ER_102605 | AP1S1 | cg24080086 | −0.308429704 | 2.87E−11 |
| 5 | 7 | 101959149 | 101959151 | ER_102722 | SH2B2 | cg27275553 | −0.285866301 | 7.82E−10 |
| 6 | 7 | 102080712 | 102080714 | ER_102733 | ORAI2 | cg22402224 | −0.18527387 | 7.92E−05 |
| 7 | 1 | 27240513 | 27240515 | ER_1029 | NR0B2 | cg16762386 | −0.224802698 | 1.46E−06 |
| 8 | 1 | 228598445 | 228598447 | ER_10389 | TRIM17 | cg13472406 | −0.249438828 | 8.25E−08 |
| 9 | 7 | 139345114 | 139345116 | ER_104192 | HIPK2 | cg14630106 | −0.216367357 | 3.82E−06 |
| 10 | 1 | 230882386 | 230882388 | ER_10488 | CAPN9 | cg27412093 | −0.512610884 | 1.61E−31 |
| 11 | 7 | 157916686 | 157916688 | ER_104936 | PTPRN2 | cg27324698 | −0.190965163 | 4.56E−05 |
| 12 | 1 | 230896050 | 230896052 | ER_10494 | CAPN9 | cg18126320 | −0.361156359 | 2.61E−15 |
| 13 | 1 | 231113418 | 231113420 | ER_10502 | TTC13 | cg00018229 | −0.215328471 | 4.26E−06 |
| 14 | 8 | 17394957 | 17394959 | ER_105282 | SLC7A2 | cg16906456 | −0.584122654 | 0 |
| 15 | 8 | 21948559 | 21948561 | ER_105469 | FAM160B2 | cg20576064 | −0.316480707 | 8.22E−12 |
| 16 | 8 | 28223456 | 28223458 | ER_105673 | ZNF395 | cg09759289 | −0.30437003 | 5.32E−11 |
| 17 | 8 | 62568171 | 62568173 | ER_106497 | ASPH | cg11390489 | −0.174035493 | 0.000211725 |
| 18 | 8 | 67425315 | 67425317 | ER_106600 | C8orf46 | cg12897502 | −0.446493874 | 1.96E−23 |
| 19 | 8 | 98881926 | 98881928 | ER_108508 | MATN2 | cg16202564 | −0.199715554 | 2.05E−05 |
| 20 | 8 | 101017844 | 101017846 | ER_108722 | RGS22 | cg16308540 | −0.424259141 | 4.35E−21 |
| 21 | 1 | 246869081 | 246869083 | ER_10884 | SCCPDH | cg14094521 | −0.6475097 | 0 |
| 22 | 8 | 102526826 | 102526828 | ER_109025 | GRHL2 | cg04225551 | −0.470255952 | 0 |
| 23 | 10 | 416314 | 416316 | ER_10930 | DIP2C | cg01117269 | −0.266239142 | 1.11E−08 |
| 24 | 10 | 579258 | 579260 | ER_10949 | DIP2C | cg23596826 | −0.190689534 | 4.83E−05 |
| 25 | 8 | 124179874 | 124179876 | ER_110727 | FAM83A | cg12002745 | −0.372495492 | 2.94E−16 |
| 26 | 8 | 124193816 | 124193818 | ER_110732 | FAM83A | cg02715629 | −0.43367174 | 4.64E−22 |
| 27 | 8 | 124194964 | 124194966 | ER_110733 | FAM83A | cg15425827 | −0.211481781 | 6.04E−06 |
| 28 | 8 | 126082719 | 126082721 | ER_111167 | KIAA0196 | cg16593865 | −0.361311348 | 3.00E−15 |
| 29 | 10 | 5538777 | 5538779 | ER_11143 | CALML5 | cg05876550 | −0.271567366 | 5.50E−09 |
| 30 | 8 | 129103557 | 129103559 | ER_111781 | PVT1 | cg19508622 | −0.35335898 | 1.46E−14 |
| 31 | 8 | 142139838 | 142139840 | ER_112470 | DENND3 | cg01287592 | −0.208632932 | 8.48E−06 |
| 32 | 8 | 142247863 | 142247865 | ER_112484 | SLC45A4 | cg27109043 | −0.18946273 | 5.40E−05 |
| 33 | 8 | 143395440 | 143395442 | ER_112563 | TSNARE1 | cg12692919 | −0.175513031 | 0.000186653 |
| 34 | 8 | 143823733 | 143823735 | ER_112619 | SLURP1 | cg20862496 | −0.621291369 | 2.14E−49 |
| 35 | 8 | 143851426 | 143851428 | ER_112624 | LYNX1 | cg17770910 | −0.204416812 | 1.29E−05 |
| 36 | 8 | 145048766 | 145048768 | ER_112777 | PLEC | cg07598407 | −0.233518914 | 5.89E−07 |
| 37 | 8 | 145086426 | 145086428 | ER_112784 | SPATC1 | cg03288742 | −0.27978868 | 1.54E−09 |
| 38 | 8 | 145721559 | 145721561 | ER_112829 | PPP1R16A | cg00994323 | −0.166529283 | 0.000395527 |
| 39 | 8 | 145728629 | 145728631 | ER_112830 | GPT | cg19352605 | −0.510988729 | 0 |
| 40 | 9 | 33443364 | 33443366 | ER_113615 | AQP3 | cg14473416 | −0.375082083 | 1.01E−16 |
| 41 | 9 | 34372820 | 34372822 | ER_113641 | KIAA1161 | cg20241254 | −0.200056757 | 1.98E−05 |
| 42 | 9 | 95834819 | 95834821 | ER_115179 | SUSD3 | cg13916469 | −0.290895198 | 3.84E−10 |
| 43 | 1 | 32039681 | 32039683 | ER_1168 | TINAGL1 | cg03211098 | −0.32389197 | 2.51E−12 |
| 44 | 1 | 3398885 | 3398887 | ER_117 | ARHGEF16 | cg22007638 | −0.482192011 | 0 |
| 45 | 9 | 129433968 | 129433970 | ER_117042 | LMX1B | cg14916924 | −0.571087528 | 0 |
| 46 | 9 | 136728553 | 136728555 | ER_117634 | VAV2 | cg14178533 | −0.239045592 | 3.13E−07 |
| 47 | 9 | 137253048 | 137253050 | ER_117681 | RXRA | cg13677043 | −0.244453224 | 1.66E−07 |
| 48 | 9 | 137258919 | 137258921 | ER_117685 | RXRA | cg13786567 | −0.22430142 | 1.64E−06 |
| 49 | 9 | 139949062 | 139949064 | ER_117897 | ENTPD2 | cg13981310 | −0.367248102 | 8.21E−16 |
| 50 | 9 | 140374284 | 140374286 | ER_117940 | PNPLA7 | cg01701837 | −0.227579527 | 1.14E−06 |
| 51 | 10 | 24496597 | 24496599 | ER_11835 | KIAA1217 | cg18829521 | −0.283127061 | 1.15E−09 |
| 52 | X | 16889682 | 16889684 | ER_118379 | RBBP7 | cg14719055 | −0.328262296 | 1.22E−12 |
| 53 | X | 18708952 | 18708954 | ER_118438 | PPEF1 | cg17198372 | −0.26765505 | 9.21E−09 |
| 54 | X | 133792598 | 133792600 | ER_120294 | PLAC1 | cg02831668 | −0.351896922 | 1.45E−14 |
| 55 | 1 | 37503349 | 37503351 | ER_1301 | GRIK3 | cg17815567 | −0.292808764 | 2.40E−10 |
| 56 | 10 | 71213994 | 71213996 | ER_13175 | TSPAN15 | cg06791973 | −0.424739404 | 3.89E−21 |
| 57 | 10 | 74020427 | 74020429 | ER_13281 | DDIT4 | cg16407699 | −0.428856044 | 0 |
| 58 | 10 | 76859205 | 76859207 | ER_13395 | DUSP13 | cg10963664 | −0.374994214 | 1.79E−16 |
| 59 | 10 | 88475770 | 88475772 | ER_13815 | LDB3 | cg19185706 | −0.20457813 | 1.27E−05 |
| 60 | 10 | 95228649 | 95228651 | ER_14079 | MYOF | cg26808637 | −0.293781665 | 2.54E−10 |
| 61 | 10 | 99331807 | 99331809 | ER_14202 | UBTD1 | cg10343327 | −0.162973315 | 0.000527081 |
| 62 | 10 | 102792834 | 102792836 | ER_14340 | SFXN3 | cg15428620 | −0.550035375 | 0 |
| 63 | 10 | 105238872 | 105238874 | ER_14449 | CALHM3 | cg04804772 | −0.200511144 | 1.83E−05 |
| 64 | 10 | 112259304 | 112259306 | ER_14629 | DUSP5 | cg09357462 | −0.47604403 | 0 |
| 65 | 10 | 121415188 | 121415190 | ER_15018 | BAG3 | cg01303372 | −0.449812197 | 0 |
| 66 | 10 | 123900860 | 123900862 | ER_15113 | TACC2 | cg24181174 | −0.165628275 | 0.000425606 |
| 67 | 10 | 126315760 | 126315762 | ER_15199 | FAM53B | cg24916358 | −0.247233418 | 1.19E−07 |
| 68 | 10 | 126700660 | 126700662 | ER_15221 | CTBP2 | cg04839409 | −0.267400497 | 9.52E−09 |
| 69 | 1 | 44300941 | 44300943 | ER_1532 | ST3GAL3 | cg09989037 | −0.203979608 | 1.35E−05 |
| 70 | 10 | 128606856 | 128606858 | ER_15340 | DOCK1 | cg26717418 | −0.286907293 | 6.76E−10 |
| 71 | 10 | 134225119 | 134225121 | ER_15443 | PWWP2B | cg23622878 | −0.185156404 | 8.00E−05 |
| 72 | 10 | 134261412 | 134261414 | ER_15450 | C10orf91 | cg15834395 | −0.370354575 | 4.46E−16 |
| 73 | 10 | 134332382 | 134332384 | ER_15463 | INPP5A | cg00149455 | −0.161112302 | 0.000611156 |
| 74 | 10 | 134420622 | 134420624 | ER_15478 | INPP5A | cg00805619 | −0.177054833 | 0.000163475 |
| 75 | 10 | 134499265 | 134499267 | ER_15480 | INPP5A | cg19649172 | −0.207945257 | 9.09E−06 |
| 76 | 11 | 1507262 | 1507264 | ER_15583 | MOB2 | cg11611244 | −0.155896803 | 0.000917668 |
| 77 | 11 | 1777551 | 1777553 | ER_15595 | CTSD | cg25050723 | −0.389507306 | 0 |
| 78 | 11 | 10764591 | 10764593 | ER_15808 | CTR9 | cg09645818 | −0.483304247 | 0 |
| 79 | 11 | 33744822 | 33744824 | ER_16397 | CD59 | cg04188351 | −0.304313667 | 5.36E−11 |
| 80 | 11 | 46374123 | 46374125 | ER_16803 | DGKZ | cg12087471 | −0.292581725 | 3.01E−10 |
| 81 | 11 | 61467285 | 61467287 | ER_17096 | DAGLA | cg07011711 | −0.222108817 | 2.08E−06 |
| 82 | 11 | 62437614 | 62437616 | ER_17173 | C11orf48 | cg02375832 | −0.162346349 | 0.000554124 |
| 83 | 11 | 64008173 | 64008175 | ER_17271 | FKBP2 | cg03764062 | −0.155263384 | 0.000963309 |
| 84 | 11 | 64034958 | 64034960 | ER_17276 | PLCB3 | cg18375707 | −0.229382466 | 9.37E−07 |
| 85 | 11 | 65582809 | 65582811 | ER_17439 | OVOL1 | cg20077042 | −0.282987867 | 1.17E−09 |
| 86 | 11 | 67914462 | 67914464 | ER_17656 | SUV420H1 | cg27606671 | −0.287097977 | 6.58E−10 |
| 87 | 11 | 69061910 | 69061912 | ER_17743 | MYEOV | cg22779330 | −0.161570899 | 0.000580565 |
| 88 | 11 | 70917158 | 70917160 | ER_17848 | SHANK2 | cg21810373 | −0.315797247 | 9.16E−12 |
| 89 | 11 | 85469036 | 85469038 | ER_18506 | SYTL2 | cg11429283 | −0.459623471 | 0 |
| 90 | 1 | 6445631 | 6445633 | ER_192 | ACOT7 | cg16429975 | −0.346507797 | 3.86E−14 |
| 91 | 11 | 120106597 | 120106599 | ER_19293 | POU2F3 | cg21886186 | −0.250931346 | 7.60E−08 |
| 92 | 12 | 48340532 | 48340534 | ER_21318 | TMEM106C | cg26115531 | −0.34357773 | 8.83E−14 |
| 93 | 12 | 51236581 | 51236583 | ER_21514 | TMPRSS12 | cg02956274 | −0.385063766 | 2.36E−17 |
| 94 | 12 | 52542741 | 52542743 | ER_21611 | KRT80 | cg00230924 | −0.278960719 | 2.03E−09 |
| 95 | 12 | 52579701 | 52579703 | ER_21620 | KRT80 | cg23345977 | −0.156387208 | 0.000883714 |
| 96 | 12 | 53300292 | 53300294 | ER_21729 | KRT8 | cg26357344 | −0.358090657 | 5.78E−15 |
| 97 | 12 | 53553085 | 53553087 | ER_21786 | CSAD | cg05242244 | −0.462318596 | 0 |
| 98 | 12 | 58160842 | 58160844 | ER_22088 | CYP27B1 | cg24722099 | −0.255597575 | 4.28E−08 |
| 99 | 12 | 63193646 | 63193648 | ER_22367 | PPM1H | cg21177112 | −0.167030751 | 0.000379657 |
| 100 | 12 | 117471699 | 117471701 | ER_25133 | FBXW8 | cg04605980 | −0.351039676 | 1.70E−14 |
| 101 | 12 | 122019524 | 122019526 | ER_25371 | KDM2B | cg03486832 | −0.15698494 | 0.0008439 |
| 102 | 12 | 122458892 | 122458894 | ER_25409 | BCL7A | cg03496157 | −0.226142681 | 1.34E−06 |
| 103 | 12 | 123446271 | 123446273 | ER_25491 | ABCB9 | cg03131767 | −0.188629804 | 5.83E−05 |
| 104 | 12 | 123449185 | 123449187 | ER_25493 | ABCB9 | cg18473642 | −0.416828462 | 0 |
| 105 | 12 | 124844873 | 124844875 | ER_25578 | NCOR2 | cg13712197 | −0.158414149 | 0.000755335 |
| 106 | 12 | 124880021 | 124880023 | ER_25592 | NCOR2 | cg03905247 | −0.219796114 | 2.66E−06 |
| 107 | 12 | 125004852 | 125004854 | ER_25616 | NCOR2 | cg13146040 | −0.17339707 | 0.000223506 |
| 108 | 13 | 34208893 | 34208895 | ER_26636 | STARD13 | cg00527307 | −0.292609379 | 3.00E−10 |
| 109 | 13 | 41590152 | 41590154 | ER_26934 | ELF1 | cg20033981 | −0.28858071 | 4.44E−10 |
| 110 | 13 | 113652068 | 113652070 | ER_28798 | MCF2L | cg06072822 | −0.416344509 | 0 |
| 111 | 13 | 113677187 | 113677189 | ER_28802 | MCF2L | cg18050804 | −0.233597269 | 5.84E−07 |
| 112 | 13 | 113980034 | 113980036 | ER_28813 | GRTP1 | cg08351085 | −0.561680883 | 8.92E−39 |
| 113 | 1 | 8824176 | 8824178 | ER_290 | RERE | cg20263853 | −0.425083186 | 0 |
| 114 | 14 | 23623479 | 23623481 | ER_29019 | SLC7A8 | cg05357695 | −0.354356515 | 1.20E−14 |
| 115 | 14 | 23623683 | 23623685 | ER_29020 | SLC7A8 | cg23543481 | −0.296853548 | 1.62E−10 |
| 116 | 14 | 38054164 | 38054166 | ER_29794 | FOXA1 | cg05555111 | −0.477419115 | 0 |
| 117 | 14 | 64932677 | 64932679 | ER_31140 | AKAP5 | cg26524899 | −0.214691628 | 4.56E−06 |
| 118 | 14 | 69263266 | 69263268 | ER_31542 | ZFP36L1 | cg20016914 | −0.251562921 | 7.04E−08 |
| 119 | 14 | 74214182 | 74214184 | ER_31871 | C14orf43 | cg22851561 | −0.432667091 | 0 |
| 120 | 14 | 74238380 | 74238382 | ER_31884 | C14orf43 | cg26217402 | −0.195733971 | 3.01E−05 |
| 121 | 14 | 76447326 | 76447328 | ER_32084 | TGFB3 | cg18298494 | −0.303016542 | 6.51E−11 |
| 122 | 14 | 88942953 | 88942955 | ER_32519 | PTPN21 | cg12215094 | −0.168486692 | 0.000336879 |
| 123 | 14 | 93412665 | 93412667 | ER_32818 | ITPK1 | cg16157895 | −0.361876948 | 2.67E−15 |
| 124 | 14 | 93474065 | 93474067 | ER_32832 | ITPK1 | cg05939041 | −0.290394125 | 4.12E−10 |
| 125 | 14 | 95047654 | 95047656 | ER_32949 | SERPINA5 | cg13984563 | −0.671013091 | 0 |
| 126 | 14 | 95078598 | 95078600 | ER_32959 | SERPINA3 | cg08057786 | −0.324660895 | 2.21E−12 |
| 127 | 14 | 95615730 | 95615732 | ER_32995 | DICER1 | cg01901579 | −0.296402122 | 1.73E−10 |
| 128 | 14 | 96692186 | 96692188 | ER_33117 | BDKRB2 | cg06616512 | −0.167100809 | 0.000377488 |
| 129 | 14 | 100055668 | 100055670 | ER_33296 | CCDC85C | cg11773456 | −0.175193161 | 0.000191832 |
| 130 | 14 | 100618490 | 100618492 | ER_33370 | DEGS2 | cg20904336 | −0.394516582 | 0 |
| 131 | 14 | 105181901 | 105181903 | ER_33695 | INF2 | cg08043200 | −0.165915618 | 0.00041579 |
| 132 | 14 | 105215643 | 105215645 | ER_33701 | ADSSL1 | cg15983585 | −0.301120862 | 6.94E−11 |
| 133 | 14 | 105434297 | 105434299 | ER_33730 | AHNAK2 | cg09796640 | −0.17137013 | 0.000265106 |
| 134 | 14 | 105992114 | 105992116 | ER_33807 | TMEM121 | cg07037750 | −0.284994395 | 8.84E−10 |
| 135 | 15 | 50519373 | 50519375 | ER_34615 | SLC27A2 | cg05028948 | −0.342165772 | 1.14E−13 |
| 136 | 15 | 63671691 | 63671693 | ER_35532 | CA12 | cg08947167 | −0.663688479 | 0 |
| 137 | 15 | 63673012 | 63673014 | ER_35533 | CA12 | cg23931734 | −0.553637039 | 0 |
| 138 | 15 | 70384231 | 70384233 | ER_35866 | TLE3 | cg02009766 | −0.624063592 | 0 |
| 139 | 15 | 74233032 | 74233034 | ER_36102 | LOXL1 | cg16581800 | −0.219977053 | 2.61E−06 |
| 140 | 15 | 80878743 | 80878745 | ER_36421 | ARNT2 | cg08371852 | −0.270986754 | 5.94E−09 |
| 141 | 15 | 89028368 | 89028370 | ER_36781 | MRPS11 | cg01608635 | −0.158819879 | 0.000731809 |
| 142 | 15 | 96866813 | 96866815 | ER_37215 | NR2F2 | cg11122899 | −0.210236502 | 6.87E−06 |
| 143 | 15 | 99236766 | 99236768 | ER_37297 | IGF1R | cg02613818 | −0.537894014 | 0 |
| 144 | 15 | 99272175 | 99272177 | ER_37301 | IGF1R | cg12402183 | −0.588333638 | 0 |
| 145 | 16 | 635622 | 635624 | ER_37540 | PIGQ | cg03804128 | −0.400189696 | 0 |
| 146 | 16 | 1232362 | 1232364 | ER_37605 | CACNA1H | cg05272807 | −0.232335435 | 6.74E−07 |
| 147 | 16 | 1275784 | 1275786 | ER_37610 | TPSG1 | cg13997068 | −0.53048809 | 5.01E−34 |
| 148 | 16 | 1353626 | 1353628 | ER_37624 | UBE2I | cg06908857 | −0.192504589 | 4.07E−05 |
| 149 | 16 | 1479500 | 1479502 | ER_37638 | CCDC154 | cg07000567 | −0.210795649 | 6.48E−06 |
| 150 | 16 | 1828145 | 1828147 | ER_37665 | SPSB3 | cg01676844 | −0.168403597 | 0.000339194 |
| 151 | 16 | 2879943 | 2879945 | ER_37757 | ZG16B | cg05461841 | −0.331448616 | 7.20E−13 |
| 152 | 16 | 3704736 | 3704738 | ER_37830 | DNASE1 | cg08778316 | −0.225124178 | 1.40E−06 |
| 153 | 16 | 3707038 | 3707040 | ER_37831 | DNASE1 | cg04332526 | −0.190279859 | 4.86E−05 |
| 154 | 16 | 4368123 | 4368125 | ER_37883 | GLIS2 | cg04123578 | −0.344446475 | 7.56E−14 |
| 155 | 16 | 4741494 | 4741496 | ER_37916 | MGRN1 | cg06125591 | −0.267451066 | 9.46E−09 |
| 156 | 16 | 4838558 | 4838560 | ER_37923 | Sep-12 | cg11353547 | −0.290559784 | 3.33E−10 |
| 157 | 16 | 14030551 | 14030553 | ER_38174 | ERCC4 | cg03927470 | −0.286422287 | 7.24E−10 |
| 158 | 16 | 14580862 | 14580864 | ER_38229 | PARN | cg03176203 | −0.328987106 | 1.09E−12 |
| 159 | 16 | 16108801 | 16108803 | ER_38301 | ABCC1 | cg00649632 | −0.199363421 | 2.12E−05 |
| 160 | 16 | 22103595 | 22103597 | ER_38518 | VWA3A | cg02843201 | −0.43583238 | 2.75E−22 |
| 161 | 16 | 24748338 | 24748340 | ER_38621 | TNRC6A | cg09018299 | −0.363523046 | 1.88E−15 |
| 162 | 16 | 24856161 | 24856163 | ER_38625 | SLC5A11 | cg04399565 | −0.17392406 | 0.0002093 |
| 163 | 16 | 28608287 | 28608289 | ER_38739 | SULT1A2 | cg00931491 | −0.449317709 | 0 |
| 164 | 16 | 72995176 | 72995178 | ER_39877 | ZFHX3 | cg16563255 | −0.209502467 | 7.77E−06 |
| 165 | 16 | 83848108 | 83848110 | ER_40473 | HSBP1 | cg08394248 | −0.313956513 | 1.22E−11 |
| 166 | 16 | 84401246 | 84401248 | ER_40523 | ATP2C2 | cg06786050 | −0.192394761 | 4.12E−05 |
| 167 | 16 | 85669461 | 85669463 | ER_40724 | KIAA0182 | cg09396032 | −0.459019682 | 0 |
| 168 | 16 | 85723488 | 85723490 | ER_40737 | GINS2 | cg08871354 | −0.29702843 | 1.58E−10 |
| 169 | 16 | 85787023 | 85787025 | ER_40750 | C16orf74 | cg07897180 | −0.272675914 | 4.75E−09 |
| 170 | 1 | 109373103 | 109373105 | ER_4085 | AKNAD1 | cg03884543 | −0.246004825 | 1.25E−07 |
| 171 | 16 | 87813068 | 87813070 | ER_40908 | KLHDC4 | cg05710032 | −0.172678581 | 0.000237498 |
| 172 | 16 | 87890566 | 87890568 | ER_40918 | SLC7A5 | cg03879320 | −0.483398404 | 0 |
| 173 | 1 | 11020630 | 11020632 | ER_411 | C1orf127 | cg21157465 | −0.239407483 | 2.76E−07 |
| 174 | 16 | 89900193 | 89900195 | ER_41112 | SPIRE2 | cg16769976 | −0.231466954 | 7.43E−07 |
| 175 | 17 | 152088 | 152090 | ER_41149 | RPH3AL | cg04897931 | −0.376348525 | 6.44E−17 |
| 176 | 17 | 1901436 | 1901438 | ER_41216 | RTN4RL1 | cg19550533 | −0.201776075 | 1.68E−05 |
| 177 | 17 | 1988011 | 1988013 | ER_41224 | SMG6 | cg14439774 | −0.176716395 | 0.000168319 |
| 178 | 17 | 3870414 | 3870416 | ER_41284 | ATP2A3 | cg25112590 | −0.33237346 | 6.16E−13 |
| 179 | 17 | 7283816 | 7283818 | ER_41423 | TNK1 | cg02503376 | −0.398328696 | 1.46E−18 |
| 180 | 17 | 14109670 | 14109672 | ER_41695 | COX10 | cg26751588 | −0.283019603 | 1.16E−09 |
| 181 | 17 | 16322481 | 16322483 | ER_41788 | TRPV2 | cg26719625 | −0.369616508 | 4.66E−16 |
| 182 | 17 | 17718524 | 17718526 | ER_41889 | SREBF1 | cg19619576 | −0.37884006 | 1.97E−17 |
| 183 | 17 | 18139505 | 18139507 | ER_41939 | LLGL1 | cg09658183 | −0.169416014 | 0.000311973 |
| 184 | 17 | 18280848 | 18280850 | ER_41950 | EVPLL | cg17549878 | −0.460071152 | 5.93E−25 |
| 185 | 17 | 27296200 | 27296202 | ER_42201 | SEZ6 | cg24778016 | −0.183419447 | 9.10E−05 |
| 186 | 17 | 29649960 | 29649962 | ER_42279 | NF1 | cg20368567 | −0.385262742 | 0 |
| 187 | 17 | 39577628 | 39577630 | ER_42572 | KRT37 | cg18068256 | −0.51802934 | 2.90E−32 |
| 188 | 17 | 39662979 | 39662981 | ER_42574 | KRT13 | cg10742225 | −0.361827589 | 2.30E−15 |
| 189 | 17 | 39678106 | 39678108 | ER_42579 | KRT19 | cg21513437 | −0.217626424 | 3.35E−06 |
| 190 | 17 | 39685911 | 39685913 | ER_42583 | KRT19 | cg08966188 | −0.41138725 | 0 |
| 191 | 17 | 39694252 | 39694254 | ER_42588 | KRT19 | cg10851010 | −0.374811464 | 1.10E−16 |
| 192 | 17 | 40932198 | 40932200 | ER_42648 | WNK4 | cg03777083 | −0.473878767 | 1.43E−26 |
| 193 | 17 | 55371726 | 55371728 | ER_43290 | MSI2 | cg26871347 | −0.576424707 | 0 |
| 194 | 17 | 55673926 | 55673928 | ER_43343 | MSI2 | cg26408224 | −0.341475332 | 1.28E−13 |
| 195 | 1 | 114218185 | 114218187 | ER_4610 | MAGI3 | cg20616821 | −0.376987343 | 5.00E−17 |
| 196 | 17 | 64940744 | 64940746 | ER_46841 | CACNG4 | cg07432111 | −0.194033617 | 3.53E−05 |
| 197 | 17 | 64954455 | 64954457 | ER_46847 | CACNG4 | cg22285671 | −0.445992326 | 0 |
| 198 | 17 | 66291421 | 66291423 | ER_46984 | ARSG | cg08815110 | −0.356520016 | 7.89E−15 |
| 199 | 17 | 73641808 | 73641810 | ER_47388 | RECQL5 | cg07251887 | −0.357417469 | 6.61E−15 |
| 200 | 17 | 73696508 | 73696510 | ER_47396 | SAP30BP | cg18563987 | −0.156918701 | 0.000848229 |
| 201 | 17 | 73872583 | 73872585 | ER_47428 | TRIM47 | cg10644544 | −0.203451408 | 1.42E−05 |
| 202 | 17 | 74684503 | 74684505 | ER_47498 | MXRA7 | cg27546012 | −0.158712948 | 0.000737942 |
| 203 | 17 | 76858247 | 76858249 | ER_47631 | TIMP2 | cg27549186 | −0.172720063 | 0.000236668 |
| 204 | 17 | 76973327 | 76973329 | ER_47643 | LGALS3BP | cg26928788 | −0.285188503 | 8.60E−10 |
| 205 | 17 | 78522608 | 78522610 | ER_47743 | RPTOR | cg02082642 | −0.211839663 | 6.12E−06 |
| 206 | 17 | 78668007 | 78668009 | ER_47754 | RPTOR | cg13102028 | −0.28719267 | 5.42E−10 |
| 207 | 17 | 78791722 | 78791724 | ER_47760 | RPTOR | cg27460531 | −0.235434315 | 4.74E−07 |
| 208 | 17 | 79018697 | 79018699 | ER_47773 | BAIAP2 | cg17027476 | −0.250472546 | 8.04E−08 |
| 209 | 17 | 80163173 | 80163175 | ER_47840 | CCDC57 | cg01777586 | −0.224436269 | 1.62E−06 |
| 210 | 17 | 80174779 | 80174781 | ER_47845 | CCDC57 | cg07013698 | −0.235217952 | 4.86E−07 |
| 211 | 18 | 74536225 | 74536227 | ER_49880 | ZNF236 | cg06398181 | −0.317765849 | 6.71E−12 |
| 212 | 19 | 1496415 | 1496417 | ER_50033 | REEP6 | cg02300825 | −0.24403709 | 1.74E−07 |
| 213 | 19 | 1907971 | 1907973 | ER_50049 | SCAMP4 | cg19254118 | −0.311880585 | 1.69E−11 |
| 214 | 19 | 3374731 | 3374733 | ER_50124 | NFIC | cg14883993 | −0.335512035 | 2.66E−13 |
| 215 | 19 | 7714474 | 7714476 | ER_50328 | STXBP2 | cg07063348 | −0.368882875 | 5.58E−16 |
| 216 | 19 | 14545200 | 14545202 | ER_50670 | PKN1 | cg13922488 | −0.210598637 | 6.95E−06 |
| 217 | 19 | 15590307 | 15590309 | ER_50728 | PGLYRP2 | cg17752089 | −0.526755523 | 1.72E−33 |
| 218 | 19 | 16045787 | 16045789 | ER_50746 | CYP4F11 | cg03190825 | −0.203840303 | 1.31E−05 |
| 219 | 19 | 35531858 | 35531860 | ER_51354 | HPN | cg21484586 | −0.269976176 | 5.90E−09 |
| 220 | 19 | 35800588 | 35800590 | ER_51381 | MAG | cg02776658 | −0.511574789 | 2.23E−31 |
| 221 | 19 | 35801214 | 35801216 | ER_51382 | MAG | cg04690840 | −0.160341682 | 0.000640132 |
| 222 | 19 | 45843825 | 45843827 | ER_51894 | KLC3 | cg03883348 | −0.288529952 | 5.38E−10 |
| 223 | 19 | 51568259 | 51568261 | ER_52287 | KLK13 | cg03307401 | −0.255470367 | 3.89E−08 |
| 224 | 19 | 56047883 | 56047885 | ER_52419 | SBK2 | cg00149708 | −0.328477521 | 8.81E−13 |
| 225 | 2 | 26947125 | 26947127 | ER_53662 | KCNK3 | cg11273176 | −0.3517283 | 1.50E−14 |
| 226 | 2 | 45998547 | 45998549 | ER_54551 | PRKCE | cg23924526 | −0.165247828 | 0.000438935 |
| 227 | 2 | 46361963 | 46361965 | ER_54598 | PRKCE | cg00518941 | −0.154901111 | 0.000990347 |
| 228 | 1 | 16074113 | 16074115 | ER_550 | TMEM82 | cg12703825 | −0.15474423 | 0.000990014 |
| 229 | 2 | 102012903 | 102012905 | ER_56390 | RFX8 | cg17654419 | −0.224242571 | 1.55E−06 |
| 230 | 2 | 106007814 | 106007816 | ER_56517 | FHL2 | cg02234235 | −0.20727681 | 9.72E−06 |
| 231 | 1 | 116219478 | 116219480 | ER_5750 | VANGL1 | cg04880253 | −0.289419108 | 4.74E−10 |
| 232 | 2 | 175499282 | 175499284 | ER_59117 | WIPF1 | cg03983223 | −0.320473352 | 4.36E−12 |
| 233 | 2 | 224625079 | 224625081 | ER_61179 | AP1S3 | cg02234653 | −0.292051154 | 3.25E−10 |
| 234 | 2 | 236447791 | 236447793 | ER_61661 | AGAP1 | cg10647547 | −0.18315725 | 9.57E−05 |
| 235 | 2 | 241807746 | 241807748 | ER_61956 | AGXT | cg17461448 | −0.350729914 | 1.80E−14 |
| 236 | 2 | 241832899 | 241832901 | ER_61959 | C2orf54 | cg01588581 | −0.342330286 | 8.11E−14 |
| 237 | 2 | 241936843 | 241936845 | ER_61974 | SNED1 | cg16937168 | −0.221441686 | 2.23E−06 |
| 238 | 2 | 242138540 | 242138542 | ER_61990 | ANO7 | cg01583021 | −0.164860403 | 0.000452908 |
| 239 | 2 | 242500483 | 242500485 | ER_62006 | BOK | cg24828208 | −0.326941071 | 1.52E−12 |
| 240 | 20 | 18036012 | 18036014 | ER_62490 | OVOL2 | cg02113429 | −0.264697876 | 1.19E−08 |
| 241 | 20 | 32447029 | 32447031 | ER_63011 | CHMP4B | cg03217337 | −0.193799476 | 3.61E−05 |
| 242 | 20 | 32888854 | 32888856 | ER_63021 | AHCY | cg00582941 | −0.255937198 | 4.10E−08 |
| 243 | 20 | 34205181 | 34205183 | ER_63067 | SPAG4 | cg27632911 | −0.253362831 | 5.64E−08 |
| 244 | 20 | 35093927 | 35093929 | ER_63095 | DLGAP4 | cg12992443 | −0.226710124 | 1.26E−06 |
| 245 | 1 | 118727833 | 118727835 | ER_6319 | SPAG17 | cg23257935 | −0.468876303 | 5.63E−26 |
| 246 | 1 | 17634542 | 17634544 | ER_634 | PADI4 | cg16091981 | −0.304565675 | 4.10E−11 |
| 247 | 20 | 44048173 | 44048175 | ER_63559 | PIGT | cg21723559 | −0.483985863 | 0 |
| 248 | 20 | 44330620 | 44330622 | ER_63571 | WFDC13 | cg17890298 | −0.372474463 | 2.95E−16 |
| 249 | 20 | 47448544 | 47448546 | ER_65321 | PREX1 | cg18112953 | −0.404365915 | 0 |
| 250 | 20 | 49345566 | 49345568 | ER_65723 | PARD6B | cg03326606 | −0.55204559 | 0 |
| 251 | 20 | 49346623 | 49346625 | ER_65724 | PARD6B | cg07803218 | −0.695503616 | 0 |
| 252 | 1 | 18006886 | 18006888 | ER_669 | ARHGEF10L | cg00172227 | −0.203444297 | 1.42E−05 |
| 253 | 20 | 60925178 | 60925180 | ER_68486 | LAMA5 | cg18668449 | −0.172436407 | 0.000242396 |
| 254 | 1 | 2036507 | 2036509 | ER_69 | PRKCZ | cg17023856 | −0.217921669 | 3.25E−06 |
| 255 | 21 | 43735411 | 43735413 | ER_69642 | TFF3 | cg14283447 | −0.64300849 | 7.33E−54 |
| 256 | 22 | 18919802 | 18919804 | ER_69990 | PRODH | cg11438552 | −0.498286049 | 1.29E−29 |
| 257 | 1 | 151554823 | 151554825 | ER_7042 | TUFT1 | cg20383521 | −0.426720988 | 0 |
| 258 | 22 | 39760058 | 39760060 | ER_70842 | SYNGR1 | cg06397161 | −0.473134386 | 0 |
| 259 | 22 | 46921966 | 46921968 | ER_71163 | CELSR1 | cg19206437 | −0.358860373 | 4.95E−15 |
| 260 | 22 | 50720287 | 50720289 | ER_71300 | PLXNB2 | cg00012194 | −0.26103668 | 2.16E−08 |
| 261 | 22 | 50738889 | 50738891 | ER_71305 | PLXNB2 | cg04089788 | −0.165338693 | 0.000435717 |
| 262 | 3 | 11643367 | 11643369 | ER_71757 | VGLL4 | cg08525922 | −0.222133443 | 2.07E−06 |
| 263 | 3 | 12994820 | 12994822 | ER_71864 | IQSEC1 | cg09361614 | −0.187690211 | 6.36E−05 |
| 264 | 3 | 13517896 | 13517898 | ER_71911 | HDAC11 | cg03190578 | −0.488500124 | 0 |
| 265 | 3 | 15687270 | 15687272 | ER_72120 | BTD | cg21634628 | −0.367472893 | 7.79E−16 |
| 266 | 1 | 19664275 | 19664277 | ER_722 | CAPZB | cg26157446 | −0.160511147 | 0.00064087 |
| 267 | 1 | 154377620 | 154377622 | ER_7267 | IL6R | cg25853020 | −0.226436213 | 1.30E−06 |
| 268 | 3 | 48590039 | 48590041 | ER_73356 | PFKFB4 | cg20732160 | −0.248525803 | 1.02E−07 |
| 269 | 1 | 155161678 | 155161680 | ER_7336 | MUC1 | cg20949223 | −0.415072063 | 3.62E−20 |
| 270 | 3 | 50639078 | 50639080 | ER_73426 | CISH | cg13519902 | −0.25711001 | 3.54E−08 |
| 271 | 3 | 52280123 | 52280125 | ER_73463 | PPM1M | cg05406954 | −0.1589276 | 0.000725678 |
| 272 | 3 | 58028596 | 58028598 | ER_73666 | FLNB | cg02026180 | −0.538338329 | 0 |
| 273 | 1 | 156096177 | 156096179 | ER_7395 | LMNA | cg08881019 | −0.275981939 | 3.05E−09 |
| 274 | 3 | 66519997 | 66519999 | ER_77324 | LRIG1 | cg09716921 | −0.507588811 | 0 |
| 275 | 3 | 66543262 | 66543264 | ER_77334 | LRIG1 | cg24150385 | −0.352144949 | 1.83E−14 |
| 276 | 3 | 133174925 | 133174927 | ER_79397 | BFSP2 | cg05618222 | −0.17660393 | 0.000166181 |
| 277 | 3 | 169758288 | 169758290 | ER_80993 | GPR160 | cg12350863 | −0.525559204 | 0 |
| 278 | 3 | 183959852 | 183959854 | ER_81693 | VWA5B2 | cg24363374 | −0.24189682 | 2.05E−07 |
| 279 | 4 | 681085 | 681087 | ER_82472 | MFSD7 | cg23681017 | −0.324124136 | 2.42E−12 |
| 280 | 4 | 686869 | 686871 | ER_82474 | MFSD7 | cg21498785 | −0.187877471 | 6.25E−05 |
| 281 | 4 | 757455 | 757457 | ER_82479 | PCGF3 | cg26690744 | −0.384333816 | 0 |
| 282 | 4 | 965629 | 965631 | ER_82491 | DGKQ | cg15639776 | −0.182120866 | 0.000104932 |
| 283 | 4 | 987651 | 987653 | ER_82493 | IDUA | cg23332689 | −0.316721564 | 7.91E−12 |
| 284 | 4 | 1986541 | 1986543 | ER_82549 | WHSC2 | cg00248861 | −0.180201515 | 0.000124297 |
| 285 | 4 | 3341176 | 3341178 | ER_82632 | RGS12 | cg21343777 | −0.390930721 | 0 |
| 286 | 4 | 48909056 | 48909058 | ER_83945 | OCIAD2 | cg26134090 | −0.19336451 | 3.76E−05 |
| 287 | 5 | 672844 | 672846 | ER_88176 | TPPP | cg22879098 | −0.173994887 | 0.000208036 |
| 288 | 5 | 7851680 | 7851682 | ER_88404 | C5orf49 | cg01035170 | −0.394008422 | 3.66E−18 |
| 289 | 5 | 43604148 | 43604150 | ER_89613 | NNT | cg09489281 | −0.217287839 | 3.28E−06 |
| 290 | 5 | 52386487 | 52386489 | ER_89737 | ITGA2 | cg08874888 | −0.21285208 | 5.51E−06 |
| 291 | 1 | 201096288 | 201096290 | ER_8996 | TMEM9 | cg06714761 | −0.236629383 | 4.13E−07 |
| 292 | 1 | 201252452 | 201252454 | ER_9013 | PKP1 | cg17463149 | −0.160895926 | 0.000612605 |
| 293 | 5 | 95226945 | 95226947 | ER_91036 | ELL2 | cg19493250 | −0.204999399 | 1.22E−05 |
| 294 | 1 | 203097233 | 203097235 | ER_9205 | ADORA1 | cg12794758 | −0.156376349 | 0.000873099 |
| 295 | 1 | 203123143 | 203123145 | ER_9215 | ADORA1 | cg23257225 | −0.17524604 | 0.000186865 |
| 296 | 5 | 138299676 | 138299678 | ER_92405 | SIL1 | cg24650915 | −0.199535932 | 2.09E−05 |
| 297 | 5 | 139034001 | 139034003 | ER_92444 | CXXC5 | cg22885332 | −0.689848806 | 0 |
| 298 | 5 | 139069448 | 139069450 | ER_92451 | CXXC5 | cg10680621 | −0.223444527 | 1.80E−06 |
| 299 | 1 | 203488763 | 203488765 | ER_9257 | OPTC | cg06122825 | −0.223553288 | 1.67E−06 |
| 300 | 1 | 203595060 | 203595062 | ER_9266 | ATP2B4 | cg21693907 | −0.164075806 | 0.000482481 |
| 301 | 5 | 148513940 | 148513942 | ER_92755 | ABLIM3 | cg16247269 | −0.330183359 | 8.90E−13 |
| 302 | 5 | 176816678 | 176816680 | ER_93670 | SLC34A1 | cg21145248 | −0.338284799 | 1.65E−13 |
| 303 | 5 | 177029443 | 177029445 | ER_93689 | B4GALT7 | cg03493123 | −0.203926933 | 1.36E−05 |
| 304 | 5 | 177547972 | 177547974 | ER_93712 | N4BP3 | cg17796323 | −0.200270092 | 1.94E−05 |
| 305 | 6 | 3139626 | 3139628 | ER_93950 | BPHL | cg00266592 | −0.183369268 | 9.39E−05 |
| 306 | 6 | 10420695 | 10420697 | ER_94256 | TFAP2A | cg17557766 | −0.250606999 | 7.91E−08 |
| 307 | 6 | 30698986 | 30698988 | ER_95075 | FLOT1 | cg16646298 | −0.158565326 | 0.000746488 |
| 308 | 6 | 31478231 | 31478233 | ER_95117 | MICB | cg12989719 | −0.294476318 | 2.29E−10 |
| 309 | 6 | 31833152 | 31833154 | ER_95141 | SLC44A4 | cg23701033 | −0.191804667 | 4.35E−05 |
| 310 | 6 | 32135026 | 32135028 | ER_95158 | EGFL8 | cg05850971 | −0.241470361 | 2.36E−07 |
| 311 | 6 | 33173133 | 33173135 | ER_95186 | HSD17B8 | cg02802514 | −0.254003888 | 5.22E−08 |
| 312 | 6 | 33288179 | 33288181 | ER_95195 | DAXX | cg03477252 | −0.24031322 | 2.70E−07 |
| 313 | 6 | 33993821 | 33993823 | ER_95239 | GRM4 | cg22971402 | −0.292034242 | 2.69E−10 |
| 314 | 6 | 34512082 | 34512084 | ER_95268 | SPDEF | cg08392123 | −0.522863026 | 0 |
| 315 | 6 | 40363183 | 40363185 | ER_95568 | LRFN2 | cg18454510 | −0.408390902 | 1.62E−19 |
| 316 | 6 | 42109175 | 42109177 | ER_95663 | C6orf132 | cg24627621 | −0.240988721 | 2.29E−07 |
| 317 | 6 | 64281603 | 64281605 | ER_96269 | PTP4A1 | cg24631428 | −0.259535604 | 2.32E−08 |
| 318 | 1 | 25070671 | 25070673 | ER_965 | CLIC4 | cg10546487 | −0.196885845 | 2.69E−05 |
| 319 | 6 | 109267292 | 109267294 | ER_97322 | ARMC2 | cg13725172 | −0.189528837 | 5.37E−05 |
| 320 | 6 | 149806731 | 149806733 | ER_98712 | ZC3H12D | cg14030904 | −0.167456328 | 0.000360227 |
| 321 | 6 | 152124814 | 152124816 | ER_98841 | ESR1 | cg08415493 | −0.636001231 | 0 |
| 322 | 6 | 152432724 | 152432726 | ER_98857 | ESR1 | cg10433043 | −0.198527203 | 2.30E−05 |
| 323 | 7 | 922050 | 922052 | ER_99284 | GET4 | cg07179981 | −0.206381595 | 1.06E−05 |
| 324 | 7 | 923983 | 923985 | ER_99285 | GET4 | cg25795026 | −0.309385232 | 2.48E−11 |
| 325 | 7 | 927933 | 927935 | ER_99286 | GET4 | cg03842205 | −0.265790218 | 1.17E−08 |
| 326 | 7 | 1501247 | 1501249 | ER_99342 | MICALL2 | cg22372849 | −0.231820141 | 7.14E−07 |
| 327 | 1 | 217886460 | 217886462 | ER_9936 | SPATA17 | cg10491122 | −0.496347538 | 0 |
| 328 | 7 | 2673407 | 2673409 | ER_99460 | TTYH3 | cg01827726 | −0.20153074 | 1.72E−05 |

In one example, the method of detecting differential methylation comprises identifying differential methylation at one or more CpG dinucleotide sequences within one or more regions set forth in rows 1-328 of Table 3 in a subject suffering from ESR1 positive breast cancer relative to a reference level of methylation for the corresponding one or more CpG dinucleotide sequences. For example, the method may comprise detecting differential methylation at a single CpG dinucleotide sequence within any one of the genomic regions defined in Table 3 in a subject suffering from ESR1 positive breast cancer relative to a reference level of methylation for the corresponding CpG dinucleotide sequence.

In another example, the method of detecting differential methylation comprises detecting differential methylation at two or more CpG dinucleotides within any genomic region defined in Table 3 in a subject suffering from ESR1 positive breast cancer relative to a reference level of methylation for the corresponding CpG dinucleotide sequences. For example, the method may comprise detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 3 in a subject suffering from ESR1 positive breast cancer relative to a reference level of methylation for the corresponding CpG dinucleotide sequences. The two or more CpG dinucleotides may be consecutive (i.e., contiguous) within a genomic region. Alternatively, the two or more CpG dinucleotides may not be consecutive (i.e., may not be contiguous) within any genomic region.

In yet another example, the method of detecting differential methylation comprises detecting differential methylation of at least one CpG dinucleotide within two or more different genomic regions set forth in Table 3 in a subject suffering from ESR1 positive breast cancer relative to a reference level of methylation for the corresponding CpG dinucleotide sequences. For example, the method may comprise detecting differential methylation at a CpG dinucleotide within two or more, or three or more, or four or more, or five or more, or six or more, or seven or more, or eight or more, or nine or more, or 10 or more different genomic regions set forth in Table 3 in a subject suffering from ESR1 positive breast cancer relative to a reference level of methylation for the corresponding CpG dinucleotide sequences.

In one example, the method of detecting differential methylation comprise detecting hypermethylation of one or more CpG dinucleotides within the 328 ESR1 binding sites set forth in Table 3 in a subject suffering from ESR1 positive breast cancer relative to a reference level of methylation for the corresponding one or more CpG dinucleotide sequences. As hypermethylation of the one or more CpG dinucleotides within the 328 ESR1 binding sites set forth in Table 3 may alter expression of a gene within which the ESR1 binding site resides or with which the ESR1 binding site is closely associated, the method may further comprise detecting expression levels of genes associated with any of the ESR1 binding sites defined in Table 3 in the subject relative to a reference level of expression for the corresponding gene(s).

In one example, identifying differential methylation at one or more CpG dinucleotide sequences within one or more regions set forth in rows 1-328 of Table 3 in a subject relative to a reference level of methylation for the corresponding one or more CpG dinucleotide sequences is indicative of the subject's likely response to endocrine therapy.

Detecting differential methylation at a single CpG dinucleotide sequence within any one of the genomic regions defined in Table 3 may be indicative of the subject's likely response to endocrine therapy.

Alternatively, detecting differential methylation at two or more CpG dinucleotides within any genomic region defined in Table 3 may be indicative of the subject's likely response to endocrine therapy. For example, detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 3 may be indicative of the subject's likely response to endocrine therapy. The two or more CpG dinucleotides may be consecutive (i.e., contiguous) within a genomic region. Alternatively, the two or more CpG dinucleotides may not be consecutive (i.e., may not be contiguous) within any genomic region.

Detecting differential methylation of at least one CpG dinucleotide within two or more different genomic regions set forth in Table 3 may be indicative of the subject's likely response to endocrine therapy. For example, detecting differential methylation at a CpG dinucleotide within two or more, or three or more, or four or more, or five or more, or six or more, or seven or more, or eight or more, or nine or more, or 10 or more different genomic regions set forth in Table 3 may be indicative of the subject's likely response to endocrine therapy.

Hypermethylation of one or more CpG dinucleotides within the 328 ESR1 binding sites set forth in Table 3 may alter expression of a gene within which the ESR1 binding site resides or with which the ESR1 binding site is closely associated. For example, hypermethylation of one or more CpG dinucleotides within the 328 ESR1 binding sites set forth in Table 3 may reduce expression of the gene(s) with which the respective ESR1 binding sites is/are most closely associated. Accordingly, expression levels of genes associated with any of the ESR1 binding sites defined in Table 3 may be indicative of the subject's likely response to endocrine therapy e.g., when that subject is suffering from ESR1 positive breast cancer.

In one example, identifying differential methylation at one or more CpG dinucleotide sequences within one or more regions set forth in rows 1-328 of Table 3 in a subject relative to a reference level of methylation for the corresponding one or more CpG dinucleotide sequences is indicative of the subject having breast cancer which is refractory to endocrine therapy.

Detecting differential methylation at a single CpG dinucleotide sequence within any one of the genomic regions defined in Table 3 may be indicative of the subject having breast cancer which is refractory to endocrine therapy.

Alternatively, detecting differential methylation at two or more CpG dinucleotides within any genomic region defined in Table 3 may be indicative of the subject having breast cancer which is refractory to endocrine therapy. For example, detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 3 may be indicative of the subject having breast cancer which is refractory to endocrine therapy. The two or more CpG dinucleotides may be consecutive (i.e., contiguous) within a genomic region. Alternatively, the two or more CpG dinucleotides may not be consecutive (i.e., may not be contiguous) within any genomic region.

Detecting differential methylation of at least one CpG dinucleotide within two or more different genomic regions set forth in Table 3 may be indicative of the subject having breast cancer which is refractory to endocrine therapy. For example, detecting differential methylation at a CpG dinucleotide within two or more, or three or more, or four or more, or five or more, or six or more, or seven or more, or eight or more, or nine or more, or 10 or more different genomic regions set forth in Table 3 may be indicative of the subject having breast cancer which is refractory to endocrine therapy.

Hypermethylation of one or more CpG dinucleotides within the 328 ESR1 binding sites set forth in Table 3 may alter expression of a gene within which the ESR1 binding site resides or with which the ESR1 binding site is closely associated. For example, hypermethylation of one or more CpG dinucleotides within the 328 ESR1 binding sites set forth in Table 3 may reduce expression of the gene(s) with which the respective ESR1 binding sites is/are most closely associated. Accordingly, expression levels of genes associated with any of the ESR1 binding sites defined in Table 3 may be indicative of the subject having breast cancer which is refractory to endocrine therapy e.g., such as ESR1 positive breast cancer which is refractory to endocrine therapy.

In one example, identifying differential methylation at one or more CpG dinucleotide sequences within one or more regions set forth in rows 1-328 of Table 3 in a subject relative to a reference level of methylation for the corresponding one or more CpG dinucleotide sequences is indicative of the likely therapeutic outcome and/or of the progression of the ESR1 positive breast cancer.

Detecting differential methylation at a single CpG dinucleotide sequence within any one of the genomic regions defined in Table 3 may be indicative of the likely therapeutic outcome and/or of the progression of the ESR1 positive breast cancer.

Alternatively, detecting differential methylation at two or more CpG dinucleotides within any genomic region defined in Table 3 may be indicative of the likely therapeutic outcome and/or of the progression of the ESR1 positive breast cancer. For example, detecting differential methylation at two or more CpG dinucleotides, or three or more CpG dinucleotides, or four or more CpG dinucleotides, or five or more CpG dinucleotides, or six or more CpG dinucleotides, or seven or more CpG dinucleotides, or eight or more CpG dinucleotides, or nine or more CpG dinucleotides, or 10 or more CpG dinucleotides, or 20 or more CpG dinucleotides, or 30 or more CpG dinucleotides, or 40 or more CpG dinucleotides, or 50 or more CpG dinucleotides within a genomic region set forth in Table 3 may be indicative of the likely therapeutic outcome and/or of the progression of the ESR1 positive breast cancer. The two or more CpG dinucleotides may be consecutive (i.e., contiguous) within a genomic region. Alternatively, the two or more CpG dinucleotides may not be consecutive (i.e., may not be contiguous) within any genomic region.

Detecting differential methylation of at least one CpG dinucleotide within two or more different genomic regions set forth in Table 3 may be indicative of the likely therapeutic outcome and/or of the progression of the ESR1 positive breast cancer. For example, detecting differential methylation at a CpG dinucleotide within two or more, or three or more, or four or more, or five or more, or six or more, or seven or more, or eight or more, or nine or more, or 10 or more different genomic regions set forth in Table 3 may be indicative of the likely therapeutic outcome and/or of the progression of the ESR1 positive breast cancer.

Hypermethylation of one or more CpG dinucleotides within the 328 ESR1 binding sites set forth in Table 3 may alter expression of a gene within which the ESR1 binding site resides or with which the ESR1 binding site is closely associated. For example, hypermethylation of one or more CpG dinucleotides within the 328 ESR1 binding sites set forth in Table 3 may reduce expression of the gene(s) with which the respective ESR1 binding sites is/are most closely associated. Accordingly, expression levels of genes associated with any of the ESR1 binding sites defined in Table 3 may be indicative of the likely therapeutic outcome and/or of the progression of the ESR1 positive breast cancer e.g., in a patient suffering from ESR1 positive breast cancer and who is receiving, or about to receive, endocrine therapy.

Particular individual CpG dinucleotides within any of the genomic regions identified in Table 1, Table 2 or Table 3 may be particularly strong predictors of a subject's likely response to endocrine therapy. For example, identifying differential methylation at one or more CpG dinucleotide sequences within one or more estrogen responsive enhancer regions as defined in Table 1 associated with, or spanning, a gene selected from DAXX, ESR1, RXRA, GET4, NCOR2, GATA3, MSI2, C8orf46 and/or ITPK1 in a subject relative to a reference level of methylation for the corresponding one or more CpG dinucleotide sequences is indicative of the subject's likely response to endocrine therapy. For example, differential methylation at one or more CpG dinucleotide sequences selected from those defined in rows 57, 111-113, 256-258, 288-289, 469-470, 805, 821-822 and 824-826 of Table 1 in a subject relative to a reference level of methylation for the corresponding one or more CpG dinucleotide sequences is indicative of the subject's likely response to endocrine therapy.

In another example, identifying differential methylation at one or more CpG dinucleotide sequences within one or more estrogen responsive enhancer regions as defined in Table 1 associated with, or spanning, a gene selected from DAXX, RXRA, NCOR2, MSI2, and/or C8orf46 in a subject relative to a reference level of methylation for the corresponding one or more CpG dinucleotide sequences is indicative of the subject's likely response to endocrine therapy. For example, differential methylation at one or more CpG dinucleotide sequences selected from those defined in rows 57, 111-113, 256-258, 460-470 and 805 of Table 1 in a subject relative to a reference level of methylation for the corresponding one or more CpG dinucleotide sequences is indicative of the subject's likely response to endocrine therapy.

In yet another example, identifying differential methylation at one or more CpG dinucleotide sequences within one or more estrogen responsive enhancer regions as defined in Table 1 associated with, or spanning, a gene selected from FOXA1, ESR1 and/or GATA3 in a subject relative to a reference level of methylation for the corresponding one or more CpG dinucleotide sequences is indicative of the subject's likely response to endocrine therapy. For example, differential methylation at one or more CpG dinucleotide sequences selected from those defined in rows 277 and 821-822 of Table 1 in a subject relative to a reference level of methylation for the corresponding one or more CpG dinucleotide sequences is indicative of the subject's likely response to endocrine therapy.

Particular individual CpG dinucleotides within any of the genomic regions identified in Table 1, Table 2 or Table 3 may have particularly strong diagnostic value in determining whether a subject suffering from ESR1-positive breast cancer is or will be refractory to endocrine therapy. For example, identifying differential methylation at one or more CpG dinucleotide sequences within one or more estrogen responsive enhancer regions as defined in Table 1 associated with, or spanning, a gene selected from DAXX, ESR1, RXRA, GET4, NCOR2, GATA3, MSI2, C8orf46 and/or ITPK1 in a subject relative to a reference level of methylation for the corresponding one or more CpG dinucleotide sequences is indicative of the subject having breast cancer which is refractory to endocrine therapy. For example, differential methylation at one or more CpG dinucleotide sequences selected from those defined in rows 57, 111-113, 256-258, 288-289, 469-470, 805, 821-822 and 824-826 of Table 1 in a subject relative to a reference level of methylation for the corresponding one or more CpG dinucleotide sequences is indicative of the subject having breast cancer which is refractory to endocrine therapy.

In another example, identifying differential methylation at one or more CpG dinucleotide sequences within one or more estrogen responsive enhancer regions as defined in Table 1 associated with, or spanning, a gene selected from DAXX, RXRA, NCOR2, MSI2, and/or C8orf46 in a subject relative to a reference level of methylation for the corresponding one or more CpG dinucleotide sequences is indicative of the subject having breast cancer which is refractory to endocrine therapy. For example, differential methylation at one or more CpG dinucleotide sequences selected from those defined in rows 57, 111-113, 256-258, 460-470 and 805 of Table 1 in a subject relative to a reference level of methylation for the corresponding one or more CpG dinucleotide sequences is indicative of the subject having breast cancer which is refractory to endocrine therapy.

In yet another example, identifying differential methylation at one or more CpG dinucleotide sequences within one or more estrogen responsive enhancer regions as defined in Table 1 associated with, or spanning, a gene selected from FOXA1, ESR1 and/or GATA3 in a subject relative to a reference level of methylation for the corresponding one or more CpG dinucleotide sequences is indicative of the subject having breast cancer which is refractory to endocrine therapy. For example, differential methylation at one or more CpG dinucleotide sequences selected from those defined in rows 277 and 821-822 of Table 1 in a subject relative to a reference level of methylation for the corresponding one or more CpG dinucleotide sequences is indicative of the subject having breast cancer which is refractory to endocrine therapy.

Particular individual CpG dinucleotides within any of the genomic regions identified in Table 1, Table 2 or Table 3 may be particularly strong predictors of a subject's likely therapeutic outcome e.g., in a subject suffering from ESR1-positive cancer receiving endocrine therapy, and/or of the progression of the ESR1 positive breast cancer e.g., in a subject suffering from ESR1-positive cancer receiving endocrine therapy. For example, identifying differential methylation at one or more CpG dinucleotide sequences within one or more estrogen responsive enhancer regions e.g., as defined in Table 1, 2 or 3, associated with, or spanning, a gene selected from DAXX, ESR1, RXRA, GET4, NCOR2, GATA3, MSI2, C8orf46 and/or ITPK1 in a subject relative to a reference level of methylation for the corresponding one or more CpG dinucleotide sequences is indicative of the likely therapeutic outcome and/or of the progression of the ESR1 positive breast cancer. For example, differential methylation at one or more CpG dinucleotide sequences selected from those defined in rows 57, 111-113, 256-258, 288-289, 469-470, 805, 821-822 and 824-826 of Table 1 in a subject relative to a reference level of methylation for the corresponding one or more CpG dinucleotide sequences is indicative of the likely therapeutic outcome and/or of the progression of the ESR1 positive breast cancer.

In another example, identifying differential methylation at one or more CpG dinucleotide sequences within one or more estrogen responsive enhancer regions e.g., as defined in Table 1, 2 or 3, associated with, or spanning, a gene selected from DAXX, RXRA, NCOR2, MSI2, and/or C8orf46 in a subject relative to a reference level of methylation for the corresponding one or more CpG dinucleotide sequences is indicative of the likely therapeutic outcome and/or of the progression of the ESR1 positive breast cancer. For example, differential methylation at one or more CpG dinucleotide sequences selected from those defined in rows 57, 111-113, 256-258, 460-470 and 805 of Table 1 in a subject relative to a reference level of methylation for the corresponding one or more CpG dinucleotide sequences is indicative of the likely therapeutic outcome and/or of the progression of the ESR1 positive breast cancer.

In another example, identifying differential methylation at one or more CpG dinucleotide sequences within one or more estrogen responsive enhancer regions as defined in Table 1 associated with, or spanning, a gene selected from FOXA1, ESR1 and/or GATA3 in a subject relative to a reference level of methylation for the corresponding one or more CpG dinucleotide sequences is indicative of the likely therapeutic outcome and/or of the progression of the ESR1 positive breast cancer. For example, differential methylation at one or more CpG dinucleotide sequences selected from those defined in rows 277 and 821-822 of Table 1 in a subject relative to a reference level of methylation for the corresponding one or more CpG dinucleotide sequences is indicative of the likely therapeutic outcome and/or of the progression of the ESR1 positive breast cancer.

In accordance with any example described herein, the method of detecting differential methylation may comprise identifying differential methylation at one or more CpG dinucleotide sequences within one or more estrogen responsive enhancer regions as defined in Table 1 associated with, or spanning, a gene selected from DAXX, ESR1, RXRA, GET4, NCOR2, GATA3, MSI2, C8orf46 and/or ITPK1 in a subject suffering from ESR1 positive breast cancer relative to a reference level of methylation for the corresponding one or more CpG dinucleotide sequences. For example, the method may comprise detecting differential methylation at one or more CpG dinucleotide sequences selected from those defined in rows 57, 111-113, 256-258, 288-289, 469-470, 805, 821-822 and 824-826 of Table 1 in a subject suffering from ESR1 positive breast cancer relative to a reference level of methylation for the corresponding one or more CpG dinucleotide sequences.

In another example, the method of detecting differential methylation may comprise identifying differential methylation at one or more CpG dinucleotide sequences within one or more estrogen responsive enhancer regions as defined in Table 1 associated with, or spanning, a gene selected from DAXX, RXRA, NCOR2, MSI2, and/or C8orf46 in a subject suffering from ESR1 positive breast cancer relative to a reference level of methylation for the corresponding one or more CpG dinucleotide sequences. For example, the method may comprise detecting differential methylation at one or more CpG dinucleotide sequences selected from those defined in rows 57, 111-113, 256-258, 460-470 and 805 of Table 1 in a subject suffering from ESR1 positive breast cancer relative to a reference level of methylation for the corresponding one or more CpG dinucleotide sequences.

In yet another example, the method of detecting differential methylation may comprise identifying differential methylation at one or more CpG dinucleotide sequences within one or more estrogen responsive enhancer regions as defined in Table 1 associated with, or spanning, a gene selected from FOXA1, ESR1 and/or GATA3 in a subject suffering from ESR1 positive breast cancer relative to a reference level of methylation for the corresponding one or more CpG dinucleotide sequences. For example, the method may comprise detecting differential methylation at one or more CpG dinucleotide sequences selected from those defined in rows 277 and 821-822 of Table 1 in a subject suffering from ESR1 positive breast cancer relative to a reference level of methylation for the corresponding one or more CpG dinucleotide sequences.

It will be understood that the methods described herein encompass determining methylation status of any combination of CpG dinucleotide sequences in any combination of genomic regions set forth in Table 1, Table 2 or Table 3, in any permutation. For example, the methods disclosed herein may comprise determining the methylation status of any one or more CpG dinucleotide sequences in any 2, or more, 3 or more, 4 or more, 5 or more, 10 or more, 20 or more, 30 or more, 40 or more, 50 or more, 60 or more, 70 or more, 80 or more, 90 or more, or 100 or more genomic regions set forth in Table 1, Table 2 or Table 3, in any permutation.

Generally, the greater the number of CpG dinucleotides assessed for methylation status, the more reliable the diagnosis and/or prognosis of the subject. Thus, the greater the number of genomic regions defined in Table 1, Table 2 or Table 3 for which methylation status is determined in the methods disclosed herein, the more reliable the diagnosis or prognosis of the subject.

Breast Cancer Subtypes

The present disclosure provides (i) methods for predicting response to endocrine therapy in a subject suffering from ESR1 positive breast cancer, (ii) methods for diagnosing ESR1 positive breast cancer which is refractory to endocrine therapy, and (iii) methods for predicting the therapeutic outcome of and/or monitoring the progression of ESR1 positive breast cancer in a subject receiving or about to receive endocrine therapy. Exemplary breast cancers which are ESR1 positive include basal breast cancer, Her2 positive breast cancer, progesterone receptor positive breast cancer, ductal carcinoma in situ, lobular carcinoma in situ, early breast cancer, invasive breast cancer, Paget's disease of the nipple, inflammatory breast cancer, locally advanced breast cancer and secondary breast cancer. Breast cancer may also be characterised according to various molecular subtypes which are typically categorized on an immunohistochemical basis. Exemplary molecular subtypes of breast cancer which are ESR1 positive are as follows:

(i) normal (ER+, PR+, HER2+, cytokeratin 5/6+, and HER1+);

(ii) luminal A (ER+ and/or PR+, HER2−); and

(iii) luminal B (ER+ and/or PR+, HER2+).

Detection of differential methylation e.g., hypermethylation, at combinations of the CpG dinucleotides within the ESR1 binding sites identified herein may be particularly useful in the diagnosis, prognosis and/or treatment management of any one or more of these known subtypes of breast cancer.

Diagnostic and/or Prognostic Assay Formats

1. Detection of Methylation of Nucleic Acid and Methods Therefor

The present inventors have identified CpG dinucleotide sequences within estrogen responsive enhancers which are differentially methylated in ESR1 positive breast cancer cells which are responsive to endocrine therapy compared to ESR1 positive breast cancer cells which are refractory to endocrine therapy. The present inventors have also shown that these differentially methylated CpG dinucleotide sequences reside within ESR1-binding sites e.g., as described in Table 1. The present inventors have also shown that a subset of these differentially methylated CpG dinucleotide sequences reside within ESR1-binding sites which are intragenic e.g., as described in Table 2. Furthermore, the present inventors have shown that a subset of these differentially methylated CpG dinucleotide sequences reside within ESR1-binding sites and that hypermethylation of those sites correlates with reduced expression of the gene with which the ESR1 binding site is most closely correlated e.g., as described in Table 3.

The inventors have demonstrated that the differentially methylated CpG dinucleotide sequences within estrogen responsive enhancers e.g., as defined in Tables 1, 2 or 3, are capable of predicting response to endocrine therapy in a subject suffering from ESR1 positive breast cancer. Accordingly, a method of predicting response to endocrine therapy in a subject suffering from ESR1 positive breast cancer shall be taken to include detecting methylation status of one or more CpG dinucleotide sequences within one or more estrogen responsive enhancers e.g., as defined in Tables 1, 2 or 3, to determine whether or not the one or more CpG dinucleotide sequences is differentially methylated relative to a reference level of methylation for the corresponding one or more CpG dinucleotide sequences, wherein differential methylation of said one or more CpG dinucleotide sequences in the subject relative to the reference level is indicative of the subject's likely response to endocrine therapy.

The inventors have also demonstrated that the differentially methylated CpG dinucleotide sequences within estrogen responsive enhancers e.g., as defined in Tables 1, 2 or 3, are capable of diagnosing ESR1 positive breast cancer which is refractory to endocrine therapy. Accordingly, a method of diagnosing ESR1 positive breast cancer which is refractory to endocrine therapy in a subject suffering from ESR1 positive breast cancer shall be taken to include detecting methylation status of one or more CpG dinucleotide sequences within one or more estrogen responsive enhancers e.g., as defined in Tables 1, 2 or 3, to determine whether or not the one or more CpG dinucleotide sequences is differentially methylated relative to a reference level of methylation for the corresponding one or more CpG dinucleotide sequences, wherein differential methylation of said one or more CpG dinucleotide sequences in the subject relative to the reference level is indicative of the subject having breast cancer which is refractory to endocrine therapy.

The inventors have also demonstrated that the differentially methylated CpG dinucleotide sequences within estrogen responsive enhancers e.g., as defined in Tables 1, 2 or 3, are capable of predicting the therapeutic outcome of and/or monitoring the progression of ESR1 positive breast cancer in a subject receiving or about to receive endocrine therapy. Accordingly, a method of predicting the therapeutic outcome of and/or monitoring the progression of ESR1 positive breast cancer in a subject receiving or about to receive endocrine therapy shall be taken to include detecting methylation status of one or more CpG dinucleotide sequences within one or more estrogen responsive enhancers e.g., as defined in Tables 1, 2 or 3, to determine whether or not the one or more CpG dinucleotide sequences is differentially methylated relative to a reference level of methylation for the corresponding one or more CpG dinucleotide sequences, wherein differential methylation of said one or more CpG dinucleotide sequences in the subject relative to the reference level is indicative of the likely therapeutic outcome and/or of the progression of the ESR1 positive breast cancer.

Suitable methods for the detection of methylation status are known in the art and/or described herein.

The term “methylation” shall be taken to mean the addition of a methyl group by the action of a DNA methyl transferase enzyme to a CpG island of nucleic acid, e.g., genomic DNA. As described herein, there are several methods known to those skilled in the art for determining the level or degree of methylation of nucleic acid. By “differential methylation” of a nucleic acid it is meant that there is a deviation in the number of methylated CpG dinucleotides at a genomic region within the subject diagnosed compared to that detected within a corresponding genomic region in a suitable control sample i.e., which provides a reference level of methylation for that genomic region. The differentially methylated nucleic acid may have an increased level of methylation within a specific or defined region of nucleic acid e.g., such as hypermethylation, or a decreased level of methylation within a specific or defined region of nucleic acid e.g., such as hypomethylation.

The term “hypermethylation” shall be taken to mean that a plurality of CpG dinucleotides in a specific or defined region of nucleic acid are methylated relative to a reference level.

The term “hypomethylation” shall be taken to mean that a plurality of CpG dinucleotides in a specific or defined region of nucleic acid are unmethylated relative to a reference level.

The present disclosure is not to be limited by a precise number of methylated residues that are considered to be (i) predictive of a likely response to endocrine therapy in a subject suffering from ESR1 positive breast cancer (ii) or diagnostic of ESR1 positive breast cancer which is refractory to endocrine therapy, or (iii) predictive of the therapeutic outcome of and/or progression of ESR1 positive breast cancer in a subject receiving or about to receive endocrine therapy, because some variation between patient samples will occur. Nor is the present disclosure to be limited by the specific positioning of the methylated residue within an estrogen responsive enhancer region.

In one example, the degree of methylation in a subject is determined for one or more CpG dinucleotides with one or more ESR1 binding sites set forth in Tables 1-3. In one example, the degree of methylation in a subject is determined for one or more CpG dinucleotides within one or more ESR1 binding sites set forth in Table 1. In one example, the degree of methylation in a subject is determined for one or more CpG dinucleotides within one or more ESR1 binding sites set forth in Table 2. In one example, the degree of methylation in a subject is determined for one or more CpG dinucleotides within one or more ESR1 binding sites set forth in Table 3. In one example, the degree of methylation in a subject is determined for one or more CpG dinucleotides within one or more estrogen responsive enhancers of a gene selected from DAXX, ESR1, RXRA, GET4, NCOR2, GATA3, MSI2, C8orf46 and/or ITPK1. In one example, the degree of methylation in a subject is determined for one or more CpG dinucleotides within one or more estrogen responsive enhancers of a gene selected from DAXX, RXRA, NCOR2, MSI2, and/or C8orf46. In one example, the degree of methylation in a subject is determined for one or more CpG dinucleotides within one or more estrogen responsive enhancers of a gene selected from FOXA1, ESR1 and/or GATA3.

a) Probe or Primer Design and/or Production

Several diagnostic and/or prognostic methods described herein use one or more probes and/or primers to detect methylation at a genomic region. Methods for designing probes and/or primers for use in, for example, PCR or hybridization are known in the art and described, for example, in Dieffenbach and Dveksler (Eds) (In: PCR Primer: A Laboratory Manual, Cold Spring Harbor Laboratories, N Y, 1995). Furthermore, several software packages are publicly available that design optimal probes and/or primers for a variety of assays, e.g. Primer 3 available from the Center for Genome Research, Cambridge, Mass., USA.

The potential use of the probe or primer should be considered during its design. For example, should the probe or primer be produced for use in, for example, a methylation specific PCR or ligase chain reaction (LCR) assay the nucleotide at the 3′ end (or 5′ end in the case of LCR) should correspond to a methylated nucleotide in a nucleic acid.

Probes and/or primers useful for detection of a marker associated with a breast cancer are assessed, for example, to determine those that do not form hairpins, self-prime or form primer dimers (e.g. with another probe or primer used in a detection assay).

Methods for producing/synthesizing a probe or primer of the present disclosure are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (e.g. by digestion of a nucleic acid with a restriction endonuclease) or by chemical synthesis. For short sequences (up to about 100 nucleotides) chemical synthesis is preferable.

Other methods for oligonucleotide synthesis include, for example, phosphotriester and phosphodiester methods (Narang, et al. Meth. Enzymol 68: 90, 1979) and synthesis on a support (Beaucage, et al Tetrahedron Letters 22: 1859-1862, 1981) as well as phosphoramidate technique, Caruthers, M. H., et al., “Methods in Enzymology,” Vol. 154, pp. 287-314 (1988), and others described in “Synthesis and Applications of DNA and RNA,” S. A. Narang, editor, Academic Press, New York, 1987, and the references cited therein.

Probes comprising locked nucleic acid (LNA) are synthesized as described, for example, in Nielsen et al, J. Chem. Soc. Perkin Trans., 1: 3423, 1997; Singh and Wengel, Chem. Commun. 1247, 1998. While, probes comprising peptide-nucleic acid (PNA) are synthesized as described, for example, in Egholm et al., Am. Chem. Soc., 114: 1895, 1992; Egholm et al., Nature, 365: 566, 1993; and Orum et al., Nucl. Acids Res., 21: 5332, 1993.

b) Methylation-Sensitive Endonuclease Digestion of DNA

In one example, the methylation status of one or more CpG dinucleotide sequences within one or more estrogen responsive enhancers e.g., as defined in Tables 1, 2 or 3, in a sample is determined using a process comprising treating the nucleic acid with an amount of a methylation-sensitive restriction endonuclease enzyme under conditions sufficient for nucleic acid to be digested and then detecting the fragments produced. Exemplary methylation-sensitive endonucleases include, for example, HpaI or HpaII.

In one example, the digestion of nucleic acid is detected by selective hybridization of a probe or primer to the undigested nucleic acid. Alternatively, the probe selectively hybridizes to both digested and undigested nucleic acid but facilitates differentiation between both forms, e.g., by electrophoresis. Suitable detection methods for achieving selective hybridization to a hybridization probe include, for example, Southern or other nucleic acid hybridization (Kawai et al., Mol. Cell. Biol. 14, 7421-7427, 1994; Gonzalgo et al., Cancer Res. 57, 594-599, 1997).

The term “selectively hybridizable” means that the probe is used under conditions where a target nucleic acid hybridizes to the probe to produce a signal that is significantly above background (i.e., a high signal-to-noise ratio). The intensity of hybridization is measured, for example, by radiolabeling the probe, e.g. by incorporating [α-³⁵5] and/or [α-³²P]dNTPs, [γ-³²P]ATP, biotin, a dye ligand (e.g., FAM or TAMRA), a fluorophore, or other suitable ligand into the probe prior to use and then detecting the ligand following hybridization.

The skilled artisan will be aware that optimum hybridization reaction conditions should be determined empirically for each probe, although some generalities can be applied. Preferably, hybridizations employing short oligonucleotide probes are performed at low to medium stringency.

For the purposes of defining the level of stringency to be used in these diagnostic and/or prognostic assays, a low stringency is defined herein as being a hybridization and/or a wash carried out in about 6×SSC buffer and/or about 0.1% (w/v) SDS at about 28° C. to about 40° C., or equivalent conditions. A moderate stringency is defined herein as being a hybridization and/or washing carried out in about 2×SSC buffer and/or about 0.1% (w/v) SDS at a temperature in the range of about 45° C. to about 65° C., or equivalent conditions.

In the case of a GC rich probe or primer or a longer probe or primer a high stringency hybridization and/or wash is preferred. A high stringency is defined herein as being a hybridization and/or wash carried out in about 0.1×SSC buffer and/or about 0.1% (w/v) SDS, or lower salt concentration, and/or at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample DNA, and/or the type of hybridization probe used and/or constituents of any buffer used in a hybridization. For example, formamide reduces the melting temperature of a probe or primer in a hybridization or an amplification reaction.

Conditions for specifically hybridizing nucleic acid, and conditions for washing to remove non-specific hybridizing nucleic acid, are understood by those skilled in the art. For the purposes of further clarification only, reference to the parameters affecting hybridization between nucleic acid molecules is found in Ausubel et al. (Current Protocols in Molecular Biology, Wiley Interscience, ISBN 047150338, 1992), which is herein incorporated by reference.

In accordance with the present example, a difference in the fragments produced for a test sample and a control sample is indicative of (i) a subject's likely response to endocrine therapy, (ii) an ESR1 positive breast cancer which will be refractory to endocrine therapy, and/or (iii) a likely therapeutic outcome and/or progression of ESR1 positive breast cancer in a subject receiving or about to receive endocrine therapy. Similarly, in cases where the control sample comprises data from a breast tumor, a breast cancer tissue or a breast cancerous cell, which is ESR1 positive and refractory to endocrine therapy, similarity, albeit not necessarily absolute identity, between the test sample and the control sample is indicative of (i) a subject's likely response to endocrine therapy, (ii) an ESR1 positive breast cancer which will be refractory to endocrine therapy, and/or (iii) a likely therapeutic outcome and/or progression of ESR1 positive breast cancer in a subject receiving or about to receive endocrine therapy.

In an alternative example, the fragments produced by the restriction enzyme are detected using an amplification system, such as, for example, polymerase chain reaction (PCR), rolling circle amplification (RCA), inverse polymerase chain reaction (iPCR), in situ PCR (Singer-Sam et al., Nucl. Acids Res. 18, 687,1990), strand displacement amplification (SDA) or cycling probe technology.

Methods of PCR are known in the art and described, for example, by McPherson et al., PCR: A Practical Approach. (series eds, D. Rickwood and B. D. Hames), IRL Press Limited, Oxford. pp 1-253, 1991 and by Dieffenbach (ed) and Dveksler (ed) (In: PCR Primer: A Laboratory Manual, Cold Spring Harbour Laboratories, N Y, 1995), the contents of which are each incorporated in their entirety by way of reference. Generally, for PCR two non-complementary nucleic acid primer molecules comprising at least about 18 nucleotides in length, and more preferably at least 20-30 nucleotides in length are hybridized to different strands of a nucleic acid template molecule at their respective annealing sites, and specific nucleic acid molecule copies of the template that intervene the annealing sites are amplified enzymatically. Amplification products may be detected, for example, using electrophoresis and detection with a detectable marker that binds nucleic acids. Alternatively, one or more of the oligonucleotides are labeled with a detectable marker (e.g. a fluorophore) and the amplification product detected using, for example, a lightcycler (Perkin Elmer, Wellesley, Mass., USA).

Strand displacement amplification (SDA) utilizes oligonucleotide primers, a DNA polymerase and a restriction endonuclease to amplify a target sequence. The oligonucleotides are hybridized to a target nucleic acid and the polymerase is used to produce a copy of the region intervening the primer annealing sites. The duplexes of copied nucleic acid and target nucleic acid are then nicked with an endonuclease that specifically recognizes a sequence at the beginning of the copied nucleic acid. The DNA polymerase recognizes the nicked DNA and produces another copy of the target region at the same time displacing the previously generated nucleic acid. The advantage of SDA is that it occurs in an isothermal format, thereby facilitating high-throughput automated analysis.

Cycling Probe Technology uses a chimeric synthetic primer that comprises DNA-RNA-DNA that is capable of hybridizing to a target sequence. Upon hybridization to a target sequence the RNA-DNA duplex formed is a target for RNaseH thereby cleaving the primer. The cleaved primer is then detected, for example, using mass spectrometry or electrophoresis.

For primers that flank, or which are adjacent to a methylation-sensitive endonuclease recognition site, it is preferred that such primers flank only those sites that are hypermethylated in the ESR1 breast cancer to ensure that a diagnostic and/or prognostic amplification product is produced. In this regard, an amplification product will only be produced when the restriction site is not cleaved i.e., when it is methylated. Accordingly, detection of an amplification product indicates that the CpG dinucleotide/s of interest is/are methylated.

This form of analysis may be used to determine the methylation status of a plurality of CpG dinucleotides within a genomic region provided that each dinucleotide is within a methylation sensitive restriction endonuclease site.

In these methods, one or more of the primers may be labeled with a detectable marker to facilitate rapid detection of amplified nucleic acid, for example, a fluorescent label (e.g. Cy5 or Cy3) or a radioisotope (e.g. ³²P).

The amplified nucleic acids are generally analyzed using, for example, non-denaturing agarose gel electrophoresis, non-denaturing polyacrylamide gel electrophoresis, mass spectrometry, liquid chromatography (e.g. HPLC or dHPLC), or capillary electrophoresis. (e.g. MALDI-TOF). High throughput detection methods, such as, for example, matrix-assisted laser desorption/ionization time of flight (MALDI-TOF), electrospray ionization (ESI), mass spectrometry (including tandem mass spectrometry, e.g. LC MS/MS), biosensor technology, evanescent fiber-optics technology or DNA chip technology (e.g., WO98/49557; WO 96/17958; Fodor et al., Science 767-773, 1991; U.S. Pat. No. 5,143,854; and U.S. Pat. No. 5,837,832, the contents of which are all incorporated herein by reference).

Alternatively, amplification of a nucleic acid may be continuously monitored using a melting curve analysis method as described herein and/or in, for example, U.S. Pat. No. 6,174,670, which is incorporated herein by reference.

c) Selective Mutagenesis of Non-Methylated DNA

In an alternative example of the present disclosure, the methylation status of a genomic region in a subject sample is determined using a process comprising treating the nucleic acid with an amount of a compound that selectively mutates a non-methylated cytosine residue within a CpG dinucleotide under conditions sufficient to induce mutagenesis.

Exemplary compounds mutate cytosine to uracil or thymidine, such as, for example, a salt of bisulfite, e.g., sodium bisulfite or potassium bisulfite (Frommer et al., Proc. Natl. Acad. Sci. USA 89, 1827-1831, 1992). Bisulfite treatment of DNA is known to distinguish methylated from non-methylated cytosine residues, by mutating cytosine residues that are not protected by methylation, including cytosine residues that are not within a CpG dinucleotide or that are positioned within a CpG dinucleotide that is not subject to methylation.

(i) Sequence Based Detection

In one example, the presence of one or more mutated nucleotides in a genomic region or the number of mutated sequences in a sample is determined by sequencing mutated DNA. One form of analysis comprises amplifying mutated nucleic acid or methylated nucleic acid using an amplification reaction described herein, for example, PCR. The amplified product is then directly sequenced or cloned and the cloned product sequenced. Methods for sequencing DNA are known in the art and include for example, the dideoxy chain termination method or the Maxam-Gilbert method (see Sambrook et al., Molecular Cloning, A Laboratory Manual (2nd Ed., CSHP, New York 1989) or Zyskind et al., Recombinant DNA Laboratory Manual, (Acad. Press, 1988)).

As the treatment of nucleic acid with a compound, such as, for example, bisulfite results in non-methylated cytosines being mutated to uracil or thymidine, analysis of the sequence determines the presence or absence of a methylated nucleotide. For example, by comparing the sequence obtained using a control sample or a sample that has not been treated with bisulfite, or the known nucleotide sequence of the region of interest with a treated sample facilitates the detection of differences in the nucleotide sequence. Any thymine residue detected at the site of a cytosine in the treated sample compared to a control or untreated sample may be considered to be caused by mutation as a result of bisulfite treatment. Suitable methods for the detection of methylation using sequencing of bisulfite treated nucleic acid are described, for example, in Frommer et al., Proc. Natl. Acad. Sci. USA 89: 1827-1831, 1992 or Clark et al., Nucl. Acids Res. 22: 2990-2997, 1994. One example of a commercially available kit for carrying out such methods is the CpGenome™ DNA modification Kit (Millipore). Other suitable kits are available from MDX Health SA (Belgium).

In another example, the presence of a mutated or non-mutated nucleotide in a bisulfite treated sample is detected using pyrosequencing, such as, for example, as described in Uhlmann et al., Electrophoresis, 23: 4072-4079, 2002. Essentially this method is a form of real-time sequencing that uses a primer that hybridizes to a site adjacent or close to the site of a cytosine that is methylated in a cancer cell. Following hybridization of the primer and template in the presence of a DNA polymerase each of four modified deoxynucleotide triphosphates are added separately according to a predetermined dispensation order. Only an added nucleotide that is complementary to the bisulfite treated sample is incorporated and inorganic pyrophosphate (PPi) is liberated. The PPi then drives a reaction resulting in production of detectable levels of light. Such a method allows determination of the identity of a specific nucleotide adjacent to the site of hybridization of the primer.

A related method for determining the sequence of a bisulfite treated nucleotide is methylation-sensitive single nucleotide primer extension (Me-SnuPE) or SNaPmeth. Suitable methods are described, for example, in Gonzalgo and Jones Nucl. Acids Res., 25: 2529-2531 or Uhlmann et al., Electrophoresis, 23: 4072-4079, 2002.

Clearly other high throughput sequencing methods are encompassed by the present disclosure. Such methods include, for example, solid phase minisequencing (as described, for example, in Syvamen et al, Genomics, 13: 1008-1017, 1992), or minisequencing with FRET (as described, for example, in Chen and Kwok, Nucleic Acids Res. 25: 347-353, 1997).

(ii) Restriction Endonuclease-Based Assay Format

In one example, the presence of a non-mutated nucleic sequence is detected using combined bisulfite restriction analysis (COBRA) essentially as described in Xiong and Laird, Nucl. Acids Res., 25: 2532-2534, 2001. This method exploits the differences in restriction enzyme recognition sites between methylated and unmethylated nucleic acid after treatment with a compound that selectively mutates a non-methylated cytosine residue, e.g., bisulfite.

Following bisulfite treatment a genomic region of interest comprising one or more CpG dinucleotides that are methylated in a ESR1 positive cancer cell, and which are included in a restriction endonuclease recognition sequence, is amplified using an amplification reaction described herein, e.g., PCR. The amplified product is then contacted with the restriction enzyme that cleaves at the site of the CpG dinucleotide for a time and under conditions sufficient for cleavage to occur. A restriction site may be selected to indicate the presence or absence of methylation. For example, the restriction endonuclease Taql cleaves the sequence TCGA, following bisulfite treatment of a non-methylated nucleic acid the sequence will be TTGA and, as a consequence, will not be cleaved. The digested and/or non-digested nucleic acid is then detected using a detection means known in the art, such as, for example, electrophoresis and/or mass spectrometry. The cleavage or non-cleavage of the nucleic acid is indicative of cancer in a subject.

Clearly, this method may be employed in either a positive read-out or negative read-out system when performing a diagnostic and/or prognostic method of the disclosure.

(iii) Positive Read-Out Assay Format

In one example, the assay format of the disclosure comprises a positive read-out system in which hypermethylated DNA from a breast cancer sample e.g., an ESR1-positive breast cancer sample, that has been treated, for example, with bisulfite is detected as a positive signal if the breast cancer is, or is likely to be, refractory to endocrine therapy. For example, non-hypermethylated DNA from a healthy or normal control subject, or non-hypermethylated DNA from a breast cancer sample e.g., an ESR1 positive breast cancer sample, is not detected or only weakly detected and is likely to be or is responsive to endocrine therapy.

In one example, the enhanced methylation in a subject sample is determined using a process comprising:

(i) treating the nucleic acid with an amount of a compound that selectively mutates a non-methylated cytosine residue under conditions sufficient to induce mutagenesis thereby producing a mutated nucleic acid;

(ii) hybridizing a nucleic acid to a probe or primer comprising a nucleotide sequence that is complementary to a sequence comprising a methylated cytosine residue under conditions such that selective hybridization to the non-mutated nucleic acid occurs; and

(iii) Detecting the Selective Hybridization.

In this context, the term “selective hybridization” means that hybridization of a probe or primer to the non-mutated nucleic acid occurs at a higher frequency or rate, or has a higher maximum reaction velocity, than hybridization of the same probe or primer to the corresponding mutated sequence. Preferably, the probe or primer does not hybridize or detectably hybridize (e.g., does not hybridize at a level significantly above background) to the non-methylated sequence carrying the mutation(s) under the reaction conditions used.

In one example, the hybridization is detected using Southern, dot blot, slot blot or other nucleic acid hybridization means (Kawai et al., Mol. Cell. Biol. 14, 7421-7427, 1994; Gonzalgo et al., Cancer Res. 57, 594-599, 1997). Subject to appropriate probe selection, such assay formats are generally described herein above and apply mutatis mutandis to the presently described selective mutagenesis approach.

In one example, a ligase chain reaction format is employed to distinguish between a mutated and non-mutated nucleic acid. Ligase chain reaction (described in EP 320,308 and U.S. Pat. No. 4,883,750) uses at least two oligonucleotide probes that anneal to a target nucleic acid in such a way that they are juxtaposed on the target nucleic acid such that they can be linked using a ligase. The probes that are not ligated are removed by modifying the hybridization stringency. In this respect, probes that have not been ligated will selectively hybridize under lower stringency hybridization conditions than probes that have been ligated. Accordingly, the stringency of the hybridization can be increased to a stringency that is at least as high as the stringency used to hybridize the longer probe, and preferably at a higher stringency due to the increased length contributed by the shorter probe following ligation. One exemplary method melts the target-probe duplex, elute the dissociated probe and confirm that is has been ligated, e.g., by determining its length using electrophoresis, mass spectrometry, nucleotide sequence analysis, gel filtration, or other means known to the skilled artisan.

Methylation specific microarrays (MSO) are also useful for differentiating between a mutated and non-mutated sequence. A suitable method is described, for example, in Adorj et al, Nucl. Acids Res., 30: e21, 2002. MSO uses nucleic acid that has been treated with a compound that selectively mutates a non-methylated cytosine residue (e.g., bisulfite) as template for an amplification reaction that amplifies both mutant and non-mutated nucleic acid. The amplification is performed with at least one primer that comprises a detectable label, such as, for example, a fluorophore, e.g., Cy3 or Cy5. The labeled amplification products are then hybridized to oligonucleotides on the microarray under conditions that enable detection of single nucleotide differences. Following washing to remove unbound amplification product, hybridization is detected using, for example, a microarray scanner. Not only does this method allow for determination of the methylation status of a large number of CpG dinucleotides, it is also semi-quantitative, enabling determination of the degree of methylation at each CpG dinucleotide analyzed. As there may be some degree of heterogeneity of methylation in a single sample, such quantification may assist in the diagnosis of cancer.

In an alternative example, the hybridization is detected using an amplification system. In methylation-specific PCR formats (MSP; Herman et al. Proc. Natl. Acad. Sci. USA 93: 9821-9826, 1992), the hybridization is detection using a process comprising amplifying the bisulfite-treated DNA. By using one or more probe or primer that anneals specifically to the unmutated sequence under moderate and/or high stringency conditions an amplification product is only produced using a sample comprising a methylated nucleotide.

Any amplification assay format described herein can be used, such as, for example, polymerase chain reaction (PCR), rolling circle amplification (RCA), inverse polymerase chain reaction (iPCR), in situ PCR (Singer-Sam et al., Nucl. Acids Res. 18, 687,1990), strand displacement amplification, or cycling probe technology.

PCR techniques have been developed for detection of gene mutations (Kuppuswamy et al., Proc. Natl. Acad. Sci. USA 88:1143-1147, 1991) and quantitation of allelic-specific expression (Szabo and Mann, Genes Dev. 9: 3097-3108, 1995; and Singer-Sam et al., PCR Methods Appl. 1: 160-163, 1992). Such techniques use internal primers, which anneal to a PCR-generated template and terminate immediately 5′ of the single nucleotide to be assayed. Such as format is readily combined with ligase chain reaction as described herein above.

Methylation-specific melting-curve analysis (essentially as described in Worm et al., Clin. Chem., 47: 1183-1189, 2001) is also contemplated by the present disclosure. This process exploits the difference in melting temperature in amplification products produced using bisulfite treated methylated or unmethylated nucleic acid. In essence, non-discriminatory amplification of a bisulfite treated sample is performed in the presence of a fluorescent dye that specifically binds to double stranded DNA (e.g., SYBR Green I). By increasing the temperature of the amplification product while monitoring fluorescence the melting properties and thus the sequence of the amplification product is determined. A decrease in the fluorescence reflects melting of at least a domain in the amplification product. The temperature at which the fluorescence decreases is indicative of the nucleotide sequence of the amplified nucleic acid, thereby permitting the nucleotide at the site of one or more CpG dinucleotides to be determined. As the sequence of the nucleic acids amplified using the present disclosure

The present disclosure also encompasses the use of real-time quantitative forms of PCR, such as, for example, TaqMan (Holland et al., Proc. Natl Acad. Sci. USA, 88, 7276-7280, 1991; Lee et al., Nucleic Acid Res. 21, 3761-3766, 1993) to perform this embodiment. For example, the MethylLight method of Eads et al., Nucl. Acids Res. 28: E32, 2000 uses a modified TaqMan assay to detect methylation of a CpG dinucleotide.

Alternatively, rather than using a labeled probe that requires cleavage, a probe, such as, for example, a Molecular Beacon™ is used (see, for example, Mhlang and Malmberg, Methods 25: 463-471, 2001). Molecular beacons are single stranded nucleic acid molecules with a stem-and-loop structure. The loop structure is complementary to the region surrounding the one or more CpG dinucleotides that are methylated in a cancer sample and not in a control sample. The stem structure is formed by annealing two “arms” complementary to each other, which are on either side of the probe (loop). A fluorescent moiety is bound to one arm and a quenching moiety that suppresses any detectable fluorescence when the molecular beacon is not bound to a target sequence is bound to the other arm. Upon binding of the loop region to its target nucleic acid the arms are separated and fluorescence is detectable. However, even a single base mismatch significantly alters the level of fluorescence detected in a sample. Accordingly, the presence or absence of a particular base is determined by the level of fluorescence detected. Such an assay facilitates detection of one or more unmutated sites (i.e. methylated nucleotides) in a nucleic acid.

As exemplified herein, another amplification based assay useful for the detection of a methylated nucleic acid following treatment with a compound that selectively mutates a non-methylated cytosine residue makes use of headloop PCR technology (e.g., as described in published PCT Application No. PCT/AU03/00244; WO 03/072810). This form of amplification uses a probe or primer that comprises a region that binds to a nucleic acid and is capable of amplifying nucleic acid in an amplification reaction whether the nucleic acid is methylated or not. The primer additionally comprises a region that is complementary to a portion of the amplified nucleic acid enabling this region of the primer to hybridize to the amplified nucleic acid incorporating the primer thereby forming a hairpin. The now 3′ terminal nucleotide/s of the annealed region (i.e. the most 5′ nucleotide/s of the primer) hybridize to the site of one or more mutated cytosine residues (i.e., unmethylated in nucleic acid from a cancer subject). Accordingly, this facilitates self-priming of amplification products from unmethylated nucleic acid, the thus formed hairpin structure blocking further amplification of this nucleic acid. In contrast, the complementary region may or may not by capable of hybridizing to an amplification product from methylated (mutated) nucleic acid, but is unable to “self-prime” thereby enabling further amplification of this nucleic acid (e.g., by the inability of the now 3′ nucleotide to hybridize to the amplification product). This method may be performed using a melting curve analysis method to determine the amount of methylated nucleic acid in a biological sample from a subject.

Other amplification based methods for detecting methylated nucleic acid following treatment with a compound that selectively mutates a non-methylated cytosine residue include, for example, methylation-specific single stranded conformation analysis (MS-SSCA) (Bianco et al., Hum. Mutat., 14: 289-293, 1999), methylation-specific denaturing gradient gel electrophoresis (MS-DGGE) (Abrams and Stanton, Methods Enzymol., 212: 71-74, 1992) and methylation-specific denaturing high-performance liquid chromatography (MS-DHPLC) (Deng et al, Chin. J. Cancer Res., 12: 171-191, 2000). Each of these methods use different techniques for detecting nucleic acid differences in an amplification product based on differences in nucleotide sequence and/or secondary structure. Such methods are clearly contemplated by the present disclosure.

(iv) Negative Read-Out Assays

In an alternative example, the assay format comprises a negative read-out system in which non-hypermethylated DNA from a healthy or normal control subject, or non-hypermethylated DNA from a breast cancer sample e.g., a ESR1 positive breast cancer sample, which is responsive to endocrine therapy is detected as a positive signal and preferably, hypermethylated DNA from a breast cancer sample e.g., an ESR1-positive breast cancer sample, which is, or is likely to be, refractory to endocrine therapy, is not detected or is only weakly detected.

In one example, the non-hypermethylated DNA is determined using a process comprising:

(i) treating the nucleic acid with an amount of a compound that selectively mutates a non-methylated cytosine residue within a CpG island under conditions sufficient to induce mutagenesis thereby producing a mutated nucleic acid;

(ii) hybridizing the nucleic acid to a probe or primer comprising a nucleotide sequence that is complementary to a sequence comprising the mutated cytosine residue under conditions such that selective hybridization to the mutated nucleic acid occurs; and

(iii) Detecting the Selective Hybridization.

In this context, the term “selective hybridization” means that hybridization of a probe or primer to the mutated nucleic acid occurs at a higher frequency or rate, or has a higher maximum reaction velocity, than hybridization of the same probe or primer to the corresponding non-mutated sequence. In one example, the probe or primer does not hybridize or detectably hybridize to the methylated sequence (or non-mutated sequence) under the reaction conditions used.

The skilled artisan will be able to adapt a positive read-out assay described above to a negative read-out assay, e.g., by producing a probe or primer that selectively hybridizes to non-mutated DNA rather than mutated DNA.

d) Methylated DNA Immunoprecipitation (MeDiP)

In another example, the methylation status of a genomic region in a subject sample is determined using a process comprising physically isolating methylated DNA (e.g., hypermethylated DNA) from hypomethylated or non-methylated DNA in a sample, followed by sequencing of the physically-separated methylated DNA. Preferably, the physical separation of methylated DNA is accomplished using Methylated DNA Immunoprecipitation (MeDiP), a technique that has been described in the art (See e.g., Weber, M. et al. (2005) Nat. Genet. 37:853-862; and Rakyan, et al. (2008) Genome Res. 18:1518-1529; which are both expressly incorporated herein by reference).

In accordance with a method of the disclosure in which MeDiP is employed to physically separate methylated DNA (e.g., hypermethylated DNA), the input nucleic acid preparation (from a subject) is denatured, incubated with an antibody directed against 5-methylcytosine and then the methylated DNA is isolated by immunoprecipitation. For example, to accomplish immunoprecipitation, the anti-5-methylcytosine antibody can be coupled to a solid support (e.g., magnetic dynabeads, microscopic agarose beads or paramagnetic beads) to allow for precipitation of the methylated DNA from solution (direct immunoprecipitation). Alternatively, a secondary antibody or reagent can be used that recognizes the anti-5-methylcytosine antibody (e.g., the constant region of the antibody) and that is coupled to a solid support, to thereby allow for precipitation of the methylated DNA from solution (indirect immunoprecipitation). For direct or indirect immunoprecipitation, other approaches known in the art for physical separation of components within a sample, such as the biotin/avidin or biotin/streptavidin systems, can be used. For example, the anti-5-methylcytosine antibody can be coupled to biotin and then avidin or streptavidin coupled to a solid support can be used to allow for precipitation of the methylated DNA from solution. It will be apparent to the ordinarily skilled artisan that other variations known in the art for causing immunoprecipitation are also suitable for use in the method of the disclosure. Thus, as used herein, the term “Methylated DNA Immunoprecipitation” or “MeDiP” is intended to encompass any and all approaches in which an antibody that discriminates between hypermethylated DNA and hypomethylated or non-methylated DNA is contacted with a nucleic acid obtained from a subject suffering from ESR1 positive breast cancer, followed by precipitation of the hypermethylated DNA (i.e., the DNA that specifically binds to the antibody) out of solution. For example, an approach in which an antibody comprising a methylated DNA binding domain (MBD) or a bispecific molecule comprised of a MBD and an antibody or part thereof e.g., Fc portion), is clearly contemplated for use in a method of the disclosure for detecting and/or physically isolating methylated DNA from a sample. Techniques using antibodies and other proteins comprising MBD for detecting methylated DNA are described in US Patent Publication US200150267263 and in BLUEPRINT Consortium (2016) Nat. Biotechnol. Doi: 10.1038/nbt.3062; both of which are expressly incorporated herein by reference.

Typically after physical separation of the hypermethylated DNA from hypomethylated or non-methylated DNA, the hypermethylated DNA is then amplified. As used herein, the term “amplified” is intended to mean that additional copies of the DNA are made to thereby increase the number of copies of the DNA, which is typically accomplished using the polymerase chain reaction (PCR). One particular method for amplification of the hypermethylated DNA is ligation mediated polymerase chain reaction (LM-PCR), which has been described previously in the art (See e.g., Ren, B. et al. (2000) Science 22:2306-2309; and Oberley, M. J. et al. (2004) Methods Enzymol. 376:315-334; the contents of both of which are expressly incorporated herein by reference). In LM-PCR, linker ends are ligated onto a sample of DNA fragments through blunt-end ligation and then oligonucleotide primers that recognize the nucleotide sequences of the linker ends are used in PCR to thereby amplify the DNA fragments to which the linkers have been ligated. Thus, in an example of the method of the disclosure in which LM-PCR is used, DNA from a subject suffering from ESR1-positive breast cancer is fragmented (e.g., into fragments of approximately 300-800 bp), and linker arms are ligated onto the fragmented DNA by blunt-end ligation, after which the hypermethylated DNA is physically separated from the hypeomethylated DNA or non-methylated DNA (e.g., by MeDiP). Then, following physical separation of the hypermethylated DNA, the recovered hypermethylated DNA is subjected to PCR using oligonucleotide primers that recognized the linker ends that have been ligated onto the DNA. This results in amplification of the hypermethylated DNA sample.

The amplified hypermethylated DNA sample may then be sequenced using standard sequencing methodologies known in the art and described herein. Sequence data can then be used to determine the methylation status of a genomic region in a subject sample. Moreover, this form of analysis may be used to determine the methylation status of a plurality of CpG dinucleotides within a genomic region simultaneously.

e) Additional Method Steps

The methods disclosed herein may further comprise one or more steps of enriching methylated DNA in a sample. Thus, the methods disclosed herein may further comprise one or more steps of isolating methylated DNA from a sample. The enrichment/isolation step may be performed prior to or concomitant with any other step in the method for detecting the level of methylation of a CpG dinucleotide sequence within an estrogen responsive enhancer region as disclosed herein.

Any suitable enriching/isolating step known in the art may be used. For example, the methods disclosed herein may comprise a step of enriching methylated DNA in a sample using a commercially available kit such as the CpG MethylQuest DNA Isolation Kit (Millipore), which provides a recombinant protein comprising the methyl binding domain (MBD) of the mouse MBD2b protein fused to a glutathione-S-transferase (GST) protein from S. japonicum via a linker containing a thrombin cleavage site, the recombinant protein being immobilized to a magnetic bead. The MBD binds to methylated CpG sites with high affinity and in a sequence-independent manner, thereby allowing enrichment of methylated DNA in a sample.

It will be appreciated that alternative or additional methods known in the art for enrichment/isolation of methylated DNA in a sample can be used in the methods disclosed herein. For example, methods of enrichment/isolation of methylated DNA in a sample are described in Hsu et al., (2014) Methods Mol Biol, 1105:61-70, Serre et al., (2010) Nucleic Acids Res, 38:391-399, Rauch and Pfeifer (2005) Lab Invest, 85:1172-1180, Nair et al., (2011) Epigenetics, 6:34-44; and Robinson et al., (2010) Genome Res, 20:1719-1729.

A method disclosed herein according to any example may also comprise selecting a patient based on the result of a method disclosed herein and performing an additional diagnostic method or recommending performance of an additional diagnostic method. For example, for a patient diagnosed as suffering from ESR1 positive breast cancer which is, or is likely to become, refractory to endocrine therapy, the additional diagnostic method may be an ultrasound or a biopsy.

2. Detection of Reduced Gene Expression

Since methylation of a nucleic acid sequence affects its expression, the present inventors have also demonstrated that the level of expression of nucleic acids within any of a number of genomic regions described herein is varied (e.g., reduced or increased) in ESR1 positive breast cancer subjects and in ESR1 positive breast cancer cell lines. Thus, the methods disclosed herein may additionally or alternatively comprise determining the level of expression of any polynucleotides overlapping, spanning or closely associated with, any of the estrogen responsive enhancer regions identified in Tables 1-3 herein. In one example, the methods disclosed herein may additionally or alternatively comprise determining the level of expression of one or more polynucleotides overlapping, spanning or closely associated with, one or more of the estrogen responsive enhancer regions defined in Table 1. In one example, the methods disclosed herein may additionally or alternatively comprise determining the level of expression of one or more polynucleotides overlapping, spanning or closely associated with, one or more of the estrogen responsive enhancer regions defined in Table 2. In one example, the methods disclosed herein may additionally or alternatively comprise determining the level of expression of one or more polynucleotides overlapping, spanning or closely associated with, one or more of the estrogen responsive enhancer regions defined in Table 3. For example, detecting a reduced level of expression of one or more polynucleotides overlapping, spanning or closely associated with, one or more of the estrogen responsive enhancer regions defined in Tables 1-3 may be (i) predictive of a likely response to endocrine therapy in a subject suffering from ESR1 positive breast cancer, (ii) or diagnostic of ESR1 positive breast cancer which is refractory to endocrine therapy, or (iii) predictive of the therapeutic outcome and/or progression of ESR1 positive breast cancer in a subject receiving or about to receive endocrine therapy.

a) Nucleic Acid Detection

In one example, the level of expression of a nucleic acid is determined by detecting the level of mRNA transcribed from genomic region described herein.

In one example, the mRNA is detected by hybridizing a nucleic acid probe or primer capable of specifically hybridizing to a transcript of a genomic region described herein to a nucleic acid in a biological sample derived from a subject and detecting the hybridization by a detection means. Preferably, the detection means is an amplification reaction, or a nucleic acid hybridization reaction, such as, for example, as described herein.

In this context, the term “selective hybridization” means that hybridization of a probe or primer to the transcript occurs at a higher frequency or rate, or has a higher maximum reaction velocity, than hybridization of the same probe or primer to any other nucleic acid. Preferably, the probe or primer does not hybridize to another nucleic acid at a detectable level under the reaction conditions used.

As transcripts of a gene or pseudogene described herein are detected using mRNA or cDNA derived therefrom, assays that detect changes in mRNA are preferred (e.g. Northern hybridization, RT-PCR, NASBA, TMA or ligase chain reaction).

Methods of RT-PCR are known in the art and described, for example, in Dieffenbach (ed) and Dveksler (ed) (In: PCR Primer: A Laboratory Manual, Cold Spring Harbour Laboratories, N Y, 1995). Essentially, this method comprises performing a PCR reaction using cDNA produced by reverse transcribing mRNA from a cell using a reverse transcriptase. Methods of PCR described supra are to be taken to apply mutatis mutandis to this embodiment of the disclosure.

Similarly LCR may be performed using cDNA. Preferably, one or more of the probes or primers used in the reaction specifically hybridize to the transcript of interest. Method of LCR are described supra and are to be taken to apply mutatis mutandis to this embodiment of the disclosure.

Methods of TMA or self-sustained sequence replication (3SR) use two or more oligonucleotides that flank a target sequence, a RNA polymerase, RNase H and a reverse transcriptase. One oligonucleotide (that also comprises a RNA polymerase binding site) hybridizes to an RNA molecule that comprises the target sequence and the reverse transcriptase produces cDNA copy of this region. RNase H is used to digest the RNA in the RNA-DNA complex, and the second oligonucleotide used to produce a copy of the cDNA. The RNA polymerase is then used to produce a RNA copy of the cDNA, and the process repeated.

NASBA systems relies on the simultaneous activity of three enzymes (a reverse transcriptase, RNase H and RNA polymerase) to selectively amplify target mRNA sequences. The mRNA template is transcribed to cDNA by reverse transcription using an oligonucleotide that hybridizes to the target sequence and comprises a RNA polymerase binding site at its 5′ end. The template RNA is digested with RNase H and double stranded DNA is synthesized. The RNA polymerase then produces multiple RNA copies of the cDNA and the process is repeated.

The present disclosure also contemplates the use of a microarray to determine the level of expression of one or more nucleic acids described herein. Such a method enables the detection of a number of different nucleic acids, thereby providing a multi-analyte test and improving the sensitivity and/or accuracy of the diagnostic assay of the disclosure.

b) Polypeptide Detection

In an alternative example, the level of expression of a genomic region is determined by detecting the level of a protein encoded by a nucleic acid within a genomic region described herein.

In this respect, the present disclosure is not necessarily limited to the detection of a protein comprising the specific amino acid sequence recited herein. Rather, the present disclosure encompasses the detection of variant sequences (e.g., having at least about 80% or 90% or 95% or 98% amino acid sequence identity) or the detection of an immunogenic fragment or epitope of said protein.

The amount, level or presence of a polypeptide is determined using any of a variety of techniques known to the skilled artisan such as, for example, a technique selected from the group consisting of, immunohistochemistry, immunofluorescence, an immunoblot, a Western blot, a dot blot, an enzyme linked immunosorbent assay (ELISA), radioimmunoassay (RIA), enzyme immunoassay, fluorescence resonance energy transfer (FRET), matrix-assisted laser desorption/ionization time of flight (MALDI-TOF), electrospray ionization (ESI), mass spectrometry (including tandem mass spectrometry, e.g. LC MS/MS), biosensor technology, evanescent fiber-optics technology or protein chip technology.

In one example, the assay used to determine the amount or level of a protein is a semi-quantitative assay. In another example, the assay used to determine the amount or level of a protein in a quantitative assay. As will be apparent from the preceding description, such an assay may require the use of a suitable control, e.g. from a normal individual or matched normal control.

Standard solid-phase ELISA or FLISA formats are particularly useful in determining the concentration of a protein from a variety of samples.

In one form such an assay involves immobilizing a biological sample onto a solid matrix, such as, for example a polystyrene or polycarbonate microwell or dipstick, a membrane, or a glass support (e.g. a glass slide). An antibody that specifically binds to a protein described herein is brought into direct contact with the immobilized biological sample, and forms a direct bond with any of its target protein present in said sample. This antibody is generally labeled with a detectable reporter molecule, such as for example, a fluorescent label (e.g. FITC or Texas Red) or a fluorescent semiconductor nanocrystal (as described in U.S. Pat. No. 6,306,610) in the case of a FLISA or an enzyme (e.g. horseradish peroxidase (HRP), alkaline phosphatase (AP) or β-galactosidase) in the case of an ELISA, or alternatively a second labeled antibody can be used that binds to the first antibody. Following washing to remove any unbound antibody the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal) in the case of an enzymatic label.

In another form, an ELISA or FLISA comprises immobilizing an antibody or ligand that specifically binds a protein described supra on a solid matrix, such as, for example, a membrane, a polystyrene or polycarbonate microwell, a polystyrene or polycarbonate dipstick or a glass support. A sample is then brought into physical relation with said antibody, and the polypeptide is bound or ‘captured’. The bound protein is then detected using a labeled antibody. For example, a labeled antibody that binds to an epitope that is distinct from the first (capture) antibody is used to detect the captured protein. Alternatively, a third labeled antibody can be used that binds the second (detecting) antibody.

In another example, the presence or level of a protein is detected in a body fluid using, for example, a biosensor instrument (e.g., BIAcore™, Pharmacia Biosensor, Piscataway, N.J.). In such an assay, an antibody or ligand that specifically binds a protein is immobilized onto the surface of a receptor chip. For example, the antibody or ligand is covalently attached to dextran fibers that are attached to gold film within the flow cell of the biosensor device. A test sample is passed through the cell. Any antigen present in the body fluid sample, binds to the immobilized antibody or ligand, causing a change in the refractive index of the medium over the gold film, which is detected as a change in surface plasmon resonance of the gold film.

In another example, the presence or level of a protein or a fragment or epitope thereof is detected using a protein and/or antibody chip. To produce such a chip, an antibody or ligand that binds to the antigen of interest is bound to a solid support such as, for example glass, polycarbonate, polytetrafluoroethylene, polystyrene, silicon oxide, gold or silicon nitride. This immobilization is either direct (e.g. by covalent linkage, such as, for example, Schiff's base formation, disulfide linkage, or amide or urea bond formation) or indirect.

To bind a protein to a solid support it is often necessary to treat the solid support so as to create chemically reactive groups on the surface, such as, for example, with an aldehyde-containing silane reagent or the calixcrown derivatives described in Lee et al, Proteomics, 3: 2289-2304, 2003. A streptavidin chip is also useful for capturing proteins and/or peptides and/or nucleic acid and/or cells that have been conjugated with biotin (e.g. as described in Pavlickova et al., Biotechniques, 34: 124-130, 2003). Alternatively, a peptide is captured on a microfabricated polyacrylamide gel pad and accelerated into the gel using microelectrophoresis as described in, Arenkov et al. Anal. Biochem. 278:123-131, 2000.

Other assay formats are also contemplated, such as flow-through immunoassays (PCT/AU2002/01684), a lateral flow immunoassay (US20040228761, US20040248322 or US20040265926), a fluorescence polarization immunoassay (FPIA) (U.S. Pat. Nos. 4,593,089, 4,492,762, 4,668,640, and 4,751,190), a homogeneous microparticles immunoassay (“HMI”) (e.g., U.S. Pat. Nos. 5,571,728, 4,847,209, 6,514,770, and 6,248,597) or a chemiluminescent microparticle immunoassay (“CMIA”).

3 Multiplex Assay Formats

The present disclosure also contemplates multiplex or multianalyte format assays to improve the accuracy or specificity of the diagnostic and/or prognostic methods described herein. Such assays may also improve the population coverage by an assay.

Methods for determining the sensitivity of an assay will be apparent to the skilled artisan. For example, an assay described herein is used to analyze a population of test subjects to determine those that will develop cancer. Post-mortem analysis is then used to determine those subjects that did actually determine breast cancer. The number of “true positives” (i.e., subjects that developed breast cancer and were positively identified using the method of the disclosure) and “true negatives” (i.e., subjects that did not develop breast cancer and were not identified using the method of the disclosure) are determined.

Sensitivity of the assay is then determined by the following formula:

No. of true positives/(No. of true positives+No. of false negatives).

In one example, a method of the disclosure has a high degree of sensitivity in predicting the likelihood of a subject suffering from ESR1 positive breast cancer responding to endocrine therapy. For example, in a test population of individuals, the method of the disclosure is able to predict that a subject will not respond to endocrine therapy, for example, in at least about 50% of subjects suffering from ESR1 positive breast cancer which do not respond to endocrine therapy, for example, in at least about 60% of subjects suffering from ESR1 positive breast cancer which do not respond to endocrine therapy, for example, in at least about 65% of subjects suffering from ESR1 positive breast cancer which do not respond to endocrine therapy, for example, in at least about 70% of subjects suffering from ESR1 positive breast cancer which do not respond to endocrine therapy, for example, in at least about 75% of subjects suffering from ESR1 positive breast cancer which do not respond to endocrine therapy, for example, in at least about 80% of subjects suffering from ESR1 positive breast cancer which do not respond to endocrine therapy, for example, in at least about 85% of subjects suffering from ESR1 positive breast cancer which do not respond to endocrine therapy, for example, in at least about 90% of subjects suffering from ESR1 positive breast cancer which do not respond to endocrine therapy, for example, in at least about 95% of subjects suffering from ESR1 positive breast cancer which do not respond to endocrine therapy.

In another example, a method of the disclosure has a high degree of sensitivity in detecting ESR1 positive breast cancer which is refractory to endocrine therapy. For example, in a test population of individuals, the method of the disclosure detects at least about 50% of subjects developing or suffering from ESR1 positive breast cancer which is refractory to endocrine therapy, for example, at least about 60% of subjects developing or suffering from ESR1 positive breast cancer which is refractory to endocrine therapy, for example, at least about 65% of subjects developing or suffering from ESR1 positive breast cancer which is refractory to endocrine therapy, for example, at least about 70% of subjects developing or suffering from ESR1 positive breast cancer which is refractory to endocrine therapy, for example, at least about 75% of subjects developing or suffering from ESR1 positive breast cancer which is refractory to endocrine therapy, for example, at least about 80% of subjects developing or suffering from ESR1 positive breast cancer which is refractory to endocrine therapy, for example, at least about 85% of subjects developing or suffering from ESR1 positive breast cancer which is refractory to endocrine therapy, for example, at least about 90% of subjects developing or suffering from ESR1 positive breast cancer which is refractory to endocrine therapy, for example, at least about 95% of subjects developing or suffering from ESR1 positive breast cancer which is refractory to endocrine therapy.

In another example, a method of the disclosure has a high degree of sensitivity in stratifying ESR1 positive breast cancer subtypes associated with prognostic profiles following endocrine therapy e.g., such as populations of ESR1 positive breast cancer patients with which are likely to respond to endocrine therapy and populations of ESR1 positive breast cancer patients with which are unlikely to respond to endocrine therapy. In this way, the method of the disclosure has a high degree of sensitivity in predicting a therapeutic outcome and/or progression of ESR1 positive breast cancer in a subject receiving or about to receive endocrine therapy. For example, in a test population of individuals having ESR1 positive breast cancer, the method of the disclosure stratifies at least about 50% of subjects having ESR1 positive breast cancer according to a disease outcome, for example, at least about 60% of subjects having ESR1 positive breast cancer according to a disease outcome, for example, at least about 70% of subjects having ESR1 positive breast cancer according to a disease outcome, for example, at least about 80% of subjects having ESR1 positive breast cancer according to a disease outcome, for example, at least about 85% of subjects having ESR1 positive breast cancer according to a disease outcome, for example, at least about 90% of subjects having ESR1 positive breast cancer according to a disease outcome, for example, at least about 95% of subjects having ESR1 positive breast cancer according to a disease outcome. A disease outcome in accordance with this example is a likelihood that the breast cancer patient will survive at least 3 years following endocrine therapy, for example, at least 5 years following endocrine therapy, for example, at least 10 years following endocrine therapy.

Specificity is determined by the following formula:

No. of true negatives/(No. of true negatives+No. of false positives).

An exemplary multiplex assay for use in a method of the disclosure comprises, for example, detecting differential methylation of one or more CpG dinucleotides in a plurality of estrogen responsive enhancer regions set forth in Tables 1-3. In one example, the method comprises detecting the level of methylation of one or more CpG dinucleotides in a plurality of the estrogen responsive enhancer regions set forth in Tables 1-3 to predict response to endocrine therapy in a subject suffering from ESR1 positive breast cancer. In another example, the method comprises detecting the level of methylation of one or more CpG dinucleotides in a plurality of estrogen responsive enhancer regions set forth in Tables 1-3 to diagnose ESR1 positive breast cancer which is refractory to endocrine therapy. In yet another example, the method comprises detecting the level of methylation of one or more CpG dinucleotides in a plurality of estrogen responsive enhancer regions set forth in Tables 1-3 to stratify and/or predict a therapeutic outcome and/or progression of ESR1 positive breast cancer in a subject receiving or about to receive endocrine therapy.

The multiplex assay of the disclosure is not to be limited to the detection of methylation at a single CpG dinucleotide within a region of interest i.e., each estrogen responsive enhancer region. Rather, the present disclosure contemplates detection of methylation at a sufficient number of CpG dinucleotides in each nucleic acid to provide a diagnosis/prognosis. For example, the disclosure contemplates detection of methylation at 1 or 2 or 3 or 4 or 5 or 7 or 9 or 10 or 15 or 20 or 25 or 30 CpG dinculeotides in each nucleic acid i.e., each estrogen responsive enhancer region. Preferably, the disclosure contemplates detection of methylation at more than 1 CpG dinculeotide in each nucleic acid i.e., each estrogen responsive enhancer region.

As will be apparent from the foregoing description, a methylation specific microarray is amenable to such high density analysis. Previously, up to 232 CpG dinucleotides have been analyzed using such a microarray (Adorján et al., Nucl. Acids Res. 30: e21, 2002).

A method of the disclosure may comprises one or more assays to determine the level of expression of a gene spanning, comprising or closely associated with at least one estrogen responsive enhancer region set forth in Tables 1-3 to predict response to endocrine therapy in a subject suffering from ESR1 positive breast cancer. A method of the disclosure may comprises one or more assays to determine the level of expression of a gene spanning, comprising or closely associated with at least one estrogen responsive enhancer region set forth in Table 1-3 to diagnose ESR1 positive breast cancer which is refractory to endocrine therapy A method of the disclosure may comprises one or more assays to determine the level of expression of a gene spanning, comprising or closely associated with at least one estrogen responsive enhancer region set forth in Tables 1-3 to stratify and/or predict a therapeutic outcome and/or progression of ESR1 positive breast cancer in a subject receiving or about to receive endocrine therapy. For example, the method may comprise detecting the level of mRNA or protein corresponding to a gene spanning, comprising or closely associated with at least one estrogen responsive enhancer region set forth in Table 1-3. Alternatively, the level of mRNA transcribed from one or more genes spanning, comprising or closely associated with at least one estrogen responsive enhancer region set forth in Table 1-3 and the level of one or more proteins expressed by the same or different genes spanning, comprising or closely associated with at least one estrogen responsive enhancer region set forth in Table 1-3 may be determined.

Each of the previously described detection techniques can be used independently of one another in the diagnostic and/or prognostic methods described. Accordingly, a single sample may be analyzed to determine the level of methylation of one or more CpG dinculeotides in one or more estrogen responsive enhancer regions and to determine the level of expression of one or more nucleic acids and/or proteins. In accordance with this example, hypermethylation of one or more CpG dinucleotides within one or more estorgen enhancer regions defined in Tables 1-3, and reduced expression of one or more genes spanning, comprising or closely associated with at least one estrogen responsive enhancer region set forth in Tables 1-3, is indicative of (i) a subject's likely response to endocrine therapy e.g., non-response to endocrine therapy, (ii) a ESR1 positive breast cancer will be refractory to endocrine therapy, and/or (iii) a likely therapeutic outcome and/or progression of ESR1 positive breast cancer in a subject receiving or about to receive endocrine therapy.

Based on the teachings provided herein, a variety of combinations of assays will be apparent to the skilled artisan.

The present disclosure also contemplates the use of a known diagnostic assay in combination with an assay described herein.

Samples

A sample useful for the method of the present disclosure is, for example, from a tissue suspected of comprising a ESR1 positive breast cancer or a ESR1 positive breast cancer cell. For example, the cell is from a region of a tissue thought to comprise a ESR1 positive breast cancer or a ESR1 positive breast cancer cell. This does not exclude cells that have originated in a particular tissue but are isolated from a remote source.

The sample may be taken from a subject suspected of having ESR1 positive breast cancer. For example, the sample may be taken from a subject having ESR1 positive breast cancer and suspected of having or being at risk of developing ESR1 positive breast cancer which is refractory to endocrine therapy. For example, the subject may have a family history of ESR1 positive breast cancer, including ESR1 positive breast cancer which is resistant, or develops resistance, to endocrine therapy. The subject may have been subjected to any other test for detecting any form of ESR1 positive breast cancer.

In one example, the sample comprises a body fluid or a derivative of a body fluid or a body secretion. For example, the body fluid is selected from the group consisting of whole blood, urine, saliva, breast milk, pleural fluid, sweat, tears and mixtures thereof. An example of a derivative of a body fluid is selected from the group consisting of plasma, serum or buffy coat fraction. In one example, the sample comprises a whole blood sample, a serum sample or a plasma sample.

In one example DNA is isolated from either; whole blood, plasma, serum, peripheral blood mononucleated cells (PBMC) or enriched epithelial cells derived from the blood of patients diagnosed with breast cancer or healthy controls. DNA may then be bisulfite converted and gene-specific methylated sequences may be detected by either; methylation specific headloop suppression PCR, MALDI-TOF mass spectrometry (sequenom) or other bisulfite based PCR assay.

Preferably, the sample comprises a nucleated cell or an extract thereof. More preferably, the sample comprises a breast cancer cell e.g., an ESR1 positive breast cancer cell, or an extract thereof.

In another example, the sample comprises nucleic acid and/or protein from a breast cancer cell e.g., a nucleic acid and/or protein from an ESR1 positive breast cancer cell. The nucleic acid and/or protein may be separate need not be isolated with a cell, but rather may be from, for example, a lysed cell.

As the present disclosure provides methods which are useful for early detection of ESR1 positive breast cancer which is refractory to endocrine therapy in the medium to long term, the term breast cancer cell is not to be limited by the stage of a cancer in the subject from which said breast cancer cell is derived (i.e. whether or not the patient is in remission or undergoing disease recurrence or whether or not the ESR1 positive breast cancer is a primary tumor or the consequence of metastases). Nor is the term “breast cancer cell”, “cancer cell” or similar to be limited by the stage of the cell cycle of said cancer cell.

In one example, the sample comprises a cell or a plurality of cells derived from a breast.

In one example, the biological sample has been isolated previously from the subject. In accordance with this example, a method of the present disclosure is performed ex vivo. In such cases, the sample may be processed or partially processed into a nucleic acid sample that is substantially free of contaminating protein. All such examples are encompassed by the present disclosure.

Methods for isolating a sample from a subject are known in the art and include, for example, surgery, biopsy, collection of a body fluid, for example, by paracentesis or thoracentesis or collection of, for example, blood or a fraction thereof. All such methods for isolating a biological sample shall be considered to be within the scope of providing or obtaining a sample.

For example, in the case of a breast cancer, a sample is collected, for example, using a fine needle aspiration biopsy, a core needle biopsy, or a surgical biopsy.

It will be apparent from the preceding description that methods provided by the present disclosure involve a degree of quantification to determine elevated or enhanced methylation of nucleic acid in tissue that is suspected of comprising a cancer cell or metastases thereof, or reduced gene expression in tissue that is suspected of comprising a cancer cell or metastases thereof. Such quantification is readily provided by the inclusion of appropriate control samples in the assays as described below.

As will be apparent to the skilled artisan, when internal controls are not included in each assay conducted, the control may be derived from an established data set.

Data pertaining to the control subjects are selected from the group consisting of:
1. a data set comprising measurements of the degree of methylation and/or gene expression for a typical population of subjects known to have ESR1 positive breast cancer which was responsive to endocrine therapy at the time of testing the subjects;
2. a data set comprising measurements of the degree of methylation and/or gene expression for the subject being tested wherein said measurements have been made previously, such as, for example, when the subject was known to be healthy or, in the case of a subject having ESR1 positive breast cancer, when the subject was at a stage in disease progression when the ESR1 positive breast cancer was responsive to endocrine therapy;
3. a data set comprising measurements of the degree of methylation and/or gene expression for a healthy individual or a population of healthy individuals;
4. a data set comprising measurements of the degree of methylation and/or gene expression for a normal individual or a population of normal individuals;
5. a data set comprising measurements of the degree of methylation and/or gene expression for an individual or a population of individuals diagnosed as having cancer other than a breast cancer characterized as being ESR1-negative subtype, or a ESR1-positive subtype which is refractory to endocrine therapy; and
6. a data set comprising measurements of the degree of methylation and/or gene expression from the subject being tested wherein the measurements are determined in a matched sample.

In a preferred example, the data comprising measurements of the degree of methylation and/or gene expression for a healthy subject, individual or population pertains to healthy breast epithelial cell(s) from the subject, individual or population.

Those skilled in the art are readily capable of determining the baseline for comparison in any diagnostic/prognostic assay of the present disclosure without undue experimentation, based upon the teaching provided herein.

In the present context, the term “typical population” with respect to subjects known to have ESR1 positive breast cancer which is responsive to endocrine therapy shall be taken to refer to a population or sample of subjects diagnosed with a specific form of ESR1 positive breast cancer that is representative of the spectrum of subjects suffering from ESR1 positive breast cancer. This is not to be taken as requiring a strict normal distribution of morphological or clinicopathopathological parameters in the population, since some variation in such a distribution is permissible. Preferably, a “typical population” will exhibit a spectrum of subtypes of ESR1 positive breast cancers at different stages of disease progression and with tumors at different stages and having different morphologies or degrees of differentiation.

In the present context, the term “healthy individual” shall be taken to mean an individual who is known not to suffer from breast cancer, such knowledge being derived from clinical data on the individual. It is preferred that the healthy individual is asymptomatic with respect to the any symptoms associated with breast cancer.

The term “normal individual” shall be taken to mean an individual having a normal level of methylation at a genomic region and/or gene expression as described herein in a particular sample derived from said individual.

As will be known to those skilled in the art, data obtained from a sufficiently large sample of the population will normalize, allowing the generation of a data set for determining the average level of a particular parameter. Accordingly, the level of methylation and/or gene expression as described herein can be determined for any population of individuals, and for any sample derived from said individual, for subsequent comparison to levels determined for a sample being assayed. Where such normalized data sets are relied upon, internal controls are preferably included in each assay conducted to control for variation.

The term “matched sample” shall be taken to mean that a control sample is derived from the same subject as the test sample is derived, at approximately the same point in time. In one example, the control sample shows little or no morphological and/or pathological indications of cancer. Matched samples are not applicable to blood-based or serum-based assays. Accordingly, it is preferable that the matched sample is from a region of the same tissue as the test sample e.g., breast tissue, such as breast epithelial tissue, however does not appear to comprise a cancer cell. For example, the matched sample does not include malignant cells or exhibit any symptom of the disease. For example, the sample comprises less than about 20% malignant cells, such as less than about 10% malignant cells, for example less than about 5% malignant cells, e.g., less than about 1% malignant cells. Morphological and pathological indications of malignant cells are known in the art and/or described herein.

In one example, the differential methylation of one or more CpG dinucleotides within one or more estrogen responsive enhancer regions defined in Tables 1-3 relative to the methylation status of a corresponding one or more CpG dinucleotides of a control is indicative of a subject's likely response to endocrine therapy. For example, hypermethylation of the one or more CpG dinucleotides is indicative that a subject having ESR1 positive breast cancer will be resistant to endocrine therapy. Alternatively, non-hypermethylation of the one or more CpG dinucleotides is indicative that a subject having ESR1 positive breast cancer which is responsive to endocrine therapy.

In another example, differential methylation of one or more CpG dinucleotides within one or more estrogen responsive enhancer regions defined in Tables 1-3 relative to the methylation status of a corresponding one or more CpG dinucleotides of a control is diagnostic of ESR1 positive breast cancer which is refractory to endocrine therapy. For example, hypermethylation of the one or more CpG dinucleotides is diagnostic of ESR1 positive breast cancer which is refractory to endocrine therapy. Alternatively, non-hypermethylation of the one or more CpG dinucleotides is diagnostic of ESR1 positive breast cancer is responsive to endocrine therapy.

In another example, differential methylation of one or more CpG dinucleotides within one or more estrogen responsive enhancer regions defined in Tables 1-3 relative to the methylation status of a corresponding one or more CpG dinucleotides of a control is predictive of the therapeutic outcome and/or likely progression of ESR1 positive breast cancer in a subject receiving or about to receive endocrine therapy. For example, hypermethylation of the one or more CpG dinucleotides is predictive that a subject suffering from ESR1 positive breast cancer who is receiving or about to receive endocrine therapy will not respond to the treatment and/or the cancer will progress to a worsening stage. Alternatively, non-hypermethylation of the one or more CpG dinucleotides is predictive that a subject suffering from ESR1 positive breast cancer who is receiving or about to receive endocrine therapy will have a good therapeutic outcome and/or the cancer will not progress to a worsening stage.

In an alternative example, the differential expression of a gene overlapping, spanning or closely associated with one or more CpG dinucleotides within one or more estrogen responsive enhancer regions defined in Tables 1-3 relative to a corresponding level of gene expression of a control is indicative of a subject's likely response to endocrine therapy. For example, reduced expression of a gene overlapping, spanning or closely associated with the one or more CpG dinucleotides is indicative that a subject having ESR1 positive breast cancer will be resistant to endocrine therapy. Alternatively, expression of a gene overlapping, spanning or closely associated with the one or more CpG dinucleotides which is not reduced is indicative that a subject having ESR1 positive breast cancer which is responsive to endocrine therapy.

In another alternative example, the differential expression of a gene overlapping, spanning or closely associated with one or more CpG dinucleotides within one or more estrogen responsive enhancer regions defined in Tables 1-3 relative to a corresponding level of gene expression of a control is diagnostic of ESR1 positive breast cancer which is refractory to endocrine therapy. For example, reduced expression of a gene overlapping, spanning or closely associated with the one or more CpG dinucleotides is diagnostic of ESR1 positive breast cancer which is refractory to endocrine therapy. Alternatively, expression of a gene overlapping, spanning or closely associated with the one or more CpG dinucleotides which is not reduced is diagnostic of ESR1 positive breast cancer is responsive to endocrine therapy.

In yet another alternative example, the differential expression of a gene overlapping, spanning or closely associated with one or more CpG dinucleotides within one or more estrogen responsive enhancer regions defined in Tables 1-3 relative to a corresponding level of gene expression of a control is predictive of the therapeutic outcome and/or likely progression of ESR1 positive breast cancer in a subject receiving or about to receive endocrine therapy. For example, reduced expression of a gene overlapping, spanning or closely associated with the one or more CpG dinucleotides is predictive that a subject suffering from ESR1 positive breast cancer who is receiving or about to receive endocrine therapy will not respond to the treatment and/or the cancer will progress to a worsening stage. Alternatively, expression of a gene overlapping, spanning or closely associated with the one or more CpG dinucleotides which is not reduced is predictive that a subject suffering from ESR1 positive breast cancer who is receiving or about to receive endocrine therapy will have a good therapeutic outcome and/or the cancer will not progress to a worsening stage.

The level(s) of differential methylation of the one or more CpG dinucleotides with the one or more estrogen responsive enhancer regions set forth in Tables 1-3 may be subjected to multivariate analysis to create an algorithm which enables the determination of an index of probability that a subject having ESR1 positive breast cancer will be resistant or responsive to endocrine therapy e.g., stratification of ESR1 positive breast cancer substypes, and/or that a subject having ESR1 positive breast cancer who is receiving or about to receive endocrine therapy will respond or is responding to endocrine therapy and/or that the ESR1 positive breast cancer will progress to a worsening stage following endocrine therapy. Hence, in one example, the present disclosure provides a rule based on the application of a comparison of levels of methylation biomarkers to control samples. In another example, the rule is based on application of statistical and machine learning algorithms. Such an algorithm uses the relationships between methylation biomarkers and disease status observed in training data (with known disease status) to infer relationships which are then used to predict the status of patients with unknown status. Practitioners skilled in the art of data analysis recognize that many different forms of inferring relationships in the training data may be used without materially changing the present disclosure.

The term “status” shall be taken to include whether or not a subject suffers from ESR1 positive breast cancer which is responsive or refractory to endocrine therapy (i.e., diagnostic status), whether or not an ESR1 positive breast cancer has responded to endocrine therapy and/or developed resistance thereto.

Analysis as described in the preceding paragraphs can also consider clinical parameters or traditional laboratory risk factors.

Information as discussed above can be combined and made more clinically useful through the use of various formulae, including statistical classification algorithms and others, combining and in many cases extending the performance characteristics of the combination beyond that of any individual data point. These specific combinations show an acceptable level of diagnostic/prognostic accuracy, and, when sufficient information from multiple markers is combined in a trained formula, often reliably achieve a high level of diagnostic/prognostic accuracy transportable from one population to another.

Several statistical and modeling algorithms known in the art can be used to both assist in biomarker selection choices and optimize the algorithms combining these choices. Statistical tools such as factor and cross-biomarker correlation/covariance analyses allow more rational approaches to panel construction. Mathematical clustering and classification tree showing the Euclidean standardized distance between the biomarkers can be advantageously used. Pathway informed seeding of such statistical classification techniques also may be employed, as may rational approaches based on the selection of individual biomarkers (e.g., such as those CpG dinucleotides within estrogen responsive enhancer regions set forth in Tables 1-3) based on their participation across in particular pathways or physiological functions or individual performance.

Ultimately, formulae such as statistical classification algorithms can be directly used to both select methylation biomarkers and to generate and train the optimal formula necessary to combine the results from multiple methylation biomarkers into a single index. Often techniques such as forward (from zero potential explanatory parameters) and backwards selection (from all available potential explanatory parameters) are used, and information criteria are used to quantify the tradeoff between the performance and diagnostic/prognostic accuracy of the panel and the number of methylation biomarkers used. The position of the individual methylation biomarkers on a forward or backwards selected panel can be closely related to its provision of incremental information content for the algorithm, so the order of contribution is highly dependent on the other constituent biomarkers in the panel.

Any formula may be used to combine methylation biomarker results into indices or indexes useful in the practice of the disclosure. As indicated herein, and without limitation, such indices may indicate, among the various other indications, the probability, likelihood, absolute or relative risk, time to or rate of disease, conversion from one to another disease states, or make predictions of future biomarker measurements of cancer. This may be for a specific time period or horizon, or for remaining lifetime risk, or simply be provided as an index relative to another reference subject population.

The actual model type or formula used may itself be selected from the field of potential models based on the performance and diagnostic accuracy characteristics of its results in a training population. The specifics of the formula itself may commonly be derived from biomarker results in the relevant training population. Amongst other uses, such formula may be intended to map the feature space derived from one or more biomarker inputs to a set of subject classes (e.g. useful in predicting class membership of subjects as normal, as having ESR1 positive breast cancer which is responsive or resistant/refractory to endocrine therapy or at risk of developing resistance to endocrine therapy), to derive an estimation of a probability function of risk using a Bayesian approach (e.g. the risk of ESR1 positive breast cancer which is resistant/refractory to endocrine therapy or at risk of developing resistance to endocrine therapy), or to estimate the class-conditional probabilities, then use Bayes' rule to produce the class probability function as in the previous case.

Following analysis and determination of an index of probability of the presence or absence of ESR1 positive breast cancer which is responsive or resistant/refractory to endocrine therapy or at risk of developing resistance to endocrine therapy, the index can be transmitted or provided to a third party, e.g., a medical practitioner for assessment. The index may be used by the practitioner to assess whether or not additional diagnostic methods are required, e.g., biopsy and histological analysis and/or other assays, or a change in treatment e.g., away from endocrine therapy, or commencement of treatment e.g., endocrine therapy.

Monitoring the Efficacy of Treatment and Disease Progression

As the methylation profile of ESR1-positive breast cancer can vary with the progression of the cancer, a subject suffering from ESR1-positive breast cancer who was previously responsive to endocrine therapy, or who has been previously identified as having a methylation profile which is indicative of responsiveness to endocrine therapy, may acquire (over time) a methylation profile which is indicative of resistance to endocrine therapy and thereby develop resistance to endocrine therapy. Accordingly, the methods described herein are useful for monitoring the progression of ESR1-positive breast cancer in a subject suffering therefrom and monitoring the efficacy of treatment. In this regard, the term “monitoring the progression of ESR1-positive breast cancer” and/or “monitoring the efficacy of treatment” includes, for example, determining whether a subject suffering from ESR1-positive breast cancer retains a methylation profile which is indicative of responsiveness to endocrine therapy or acquires a methylation profile which is indicative of resistance to endocrine therapy. For example, the method comprises determining differential methylation of one or more CpG dinucleotides with the one or more estrogen responsive enhancer regions set forth in Table 1, Table 2 and/or Table 3 in a sample from a subject relative to a reference level of methylation for the corresponding one or more CpG dinucleotides previously determined for the subject or a control sample.

In one example, an increase in methylation at one or more the CpG dinucleotides in the sample compared to the previously obtained sample may indicate that the ESR1-positive breast cancer has progressed to a worsening stage e.g., by acquiring resistance to endocrine therapy. In such circumstances, alternative or additional treatment of the breast cancer may be desired.

In another example, a decrease in methylation at one or more the CpG dinucleotides in the sample compared to the previously obtained sample may indicate that the ESR1-positive breast cancer has improved i.e., the subject is responding to treatment, and/or remains or has become responsive to endocrine therapy. For example, in circumstances where the subject has retained a methylation profile which is indicative of responsiveness to endocrine therapy and is already undergoing endocrine therapy, it may be desirable to continue endocrine therapy. For example, in circumstances where the subject was previously determined to have a methylation profile which is indicative of resistance to endocrine therapy and is therefore not undergoing endocrine therapy, it may be desirable to commence endocrine therapy.

Clearly, the detection of one or more additional biomarkers other than those set forth in Tables 1-3 is encompassed by this example of the disclosure.

Methods for detecting markers are described herein and are to be taken to apply mutatis mutandis to this example of the disclosure.

Methods of Treatment

The present disclosure additionally provides a method of treating ESR1-positive breast cancer. Such a method comprises, for example, diagnosing ESR1-positive breast cancer using a method of the disclosure described in any one or more examples described herein and, based on whether the subject is determined as being responsive or resistant to endocrine therapy, administering a suitable therapeutic compound or performing surgery or recommending treatment with a suitable therapeutic compound or recommending performance of surgery. For example, if, after performing the method of diagnosis or prognosis of the disclosure, the subject is determined as being responsive to endocrine therapy, the method may comprise commencing endocrine therapy e.g., by administering a therapeutic compound which blocks, alters or removes the activity of estrogen and/or progesterone, or recommending that the subject commence endocrine therapy. In another example, if, after performing the method of diagnosis or prognosis of the disclosure, the subject is determined as being resistance/refractory to endocrine therapy, the method may comprise commencing treatment other than endocrine therapy e.g., chemotherapy or radiotherapy and/or performing surgery, or recommending that the subject commences treatment other than endocrine therapy e.g., chemotherapy or radiotherapy, and/or recommending surgery.

Drugs suitable for use in endocrine therapy are well known in the art, and include, for example, anastrozole, exemestane, fulvestrant, goserelin, letrozole, leuprorelin, leuprolide acetate, megestrol, palbociclib, tamoxifen and toremifene.

Chemotherapeutic drugs suitable for treatment of breast cancer are known in the art, but may include, for example, docetaxel, paclitaxel, platinum agents (cisplatin, carboplatin), vinorelbine, capecitabine, liposomal doxorubicin, gemcitabine, mitoxantrone, ixabepilone, albumin-bound paclitaxel and eribulin.

Kits

The present disclosure additionally provides a kit for use in a method of the disclosure. In one embodiment, the kit comprises:

(i) one or more probes or primers (or isolated antibodies or ligands) that specifically hybridize to a biomarker (a CpG dinucleotide) described herein according to any example; and

(ii) detection means.

In another example, a kit additionally comprises a reference sample. Such a reference sample may for example, be a polynucleotide sample derived from a sample isolated from one or more subjects suffering from breast cancer. Alternatively, a reference sample may comprise a sample isolated from one or more normal healthy individuals.

In one example, the kit comprises a probe or primer. In one example, the probe or primer that is capable of selectively hybridizing to a CpG dinucleotide of an estrogen responsive enhancer region described herein according to any example.

In those cases where the probe is not already available, they must be produced. Apparatus for such synthesis is presently available commercially and techniques for synthesis of various nucleic acids are available in the literature. Methods for producing probes or primers are known in the art and/or described herein.

In one example, a probe or primer selectively hybridizes to a CpG dinucleotide of a estrogen responsive enhancer region set forth in Tables 1-3 that is selectively mutated by, for example, bisulphite treatment if the residue is not methylated. In another example, a probe or primer selectively hybridizes to a CpG dinucleotide of a genomic region set forth in Tables 1-3 that can be methylated in a ESR1 positive breast cancer cell.

The kit may further comprise instructions for the detection of methylation levels of any of the target genes disclosed herein and for the comparison of those methylation levels with a reference level. The instructions may provide one or a series of cut-off values demarcating the likelihood of risk of a subject having ESR1 positive breast cancer which is responsive or resistance to endocrine therapy.

The present disclosure additionally provides a kit or an article of manufacture comprising a compound for therapeutic treatment of ESR1 positive breast cancer packaged with instructions to perform a method substantially as described herein according to any example of the disclosure. For example, if the ESR1 positive breast cancer is determined as being responsive to endocrine therapy, the kit may comprise a therapeutic compound which blocks, alters or removes the activity of estrogen and/or progesterone e.g., anastrozole, exemestane, fulvestrant, goserelin, letrozole, leuprorelin, leuprolide acetate, megestrol, palbociclib, tamoxifen or toremifene. If, on the other hand, the ESR1 positive breast cancer is determined as being resistant to endocrine therapy, the kit may comprise a chemotherapeutic drug known in the art for treatment of breast cancer e.g., docetaxel, paclitaxel, platinum agents (cisplatin, carboplatin), vinorelbine, capecitabine, liposomal doxorubicin, gemcitabine, mitoxantrone, ixabepilone, albumin-bound paclitaxel or eribulin.

Knowledge-Based Systems

Knowledge-based computer software and hardware for implementing an algorithm of the disclosure also form part of the present disclosure. Such computer software and/or hardware are useful for performing a method of the disclosure. Thus, the present disclosure also provides software or hardware programmed to implement an algorithm that processes data obtained by performing the method of the disclosure via an univariate or multivariate analysis to provide a disease index value and provide or permit a diagnosis of ESR1 positive breast cancer which is responsive or resistance to endocrine therapy and/or for treatment management to determine progression or status of ESR1 positive breast cancer throughout treatment to determine whether there is likely to be a change in responsiveness or resistance to endocrine therapy, with the results of the disease index value in comparison with predetermined values.

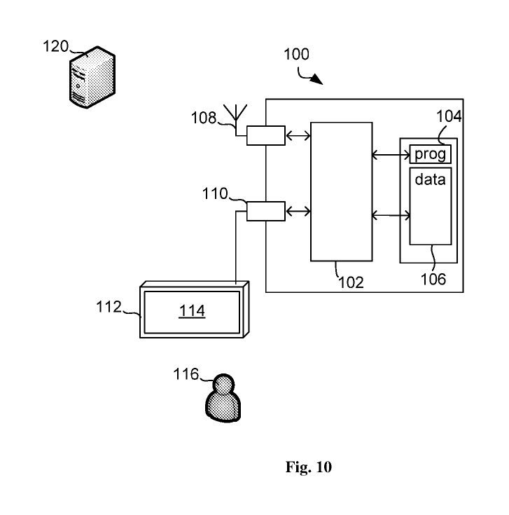

FIG. 10 illustrates a computer system 100 for predicting response to endocrine therapy in a subject suffering from estrogen receptor 1 (ESR1) positive breast cancer. The computer system 100 comprises a processor 102 connected to a program memory 104, a data memory 106, a communication port 108 and a user port 110. The program memory 104 is a non-transitory computer readable medium, such as a hard drive, a solid state disk or CD-ROM. Software, that is, an executable program stored on program memory 104 causes the processor 102 to perform the methods disclosed herein. For example, processor 102 determines the methylation status of one or more CpG dinucleotide sequences within one or more estrogen responsive enhancers in the subject; and identifies differential methylation of said one or more CpG dinucleotide sequences in the subject relative to data for a reference level of methylation for the corresponding one or more CpG dinucleotide sequences. Differential methylation of said one or more CpG dinucleotide sequences in the subject relative to the reference level is indicative of the subject's likely response to endocrine therapy

As used in the context of a computer system 100 of the disclosure, the term “determines the methylation status”, “determining the methylation status” or similar refers to calculating, retrieving or receiving one or more data values indicative of the methylation status of the one or more CpG dinucleotide sequences in the subject. This also applies to related terms.

The processor 102 may then store the methylation status on data store 106, such as on RAM or a processor register. Processor 102 may also send the determined methylation status via communication port 108 to a server, such as a pathology server.

The processor 102 may receive data, such as sequencing data, from data memory 106 as well as from the communications port 108 and the user port 110, which is connected to a display 112 that shows a visual representation 114 of the predicted response to a user 116, such as a clinician. In one example, the processor 102 receives sequencing data from a sequencing machine via communications port 108, such as by using a local area network. Although communications port 108 and user port 110 are shown as distinct entities, it is to be understood that any kind of data port may be used to receive methylation status data, such as a network connection, a memory interface, a pin of the chip package of processor 102, or logical ports, such as IP sockets or parameters of functions stored on program memory 104 and executed by processor 102. These parameters may be stored on data memory 106 and may be handled by-value or by-reference, that is, as a pointer, in the source code.

The processor 102 may receive sequencing data through all these interfaces, which includes memory access of volatile memory, such as cache or RAM, or non-volatile memory, such as an optical disk drive, hard disk drive, storage server or cloud storage. The computer system 100 may further be implemented within a cloud computing environment, such as a managed group of interconnected servers hosting a dynamic number of virtual machines.

It is to be understood that any receiving step may be preceded by the processor 102 determining or computing the data that is later received. For example, the processor 102 determines the methylation status and stores the methylation status in data memory 106, such as RAM or a processor register. The processor 102 then requests the data from the data memory 106, such as by providing a read signal together with a memory address. The data memory 106 provides the data as a voltage signal on a physical bit line and the processor 102 receives the methylation status via a memory interface.

For example, processor 102 may receive sequencing data in the form of a file stored on a file system that is remote (cloud) or local including network attached storage (NAS) or server attached storage (SAN). Processor 102 analyses the sequencing data and identifies the presence of methylated cytosine nucleotides (5-methylcytosine or 5-MeC) and/or cytosine-to-uracil converted nucleotides (optionally identified as thymine nucleotides). Processor 102 may identify cytosine nucleotides which are methylated by comparing the received sequencing data to a reference and determining those cytosine nucleotides which are methylated and/or those cytosine nucleotides that have are not methylated (for example, those cytosines which have not been deaminated as a result of bisulphite treatment and thereby converted to uracil). Processor 102 stores the result of this identification in a separate file on the file system that may be the same or different to the file system on which the sequencing data is stored.

It is to be understood that throughout this disclosure unless stated otherwise, methylation status, sequences, methylation, level, patient, subject and the like may refer to data structures, which are physically stored on data memory 106 or processed by processor 102. Further, for the sake of brevity when reference is made to particular variable names, such as “differential methylation” or “methylation status” this can be understood to refer to values of variables stored as physical data in computer system 100.

The method for predicting response to endocrine therapy may be understood as a blueprint for the software program and may be implemented step-by-step, such that each step is represented by a function in a programming language, such as C++ or Java. The resulting source code may then be compiled and stored as computer executable instructions on program memory 104 or provided as executable source code such as PHP or Python.

Processor 102 may generate an output to indicate the predicted response to endocrine therapy. This output may comprise an electronic document, such as a PDF document. This output may also be rendered on a website that is remotely accessible by the clinician. Generating the output may then comprise generating HTML code and storing the HTML code on the data store of a webserver. This generation of the HTML code may occur dynamically and triggered by the clinician requesting the information. The predicted response to endocrine therapy may be stored on a database, such as a subject database, associated with the subject. The system 100 may be implemented using an Angular front-end for user interface generation and Flask backend for database management.

It should be understood that the techniques of the present disclosure might be implemented using a variety of technologies. For example, the methods described herein may be implemented by a series of computer executable instructions residing on a suitable computer readable medium. Suitable computer readable media may include volatile (e.g. RAM) and/or non-volatile (e.g. ROM, disk) memory, carrier waves and transmission media. Exemplary carrier waves may take the form of electrical, electromagnetic or optical signals conveying digital data steams along a local network or a publically accessible network such as the internet.

It should also be understood that, unless specifically stated otherwise as apparent from the following discussion, it is appreciated that throughout the description, discussions utilizing terms such as “estimating” or “processing” or “computing” or “calculating”, “optimizing” or “determining” or “displaying” or “maximising” or the like, in the context of a computer system 100, refer to the action and processes of a computer system, or similar electronic computing device, that processes and transforms data represented as physical (electronic) quantities within the computer system's registers and memories into other data similarly represented as physical quantities within the computer system memories or registers or other such information storage, transmission or display devices.

In one example, a method of the disclosure may be used in existing knowledge-based architecture or platforms associated with pathology services. For example, results from a method described herein are transmitted via a communications network (e.g. the internet) to a processing system in which an algorithm is stored and used to generate a predicted posterior probability value which translates to the index of disease probability (e.g., ESR1 positive breast cancer which is responsive to endocrine therapy or resistant to endocrine therapy) or responsiveness to treatment, which is then forwarded to an end user in the form of a diagnostic or predictive report.

The method of the disclosure may, therefore, be in the form of a kit or computer-based system which comprises the reagents necessary to detect the concentration of the biomarkers and the computer hardware and/or software to facilitate determination and transmission of reports to a clinician.

The assay of the present disclosure permits integration into existing or newly developed pathology architecture or platform systems. For example, the present disclosure contemplates a method of allowing a user to determine the status of a subject with respect to ESR1-positive breast cancer, the method including:

(a) receiving data in the form of levels of differential methylation of one or more CpG dinucleotides within one or more estrogen responsive enhancer regions set forth in Tables 1-3 for a test sample relative to a reference level of methylation, optionally in combination with another marker of breast cancer e.g., ESR1-positive breast cancer;

(b) processing the subject data via univariate and/or multivariate analysis to provide a disease index value;

(c) determining the status of the subject in accordance with the results of the disease index value in comparison with predetermined values; and

(d) transferring an indication of the status of the subject to the user via the communications network reference to the multivariate analysis includes an algorithm which performs the multivariate analysis function.

In one example, the method additionally includes:

(a) having the user determine the data using a remote end station; and

(b) transferring the data from the end station to the base station via the communications network.

The base station can include first and second processing systems, in which case the method can include:

(a) transferring the data to the first processing system;

(b) transferring the data to the second processing system; and

(c) causing the first processing system to perform the univariate or multivariate analysis function to generate the disease index value.

The method may also include:

(a) transferring the results of the univariate or multivariate analysis function to the first processing system; and

(b) causing the first processing system to determine the status of the subject.

In this case, the method also includes at least one of:

(a) transferring the data between the communications network and the first processing system through a first firewall; and

(b) transferring the data between the first and the second processing systems through a second firewall.

The second processing system may be coupled to a database adapted to store predetermined data and/or the univariate or multivariate analysis function, the method include:

(a) querying the database to obtain at least selected predetermined data or access to the multivariate analysis function from the database; and

(b) comparing the selected predetermined data to the subject data or generating a predicted probability index.

The second processing system can be coupled to a database, the method including storing the data in the database.

The method can also include having the user determine the data using a secure array, the secure array of elements capable of determining the level of biomarker and having a number of features each located at respective position(s) on the respective code. In this case, the method typically includes causing the base station to:

(a) determine the code from the data;

(b) determine a layout indicating the position of each feature on the array; and

(c) determine the parameter values in accordance with the determined layout, and the data.

The method can also include causing the base station to:

(a) determine payment information, the payment information representing the provision of payment by the user; and

(b) perform the comparison in response to the determination of the payment information.

The present disclosure also provides a base station for determining the status of a subject with respect to a ESR1 positive breast cancer, the base station including:

(a) a store method;

(b) a processing system, the processing system being adapted to:
(i) receive subject data from the user via a communications network;
(ii) determining the status of the subject in accordance with the results of the algorithmic function including the comparison; and

(c) output an indication of the status of the subject to the user via the communications network.

The processing system can be adapted to receive data from a remote end station adapted to determine the data.

The processing system may include:

(a) a first processing system adapted to:
(i) receive the data; and
(ii) determine the status of the subject in accordance with the results of the univariate or multivariate analysis function including comparing the data; and

(b) a second processing system adapted to:
(i) receive the data from the processing system;
(ii) perform the univariate or multivariate analysis function including the comparison; and
(iii) transfer the results to the first processing system.

The base station typically includes:

(a) a first firewall for coupling the first processing system to the communications network; and

(b) a second firewall for coupling the first and the second processing systems.

The processing system can be coupled to a database, the processing system being adapted to store the data in the database.

The present disclosure is now described further in the following non-limiting examples.

### EXAMPLES

### Example 1—DNA Methylation Profiling of Enhancer Loci in Endocrine Resistant Cells

To interrogate DNA methylation remodelling as a critical component of acquired endocrine resistance, we performed methylation profiling in duplicate using the Infinium HumanMethylation 450 beadchip, on ESR1-positive hormone sensitive MCF7 cells, and three different well characterised endocrine resistant MCF7-derived cell lines; tamoxifen-resistant (TAMR)10, fulvestrant-resistant (FASR)11 and estrogen deprivation resistant (MCF7X)12 cells.

Cell Culture and HumanMethylation450K Array

MCF7 breast cancer cells and the corresponding endocrine resistant sub cell lines were provided by Dr Julia Gee (Cardiff University, UK). Briefly, MCF7 cells were maintained in RPMI-1640 based medium containing 5% (v/v) fetal calf serum (FCS). Tamoxifen-resistant MCF7 (TAMR) cells were generated by the long-term culture of MCF7 cells in phenol-red-free RPMI medium containing 5% charcoal stripped FCS and 4-OH-tamoxifen (1×10⁻⁷ M) (TAM). Fulvestrant-resistant MCF-7 (FASR) cells were generated by the long-term culture of MCF7 cells in phenol-red-free RPMI medium containing 5% charcoal stripped FCS and fulvestrant (1×10⁻⁷ M) (FAS). Long-term estrogen deprived MCF7 (MCF7X) cells were generated by the long-term culture of MCF7 cells in phenol-red-free RPMI medium containing 5% charcoal stripped FCS. Endocrine resistant sub lines were established and characterised following 6 months endocrine challenge/estrogen deprivation exposure^{10, 11, 12}. All cell lines were authenticated by short-tandem repeat (STR) profiling (Cell Bank, Australia) and cultured for less than 6 months after authentication. Genomic DNA was extracted using the Qiagen DNeasy Blood and Tissue kit according to manufacturer's instructions. HumanMethylation450K arrays were carried out by the Australian Genome Research Facility (AGRF) (Melbourne, Australia). Cell line HumanMethylation450K array data is available online at GEO (GSE69118).

HM450 Analysis

Two biological replicates per condition—MCF7, TAMR, MCF7X, or FASR—were profiled on Illumina's HumanMethylation450K array. Raw HM450 data was pre-processed and background normalized with the Biconductor minfi package (Aryee et al., (2014) Bioinformatics 30:1363-1369) using preprocesslllumina (bg.correct=TRUE, normalize=“controls”, reference=1); resulting M-Values were used for statistical analyses and I3-Values for heatmap visualizations and clustering. Differential methylation analysis of the pre-processed data was performed using the Bioconductor limma package.

Results

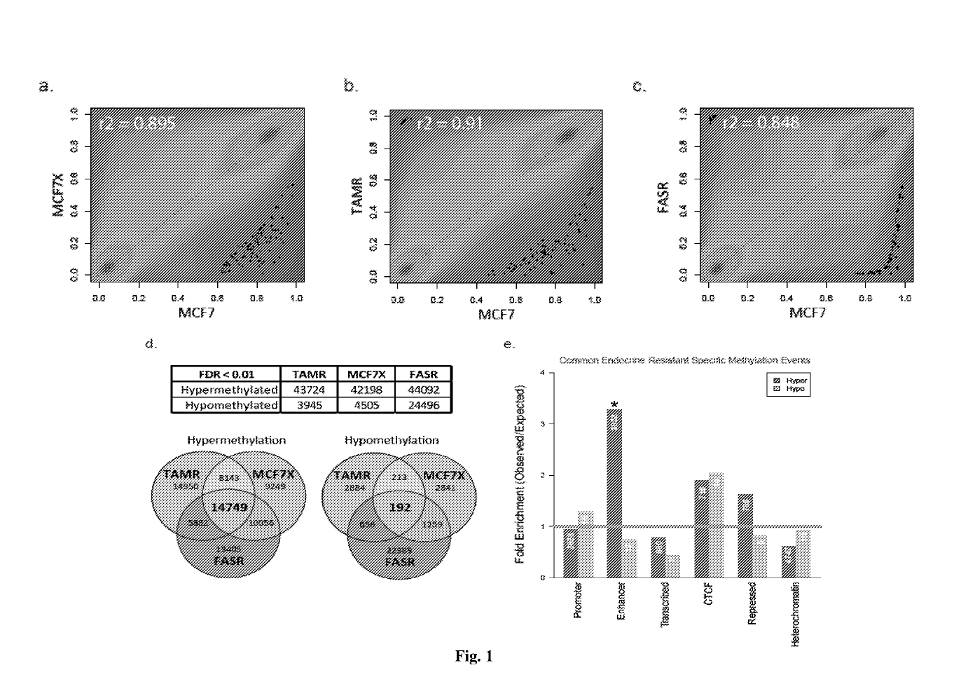

Density plots showing the correlation between the DNA methylation profile of parent MCF7 cells and individual endocrine resistant cell lines indicate that the MCF7X and TAMR cells, which are both ESR1-positive (Knowlden et al., (2003) Endocrinology 144:1032-1044; Staka et al., (2005) Endocr Relat Cancer 12:S85-97), predominantly gained DNA methylation as indicated by the increased density of points above the trend line. In contrast, FASR cells, which are ESR1-negative (McClelland et al., (2001) Endocrinology, 142:2776-2788), exhibited both hyper and hypomethylation events relative to parent MCF7 cells as indicated by a symmetrical density distribution (FIG. 1a-c). We first sought to identify the common differential DNA methylation events present in each of the three uniquely derived endocrine resistant cell models by carrying out paired analyses (i.e. each endocrine resistant cell line vs MCF7 parent control) and overlapping the data (FIG. 1d). We found that across the individual resistant cell lines 14,749 CpG probes were commonly hypermethylated (FDR<0.01) whereas only 192 probes exhibited shared hypomethylation (FDR<0.01) (FIG. 1d).

### Example 2—Characterisation of Functional Genomic Location of Differential Methylation at Enhancer Loci in Endocrine Resistant Cells

To comprehensively characterise the functional genomic location of differential methylation observed in the endocrine resistant cell models we used ChromHMM segmentation of the MCF7 genome as previously described in Taberlay et al., (2014) Genome Research, 24(9):1421-32.

Genomic Segmentation and Annotation

The ChromHMM segmentation of the MCF7 genome was obtained from Taberlay et al., (2014). Enhancer (“Enhancer” and “Enhancer+CTCF”) and Promoter categories (“Promoter”, “Promoter+CTCF”, and “Poised Promoter”) were collapsed into a single “Enhancer” and “Promoter” state respectively for the purposes of our analysis. RefSeq transcript annotations were obtained from UCSC genome browser (Kent et al. (2002) Genome Research 12:996-1006 (2002); Meyer et al. (2013) Nucleic Acids Res. 41:D64-69). Strikingly, significant enrichment of commonly hypermethylated probes was exclusively observed in enhancer regions of the genome (n=3932 probes, p<<0.0001; hypergeometric test) (FIG. 1e).

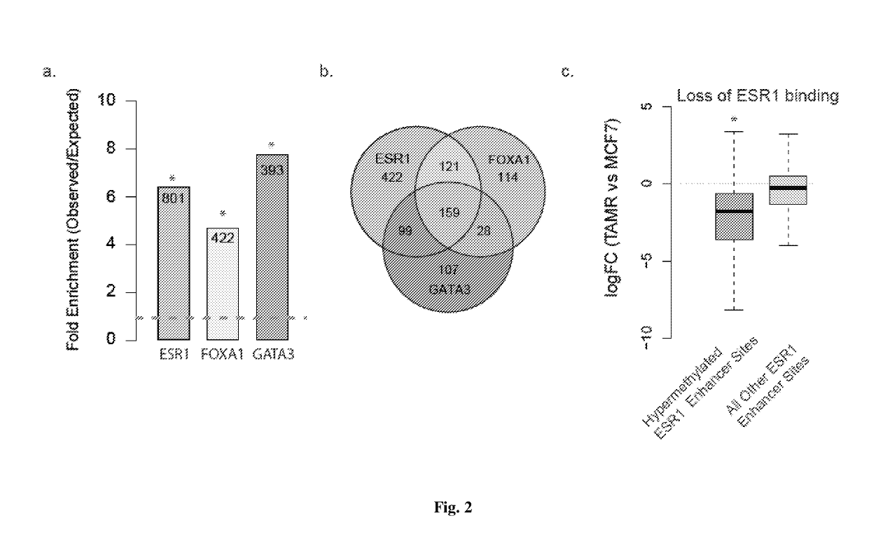

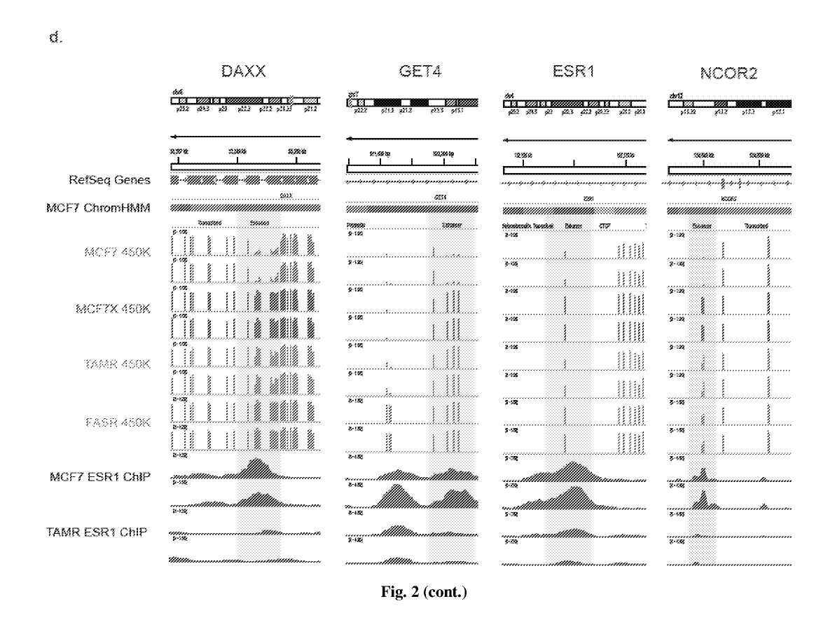

We next sought to determine whether the enhancer regions identified as being more heavily methylated in all endocrine resistance models were regulated by the estrogen receptor in the parental MCF7 cells. Using reprocessed, publically available ChIPSeq data for MCF7 ESR1 (Ross-Innes et al. (2012) Nature 481:389-393), GATA3 (Theodorou et al., (2013) Genome Res. 23:12-22) and FOXA1 (Hurtado et al., (2011) Nat. Genet. 43:27-33) (two transcription factors closely associated with ESR1-activity), we found that enhancer-specific CpG hypermethylated probes were enriched in ESR1 binding sites by approximately 6 fold, FOXA1 binding sites by 5 fold and GATA3 binding sites by 8 fold (p<<0.0001; hypergeometric test) (FIG. 2a). The greatest number of hypermethylated enhancer probes were found to overlap ESR1 binding sites (n=801), which represents approximately 20% of all hypermethylated probes in enhancer regions. Significantly, 47% (379 out of 801) of the hypermethylated enhancer probes that were located within an ESR1 binding site were also located within a FOXA1 and/or GATA3 binding site (FIG. 2b) which is particularly noteworthy since these transcription factors cooperatively modulate ESR1-transcriptional networks by forming a functional enhanceosome.

### Example 3—Enhancer DNA Hypermethylation and Diminished ESR1 Binding

Having defined a subset of ESR1 binding sites that overlap enhancer regions that contain hypermethylated loci in multiple models of endocrine resistance (n=856 sites—Table 1), we sought to determine whether DNA methylation affected the intensity of ESR1 binding at these sites.

ChIP-Seq Data Acquisition and Analysis

Using MCF7 and TAMR ESR1 ChIP data previously described by Ross-Innes et al. (2012) Nature 481:389-393, we compared the change in ESR1 binding signal intensity at ESR1-enhancer sites that contained (a) hypermethylated probe(s) to that of all other ESR1-enhancer sites. Reads were mapped to genome build HG19 (GRCh37) with bowtie and mismatched (>3 mismatched bases), multiple mapping and duplicate reads were excluded from downstream analysis. ESR1 enrichment peaks were identified with the HOMER software suite (Heinz et al. (2010) Molecular cell 38:576-589) using the findPeaks utility (-style factor -fragLength 200 -size 300 -F 0 -L 0 -C 0 -poisson 1e⁻⁰⁶) on each experiment separately. The resulting peaks were merged to produce a ground set of 120,735 regions for subsequent analysis. Active ESR1 regions were identified in MCF7 by comparing the distribution of reads overlapping the ground set of ESR1 regions in the three MCF7 ESR1 experiments (GSM798423, GSM798424, and GSM798425) and MCF7 input experiment (GSM798440) with edegR (Robinson et al., (2010) Bioinformatics 26:139-140). This yielded 54,265 active ESR1 regions in MCF7 (FDR<0.05). A similar strategy was applied to TAMR data to yield 49,511 ESR1 regions in TAMR cells. Regions of differential ESR1 binding were identified by comparing the distribution of sequence reads in MCF7 and TAMR across the ground set of ESR1 regions using edgeR and potential variation in copy number was accounted for using DiffBind (Ross-Innes et al. (2012) Nature 481:389-393). This analysis resulted in 24,711 regions with statistical significant gain (FDR 5%) and 32,343 regions with statistical significant loss (FDR 5%) of ESR1 binding in TAMR cells as compared to MCF7 cells. ESR1 peaks overlapping HM450 probes were assigned to the nearest RefSeq transcript (<20 kb distance) for the purposes of gene expression analysis. Raw MCF7 GATA3 and FOXA1 ChIP-Seq data was obtained from Theodorou et al., (2013) Genome Res. 23:12-22 and Hurtado et al., (2011) Nat Genet 43:27-33 respectively. Data were processed in the same manner as outlined for ESR1 ChIP-seq previously described.

Results

At methylated ESR1-enhancer sites there was a 2.29 log fold reduction in ESR1 binding in TAMR compared to MCF7 cells. In contrast, at all other ESR1-enhancer binding sites, there was a 0.52 log fold reduction in ESR1 binding in TAMR compared to MCF7 cells. Thus, increased methylation at ESR1-enhancer sites is associated with reduction in ESR1 binding (p<<0.0001; t-test) (FIG. 2c). Four illustrative examples show the loss of ESR1 binding in the TAMR cells at enhancer regions that are more heavily methylated in the endocrine resistant versus the parent MCF7 (FIG. 2d). The examples include enhancer regions located within the gene body of death associated protein 6 (DAXX), golgi to ER traffic protein 4 homolog (GET4) (a member of the BAG6-UBL4A-GET4 DNA damage response/cell death complex), ESR1 itself and nuclear receptor co-repressor 2 (NCOR2) (FIG. 2d).

### Example 4—Enhancer DNA Hypermethylation and Related Gene Expression

Since the vast majority of ESR1-enhancer binding sites identified as hypermethylated in the endocrine resistant cell lines compared to the parent MCF7 cells were intragenic i.e. 617 out of 856, 72% with at least partial overlap (Table 2), we next sought to determine if the DNA methylation of these regions correlated with the expression of the genes in which they were located (or closest TSS if intergenic) in human breast cancer.

TCGA Data Acquisition

DNA methylation analysis utilized clinical data available through the TCGA Breast Invasive Carcinoma (BRCA) cohort (TCGA (2012) Nature 490:61-70). Raw HM450 methylation data (level 1) were obtained from the TCGA data portal (normal samples=97, ESR1 positive tumours=353 and ESR1 negative=105). ESR1 positive tumours were further divided into luminal A (lumA=301) and luminal B (lumB=52) populations using progesterone receptor (PR) expression, such that lumA were ESR1+/PR+ and lumB were ESR1+/PR−. Processed RNA-Seq expression data (level 3) were obtained from TCGA data portal (588 ESR1 positive tumours with 73 matched normals and 174 ESR1 negative samples with 19 matched normals).

Gene Set Enrichment Analysis of TCGA Data

GSEA was performed against the Molecular Signatures Database v4.0 (MSigDB) (Subramanian et al. (2005) PNAS, 102:15545-15550) C2 Collection. Enrichment was assessed by hypergeometric testing as implemented in the R stats package.

Results

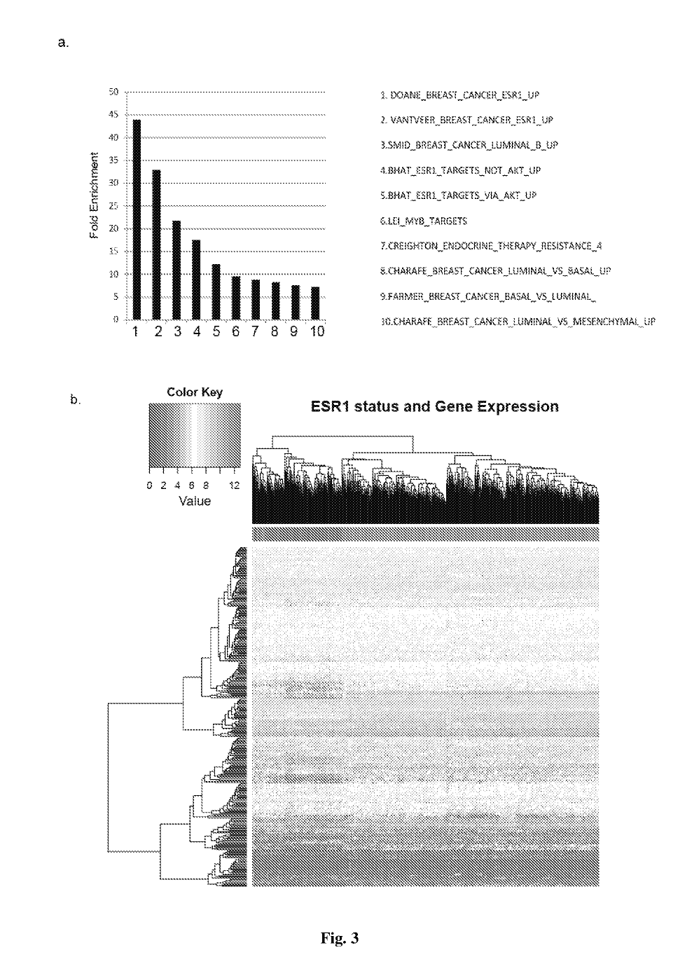

Using RNA-seq and HM450 methylation data derived from TCGA breast cohort (n=459 patients), we determined that out of the 856 ESR1-enhancer binding sites of interest, hypermethylation of 328 sites (i.e. 38% of ESR1-enhancer sites) correlated with the reduced expression of the genes with which they were most closely associated (Spearman's correlation coefficient; p<0.001) (Table 3). The 328 ESR1-enhancer binding sites represented 291 unique genes (including those presented in FIG. 2d). Gene set enrichment analysis revealed that these genes were over-represented in gene sets up-regulated by ESR1 activation, down-regulated in the acquisition of endocrine resistance and gene sets lowly expressed in basal vs luminal disease, thus suggesting that such genes were critical drivers of estrogen-driven tumours (FIG. 3a). Interestingly, using unsupervised clustering analysis, this gene set (n=291) stratifies ESR1-positive and ESR1-negative breast cancer patients (FIG. 3b). Cumulatively, this indicates that the methylation events occurring throughout the acquisition of endocrine resistance are serving to facilitate an estrogen-independent phenotype reflective of a breast cancer subtype that is refractory to endocrine therapy.

### Example 5—ESR1-Enhancer Methylation Defines Breast Cancer Subtype

We next sought to determine whether ESR1-enhancer hypermethylation was indicative of breast cancer subtype. We assessed the median methylation of all hypermethylated ESR1-enhancer probes (n=801) in TCGA normal (n=97), luminal A (n=301), luminal B (n=52) and ESR1-negative (n=105) patient HM450 data.

Clinical Sample Acquisition and DNA Extraction

Formalin-fixed, paraffin-embedded (FFPE) breast cancer samples were obtained from the St. George Hospital, Kogarah, Australia. The de-identified haematoxylin-eosin stained sections were reviewed by a pathologist and representative tumour areas were marked and blocks were cored accordingly. Genomic DNA was extracted using the Qiagen AllPrep DNA/RNA FFPE kit according to the manufacturer's instructions.

Multiplex Bisulfite-PCR Resequencing of Clinical FFPE DNA

Bisulfite DNA conversions were performed using a manual protocol. For each conversion approximately 100 ng was bisulfite converted at a time. Conversion took place at 80° C. for 45 min in the presence of 0.3M NaOH, 3.75 mM quinone, and 2.32M sodium metabisulfite, as per the methodology described in Clark et al., (2006) Nat. Protoc. 1:2353-2364. The multiplex bisulfite PCR reaction was performed as detailed in Korbie et al., (2015) Clinical Epigenetics 7:28. Briefly, Promega HotStart GoTaq with Flexi-buffer (M5005) was used with the following components at the indicated concentrations: 5X green (1×), CES 5×, (0.5×, as described in Ralser et al., (2006) Biochem Biophys Res Commun 347:747-751), MgCl₂ (4.5 mM), dNTP's (200 μM each), primers (forward and reverse at 100 mM), Hot Start Taq (0.025 U μL⁻¹), DNA (2 ng μL⁻¹). All primers used are listed in Table 4.

TABLE 4
rimer sequences for multiplex bisulfite-PCR resequencing of clinical FFPE DNA

| Non-CpG | | |
|---|---|---|
| Primers | Primer sequence | Fusion primer sequences |
| GATA3_ct_f2 | GAtAGAttAGAGGtAGtAAGGAA | ACACTGACGACATGGTTCTACAGAtAGAttAGAGGtAGtAAGGAA |
| GATA3_ct_r2 | CTTTTCAaAAACACCTTaAAAaCTA | TACGGTAGCAGAGACTTGGTCTCTTTTCAaAAACACCTTaAAAaCTA |
| ESR1_ct_f1 | TTGtAGGGTTTAGGATGAAGT | ACACTGACGACATGGTTCTACATTGtAGGGTTTAGGATGAAGT |
| ESR1_ct_r1 | CTTTACAATCTCTCTTTTTCCATT | TACGGTAGCAGAGACTTGGTCTCTTTACAATCTCTCTTTTTCCATT |
| ESR1_ct_f2 | GGTGTGGAAGGtAAGGGAA | ACACTGACGACATGGTTCTACAGGTGTGGAAGGtAAGGGAA |
| ESR1_ct_r2 | CTaaaCATTaCAaaCTTaTTCAAATAT | TACGGTAGCAGAGACTTGGTCTCTaaaCATTaCAaaCTTaTTCAAATAT |
| GET4_ct_f1 | GTTGGTGTttTTGGATATGTG | ACACTGACGACATGGTTCTACAGTTGGTGTttTTGGATATGTG |
| GET4_ct_r1 | CCATCCATaaaaCAAaaTCAaCT | TACGGTAGCAGAGACTTGGTCTCCATCCATaaaaCAAaaTCAaCT |
| ITPK1_ct_f1 | GAAAGtTGGtTTTtTGGttTtAGT | ACACTGACGACATGGTTCTACAGAAAGtTGGtTTTtTGGttTtAGT |
| ITPK1_ct_r2 | CATCATCATCAACAACCAaACA | TACGGTAGCAGAGACTTGGTCTCATCATCATCAACAACCAaACA |
| MSI2_ct_f2 | GAGtATtTGGtTTTtATTTTTAAGTG | ACACTGACGACATGGTTCTACAGAGtATtTGGtTTTtATTTTTAAGTG |
| MSI2_ct_r2 | CCCAAaAATAAaCTCAACTCCTT | TACGGTAGCAGAGACTTGGTCTCCCAAaAATAAaCTCAACTCCTT |
| C8orf46_ga_f1 | CCAaCATCAaAaAAaaaAaCACC | ACACTGACGACATGGTTCTACACCAaCATCAaAaAAaaaAaCACC |
| C8orf46_ga_r1 | GGGtAGATTGAtTtTGtAGtTG | TACGGTAGCAGAGACTTGGTCTGGGtAGATTGAtTtTGtAGtTG |
| DAXX_ga_f2 | aCATATTTaaAaATaACCTCATCCA | ACACTGACGACATGGTTCTACAaCATATTTaaAaATaACCTCATCCA |
| DAXX_ga_r2 | ttTTtAAGGGtTGAGTGtTtTGA | TACGGTAGCAGAGACTTGGTCTttTTtAAGGGtTGAGTGtTtTGA |
| NCOR2_ga_f1 | CTCCCAaAaCCACACCCT | ACACTGACGACATGGTTCTACACTCCCAaAaCCACACCCT |
| NCOR2_ga_r1 | TTTTGGAGGtAAAGttAGTGG | TACGGTAGCAGAGACTTGGTCTTTTTGGAGGtAAAGttAGTGG |
| RXRA_ga_f1 | aAaCTTTTaaTaTaCTaCCCACC | ACACTGACGACATGGTTCTACAaAaCTTTTaaTaTaCTaCCCACC |
| RXRA_ga_r1 | GATGAGTtAGATGGtAGGG | TACGGTAGCAGAGACTTGGTCTGATGAGTtAGATGGtAGGG |

Cycling conditions were: 94° C., 5 mins; 12 cycles of (95° C., 20 s; 60° C., 1 min); 12 cycles of (94° C., 20 s; 65° C., 1 min 30 s); 65° C., 3 mins, 10° C. hold. Agencourt XP beads were using to clean-up and concentrate the multiplex reaction for subsequent barcoding (i.e., addition of Illumina p5/p7 sequences and sample specific DNA barcodes). The barcoding PCR used the following reagents at the indicated final concentrations in a 100 μl reaction: 1× GoTaq Green Flexi buffer; 0.25×CES; 4.5 mM MgCl₂; 200 μM dNTPs; 0.05 U μL⁻¹ HotStart Taq; 25 μL of pooled template after Agencourt XP bead cleanup; and 20 μl MiSeq (Fluidigm PN FLD-100-3771). Cycling conditions were: 94° C., 5 mins; 9 cycles of (97° C., 15 s; 60° C., 30 s; 72° C., 2 mins); 72° C., 2 mins; 6° C., 5 mins. MiSeq sequencing was performed used the MiSeq Reagent Kit v2, 300 cycle; PN MS-102-2002. Bioinformatic analysis started with adaptor trimming using Trim galore (options: --length 100). Mapping used the Bismark methylation mapping program (Krueger et al., (2011) Bioinformatics 27:1571-1572) running Bowtie2 (Langmead and Salzberg (2012) Nat. Methods, 9:357-359) (options: --bowtie2 -N 1 -L 15 --bam -p 2 --score L, −0.6, −0.6 --non_directional; bismark_methylation_extractor -s -merge_non_CpG -comprehensive --cytosine_report). To reduce computational overhead mapping took place against only those genomic regions which were being investigated, plus an additional 100 bp-1 kb of flanking sequence.

Results

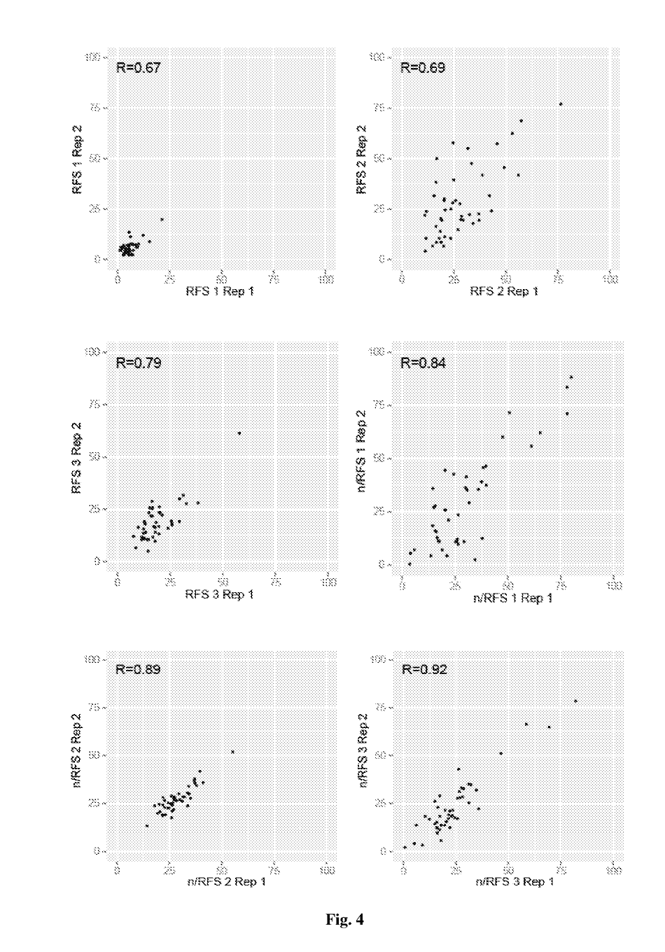

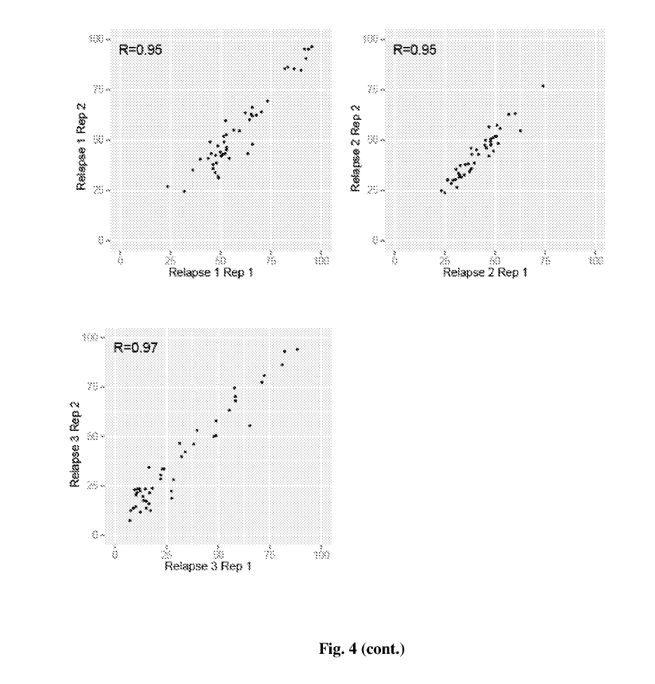

In normal breast tissue (which is reported to be approximately 7% ESR1-positive e.g., as described in Petersen et al, (1987) Cancer Research 47:5748-5751), the median methylation of the ESR1-enhancer sites was highest, while median DNA methylation was significantly reduced in luminal A disease (p<<0.0001; Mann-Whitney U test), which is indicative of its endocrine-responsive state. Interestingly, median ESR1-enhancer methylation was greater in luminal B patients compared to luminal A patients (p=0.017; Mann-Whitney U test), who are almost twice as likely to acquire endocrine resistance. In ESR1-negative disease, median methylation was higher than in luminal disease (vs luminal A, p<<0.0001; vs luminal B, p<<0.0001; Mann-Whitney U test) (FIG. 4a). A heatmap highlights the hypomethylated status of the ESR1-enhancer sites in luminal A disease relative to normal breast tissue and the other breast cancer subtypes (FIG. 4b). This trend is clearly illustrated at the DAXX enhancer region in which each CpG within the ESR1 binding site was hypomethylated in luminal A disease compared to normal tissue and luminal B and ESR1-negative cancer (FIG. 4c). Critically, no such variability was apparent at the DAXX promoter region (1000 bp upstream and 100 bp downstream of the transcription start site) (FIG. 4c), suggesting a significant regulatory effect of increased methylation at the enhancer locus.

### Example 6—ESR1-Enhancer Hypermethylation Predicts Endocrine Failure

Given that ESR1-enhancer hypermethylation is prevalent in acquired endocrine resistance in vitro (FIG. 1e and FIGS. 2a-d) and in molecular sub-classifications of breast cancer that are intrinsically less responsive to endocrine therapy (FIGS. 4a-c), we next sought to determine the methylation status of a panel of these loci in ESR1-positive (luminal A) breast cancer samples from patients with different outcomes.

Primary samples were sourced from patients that received endocrine therapy for five years and either experienced relapse-free survival (RFS) (>14 years) or those that had relapsed (<6 years), defined as no relapse-free survival (n/RFS). Matched local relapse samples were also compared to the primary n/RFS patient samples. All patients received the same endocrine therapy (tamoxifen) Patient data is provide in Table 5).

TABLE 5
Patient details

| | | | | | Histological | Ellis | | Mol | ER | PR |
|---|---|---|---|---|---|---|---|---|---|---|
| PatientID | AgeAtOperation | SizeOfTumour | VascularInvasion | HistType | Grade | Grade | Margin | subtype | Status | Status |
| RFS | | | | | | | | | | |
| 1 | 44 | 20 | No | Ductal | High | 3 | Clear | Luminal A | 2 | 1 |
| 2 | 72 | 19 | No | Lobular | Medium | 2 | Clear | Luminal A | 2 | 1 |
| 3 | 50 | 20 | Yes | Ductal | High | 3 | Clear | Luminal A | 2 | 2 |
| n/RFS | | | | | | | | | | |
| 1 | 38 | 15 | No | Ductal | High | 3 | Clear | Luminal A | 2 | 2 |
| 2 | 37 | 16 | No | Lobular | High | 3 | Clear | Luminal A | 2 | 2 |
| 3 | 45 | 30 | No | Ductal | High | 3 | Clear | Luminal A | 2 | 2 |

| | | | | Local | | Date of | Date of |
|---|---|---|---|---|---|---|---|
| | Adj | Adj | Localised | Recurrence | Date of | last | local |
| PatientID | Tamoxifen | chemo | Radiotherapy | in Breast | Diagnosis | follow up | Recurrence |
| RFS | | | | | | | |
| 1 | Adjuvant | Adjuvant | Y | N | July 1998 | September 2012 | — |
| 2 | Adjuvant | N | Y | N | November 1998 | June 2013 | — |
| 3 | Adjuvant | Adjuvant | Y | N | February 1998 | January 2014 | — |
| n/RFS | | | | | | | |
| 1 | Adjuvant | Adjuvant | Y | Y | December 1999 | — | November 2005 |
| 2 | Adjuvant | Adjuvant | Y | Y | September 2001 | — | September 2004 |
| 3 | Adjuvant | Adjuvant | Y | Y | June 1998 | — | June 2002 |

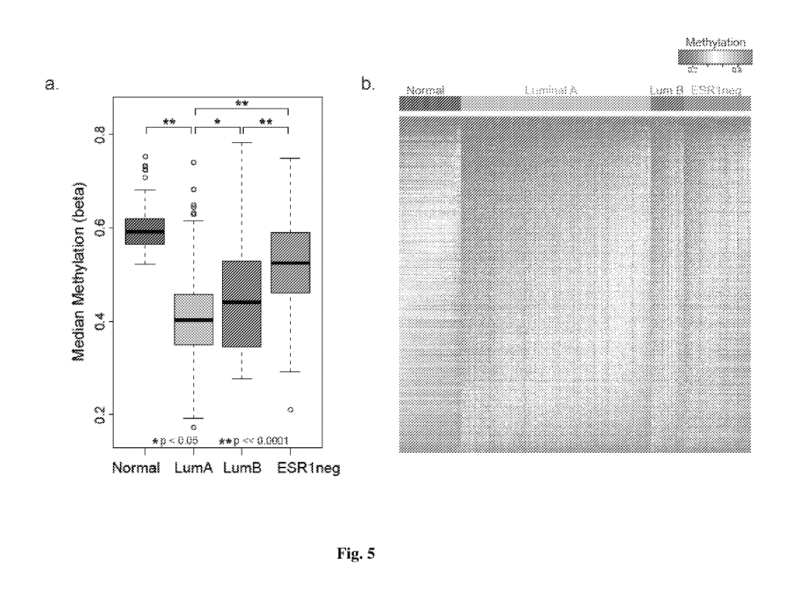

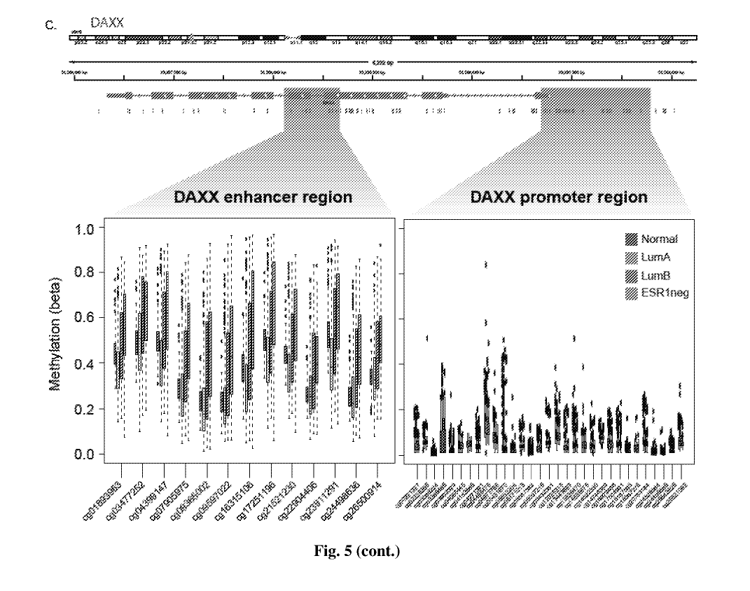

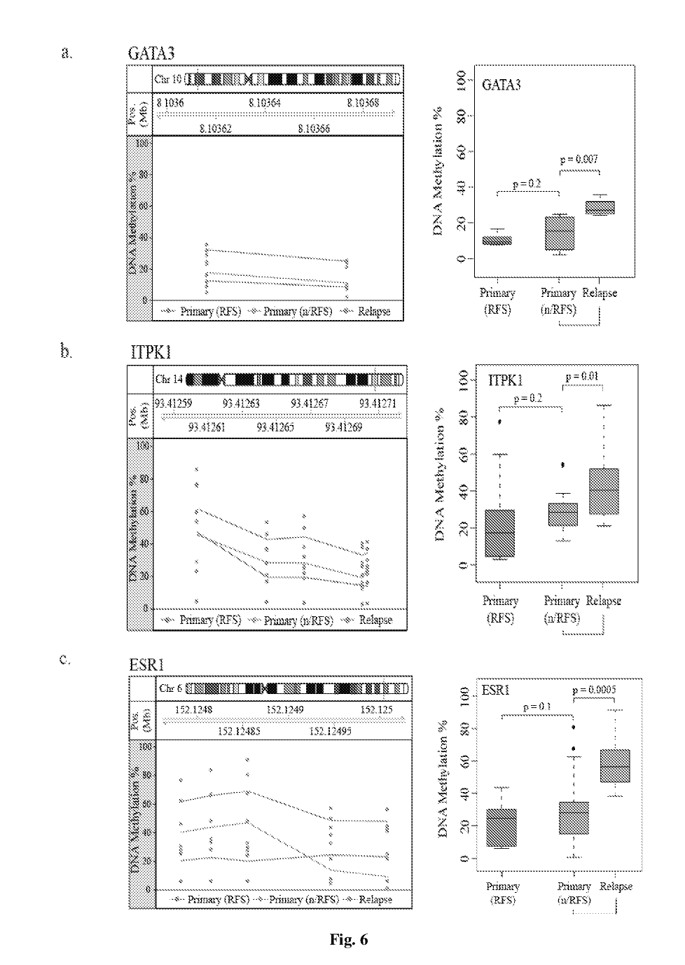

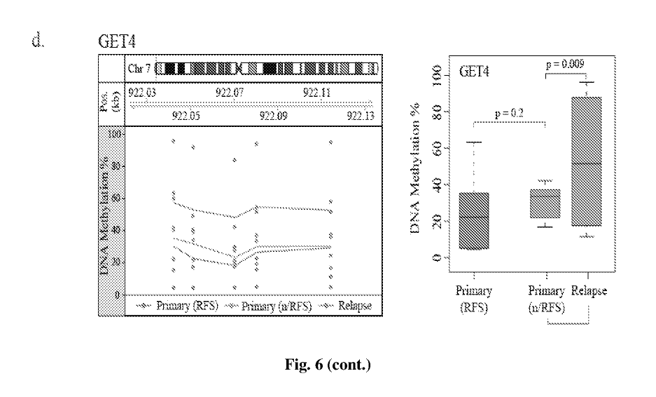

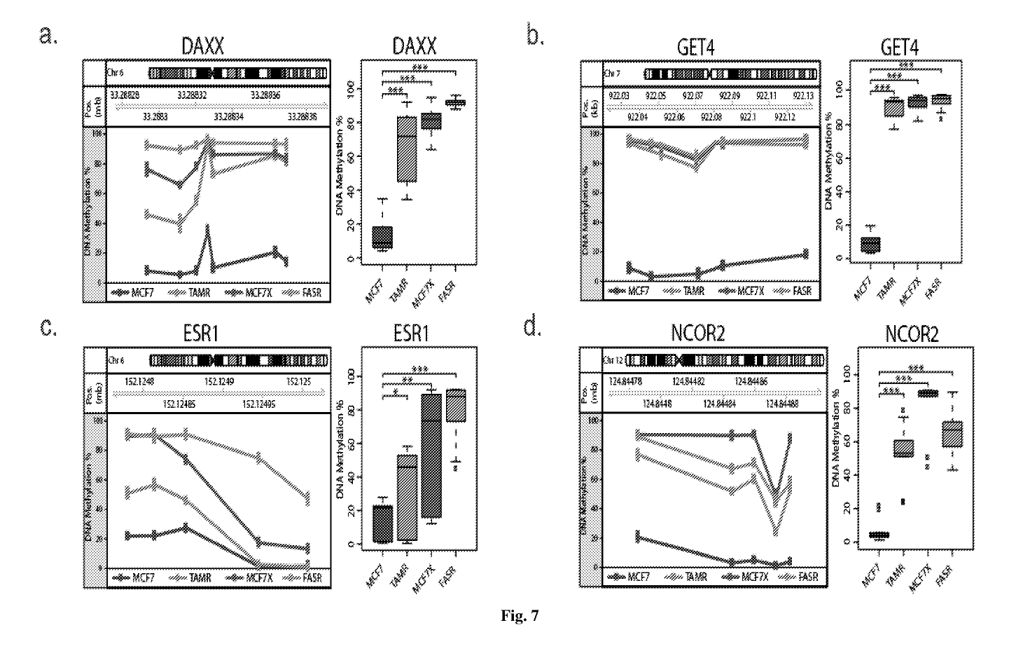

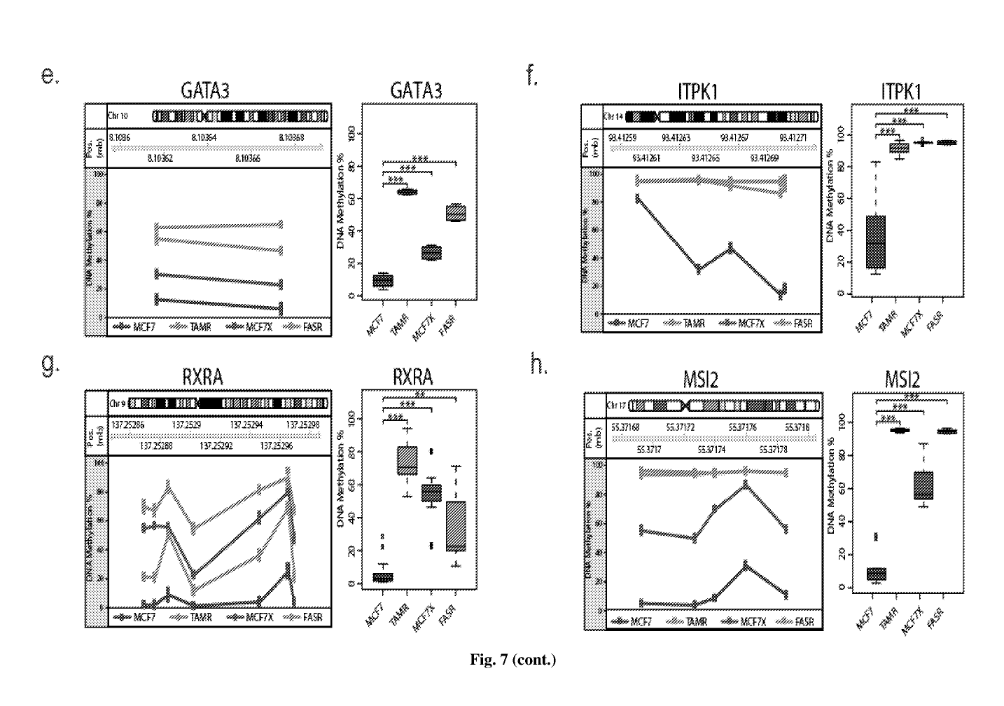

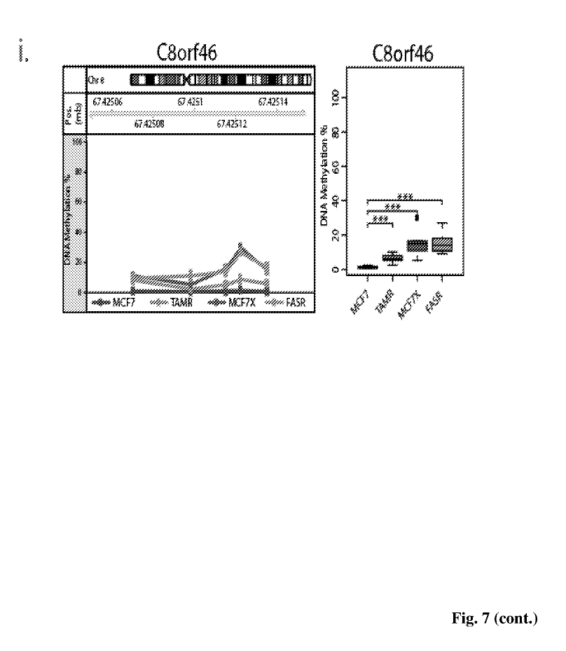

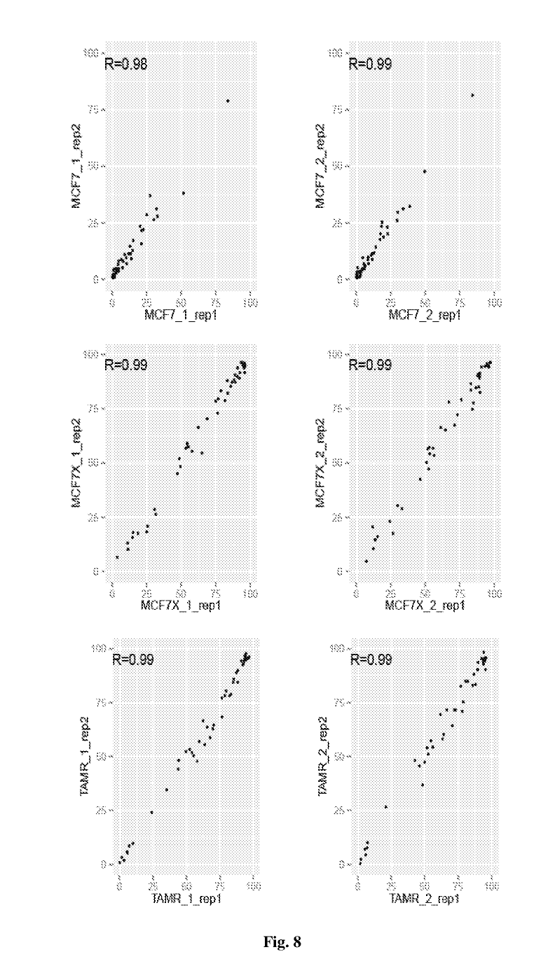

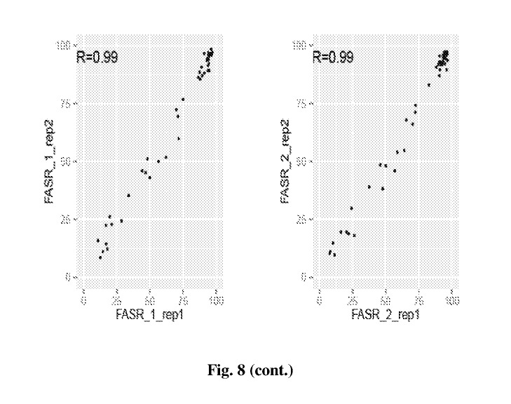

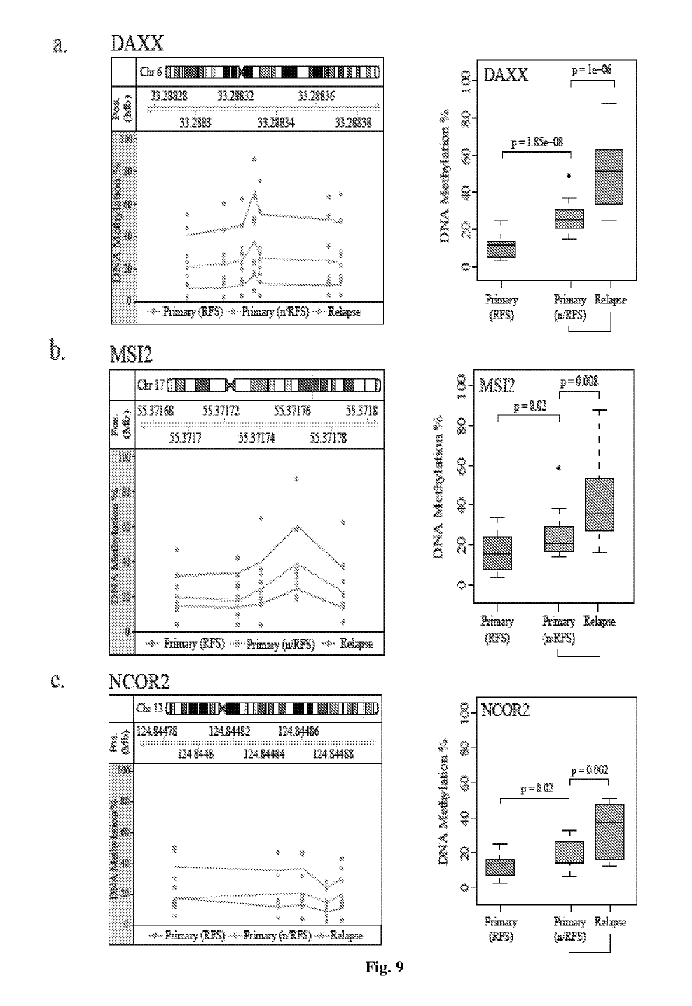

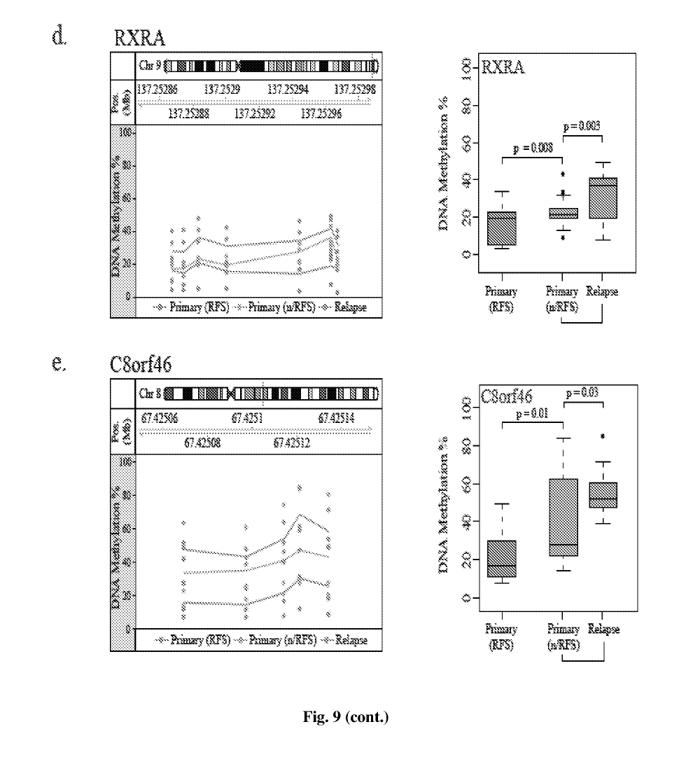

Using a multiplex bisulphite-PCR resequencing methodology specifically devised for FFPE derived DNA (Korbie et al., (2015) Clinical Epigenetics 7:28), the methylation of multiple CpG sites across a panel of 9 estrogen-responsive enhancer regions was interrogated (technical duplicate correlates for all amplicons investigated are shown in FIG. 4). These enhancer regions included those located within DAXX, MSI2, NCOR2, RXRA and C8orf46 (FIG. 5a-e) and enhancer regions located within GATA3, ITPK1, ESR1 and GET4 (FIG. 6a-d). The assay was repeated with DNA extracted from biological duplicates of the endocrine resistant cell lines and the parent MCF7 cells to ensure its viability (FIG. 7a-i; technical duplicate correlates for all amplicons investigated are shown in FIG. 8). The average methylation levels detected at all enhancer loci were significantly higher in the recurrent tumours compared to the matched primary (n/RFS) tumours (DAXX; p<0.0001, ESR1; p<0.0005, RXRA; p<0.005, GET4, NCOR2, GATA3, MSI2; p<0.01, C8orf46, ITPK1; p<0.05; t-test), confirming that DNA methylation at ESR1-responsive enhancers is acquired in resistant disease (FIGS. 6 and 9). The difference in DNA methylation between RFS and n/RFS primary tumours was less considerable, although a statistically significant difference was observed for DAXX; p<0.0001, RXRA; p<0.01, C8orf46; p=0.01, NCOR2 and MSI2 (p<0.05; t-test) enhancer regions (FIG. 9).

### Conclusions Drawn from Examples 1-6

The results provided herein support a model whereby ESR1-responsive enhancer DNA methylation is a fundamental unifying characteristic that defines endocrine sensitivity in breast cancer. This study is the first to combine in depth MCF7 ChromHMM annotation and genome wide methylation data from multiple resistance models to more comprehensively characterise global differential methylation across diverse genomic regions. This study shows for the first time that the methylation status of enhancers is associated with the inhibition of ESR1 binding in vitro and with the reduced expression of critical regulators and effectors of ESR1-activity in human disease. The identification of ESR1-responsive enhanceosome hypermethylation is both novel and considerably pertinent in the context of endocrine resistance, since genome wide positional analyses defining the set of cis-regulatory elements that recruit ESR1 in breast cancer cells have revealed its predominant recruitment to enhancers as opposed to promoter regions. In this study, the majority of ESR1-regulated enhancer regions identified as hypermethylated in the resistant cells were located within gene bodies. Strikingly, hypermethylation of these enhancer regions was frequently correlated with reduced expression of the host gene. Examples of genes whose expression inversely correlated with ESR1-enhancer DNA methylation include DAXX and GET4, each of which are reported to have roles in apoptosis. It is possible that the loss of expression of genes associated with pro-apoptotic functions facilitates the progression of endocrine resistance by reducing the efficacy of apoptotic signalling pathways activated by endocrine therapies.

Importantly, the ESR1-responsive enhancer hypermethylation events identified in the endocrine-resistant cell lines were also differentially methylated in endocrine sensitive and endocrine-resistant breast cancer patient samples. Therefore, ESR1-responsive enhancer methylation status may be reflective of endocrine dependence and could be used to stratify patients as responders to endocrine therapy. For example, NCOR2, a gene whose expression has previously been associated with metastasis free survival in 620 lymph node-negative patients with ESR1-positive breast cancer, was shown to negatively correlate with ESR1-enhancer methylation. In the present study, NCOR2 enhancer methylation was significantly higher in the poor (non relapse-free) prognosis patients, compared to the good (relapse-free) prognosis primary luminal A breast cancer patients.

1. A method for predicting response to endocrine therapy in a subject suffering from estrogen receptor 1 (ESR1) positive breast cancer, said method comprising:
(i) determining the methylation status of one or more CpG dinucleotide sequences within one or more estrogen responsive enhancers in the subject; and
(ii) identifying differential methylation of said one or more CpG dinucleotide sequences in the subject relative to a reference level of methylation for the corresponding one or more CpG dinucleotide sequences;
wherein differential methylation of said one or more CpG dinucleotide sequences in the subject relative to the reference level is indicative of the subject's likely response to endocrine therapy.

2. The method according to claim 1, wherein increased methylation at the one or more CpG dinucleotide sequences within the one or more estrogen responsive enhancers relative to the reference level is indicative of the ESR1-positive breast cancer being refractory to endocrine therapy.

3. A method for predicting the therapeutic outcome of and/or monitoring the progression of estrogen receptor 1 (ESR1) positive breast cancer in a subject receiving or about to receive endocrine therapy, said method comprising:
(i) determining the methylation status of one or more CpG dinucleotide sequences within one or more estrogen responsive enhancers in the subject; and
(ii) identifying differential methylation of said one or more CpG dinucleotide sequences in the subject relative to a reference level of methylation for the corresponding one or more CpG dinucleotide sequences;
wherein differential methylation identified at (ii) is indicative of the likely therapeutic outcome and/or of the progression of the ESR1 positive breast cancer.

4. The method according to claim 3, wherein increased methylation at the one or more CpG dinucleotide sequences within the one or more estrogen responsive enhancers relative to the reference level is indicative of the ESR1-positive breast cancer being refractory to endocrine therapy and/or that the subject is not responding to the endocrine therapy.

5. The method according to any one of claims 1 to 4, comprising determining whether the ESR1-positive breast cancer is a luminal A breast cancer subtype or a luminal B breast cancer subtype.

6. The method according to any one of claims 1 to 5, wherein the one or more CpG dinucleotide sequences are within one or more ESR1 binding sites.

7. The method according to claim 6, wherein the one or more CpG dinucleotide sequences are within one or more ESR1-binding sites as defined in Table 1.

8. The method according to claim 6 or claim 7, wherein the one or more CpG dinucleotide sequences are within one or more ESR1-binding sites as defined in Table 2.

9. The method according to any one of claims 6 to 8, wherein the one or more CpG dinucleotide sequences are within one or more ESR1-binding sites as defined in Table 3.

10. The method according to any one of claims 1 to 5, wherein methylation status is determined at one or more CpG dinucleotide sequences within an estrogen responsive enhancer of a gene selected from DAXX, ESR1, RXRA, GET4, NCOR2, GATA3, MSI2, C8orf46 and/or ITPK1.

11. The method according to claim 10, wherein methylation status is determined at one or more CpG dinucleotide sequences within an estrogen responsive enhancer within a gene selected from DAXX, ESR1, RXRA, GET4, NCOR2, GATA3, MSI2, C8orf46 and/or ITPK1.

12. The method according to claim 11, wherein methylation status is determined at one or more CpG dinucleotide sequences selected from those defined in rows 57, 111-113, 256-258, 288-289, 469-470, 805, 821-822 and 824-826 of Table 1.

13. The method according to any one of claims 1 to 5, wherein methylation status is determined at one or more CpG dinucleotide sequences within an estrogen responsive enhancer of a gene selected from DAXX, RXRA, NCOR2, MSI2, and/or C8orf46.

14. The method according to claim 13, wherein methylation status is determined at one or more CpG dinucleotide sequences within an estrogen responsive enhancer within a gene selected from DAXX, RXRA, NCOR2, MSI2, and/or C8orf46

15. The method according to any one of claims 1 to 5, wherein methylation status is determined at one or more CpG dinucleotide sequences within an estrogen responsive enhancer of a gene selected from FOXA1, ESR1 and/or GATA3.

16. The method according to any one of claims 1 to 15, wherein methylation status of one or more CpG dinucleotide sequences within the one or more estrogen responsive enhancers is determined by one or more techniques selected from the group consisting of a nucleic acid amplification, polymerase chain reaction (PCR), methylation specific PCR, bisulfite pyrosequencing, single-strand conformation polymorphism (SSCP) analysis, restriction analysis, microarray technology, and proteomics.

17. The method according to any one of claims 1 to 16, wherein methylation status of one or more CpG dinucleotide sequences within the one or more estrogen responsive enhancers in the subject is determined by one or more of the following:
(i) performing methylation-sensitive endonuclease digestion of DNA from the subject;
(ii) treating nucleic acid from the subject with an amount of a compound that selectively mutates non-methylated cytosine residues in nucleic acid under conditions sufficient to induce mutagenesis thereof and produce a mutant nucleic acid and amplifying the mutant nucleic acid using at least one primer that selectively hybridizes to the mutant nucleic acid;
(iii) treating nucleic acid from the subject with an amount of a compound that selectively mutates non-methylated cytosine residues in nucleic acid under conditions sufficient to induce mutagenesis thereof and produce a mutant nucleic acid, hybridizing a nucleic acid probe or primer capable of specifically hybridizing to the mutant nucleic acid and detecting the hybridized probe or primer;
(iv) treating nucleic acid from the subject with an amount of a compound that selectively mutates non-methylated cytosine residues in nucleic acid under conditions sufficient to induce mutagenesis thereof and produce a mutant nucleic acid, amplifying the mutant nucleic acid with promoter-tagged primers, transcribing the mutant nucleic acid in vitro to produce a transcript, subjecting the transcript to an enzymatic base-specific cleavage, and determining differences in mass and/or size of any cleaved fragments resulting from mutated cysteine residues, such as by MALDI-TOF mass spectrometry; and
(v) treating nucleic acid from the subject with an amount of a compound that selectively mutates non-methylated cytosine residues in nucleic acid under conditions sufficient to induce mutagenesis thereof, thereby producing a mutant nucleic acid, and determining the nucleotide sequence of the mutant nucleic acid.

18. The method according to claim 17, wherein the compound that selectively mutates non-methylated cytosine residues is a salt of bisulphite.

19. The method according to any one of claims 1 to 18, wherein the methylation status of one or more CpG dinucleotide sequences within the one or more estrogen responsive enhancers is determined in a test sample from the subject comprising tissue and/or a body fluid comprising, or suspected of comprising, a breast cancer cell or components of a breast cancer cell.

20. The method according to claim 19, wherein the sample comprises tissue, a cell and/or an extract thereof taken from a breast or lymph node.

21. The method according to claim 19, wherein the body fluid is selected from the group consisting of whole blood, a fraction of blood such as blood serum or plasma, urine, saliva, breast milk, pleural fluid, sweat, tears and mixtures thereof.

22. The method of any one of claims 1 to 21, wherein the reference level of methylation is a level of methylation determined for one or more CpG dinucleotide sequences within a corresponding estrogen responsive enhancer of a sample selected from the group consisting of:
(i) a sample from a normal or healthy tissue;
(ii) a sample comprising a non-cancerous cell;
(iii) a sample comprising a cancerous cell other than a breast cancer cell characterized as being ESR1-negative subtype;
(iv) a sample comprising a cancerous cell other than a breast cancer cell characterized as being a ESR1-positive subtype which is refractory to endocrine therapy;
(v) an extract of any one of (i) to (iv);
(vi) a data set comprising levels of methylation for the one or more CpG dinucleotide sequences within the corresponding estrogen responsive enhancer in a normal or healthy individual or a population of normal or healthy individuals;
(vii) a data set comprising levels of methylation for the one or more CpG dinucleotide sequences within the corresponding estrogen responsive enhancer in an individual or a population of individuals having cancer other than ESR1-negative breast cancer subtype;
(viii) a data set comprising levels of methylation for the one or more CpG dinucleotide sequences within the corresponding estrogen responsive enhancer in an individual or a population of individuals having cancer other than a ESR1-positive breast cancer subtype which is refractory to endocrine therapy; and
(ix) a data set comprising levels of methylation for the one or more CpG dinucleotide sequences within the corresponding estrogen responsive enhancer in the subject being tested wherein the levels of methylation are determined for a matched sample having normal cells.

23. A kit for predicting response to endocrine therapy in a subject suffering from estrogen receptor 1 (ESR1) positive breast cancer, said kit comprising:
(i) one or more reagents configured to determine the methylation status of one or more CpG dinucleotide sequences within one or more estrogen responsive enhancers in the subject; and
(ii) a reference material which provides a reference level of methylation of the corresponding one or more CpG dinucleotide sequences.

24. A kit for predicting the therapeutic outcome of and/or monitoring the progression of estrogen receptor 1 (ESR1) positive breast cancer in a subject receiving or about to receive endocrine therapy, said kit comprising:
(i) one or more reagents configured to determine methylation status of one or more CpG dinucleotide sequences within one or more estrogen responsive enhancers in the subject; and
(ii) a reference material which provides a reference level of methylation of the corresponding one or more CpG dinucleotide sequences.

25. The kit according to claim 23 or claim 24, wherein the one or more reagents is/are configured to determine methylation status of one or more CpG dinucleotide sequences within one or more ESR1 binding sites as defined in Table 1.

26. The kit according to any one of claims 23 to 25, wherein the reference level of methylation is a level of methylation determined for one or more CpG dinucleotide sequences within a corresponding genomic region of a sample selected from the group consisting of:
(i) a sample from a normal or healthy tissue;
(ii) a sample comprising a non-cancerous cell;
(iii) a sample comprising a cancerous cell other than a breast cancer cell characterized as being ESR1-negative subtype;
(iv) a sample comprising a cancerous cell other than a breast cancer cell characterized as being a ESR1-positive subtype which is refractory to endocrine therapy;
(v) an extract of any one of (i) to (iv);
(vi) a data set comprising levels of methylation for the one or more CpG dinucleotide sequences within the corresponding estrogen responsive enhancer in a normal or healthy individual or a population of normal or healthy individuals;
(vii) a data set comprising levels of methylation for the one or more CpG dinucleotide sequences within the corresponding estrogen responsive enhancer in an individual or a population of individuals having cancer other than ESR1-negative breast cancer subtype;
(viii) a data set comprising levels of methylation for the one or more CpG dinucleotide sequences within the corresponding estrogen responsive enhancerin an individual or a population of individuals having cancer other than a ESR1-positive breast cancer subtype which is refractory to endocrine therapy; and
(ix) a data set comprising levels of methylation for the one or more CpG dinucleotide sequences within the corresponding estrogen responsive enhancer in the subject being tested wherein the levels of methylation are determined for a matched sample having normal cells.

27. The kit according to claim 23 or 25 or 26, when used in the method of any one of claims 1, 2 or 5 to 22.

28. The kit according to any one of claims 24 to 26, when used in the method of any one of claims 3 to 22.

29. Use of one or more reagents in the preparation of a medicament for predicting response to endocrine therapy in a subject suffering from estrogen receptor 1 (ESR1) positive breast cancer, wherein the one or more reagents is/are configured to determine methylation status of one or more CpG dinucleotide sequences within one or more estrogen responsive enhancers in the subject.

30. Use of one or more reagents in the preparation of a medicament for predicting the therapeutic outcome of and/or monitoring the progression of estrogen receptor 1 (ESR1) positive breast cancer in a subject receiving or about to receive endocrine therapy, wherein the one or more reagents is/are configured to determine methylation status of one or more CpG dinucleotide sequences within one or more estrogen responsive enhancers in the subject.

31. The use according to claim 29 or claim 30, wherein the one or more reagents is/are configured to determine methylation status of one or more CpG dinucleotide sequences within one or more ESR1 binding sites as defined in Table 1.


SEQUENCE LISTING (40 sequences)

SEQ ID NO: 1 - DNA, 23 bp, Artificial sequence
  GATA3_ct_f2 primer sequence
gatagattagaggtagtaaggaa

SEQ ID NO: 2 - DNA, 45 bp, Artificial sequence
  GATA3_ct_f2 fusion primer sequence
acactgacgacatggttctacagatagattagaggtagtaaggaa

SEQ ID NO: 3 - DNA, 25 bp, Artificial sequence
  GATA3_ct_r2 primer sequence
cttttcaaaaacaccttaaaaacta

SEQ ID NO: 4 - DNA, 47 bp, Artificial sequence
  GATA3_ct_r2 fusion primer sequence
tacggtagcagagacttggtctcttttcaaaaacaccttaaaaacta

SEQ ID NO: 5 - DNA, 21 bp, Artificial sequence
  ESR1_ct_f1 primer sequence
ttgtagggtttaggatgaagt

SEQ ID NO: 6 - DNA, 43 bp, Artificial sequence
  ESR1_ct_f1 fusion primer sequence
acactgacgacatggttctacattgtagggtttaggatgaagt

SEQ ID NO: 7 - DNA, 24 bp, Artificial sequence
  ESR1_ct_r1 primer sequence
ctttacaatctctctttttccatt

SEQ ID NO: 8 - DNA, 46 bp, Artificial sequence
  ESR1_ct_r1 fusion primer sequence
tacggtagcagagacttggtctctttacaatctctctttttccatt

SEQ ID NO: 9 - DNA, 19 bp, Artificial sequence
  ESR1_ct_f2 primer sequence
ggtgtggaaggtaagggaa

SEQ ID NO: 10 - DNA, 41 bp, Artificial sequence
  ESR1_ct_f2 fusion primer sequence
acactgacgacatggttctacaggtgtggaaggtaagggaa

SEQ ID NO: 11 - DNA, 27 bp, Artificial sequence
  ESR1_ct_r2 primer sequence
ctaaacattacaaacttattcaaatat

SEQ ID NO: 12 - DNA, 49 bp, Artificial sequence
  ESR1_ct_r2 fusion primer sequence
tacggtagcagagacttggtctctaaacattacaaacttattcaaatat

SEQ ID NO: 13 - DNA, 21 bp, Artificial sequence
  GET4_ct_f1 primer sequence
gttggtgtttttggatatgtg

SEQ ID NO: 14 - DNA, 43 bp, Artificial sequence
  GET4_ct_f1 fusion primer sequence
acactgacgacatggttctacagttggtgtttttggatatgtg

SEQ ID NO: 15 - DNA, 23 bp, Artificial sequence
  GET4_ct_r1 primer sequence
ccatccataaaacaaaatcaact

SEQ ID NO: 16 - DNA, 45 bp, Artificial sequence
  GET4_ct_r1 fusion primer sequence
tacggtagcagagacttggtctccatccataaaacaaaatcaact

SEQ ID NO: 17 - DNA, 24 bp, Artificial sequence
  ITPK1_ct_f1 primer sequence
gaaagttggttttttggttttagt

SEQ ID NO: 18 - DNA, 46 bp, Artificial sequence
  ITPK1_ct_f1 fusion primer sequence
acactgacgacatggttctacagaaagttggttttttggttttagt

SEQ ID NO: 19 - DNA, 22 bp, Artificial sequence
  ITPK1_ct_r2 primer sequence
catcatcatcaacaaccaaaca

SEQ ID NO: 20 - DNA, 44 bp, Artificial sequence
  ITPK1_ct_r2 fusion primer sequence
tacggtagcagagacttggtctcatcatcatcaacaaccaaaca

SEQ ID NO: 21 - DNA, 26 bp, Artificial sequence
  MSI2_ct_f2 primer sequence
gagtatttggtttttatttttaagtg

SEQ ID NO: 22 - DNA, 48 bp, Artificial sequence
  MSI2_ct_f2 fusion primer sequence
acactgacgacatggttctacagagtatttggtttttatttttaagtg

SEQ ID NO: 23 - DNA, 23 bp, Artificial sequence
  MSI2_ct_r2 primer sequence
cccaaaaataaactcaactcctt

SEQ ID NO: 24 - DNA, 45 bp, Artificial sequence
  MSI2_ct_r2 fusion primer sequence
tacggtagcagagacttggtctcccaaaaataaactcaactcctt

SEQ ID NO: 25 - DNA, 23 bp, Artificial sequence
  C8orf46_ga_f1 primer sequence
ccaacatcaaaaaaaaaaacacc

SEQ ID NO: 26 - DNA, 45 bp, Artificial sequence
  C8orf46_ga_f1 fusion primer sequence
acactgacgacatggttctacaccaacatcaaaaaaaaaaacacc

SEQ ID NO: 27 - DNA, 22 bp, Artificial sequence
  C8orf46_ga_r1 primer sequence
gggtagattgattttgtagttg

SEQ ID NO: 28 - DNA, 44 bp, Artificial sequence
  C8orf46_ga_r1 fusion primer sequence
tacggtagcagagacttggtctgggtagattgattttgtagttg

SEQ ID NO: 29 - DNA, 25 bp, Artificial sequence
  DAXX_ga_f2 primer sequence
acatatttaaaaataacctcatcca

SEQ ID NO: 30 - DNA, 47 bp, Artificial sequence
  DAXX_ga_f2 fusion primer sequence
acactgacgacatggttctacaacatatttaaaaataacctcatcca

SEQ ID NO: 31 - DNA, 23 bp, Artificial sequence
  DAXX_ga_r2 primer sequence
tttttaagggttgagtgttttga

SEQ ID NO: 32 - DNA, 45 bp, Artificial sequence
  DAXX_ga_r2 fusion primer sequence
tacggtagcagagacttggtcttttttaagggttgagtgttttga

SEQ ID NO: 33 - DNA, 18 bp, Artificial sequence
  NCOR2_ga_f1 primer sequence
ctcccaaaaccacaccct

SEQ ID NO: 34 - DNA, 40 bp, Artificial sequence
  NCOR2_ga_f1 fusion primer sequence
acactgacgacatggttctacactcccaaaaccacaccct

SEQ ID NO: 35 - DNA, 21 bp, Artificial sequence
  NCOR2_ga_r1 primer sequence
ttttggaggtaaagttagtgg

SEQ ID NO: 36 - DNA, 43 bp, Artificial sequence
  NCOR2_ga_r1 fusion primer sequence
tacggtagcagagacttggtctttttggaggtaaagttagtgg

SEQ ID NO: 37 - DNA, 23 bp, Artificial sequence
  RXRA_ga_f1 primer sequence
aaacttttaatatactacccacc

SEQ ID NO: 38 - DNA, 45 bp, Artificial sequence
  RXRA_ga_f1 fusion primer sequence
acactgacgacatggttctacaaaacttttaatatactacccacc

SEQ ID NO: 39 - DNA, 19 bp, Artificial sequence
  RXRA_ga_r1 primer sequence
gatgagttagatggtaggg

SEQ ID NO: 40 - DNA, 41 bp, Artificial sequence
  RXRA_ga_r1 fusion primer sequence
tacggtagcagagacttggtctgatgagttagatggtaggg